(19) United States
(12) Patent Application Publication
(10) Pub. No.: US 2017/0084845 A1
(43) Pub. Date: Mar. 23, 2017

(54) ORGANIC OPTOELECTRONIC DEVICE AND DISPLAY DEVICE

(71) Applicant: SAMSUNG SDI CO., LTD., Yongin-si (KR)

(72) Inventors: Jun Seok KIM, Suwon-si (KR); Changwoo KIM, Suwon-si (KR); Eun Sun YU, Suwon-si (KR); HANILL LEE, Suwon-si (KR); Sung-Hyun JUNG, Suwon-si (KR); Sujin HAN, Suwon-si (KR); Jonghoon KO, Suwon-si (KR); Yongtak YANG, Suwon-si (KR); Suyong LIM, Suwon-si (KR); Jongwoo CHOI, Suwon-si (KR)

(21) Appl. No.: 15/270,332

(22) Filed: Sep. 20, 2016

(30) Foreign Application Priority Data
Sep. 21, 2015 (KR) 10-2015-0133056

(51) Int. Cl. H01L 51/00; C07D 251/24; C07D 401/14; C09K 11/02; C07D 471/04; C07D 403/04; C07C 211/61; C09K 11/06; C07D 209/86

(52) U.S. Cl. CPC H01L 51/0067; C09K 11/06; H01L 51/0085; C07D 251/24; C07D 401/14; H01L 51/0072; C07D 209/86; C07D 471/04; C07D 403/04; C07C 211/61; H01L 51/006; C09K 11/025; H01L 51/5004

(57) ABSTRACT

An organic optoelectronic device includes an anode and a cathode facing each other, an emission layer between the anode and the cathode, a hole transport layer between the anode and the emission layer, and a hole transport auxiliary layer between the hole transport layer and the emission layer, wherein the emission layer includes at least one first compound represented by Chemical Formula 1; and at least one second compound represented by Chemical Formula 2, and the hole transport auxiliary layer includes at least one third compound represented by Chemical Formula 3. A display device including the organic optoelectronic device is provided Chemical Formulae 1 to 3 are the same as the detailed description.

### CROSS-REFERENCE TO RELATED APPLICATION

This application claims priority to and the benefit of Korean Patent Application No. 10-2015-0133056 filed in the Korean Intellectual Property Office on Sep. 21, 2015, the entire contents of which are incorporated herein by reference.

### BACKGROUND OF THE INVENTION

### (a) Field of the Invention

An organic optoelectronic device and a display device are disclosed.

### (b) Description of the Related Art

An organic optoelectronic device is a device that converts electrical energy into photoenergy, and vice versa.

An organic optoelectronic device may be classified as follows in accordance with its driving principles. One is an optoelectronic device where excitons are generated by photoenergy, separated into electrons and holes, and are transferred to different electrodes to generate electrical energy, and the other is a light emitting device where a voltage or a current is supplied to an electrode to generate photoenergy from electrical energy.

Examples of the organic optoelectronic device may be an organic photoelectric device, an organic light emitting diode, an organic solar cell, and an organic photo conductor drum.

Of these, an organic light emitting diode (OLED) has recently drawn attention due to an increase in demand for flat panel displays. The organic light emitting diode converts electrical energy into light by applying current to an organic light emitting material and has a structure in which an organic layer is interposed between an anode and a cathode.

A green organic light emitting diode having a long life-span is considered to be one of the critical factors for realizing a long life-span full color display. Accordingly, development of a long life-span green organic light emitting diode is being actively researched. In order to solve this problem, a long life-span green organic light emitting diode is provided in this invention.

### SUMMARY OF THE INVENTION

An embodiment provides a composition for an organic optoelectronic device having high efficiency.

Another embodiment provides a display device including the organic optoelectronic device.

According to an embodiment, an organic optoelectronic device includes an anode and a cathode facing each other, an emission layer between the anode and the cathode, a hole transport layer between the anode and the emission layer, and a hole transport auxiliary layer between the hole transport layer and the emission layer,

wherein the emission layer includes at least one first compound represented by Chemical Formula 1; and at least one second compound represented by Chemical Formula 2, and

the hole transport auxiliary layer includes at least one third compound represented by Chemical Formula 3.

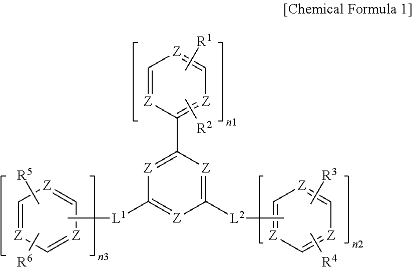

In Chemical Formula 1,

Z is independently N, C, or CR^{a},

at least one of Z is N,

R¹ to R⁶ and R^{a} are independently hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof,

R³ to R⁶ are independently present or adjacent groups are linked to each other to provide a ring,

L¹ and L² are independently a single bond, a substituted or unsubstituted C6 to C30 arylene group, a substituted or unsubstituted C2 to C30 heteroarylene group, or a combination thereof,

n1 is one of integers of 0 to 3, and

n2 and n3 are independently one of integers of 1 to 5;

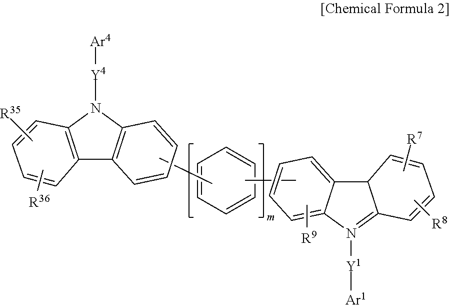

wherein, in Chemical Formula 2,

Y′ and Y⁴ are independently a single bond, a substituted or unsubstituted C6 to C30 arylene group, a substituted or unsubstituted C2 to C30 heteroarylene group, or a combination thereof,

Ar¹ and Ar⁴ are independently a substituted or unsubstituted C6 to C30 aryl group, a substituted or unsubstituted C2 to C30 heterocyclic group, or a combination thereof,

R⁷ to R⁹, R³⁵, and R³⁶ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, and

m is one of integers of 0 to 4,

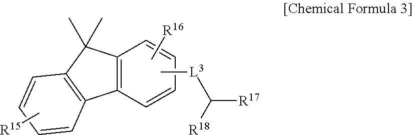

wherein, in Chemical Formula 3,

R¹⁵ to R¹⁸ are independently, hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof,

L³ is a single bond, a substituted or unsubstituted C6 to C30 arylene group, a substituted or unsubstituted C2 to C30 heteroarylene group, or a combination thereof, and

“substituted” of Chemical Formulae 1 to 3 refers to replacement of at least one hydrogen by a deuterium, a halogen, a hydroxyl group, an amino group, C1 to C30 amine group, a nitro group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a fluoro group, a C1 to C10 trifluoroalkyl group, or a cyano group.

According to another embodiment, a display device including the organic optoelectronic device is provided.

An organic optoelectronic device having high efficiency may be realized.

### BRIEF DESCRIPTION OF THE DRAWINGS

FIGS. 1 and 2 are schematic cross-sectional views showing organic optoelectronic devices according to example embodiments.

### DETAILED DESCRIPTION

Hereinafter, embodiments of the present disclosure are described in detail. However, these embodiments are exemplary, the present invention is not limited thereto and the present invention is defined by the scope of claims.

In the present specification, when a definition is not otherwise provided, the term “substituted” refers to replacement of at least one hydrogen of a substituent or a compound by deuterium, a halogen, a hydroxyl group, an amino group, a C1 to C30 amine group, a nitro group, a C1 to C40 silyl group, a C1 to C30 alkyl group, a C1 to C10 alkylsilyl group, a C3 to C30 cycloalkyl group, a C2 to C30 heterocycloalkyl group, a C6 to C30 aryl group, a C2 to C30 heterocyclic group, a C1 to C20 alkoxy group, a fluoro group, a C1 to C10 trifluoroalkyl group such as a trifluoromethyl group, or a cyano group.

In addition, two adjacent substituents of the substituted C1 to C20 amine group, the substituted C3 to C40 silyl group, the substituted C1 to C30 alkyl group, the substituted C1 to C10 alkylsilyl group, the substituted C3 to C30 cycloalkyl group, the substituted C2 to C30 heterocycloalkyl group, the substituted C6 to C30 aryl group, the substituted C2 to C30 heterocyclic group, or the substituted C1 to C20 alkoxy group may be fused to form a ring. For example, the substituted C6 to C30 aryl group may be fused with another adjacent substituted C6 to C30 aryl group to form a substituted or unsubstituted fluorene ring.

In the present specification, when specific definition is not otherwise provided, “hetero” refers to one including 1 to 3 hetero atoms selected from the group consisting of N, O, S, P, and Si, and remaining carbons in one compound or substituent.

In the present specification, when a definition is not otherwise provided, “alkyl group” refers to an aliphatic hydrocarbon group. The alkyl group may be “a saturated alkyl group” without any double bond or triple bond.

The alkyl group may be a C1 to C30 alkyl group. More specifically, the alkyl group may be a C1 to C20 alkyl group or a C1 to C10 alkyl group. For example, a C1 to C4 alkyl group may have 1 to 4 carbon atoms in an alkyl chain which may be selected from methyl, ethyl, propyl, iso-propyl, n-butyl, iso-butyl, sec-butyl, and t-butyl.

Specific examples of the alkyl group may be a methyl group, an ethyl group, a propyl group, an isopropyl group, a butyl group, an isobutyl group, a t-butyl group, a pentyl group, a hexyl group, a cyclopropyl group, a cyclobutyl group, a cyclopentyl group, a cyclohexyl group, and the like.

In the present specification, “aryl group” refers to a group including at least one hydrocarbon aromatic moiety, and

all the elements of the hydrocarbon aromatic moiety have p-orbitals which form conjugation, for example a phenyl group, a naphthyl group, and the like,

two or more hydrocarbon aromatic moieties may be linked by a sigma bond and may be, for example a biphenyl group, a terphenyl group, a quarterphenyl group, and the like, and

two or more hydrocarbon aromatic moieties are fused directly or indirectly to provide a non-aromatic fused ring. For example, it may be a fluorenyl group.

The aryl group may include a monocyclic, polycyclic or fused ring polycyclic (i.e., rings sharing adjacent pairs of carbon atoms) functional group.

In the present specification, “heterocyclic group” is a generic concept of a heteroaryl group, and may include at least one hetero atom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, a “heteroaryl group” may refer to aryl group including at least one hetero atom selected from N, O, S, P, and Si instead of carbon (C). Two or more heteroaryl groups are linked by a sigma bond directly, or when the C2 to C60 heteroaryl group includes two or more rings, the two or more rings may be fused. When the heteroaryl group is a fused ring, each ring may include 1 to 3 hetero atoms.

Specific examples of the heteroaryl group may be a pyridinyl group, a pyrimidinyl group, a pyrazinyl group, a pyridazinyl group, a triazinyl group, a quinolinyl group, an isoquinolinyl group, and the like.

More specifically, the substituted or unsubstituted C6 to C30 aryl group and/or the substituted or unsubstituted C2 to C30 heterocyclic group may be a substituted or unsubstituted phenyl group, a substituted or unsubstituted naphthyl group, a substituted or unsubstituted anthracenyl group, a substituted or unsubstituted phenanthryl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted chrysenyl group, a substituted or unsubstituted triphenylenyl group, a substituted or unsubstituted perylenyl group, a substituted or unsubstituted fluorenyl group, a substituted or unsubstituted indenyl group, a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridinyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, or a substituted or unsubstituted dibenzothiophenyl group, or a combination thereof, but are not limited thereto.

In the present specification, a single bond refers to a direct bond not by carbon or a hetero atom except carbon, and specifically the meaning that L is a single bond means that a substituent linked to L directly bonds with a central core. That is, in the present specification, the single bond does not refer to methylene that is bonded via carbon.

In the specification, hole characteristics refer to an ability to donate an electron to form a hole when an electric field is applied and that a hole formed in the anode may be easily injected into the emission layer, and a hole formed in an emission layer may be easily transported into an anode and transported in the emission layer due to conductive characteristics according to a highest occupied molecular orbital (HOMO) level.

In addition, electron characteristics refer to an ability to accept an electron when an electric field is applied and that an electron formed in a cathode may be easily injected into the emission layer, and an electron formed in an emission layer may be easily transported into a cathode and transported in the emission layer due to conductive characteristics according to a lowest unoccupied molecular orbital (LUMO) level.

Hereinafter, an organic optoelectronic device according to an embodiment is described.

An organic optoelectronic device according to an embodiment includes an anode and a cathode facing each other,

an emission layer between the anode and the cathode,

a hole transport layer between the anode and the emission layer, and

a hole transport auxiliary layer between the hole transport layer and the emission layer,

the emission layer includes at least one first compound represented by Chemical Formula 1; and at least one second compound represented by Chemical Formula 2, and

the hole transport auxiliary layer includes at least one third compound represented by Chemical Formula 3.

The organic optoelectronic device may be any device to convert electrical energy into photoenergy and vice versa without particular limitation, and may be, for example an organic photoelectric device, an organic light emitting diode, an organic solar cell, and an organic photo conductor drum.

Herein, an organic light emitting diode as one example of an organic optoelectronic device is described, but the present invention is not limited thereto, and may be applied to other organic optoelectronic device in the same way.

In the drawings, the thickness of layers, films, panels, regions, etc., are exaggerated for clarity. Like reference numerals designate like elements throughout the specification. It will be understood that when an element such as a layer, film, region, or substrate is referred to as being “on” another element, it can be directly on the other element or intervening elements may also be present. In contrast, when an element is referred to as being “directly on” another element, there are no intervening elements present.

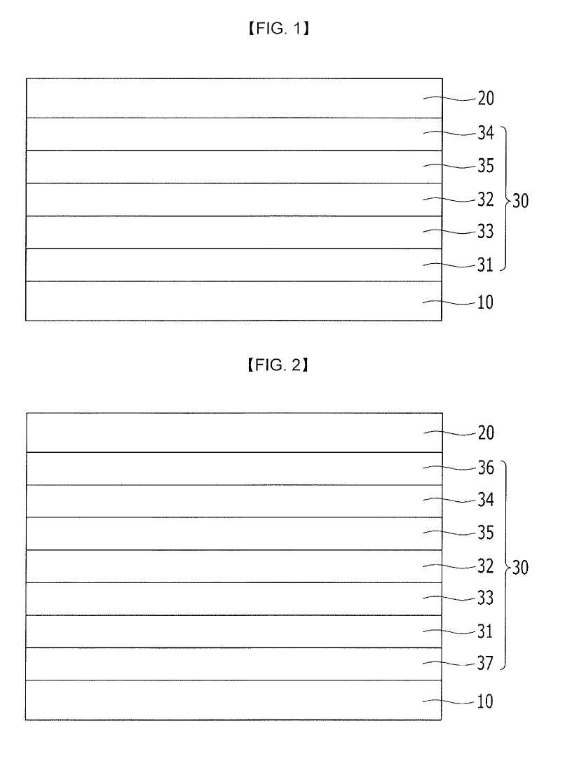

FIG. 1 is a schematic cross-sectional view of an organic optoelectronic device according to an embodiment.

Referring to FIG. 1, an organic optoelectronic device according to an embodiment includes an anode 10 and a cathode 20 and an organic layer 30 between the anode 10 and the cathode 20.

The anode 10 may be made of a conductor having a large work function to help hole injection, and may be for example metal, metal oxide and/or a conductive polymer. The anode 10 may be, for example a metal nickel, platinum, vanadium, chromium, copper, zinc, gold, and the like or an alloy thereof; metal oxide such as zinc oxide, indium oxide, indium tin oxide (ITO), indium zinc oxide (IZO), and the like; a combination of metal and oxide such as ZnO and Al or SnO₂ and Sb; a conductive polymer such as poly(3-methylthiophene), poly(3,4-(ethylene-1,2-dioxy)thiophene) (PEDT), polypyrrole, and polyaniline, but is not limited thereto.

The cathode 20 may be made of a conductor having a small work function to help electron injection, and may be for example metal, metal oxide and/or a conductive polymer. The cathode 20 may be for example a metal or an alloy thereof such as magnesium, calcium, sodium, potassium, titanium, indium, yttrium, lithium, gadolinium, aluminum silver, tin, lead, cesium, barium, and the like; a multi-layer structure material such as LiF/Al, LiO₂/Al, LiF/Ca, LiF/Al, and BaF₂/Ca, but is not limited thereto.

The organic layer 30 includes a hole transport layer 31, an emission layer 32, and a hole transport auxiliary layer 33 between the hole transport layer 31 and the emission layer 32.

Referring to FIG. 2, the organic layer 30 may further include a hole injection layer 37 between the hole transport layer 31 and the anode 10, and an electron injection layer 36 between the electron transport layer 34 and the cathode 20.

The hole injection layer 37 between the hole transport layer 31 and the anode 10 the improves interface characteristics an organic material used as the hole transport layer 31 and ITO used as the anode 10 and is coated on the ITO to smooth uneven upper surface of ITO. For example, the hole injection layer 37 may be selected from materials having a median value between work functions of the ITO and HOMO of the hole transport layer 31 to adjust a difference between the work functions of the ITO and the HOMO of the hole transport layer 31 and particularly. materials having appropriate conductivity. The materials forming the hole injection layer 37 of the present invention may be N4,N4′-diphenyl-N4,N4′-bis(9-phenyl-9H-carbazol-3-yl)biphenyl-4,4′-diamine, but is not limited thereto. A conventional material of the hole injection layer 37 may be also used together, for example, copper phthlalocyanine (CuPc), aromatic amines such as N,N′-dinaphthyl-N,N′-phenyl-(1,1′-biphenyl)-4,4′-diamine (NPD), 4,4′,4″-tris[methylphenyl(phenyl)amino] triphenyl amine (m-MTDATA), 4,4′,4″-tris[1-naphthyl(phenyl)amino] triphenyl amine (1-TNATA), 4,4′,4″-tris[2-naphthyl(phenyl)amino]triphenyl amine (2-TNATA), 1,3,5-tris[N-(4-diphenylaminophenyl)phenylamino] benzene (p-DPA-TDAB), a compound such 4,4′-bis[N-[4-{N,N-bis(3-methylphenyl)amino}phenyl]-N-phenylamino]biphenyl (DNTPD), hexaazatriphenylene-hexacarbonitirile (HAT-CN), and the like, a conductive polymer such as a polythiophene derivative of poly(3,4-ethylenedioxythiophene)-poly(styrnesulfonate) (PEDOT). The hole injection layer 37 may be coated with a thickness, for example of 10 to 300 Å on ITO as an anode.

The electron injection layer 36 is disposed on the electron transport layer and thus, facilitates injection of electrons from a cathode and ultimately improves power efficiency and may, for example, include LiF, Liq, NaCl, CsF, Li₂O, BaO and the like, which are conventionally used in a related art.

The hole transport layer 31 facilitates hole transport from the anode 10 to the emission layer 32 and may be, for example, formed of an amine compound but is not limited thereto.

The amine compound may include, for example at least one aryl group and/or heteroaryl group. The amine compound may be, for example represented by Chemical Formula a or b but is not limited thereto.

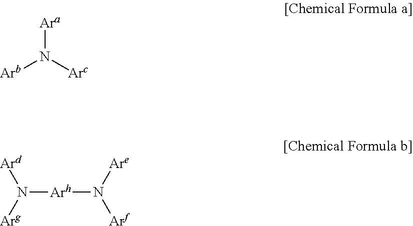

In Chemical Formula a or Chemical Formula b.

Ar^{a} to Ar^{g} are independently hydrogen, deuterium, a substituted or unsubstituted C1 to C20 alkyl group, a substituted or unsubstituted C6 to C30 aryl group, a substituted or unsubstituted C2 to C30 heteroaryl group, or a combination thereof,

at least one of Ar^{a} to Ar^{c} and at least one of Ar^{d} to Ar^{g} are a substituted or unsubstituted C6 to C30 aryl group, a substituted or unsubstituted C2 to C30 heteroaryl group, or a combination thereof, and

Ar^{h} is a single bond, a substituted or unsubstituted C1 to C20 alkylene group, a substituted or unsubstituted C6 to C30 arylene group, a substituted or unsubstituted C2 to C30 heteroarylene group or a combination thereof.

The electron transport layer 34 easily transports electrons from the cathode 20 to the emission layer 32 and may be formed of an organic compound containing an electron-accepting functional group (an electron-withdrawing group), a metal compound well accepting electrons, or a mixture thereof. For example, the electron transport layer material may include aluminum trihydroxyquinoline (Alq₃), a 1,3,4-oxadiazole derivative of 2-(4-biphenylyl)-5-phenyl-1,3,4-oxadiazole (PBD), a quinoxaline derivative of 1,3,4-tris[(3-penyl-6-trifluoromethyl)quinoxaline-2-yl] benzene (TPQ), a triazole derivative and a triazine derivative of 8-(4-(4-(naphthalen-2-yl)-6-(naphthalen-3-yl)-1,3,5-triazin-2-yl)phenyl)quinoline), and the like, but is not limited thereto.

In addition, the electron transport layer may include an organometallic compound represented by Chemical Formula c alone or as a mixture with the electron transport layer material.

Yₘ-M-(OA)ₙ [Chemical Formula c]

In Chemical Formula c,

Y includes a moiety where one selected from C, N, O and S directly bonds with M to form a single bond and a moiety where one selected from C, N, O and S forms a coordination bond with M, and is a chelated ligand with the single bond and a coordination bond,

the M is an alkali metal, an alkali earth metal, aluminum (Al), or boron (B) atom, and the OA is a monovalent ligand being capable of forming a single bond or a coordination bonding with the M,

the O is oxygen,

A is selected from a substituted or unsubstituted C1 to C30 alkyl group, a substituted or unsubstituted C5 to C50 aryl group, a substituted or unsubstituted C2 to C30 alkenyl group, a substituted or unsubstituted C2 to C20 alkynyl group, a substituted or unsubstituted C3 to C30 cycloalkyl group, a substituted or unsubstituted C5 to C30 cycloalkenyl group, and a substituted or unsubstituted C2 to C50 heteroaryl group having heterogeneous atom of O, N or S,

when the M is a metal selected from an alkali metal, m=1 and n=0,

when the M is a metal selected from an alkali earth metal, m=1 and n=1, or m=2, and n=0,

when the M is boron or aluminum, m is one of 1 to 3, and n is one of 0 to 2, satisfying m+n=3; and

the ‘substituted’ of the ‘substituted or unsubstituted’ refers to that at least one hydrogen is replaced by one or more substituent selected from deuterium, a cyano group, a halogen, a hydroxy group, a nitro group, an alkyl group, an alkoxy group, an alkylamino group, an arylamino group, hetero an arylamino group, an alkylsilyl group, an arylsilyl group, an aryloxy group, an aryl group, a heteroaryl group, germanium, phosphorus, and boron.

In the present invention, each Y is the same or different, and are independently one selected from Chemical Formula c1 to Chemical Formula c39, but is not limited thereto.

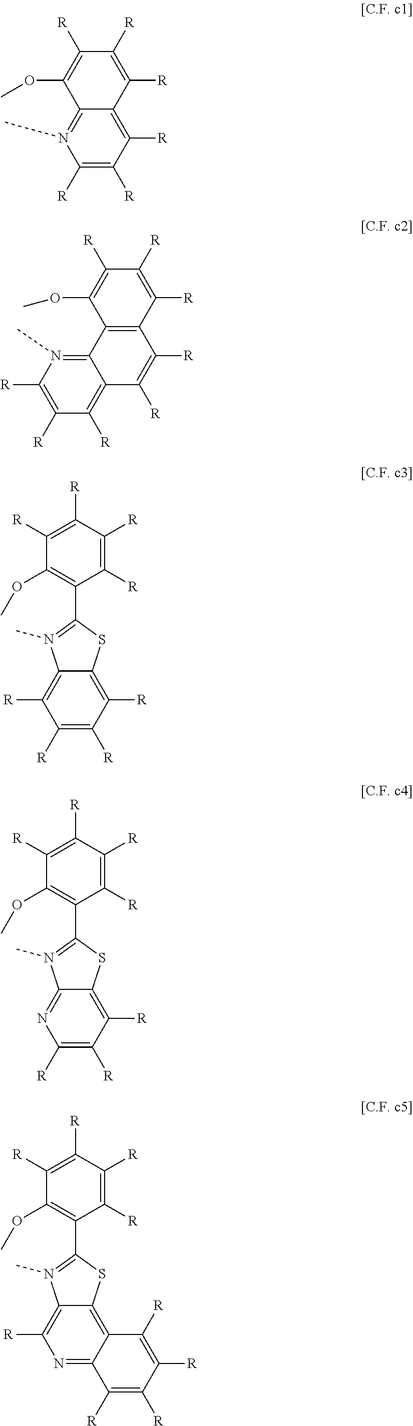

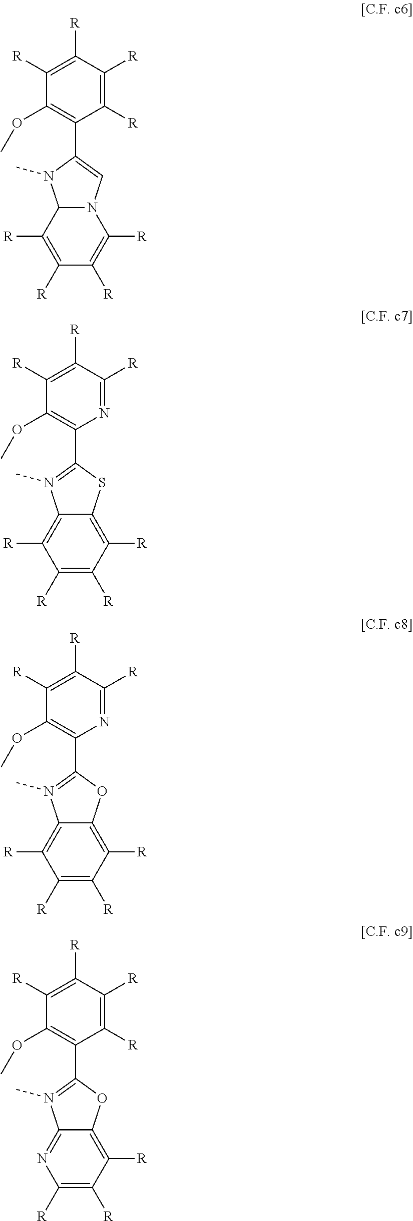

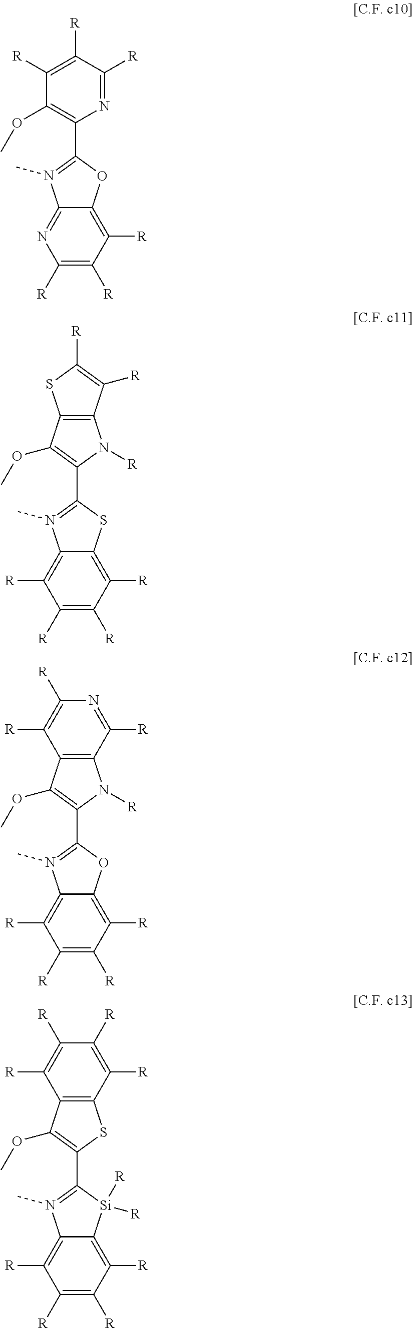

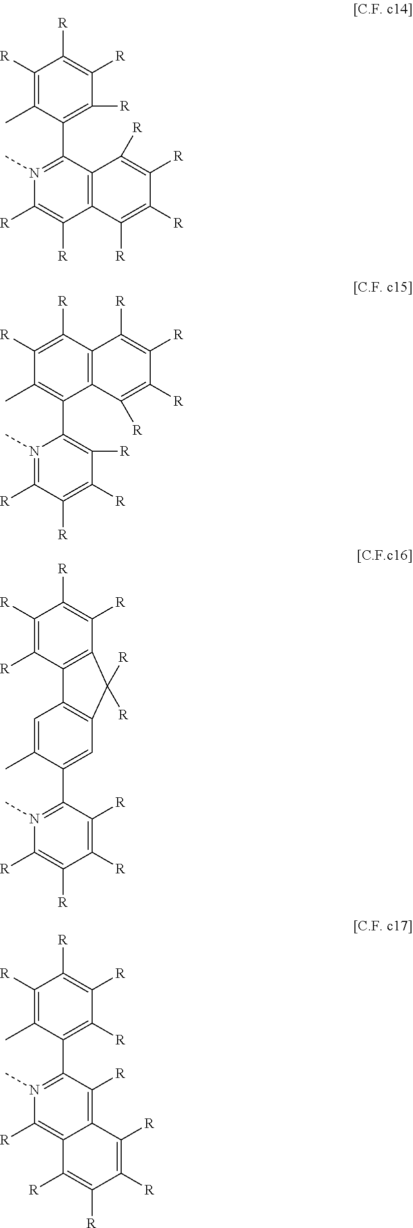

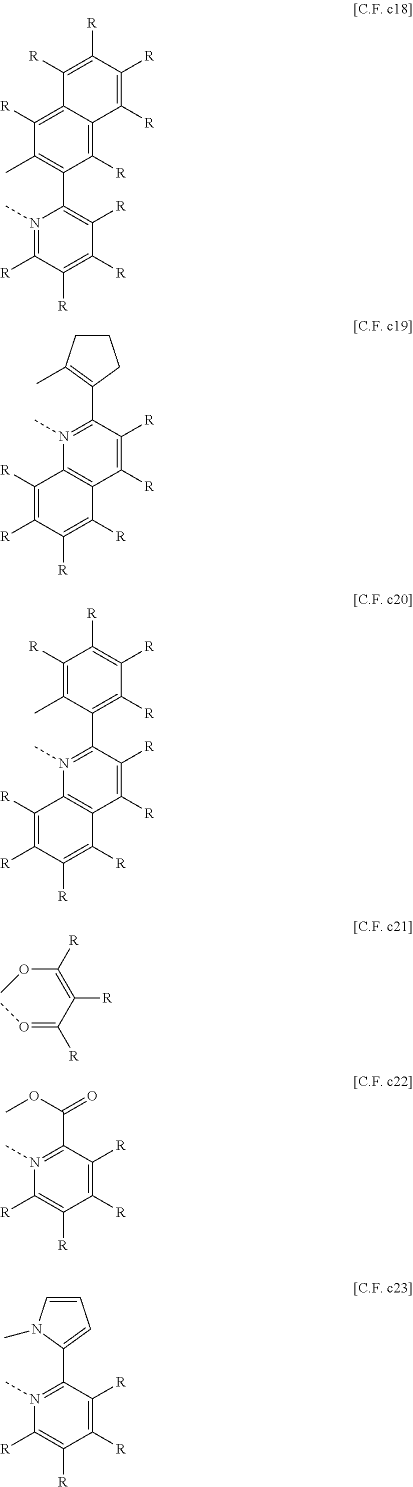

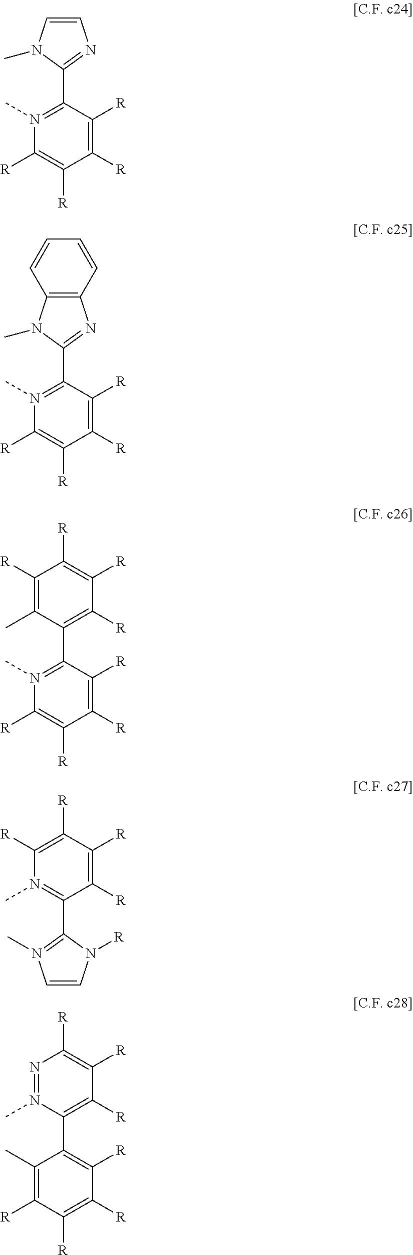

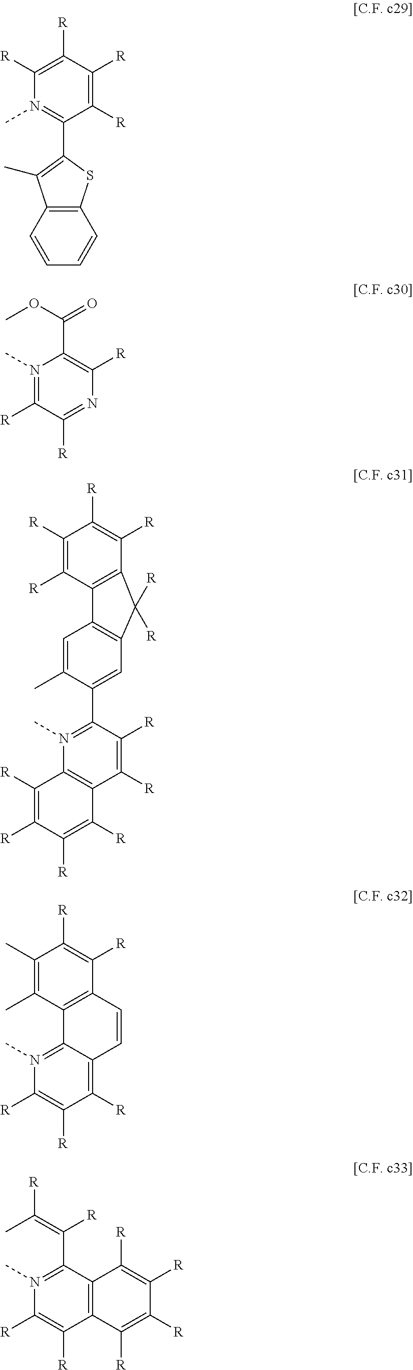

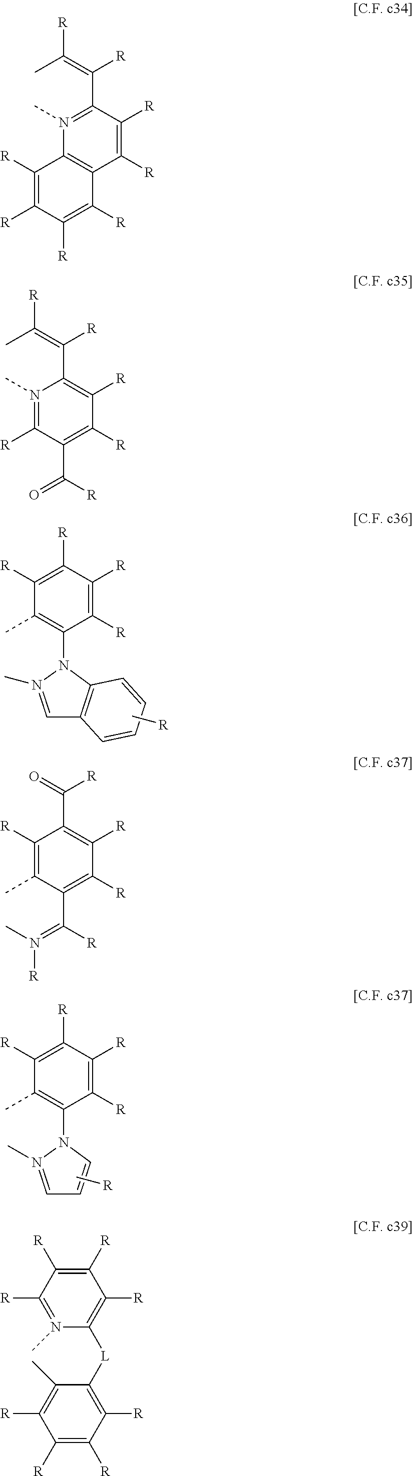

In Chemical Formulae c1 to c39,

R is the same or different and is each independently selected from hydrogen, deuterium, halogen, a cyano group, a substituted or unsubstituted C1 to C30 alkyl group, a substituted or unsubstituted C6 to C30 aryl group, a substituted or unsubstituted C3 to C30 heteroaryl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C1 to C30 alkylamino group, a substituted or unsubstituted C1 to C30 alkylsilyl group, a substituted or unsubstituted C6 to C30 arylamino group, and a substituted or unsubstituted C6 to C30 arylsilyl group, and R is linked to an adjacent substitutent through alkylene or alkenylene to form a spiroring or a fused ring.

The emission layer 32 is an organic layer emitting light and includes a host and a dopant when a doping system is adopted. Herein, the host mainly promotes a recombination of electrons and holes and holds excitons in an emission layer, while the dopant efficiently emits light from the excitons obtained from the recombination.

The emission layer 32 includes at least two kinds of hosts and dopants, and the hosts include a first compound having bipolar characteristics in which electron characteristics are relatively strong and a second compound having bipolar characteristics in which hole characteristics are relatively strong.

The first compound is a compound having bipolar characteristics in which electron characteristics are relatively strong and may be represented by Chemical Formula 1.

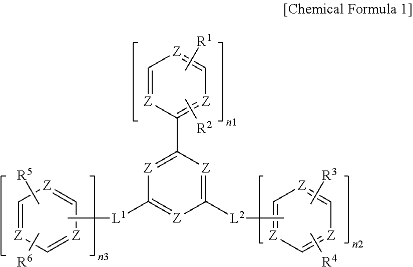

In Chemical Formula 1,

Z is independently N, C, or CR^{a},

at least one of Z is N,

R¹ to R⁶ and R^{a} are independently hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof,

R³ to R⁶ are independently present or adjacent groups are linked to each other to provide a ring,

L¹ and L² are independently a single bond, a substituted or unsubstituted C6 to C30 arylene group, a substituted or unsubstituted C2 to C30 heteroarylene group, or a combination thereof,

n1 is one of integers of 0 to 3, and

n2 and n3 are independently one of integers of 1 to 5.

The first compound may have a structure of easily receiving electrons when an electric field is applied thereto due to at least one nitrogen-containing ring and an injection amount of electrons increases, and thus decreases a driving voltage and increases efficiency of an organic optoelectronic device including the first compound.

Specifically, the first compound includes a plurality of substituted or unsubstituted aryl group moiety easily accepting holes and a nitrogen-containing ring moiety easily accepting electrons, and thus may form a bipolar structure and balance flows of the holes and the electrons and accordingly improve efficiency of an organic optoelectronic device, and

the first compound may appropriately localize the plurality of substituted or unsubstituted aryl group moiety easily accepting holes and the nitrogen-containing ring moiety easily accepting electrons in the compound having the bipolar structure and control a flow of a conjugated system, and thus show excellent bipolar characteristics and improve life-span of the organic optoelectronic device.

For example, the first compound may be represented by Chemical Formula 1-I or 1-II in accordance with arrangements of an aryl group moiety and a nitrogen-containing ring moiety.

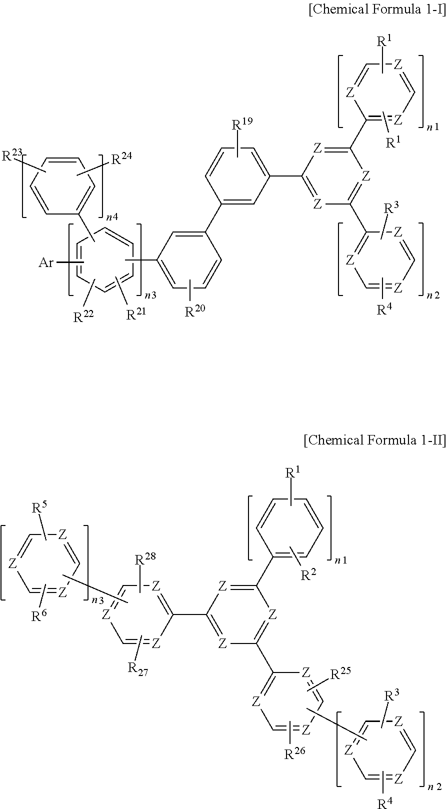

In Chemical Formulae 1-I and 1-II, Z, R¹ to R⁶, and n1 to n3 are the same as defined above,

R¹⁹ to R²⁸ are independently hydrogen, deuterium, a substituted or unsubstituted C1 to C10 alkyl group, a substituted or unsubstituted C6 to C12 aryl group, a substituted or unsubstituted C2 to C30 heteroaryl group, or a combination thereof,

Ar is independently a substituted or unsubstituted C6 to C30 aryl group, a substituted or unsubstituted C2 to C30 heterocyclic group, or a combination thereof,

n4 is an integer ranging from 0 to 2, and

“substituted” is the same as defined above.

The first compound represented by Chemical Formulae 1-I and 1-II has at least one kink structure as a center of an arylene group and/or a heteroarylene group, which is particularly desirable for performance.

The kink structure is a structure that two linking moieties of arylene groups and/or heteroarylene groups are not linear. For example, as for phenylene, ortho phenylene (o-phenylene) and meta phenylene (m-phenylene) have a kink structure where linking moieties do not form a linear structure, while para phenylene (p-phenylene) has no kink structure because where linking moieties form a linear structure.

For example, Chemical Formula 1-I according to an embodiment may be represented by one of Chemical Formulae 1-IA to 1-IC having a kink structure.

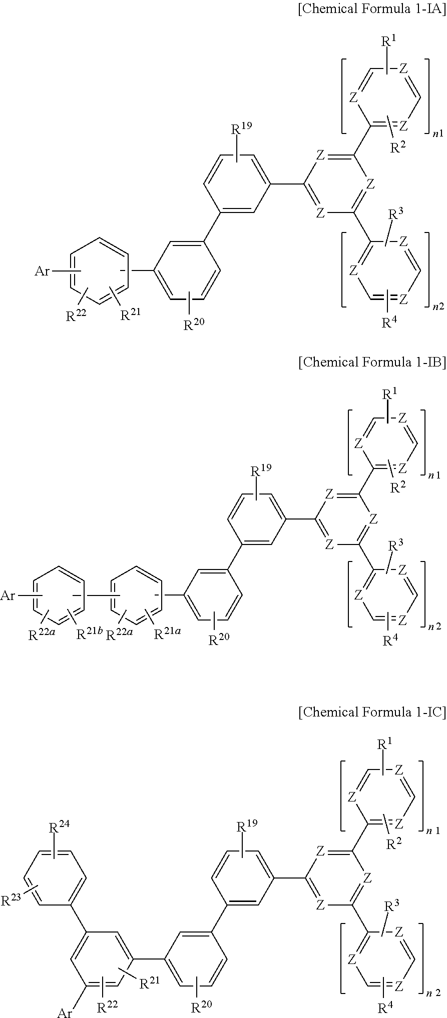

In Chemical Formulae 1-IA to 1-IC, Z, R¹ to R⁴, R¹⁹ to R²⁴, n1 and n2 are the same as above, and R^{21a} and R^{21b} are the same as R²¹, R ^{21a} and R^{22b} are the same as R²²,

Ar is a substituted or unsubstituted C6 to C30 aryl group, a substituted or unsubstituted C2 to C30 heterocyclic group, or a combination thereof, and

“substituted” is the same as defined above.

Specifically, Chemical Formula 1-IA may be represented by Chemical Formula 1-I-1a or 1-I-2a in accordance with a substitution position of Ar, but is not limited thereto.

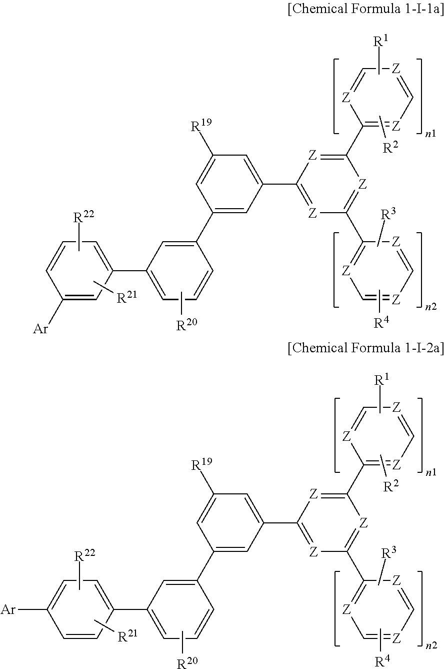

Specifically, Chemical Formula 1-IB may be represented by Chemical Formulae 1-I-1b to 1-I-7b in accordance with linking groups of the aryl group moiety and a substitution position of Ar, but is not limited thereto.

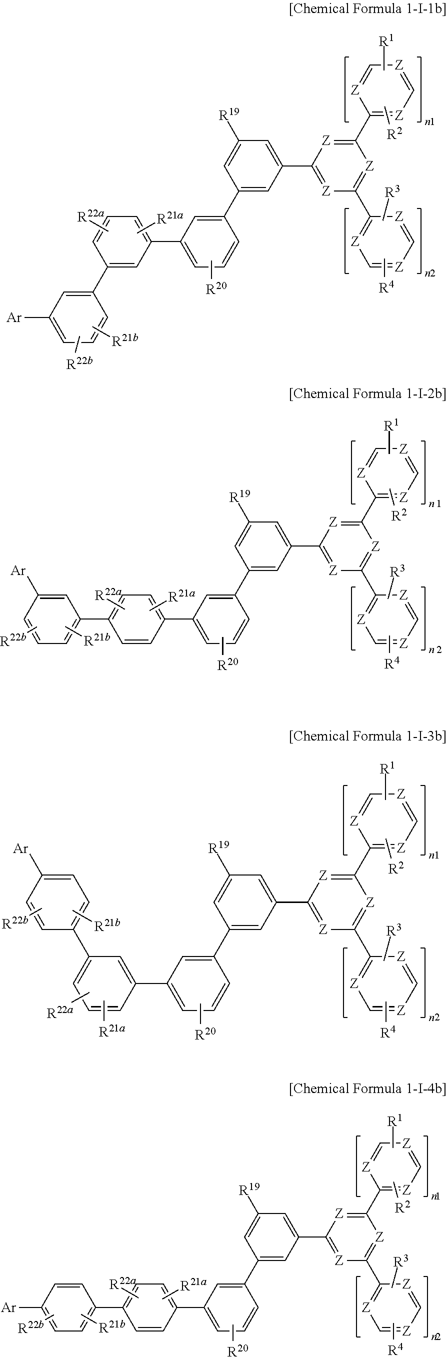

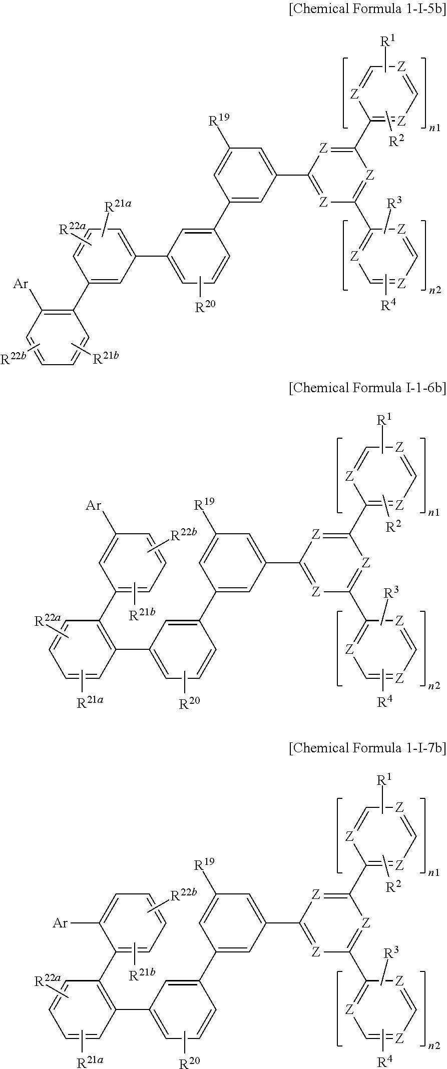

Specifically, Chemical Formula 1-IC may be represented by Chemical Formula 1-I-1c wherein a linking position of R¹⁹ is fixed, but is not limited thereto.

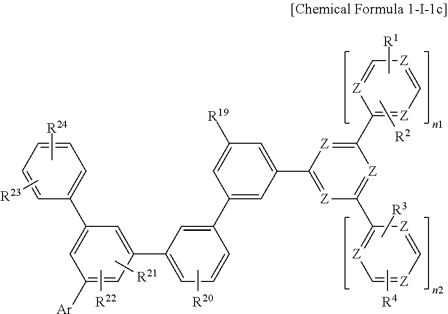

In Chemical Formulae 1-I-1a to 1-I-2a, 1-I-1b to 1-I-7b and 1-I-1c, Z, R¹ to R⁴, R¹⁹ to R²⁴, R^{21a}, R^{21b}, R^{22a}, R^{22b}, n1, n2, and Ar are the same as described above.

On the other hand, n1 of Chemical Formula 1-I may be one of integers of 0 to 3 and n2 may be integers of 1 to 5. Specifically, n1 may be one of integers of 0 to 2, n2 may be one of integers of 1 to 3, for example n1 may be an integer of 0 or 1 and n2 may be an integer of 1 and Chemical Formula 1-I may be represented by Chemical Formulae 1-I-c or 1-I-d but is not limited thereto.

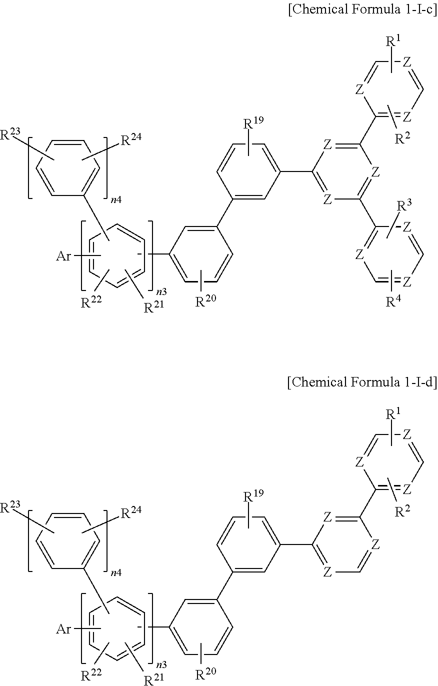

In Chemical Formulae 1-I-c and 1-I-d, Z, R¹ to R⁴, R¹⁹ to R²⁴, n1 to n4, and Ar are the same as described above.

The Ar may be, for example, a substituted or unsubstituted phenyl group, a substituted or unsubstituted biphenyl group, a substituted or unsubstituted terphenyl group, a substituted or unsubstituted naphthyl group, a substituted or unsubstituted pyridinyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted phenanthrolinyl group, or a substituted or unsubstituted quinazolinyl group.

More specifically, the Ar may be selected from substituted or unsubstituted groups of Group 1, but is not limited thereto.

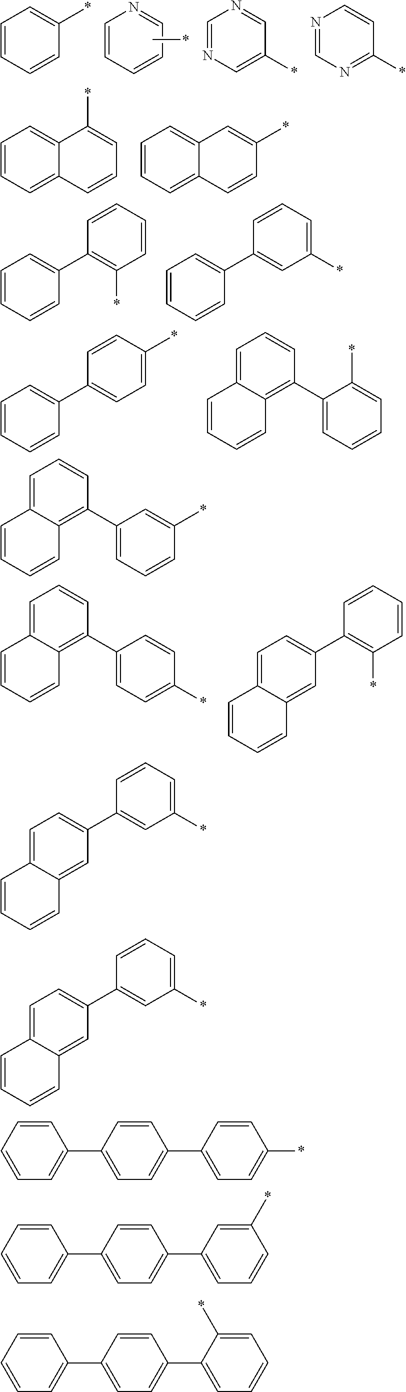

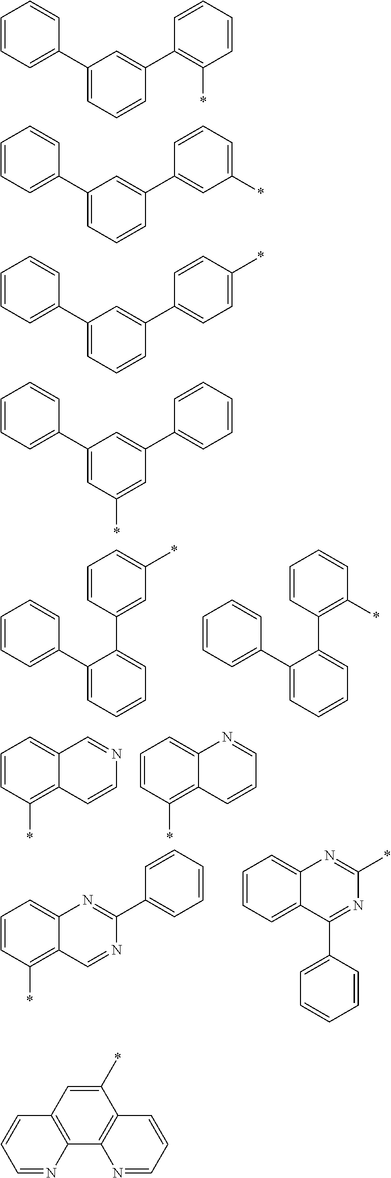

In Group 1, * is a linking point.

Chemical Formula 1-I may be, for example represented by one of Chemical Formulae 1-I-e to 1-I-m in accordance with a position and number of nitrogen, but is not limited thereto.

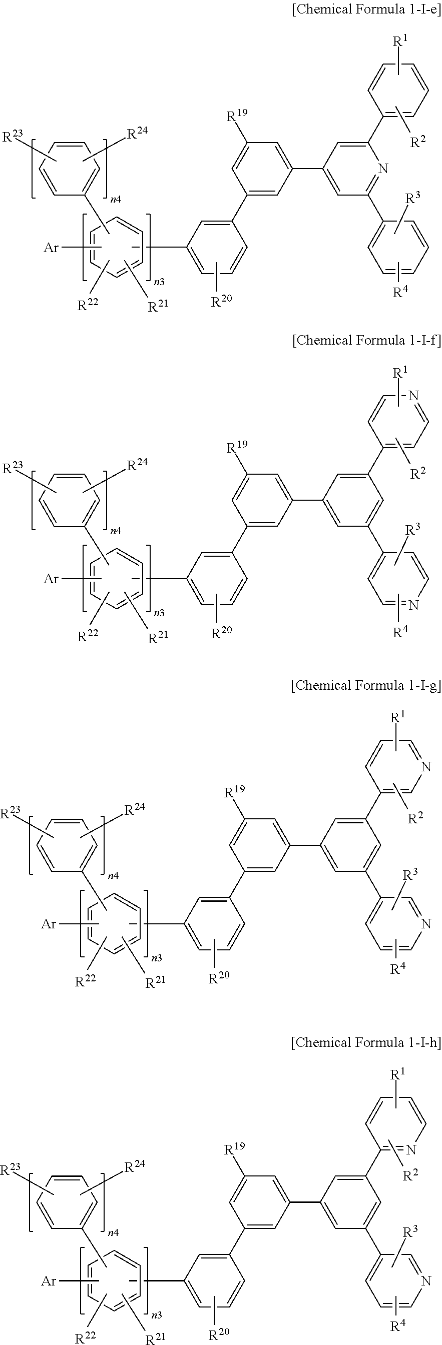

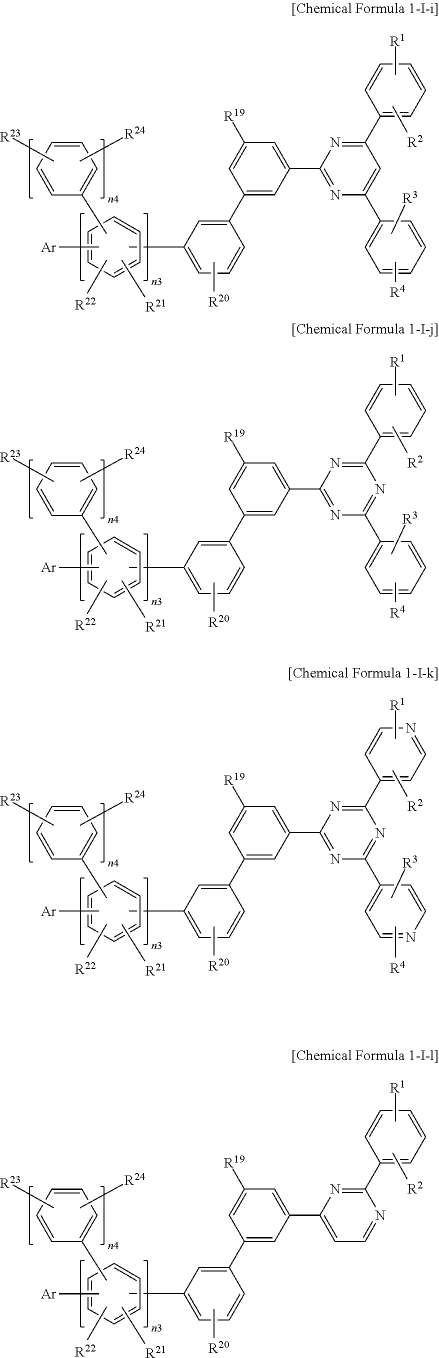

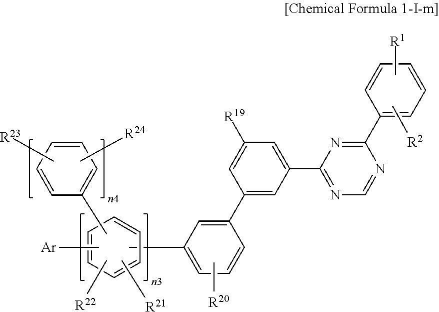

In Chemical Formulae 1-I-e to 1-I-m, R¹ to R⁴, R¹⁹ to R²⁴, n3, n4, and Ar are the same as described above.

For example, Chemical Formula 1-I according to another embodiment may be represented by Chemical Formula 1-IIA or 1-IIB having a kink structure.

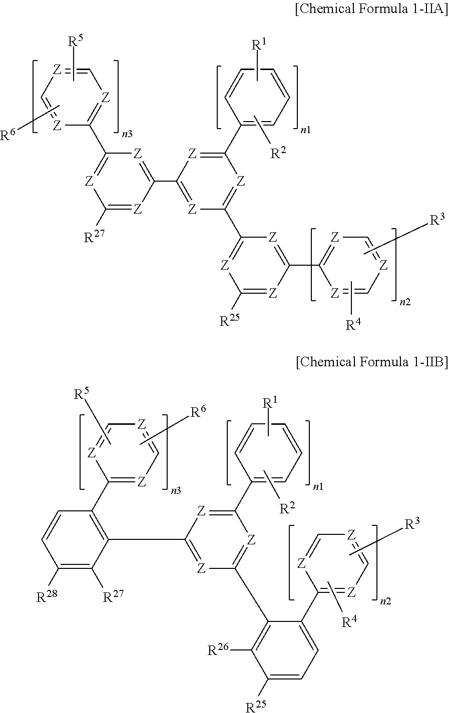

In Chemical Formulae 1-IIA and 1-IIB, Z, R¹ to R⁶, R²⁵ to R²⁸, and n1 to n3 are the same as above,

specifically, R¹ and R² of Chemical Formula 1-II may be hydrogen, deuterium, or a substituted or unsubstituted C6 to C12 aryl group. For example, they are all hydrogen, but are not limited thereto.

Specifically, R³ to R⁶ of Chemical Formula 1-II may independently be hydrogen, a substituted or unsubstituted phenyl group, a substituted or unsubstituted biphenyl group, a substituted or unsubstituted terphenyl group, a substituted or unsubstituted naphthyl group, a substituted or unsubstituted pyridyl group, a substituted or unsubstituted pyrimidinyl group, a substituted or unsubstituted triazinyl group, a substituted or unsubstituted quinolinyl group, a substituted or unsubstituted isoquinolinyl group, a substituted or unsubstituted phenanthrolinyl group, or a substituted or unsubstituted quinazolinyl group. For example, they may be selected from substituted or unsubstituted groups of Group 1, but are not limited thereto.

Specifically, R²⁵ to R²⁸ of Chemical Formula 1-II may independently be hydrogen, a substituted or unsubstituted phenyl group, a substituted or unsubstituted biphenyl group, a substituted or unsubstituted naphthyl group, or a substituted or unsubstituted pyridyl group. For example, they may be selected from substituted or unsubstituted groups of Group 1.

Herein, “substituted” is the same as defined above.

Chemical Formula 1-II may be, for example represented by one of Chemical Formulae 1-II-a to 1-II-h in accordance with a position and number of nitrogen, but is not limited thereto.

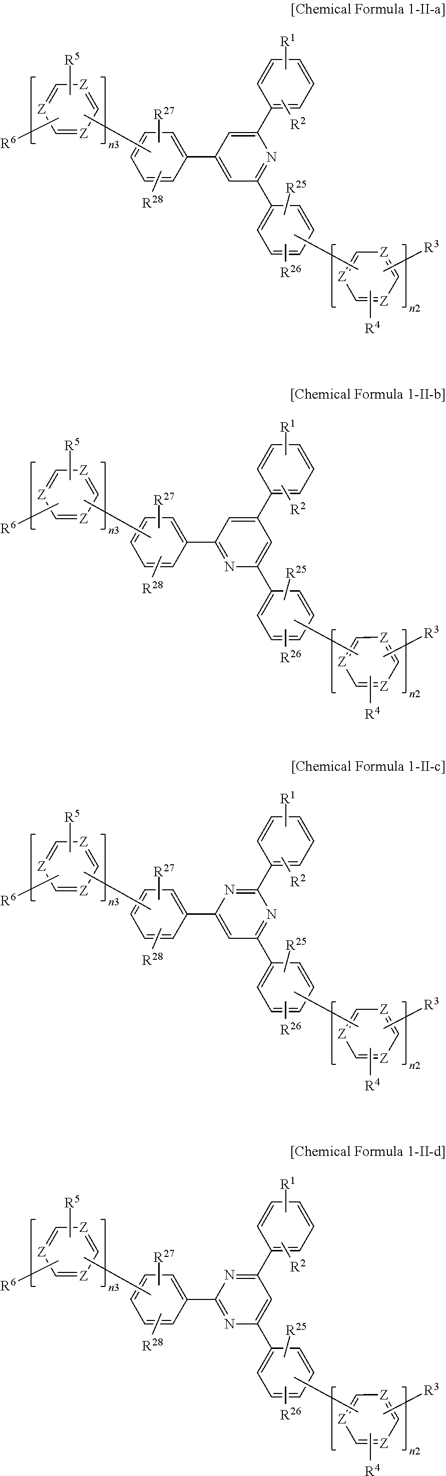

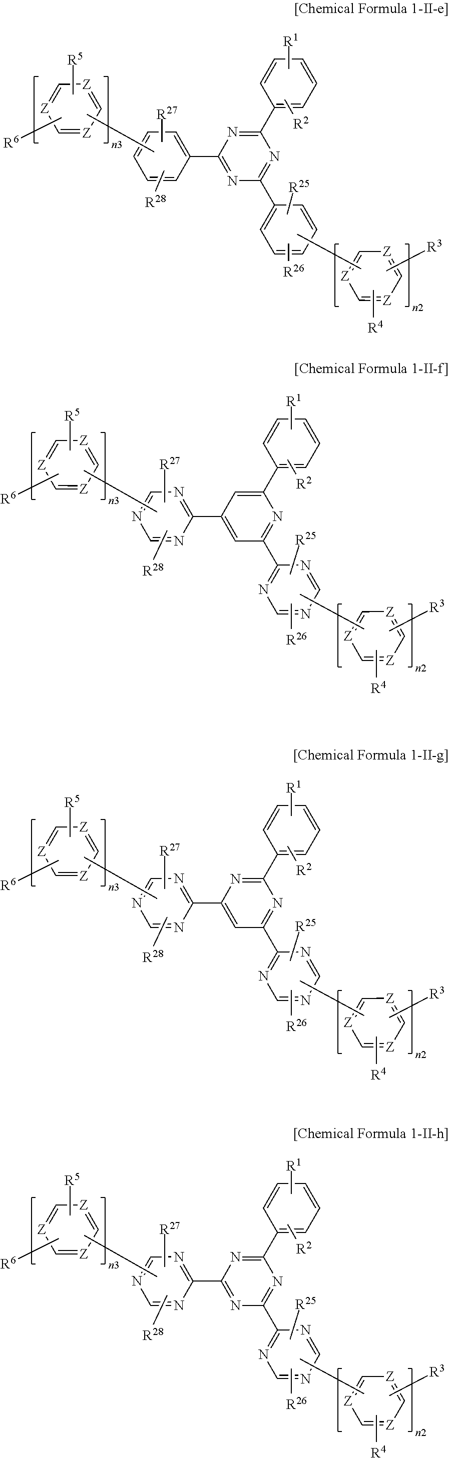

In Chemical Formula 1-II-a to Chemical Formula 1-II-h, R¹ to R⁶, R²⁵ to R²⁸, and n2 and n3 are the same as described above.

The first compound may have a structure of easily receiving electrons when an electric field is applied thereto due to the at least one nitrogen-containing ring and thus, decrease a driving voltage of an organic optoelectronic device including the first compound.

The first compound represented by Chemical Formula 1 may be, for example compounds of Group A, but is not limited thereto.

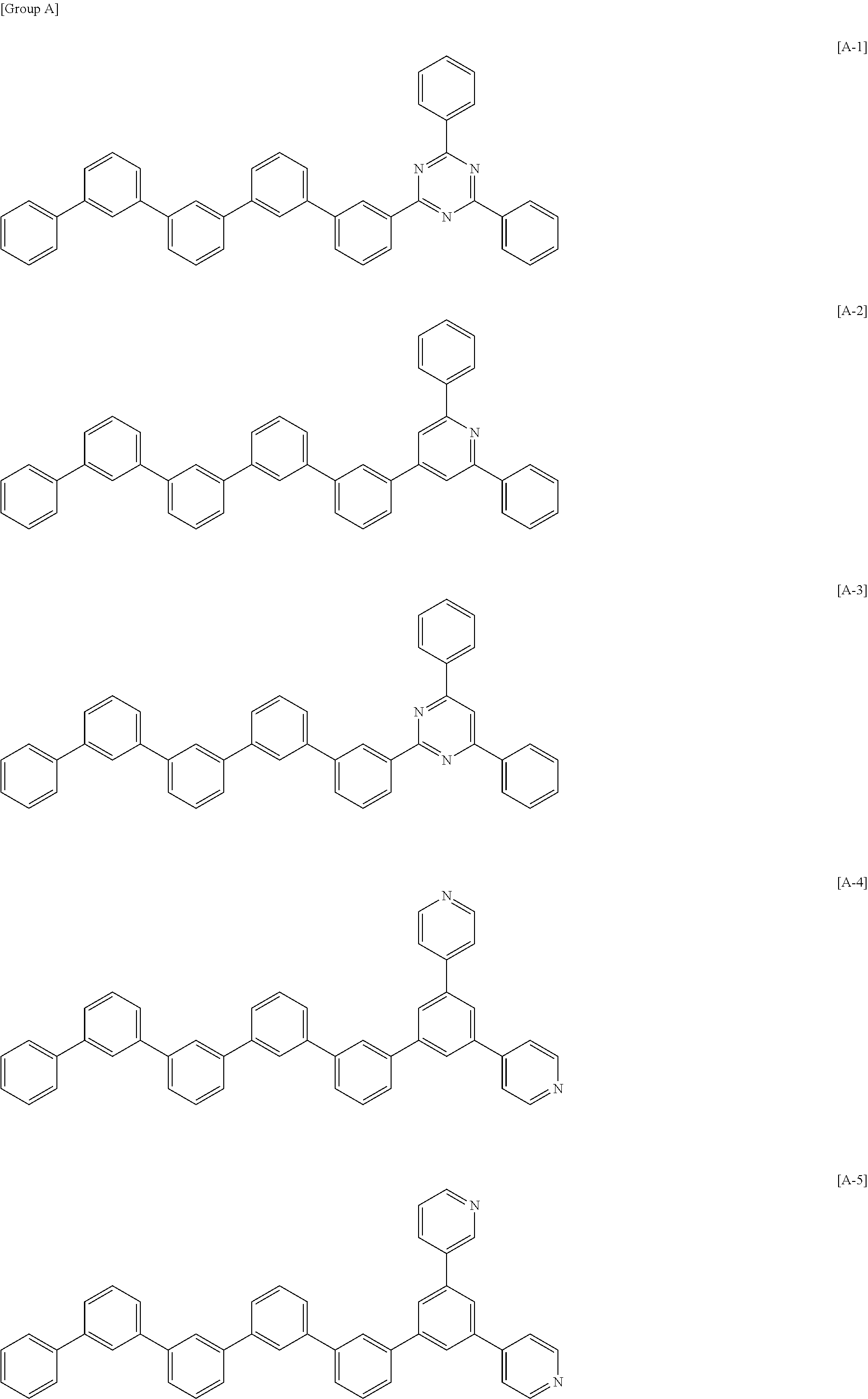 [SMILES: c1ccc(-c2cccc(-c3cccc(-c4cccc(-c5cccc(-c6nc(-c7ccccc7)nc(-c7ccccc7)n6)c5)c4)c3)c2)cc1 ; c1ccc(-c2cccc(-c3cccc(-c4cccc(-c5cccc(-c6cc(-c7ccccc7)nc(-c7ccccc7)c6)c5)c4)c3)c2)cc1 ; c1ccc(-c2cccc(-c3cccc(-c4cccc(-c5cccc(-c6nc(-c7ccccc7)cc(-c7ccccc7)n6)c5)c4)c3)c2)cc1 ; c1ccc(-c2cccc(-c3cccc(-c4cccc(-c5cccc(-c6cc(-c7ccncc7)cc(-c7ccncc7)c6)c5)c4)c3)c2)cc1 ; c1ccc(-c2cccc(-c3cccc(-c4cccc(-c5cccc(-c6cc(-c7ccncc7)cc(-c7cccnc7)c6)c5)c4)c3)c2)cc1]

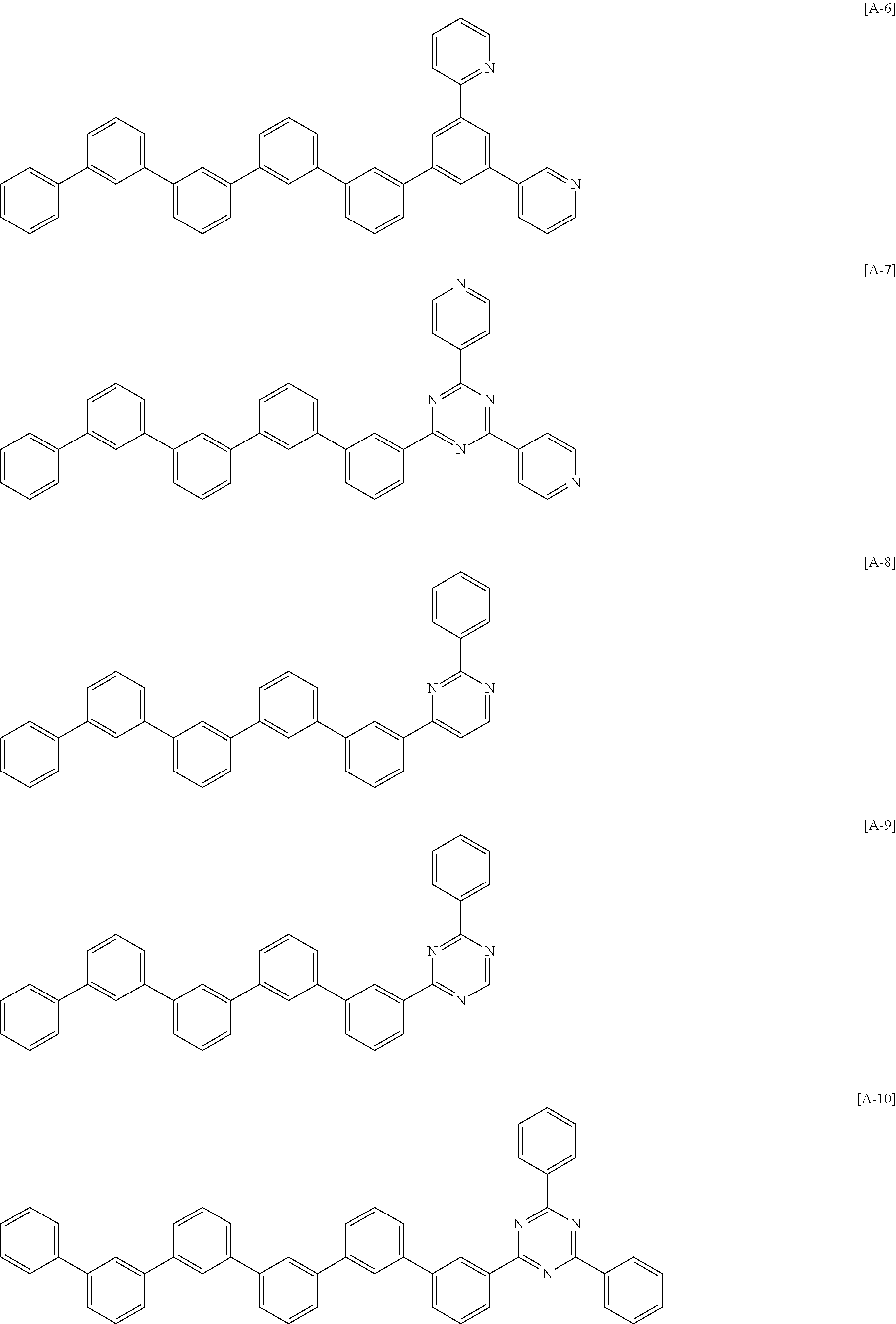 [SMILES: c1ccc(-c2cccc(-c3cccc(-c4cccc(-c5cccc(-c6cc(-c7cccnc7)cc(-c7ccccn7)c6)c5)c4)c3)c2)cc1 ; c1ccc(-c2cccc(-c3cccc(-c4cccc(-c5cccc(-c6nc(-c7ccncc7)nc(-c7ccncc7)n6)c5)c4)c3)c2)cc1 ; c1ccc(-c2cccc(-c3cccc(-c4cccc(-c5cccc(-c6ccnc(-c7ccccc7)n6)c5)c4)c3)c2)cc1 ; c1ccc(-c2cccc(-c3cccc(-c4cccc(-c5cccc(-c6ncnc(-c7ccccc7)n6)c5)c4)c3)c2)cc1 ; c1ccc(-c2cccc(-c3cccc(-c4cccc(-c5cccc(-c6cccc(-c7nc(-c8ccccc8)nc(-c8ccccc8)n7)c6)c5)c4)c3)c2)cc1]

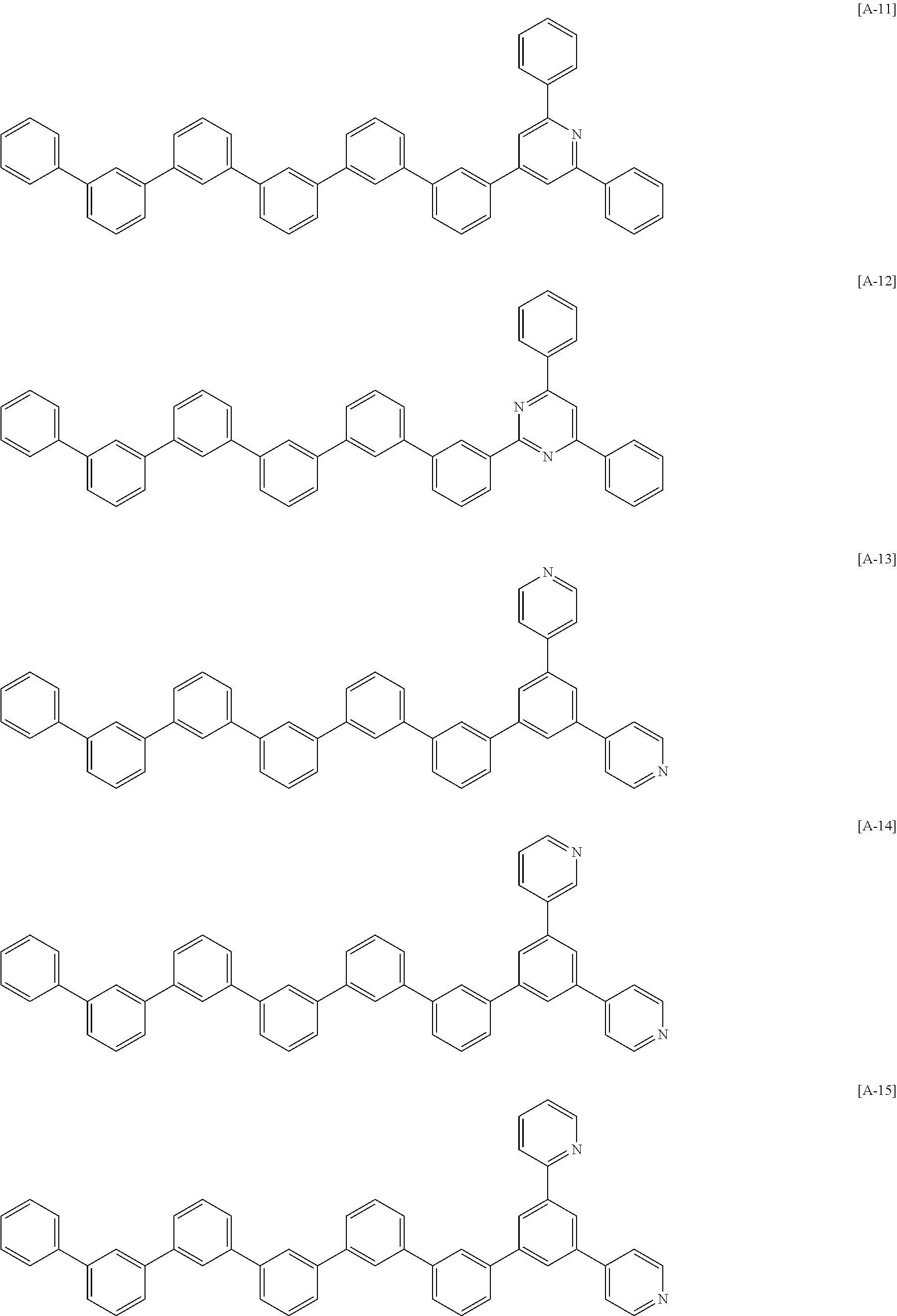 [SMILES: c1ccc(-c2cccc(-c3cccc(-c4cccc(-c5cccc(-c6cccc(-c7cc(-c8ccccc8)nc(-c8ccccc8)c7)c6)c5)c4)c3)c2)cc1 ; c1ccc(-c2cccc(-c3cccc(-c4cccc(-c5cccc(-c6cccc(-c7nc(-c8ccccc8)cc(-c8ccccc8)n7)c6)c5)c4)c3)c2)cc1 ; c1ccc(-c2cccc(-c3cccc(-c4cccc(-c5cccc(-c6cccc(-c7cc(-c8ccncc8)cc(-c8ccncc8)c7)c6)c5)c4)c3)c2)cc1 ; c1ccc(-c2cccc(-c3cccc(-c4cccc(-c5cccc(-c6cccc(-c7cc(-c8ccncc8)cc(-c8cccnc8)c7)c6)c5)c4)c3)c2)cc1 ; c1ccc(-c2cccc(-c3cccc(-c4cccc(-c5cccc(-c6cccc(-c7cc(-c8ccncc8)cc(-c8ccccn8)c7)c6)c5)c4)c3)c2)cc1]

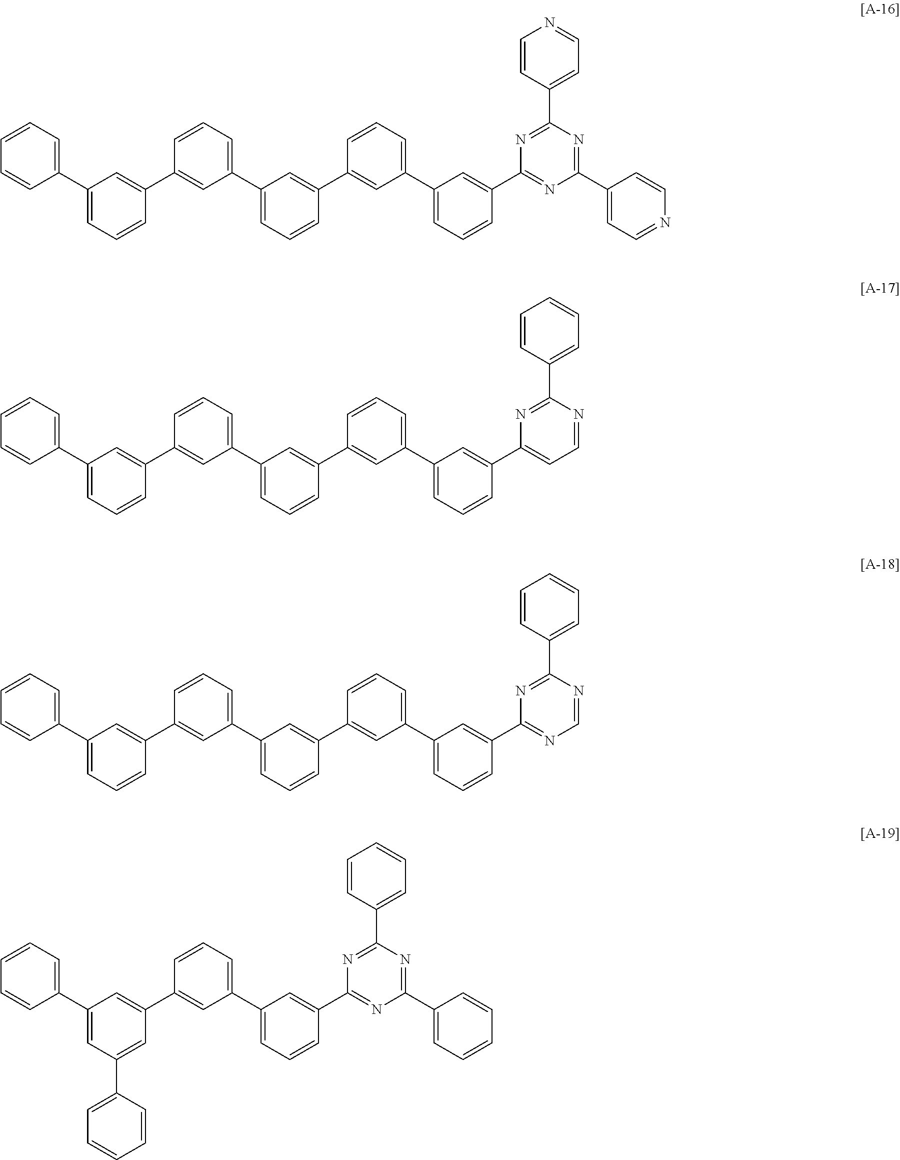 [SMILES: c1ccc(-c2cccc(-c3cccc(-c4cccc(-c5cccc(-c6cccc(-c7nc(-c8ccncc8)nc(-c8ccncc8)n7)c6)c5)c4)c3)c2)cc1 ; c1ccc(-c2cccc(-c3cccc(-c4cccc(-c5cccc(-c6cccc(-c7ccnc(-c8ccccc8)n7)c6)c5)c4)c3)c2)cc1 ; c1ccc(-c2cccc(-c3cccc(-c4cccc(-c5cccc(-c6cccc(-c7ncnc(-c8ccccc8)n7)c6)c5)c4)c3)c2)cc1 ; c1ccc(-c2cc(-c3ccccc3)cc(-c3cccc(-c4cccc(-c5nc(-c6ccccc6)nc(-c6ccccc6)n5)c4)c3)c2)cc1]

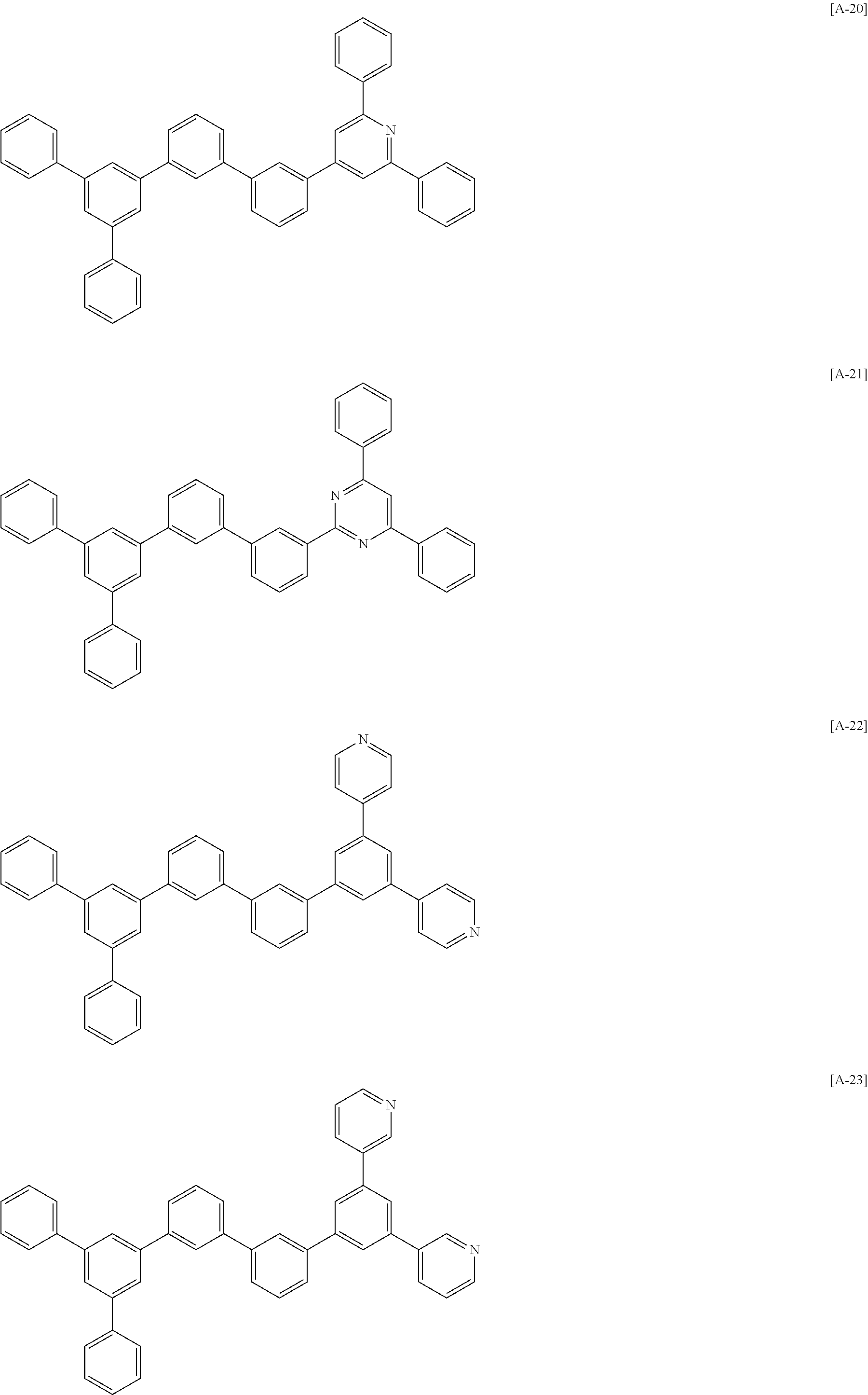 [SMILES: c1ccc(-c2cc(-c3ccccc3)cc(-c3cccc(-c4cccc(-c5cc(-c6ccccc6)nc(-c6ccccc6)c5)c4)c3)c2)cc1 ; c1ccc(-c2cc(-c3ccccc3)cc(-c3cccc(-c4cccc(-c5nc(-c6ccccc6)cc(-c6ccccc6)n5)c4)c3)c2)cc1 ; c1ccc(-c2cc(-c3ccccc3)cc(-c3cccc(-c4cccc(-c5cc(-c6ccncc6)cc(-c6ccncc6)c5)c4)c3)c2)cc1 ; c1ccc(-c2cc(-c3ccccc3)cc(-c3cccc(-c4cccc(-c5cc(-c6cccnc6)cc(-c6cccnc6)c5)c4)c3)c2)cc1]

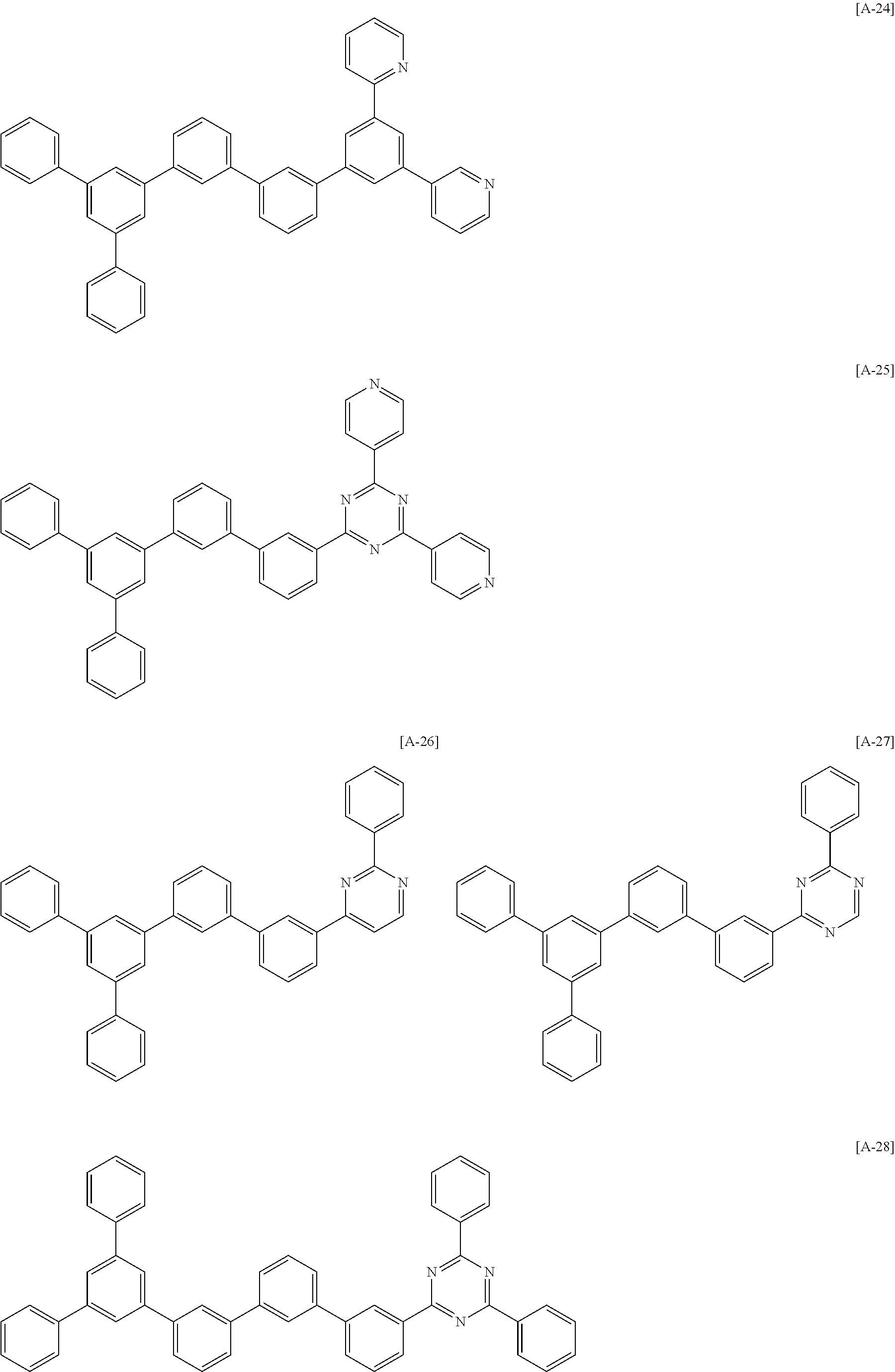 [SMILES: c1ccc(-c2cc(-c3ccccc3)cc(-c3cccc(-c4cccc(-c5cc(-c6cccnc6)cc(-c6ccccn6)c5)c4)c3)c2)cc1 ; c1ccc(-c2cc(-c3ccccc3)cc(-c3cccc(-c4cccc(-c5nc(-c6ccncc6)nc(-c6ccncc6)n5)c4)c3)c2)cc1 ; c1ccc(-c2cc(-c3ccccc3)cc(-c3cccc(-c4cccc(-c5ccnc(-c6ccccc6)n5)c4)c3)c2)cc1 ; c1ccc(-c2cc(-c3ccccc3)cc(-c3cccc(-c4cccc(-c5ncnc(-c6ccccc6)n5)c4)c3)c2)cc1 ; c1ccc(-c2cc(-c3ccccc3)cc(-c3cccc(-c4cccc(-c5cccc(-c6nc(-c7ccccc7)nc(-c7ccccc7)n6)c5)c4)c3)c2)cc1]

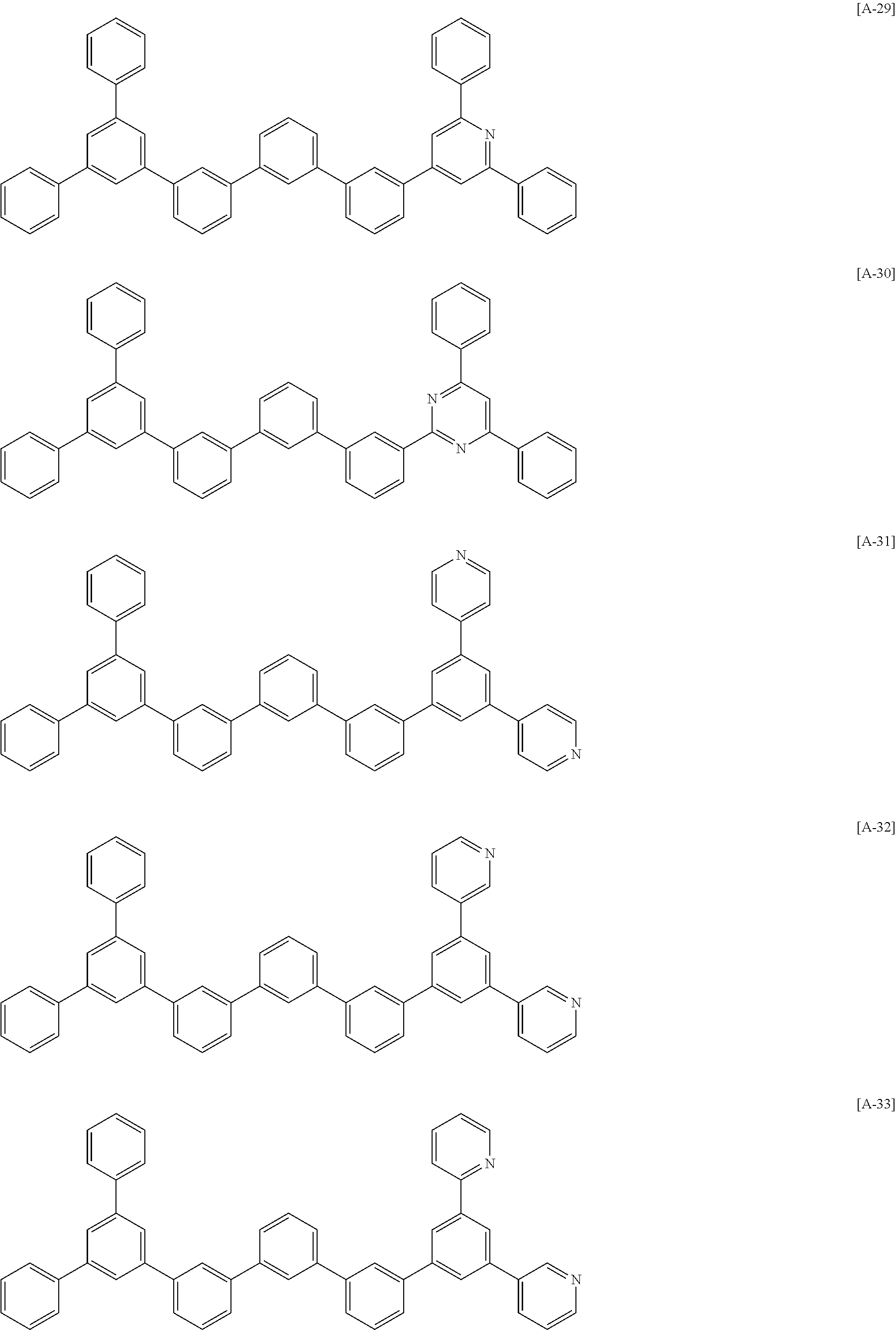 [SMILES: c1ccc(-c2cc(-c3ccccc3)cc(-c3cccc(-c4cccc(-c5cccc(-c6cc(-c7ccccc7)nc(-c7ccccc7)c6)c5)c4)c3)c2)cc1 ; c1ccc(-c2cc(-c3ccccc3)cc(-c3cccc(-c4cccc(-c5cccc(-c6nc(-c7ccccc7)cc(-c7ccccc7)n6)c5)c4)c3)c2)cc1 ; c1ccc(-c2cc(-c3ccccc3)cc(-c3cccc(-c4cccc(-c5cccc(-c6cc(-c7ccncc7)cc(-c7ccncc7)c6)c5)c4)c3)c2)cc1 ; c1ccc(-c2cc(-c3ccccc3)cc(-c3cccc(-c4cccc(-c5cccc(-c6cc(-c7cccnc7)cc(-c7cccnc7)c6)c5)c4)c3)c2)cc1 ; c1ccc(-c2cc(-c3ccccc3)cc(-c3cccc(-c4cccc(-c5cccc(-c6cc(-c7cccnc7)cc(-c7ccccn7)c6)c5)c4)c3)c2)cc1]

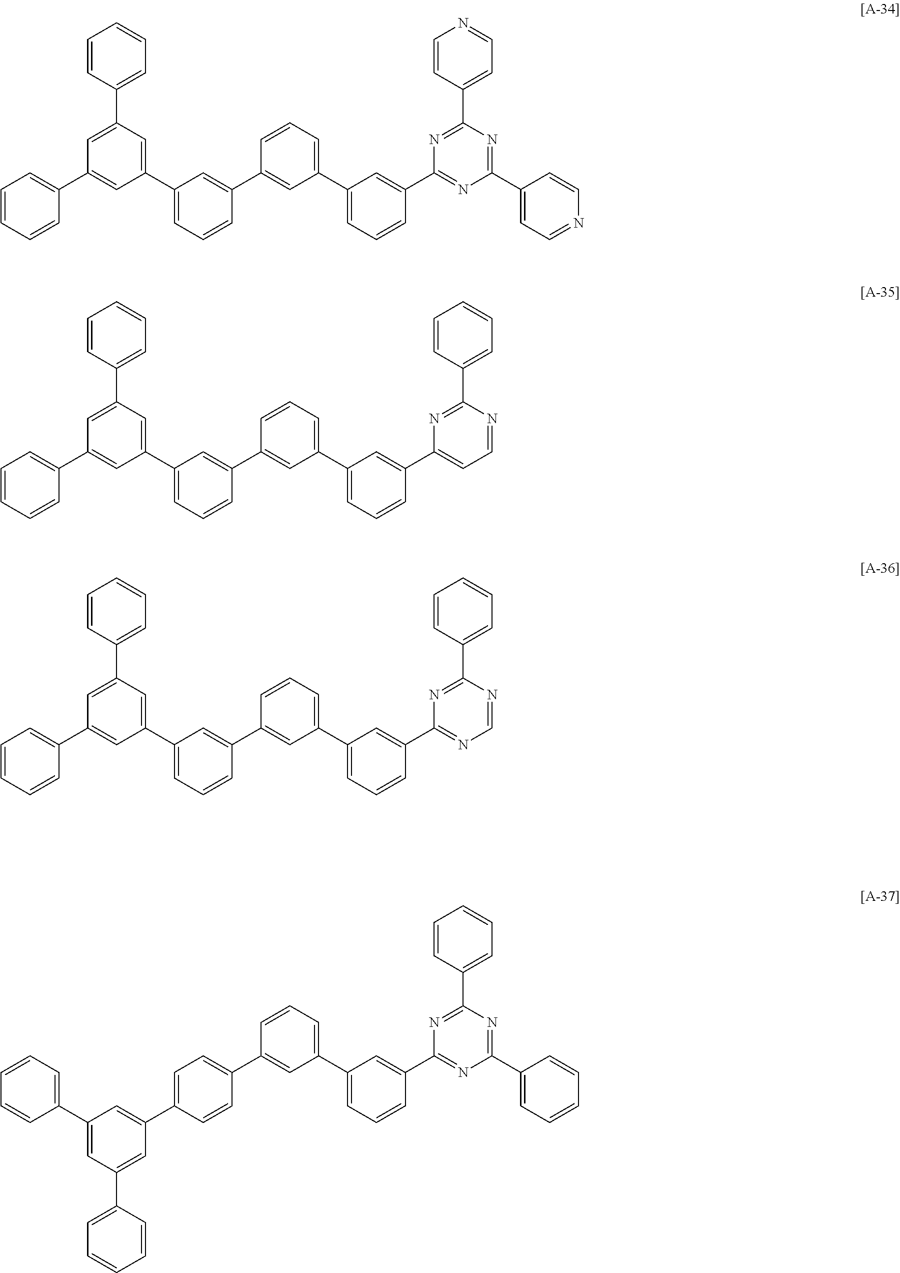 [SMILES: c1ccc(-c2cc(-c3ccccc3)cc(-c3cccc(-c4cccc(-c5cccc(-c6nc(-c7ccncc7)nc(-c7ccncc7)n6)c5)c4)c3)c2)cc1 ; c1ccc(-c2cc(-c3ccccc3)cc(-c3cccc(-c4cccc(-c5cccc(-c6ccnc(-c7ccccc7)n6)c5)c4)c3)c2)cc1 ; c1ccc(-c2cc(-c3ccccc3)cc(-c3cccc(-c4cccc(-c5cccc(-c6ncnc(-c7ccccc7)n6)c5)c4)c3)c2)cc1 ; c1ccc(-c2cc(-c3ccccc3)cc(-c3ccc(-c4cccc(-c5cccc(-c6nc(-c7ccccc7)nc(-c7ccccc7)n6)c5)c4)cc3)c2)cc1]

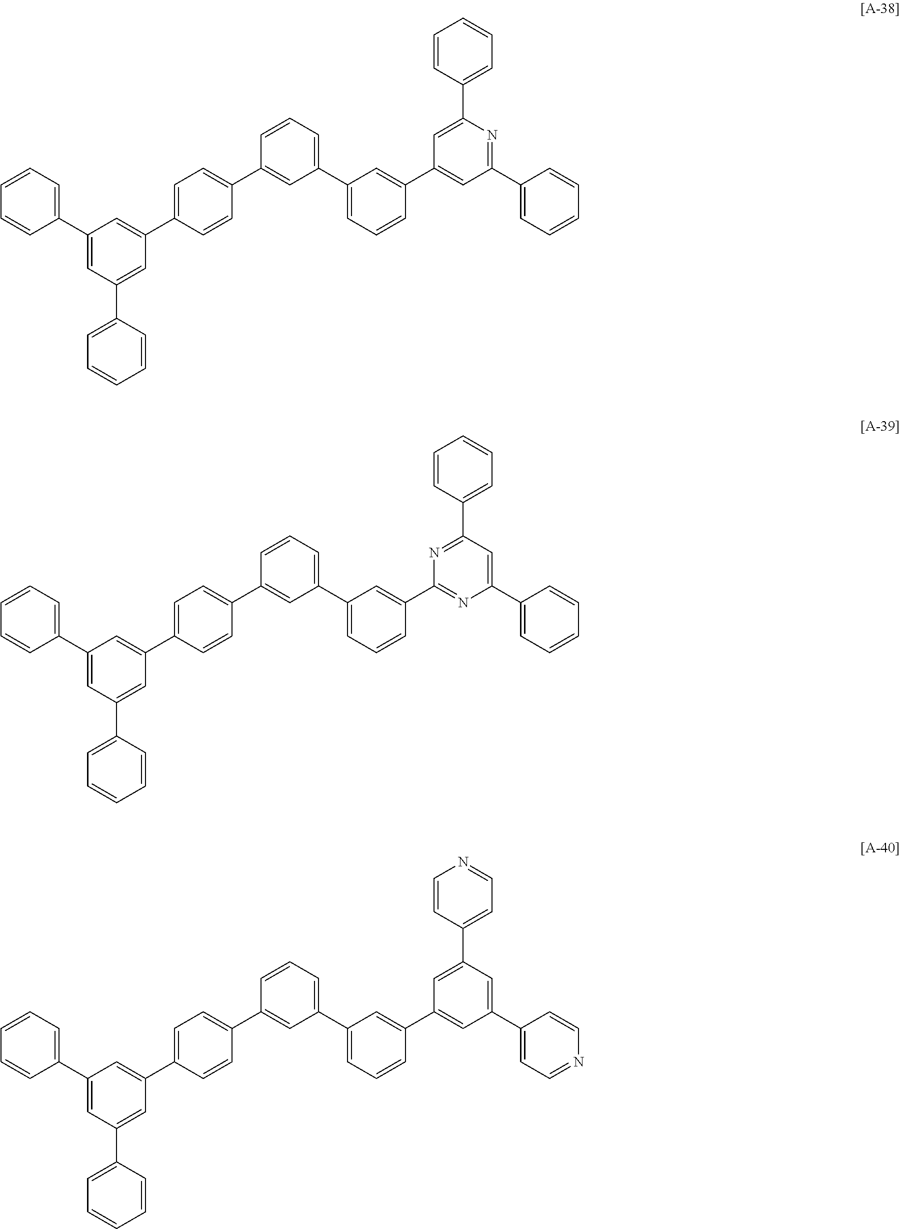 [SMILES: c1ccc(-c2cc(-c3ccccc3)cc(-c3ccc(-c4cccc(-c5cccc(-c6cc(-c7ccccc7)nc(-c7ccccc7)c6)c5)c4)cc3)c2)cc1 ; c1ccc(-c2cc(-c3ccccc3)cc(-c3ccc(-c4cccc(-c5cccc(-c6nc(-c7ccccc7)cc(-c7ccccc7)n6)c5)c4)cc3)c2)cc1 ; c1ccc(-c2cc(-c3ccccc3)cc(-c3ccc(-c4cccc(-c5cccc(-c6cc(-c7ccncc7)cc(-c7ccncc7)c6)c5)c4)cc3)c2)cc1]

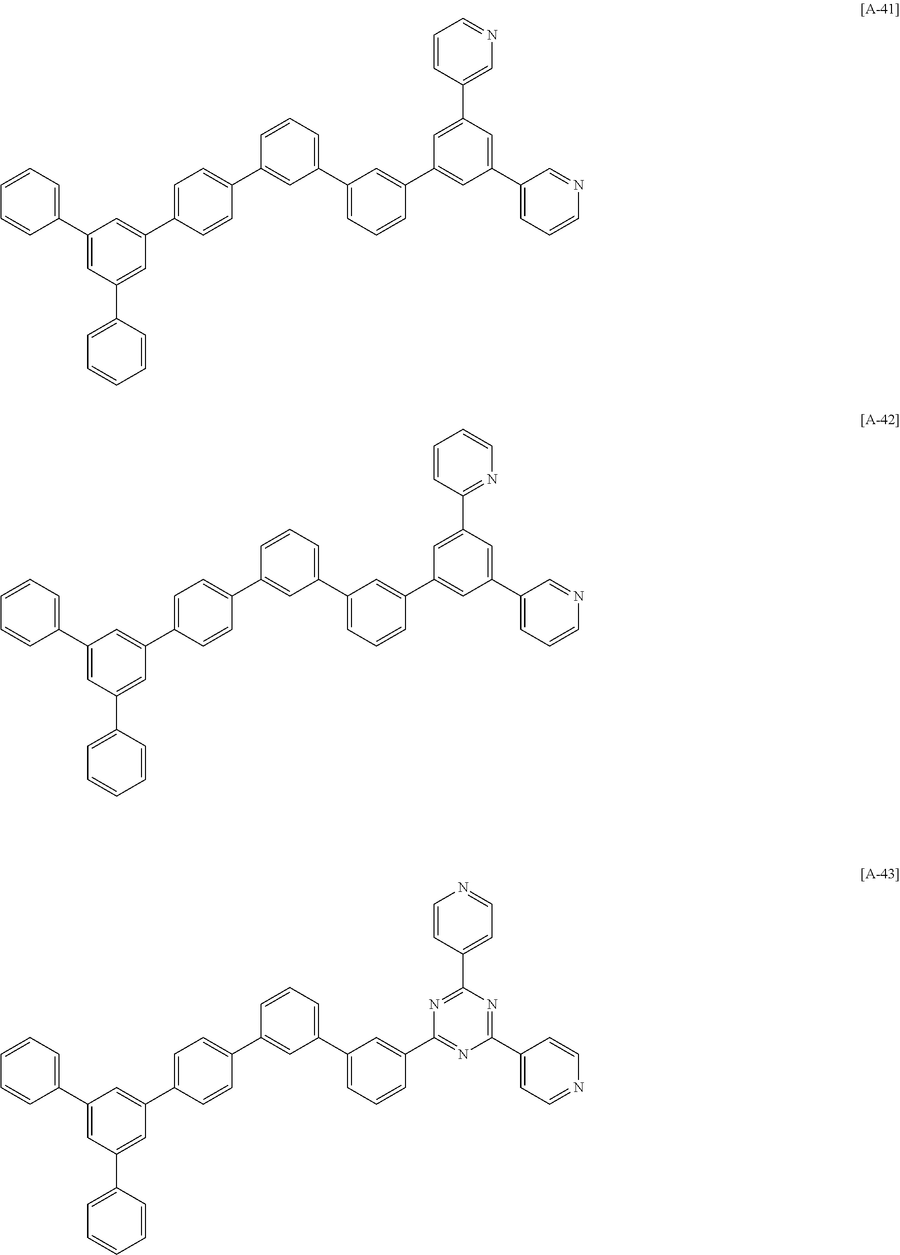 [SMILES: c1ccc(-c2cc(-c3ccccc3)cc(-c3ccc(-c4cccc(-c5cccc(-c6cc(-c7cccnc7)cc(-c7cccnc7)c6)c5)c4)cc3)c2)cc1 ; c1ccc(-c2cc(-c3ccccc3)cc(-c3ccc(-c4cccc(-c5cccc(-c6cc(-c7cccnc7)cc(-c7ccccn7)c6)c5)c4)cc3)c2)cc1 ; c1ccc(-c2cc(-c3ccccc3)cc(-c3ccc(-c4cccc(-c5cccc(-c6nc(-c7ccncc7)nc(-c7ccncc7)n6)c5)c4)cc3)c2)cc1]

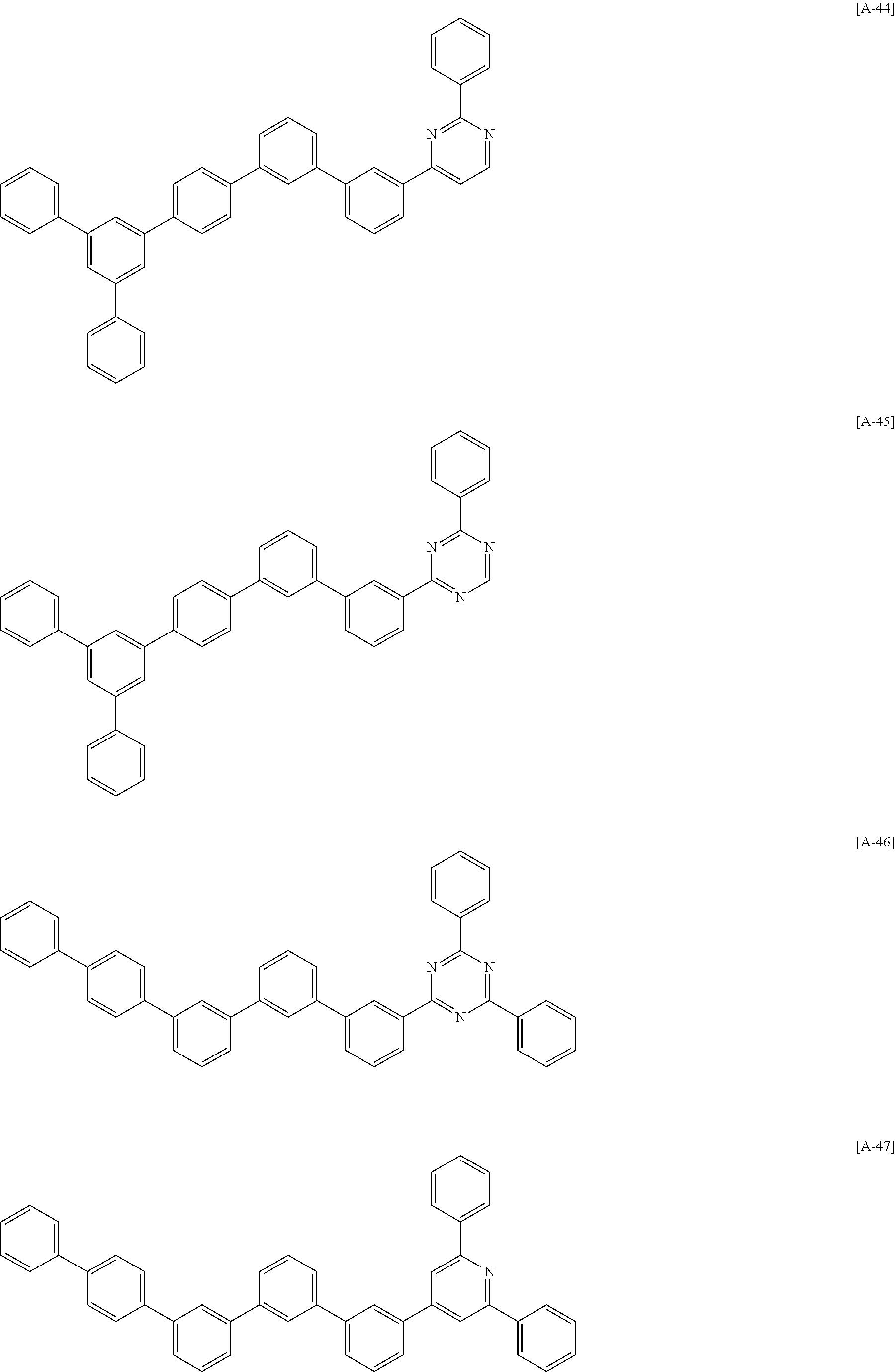 [SMILES: c1ccc(-c2cc(-c3ccccc3)cc(-c3ccc(-c4cccc(-c5cccc(-c6ccnc(-c7ccccc7)n6)c5)c4)cc3)c2)cc1 ; c1ccc(-c2cc(-c3ccccc3)cc(-c3ccc(-c4cccc(-c5cccc(-c6ncnc(-c7ccccc7)n6)c5)c4)cc3)c2)cc1 ; c1ccc(-c2ccc(-c3cccc(-c4cccc(-c5cccc(-c6nc(-c7ccccc7)nc(-c7ccccc7)n6)c5)c4)c3)cc2)cc1 ; c1ccc(-c2ccc(-c3cccc(-c4cccc(-c5cccc(-c6cc(-c7ccccc7)nc(-c7ccccc7)c6)c5)c4)c3)cc2)cc1]

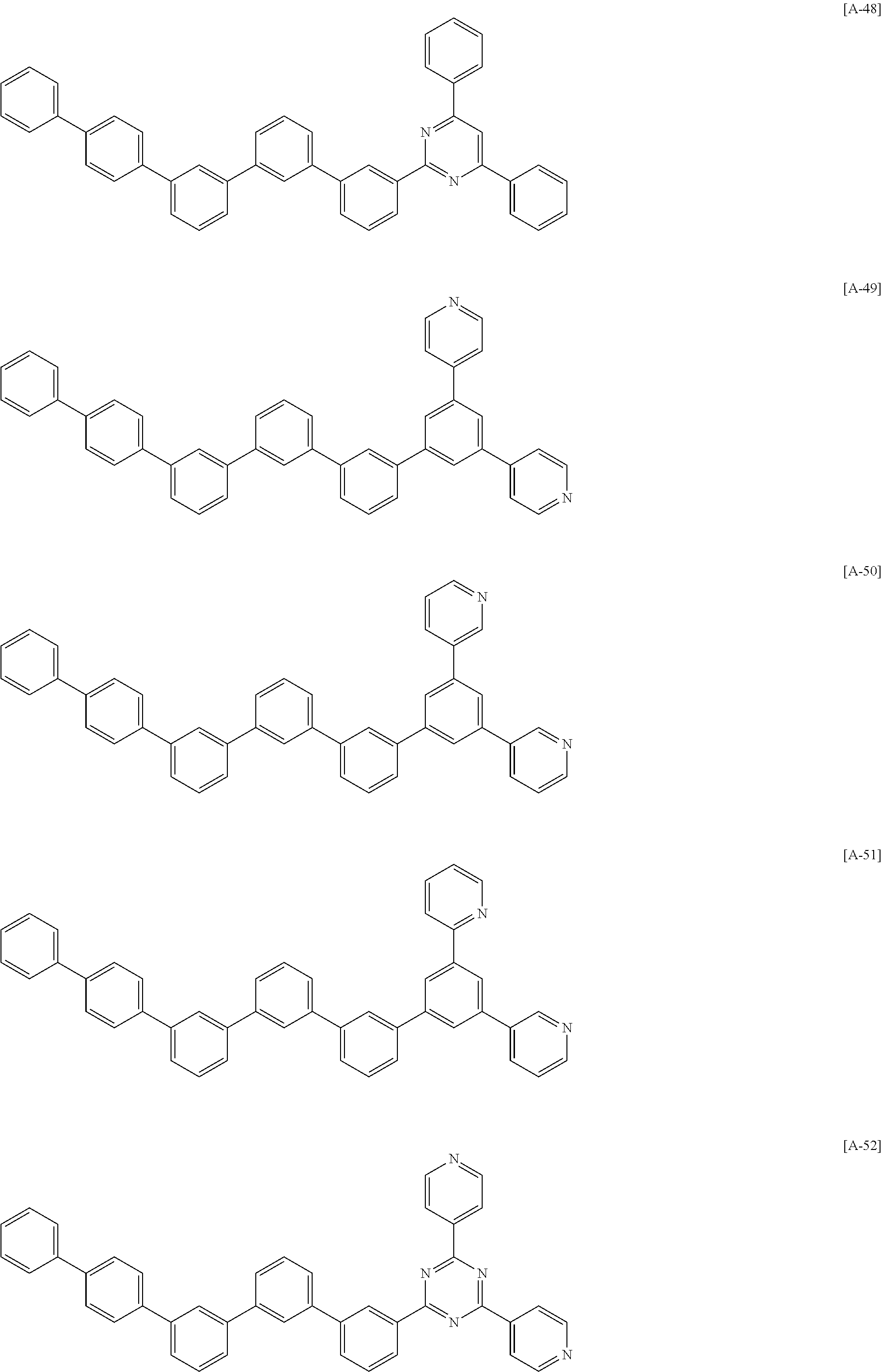 [SMILES: c1ccc(-c2ccc(-c3cccc(-c4cccc(-c5cccc(-c6nc(-c7ccccc7)cc(-c7ccccc7)n6)c5)c4)c3)cc2)cc1 ; c1ccc(-c2ccc(-c3cccc(-c4cccc(-c5cccc(-c6cc(-c7ccncc7)cc(-c7ccncc7)c6)c5)c4)c3)cc2)cc1 ; c1ccc(-c2ccc(-c3cccc(-c4cccc(-c5cccc(-c6cc(-c7cccnc7)cc(-c7cccnc7)c6)c5)c4)c3)cc2)cc1 ; c1ccc(-c2ccc(-c3cccc(-c4cccc(-c5cccc(-c6cc(-c7cccnc7)cc(-c7ccccn7)c6)c5)c4)c3)cc2)cc1 ; c1ccc(-c2ccc(-c3cccc(-c4cccc(-c5cccc(-c6nc(-c7ccncc7)nc(-c7ccncc7)n6)c5)c4)c3)cc2)cc1]

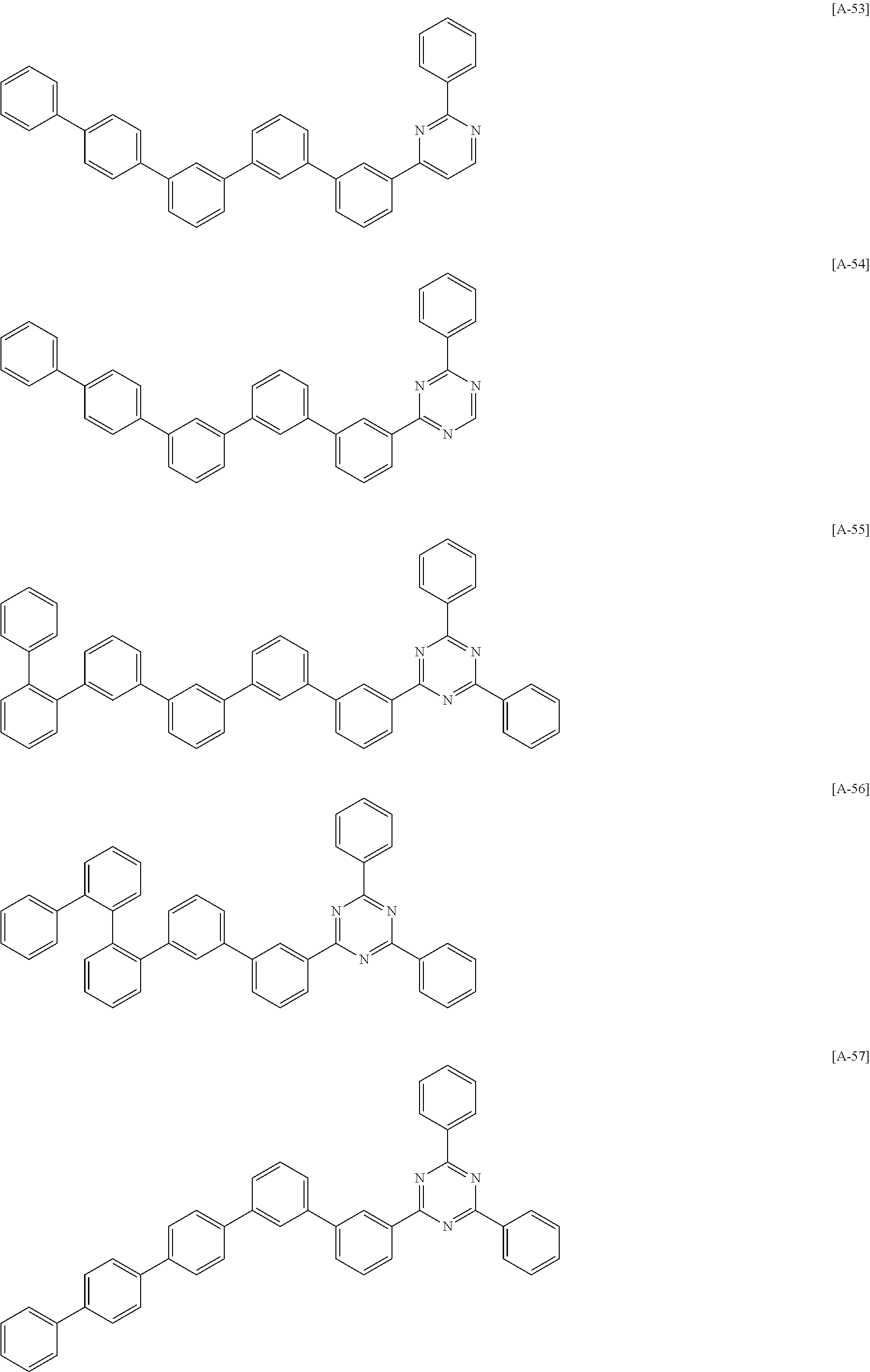 [SMILES: c1ccc(-c2ccc(-c3cccc(-c4cccc(-c5cccc(-c6ccnc(-c7ccccc7)n6)c5)c4)c3)cc2)cc1 ; c1ccc(-c2ccc(-c3cccc(-c4cccc(-c5cccc(-c6ncnc(-c7ccccc7)n6)c5)c4)c3)cc2)cc1 ; c1ccc(-c2nc(-c3ccccc3)nc(-c3cccc(-c4cccc(-c5cccc(-c6cccc(-c7ccccc7-c7ccccc7)c6)c5)c4)c3)n2)cc1 ; c1ccc(-c2nc(-c3ccccc3)nc(-c3cccc(-c4cccc(-c5ccccc5-c5ccccc5-c5ccccc5)c4)c3)n2)cc1 ; c1ccc(-c2ccc(-c3ccc(-c4cccc(-c5cccc(-c6nc(-c7ccccc7)nc(-c7ccccc7)n6)c5)c4)cc3)cc2)cc1]

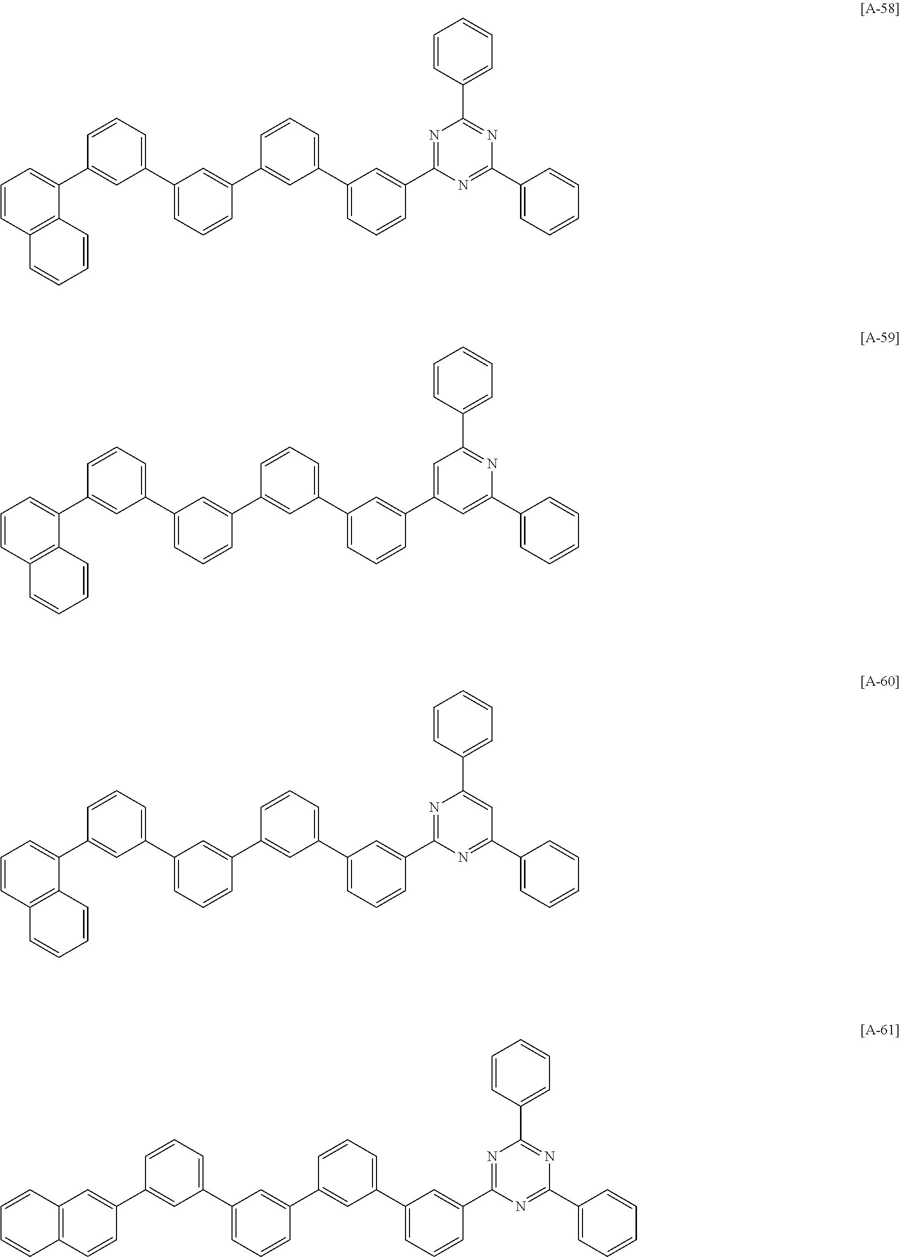 [SMILES: c1ccc(-c2nc(-c3ccccc3)nc(-c3cccc(-c4cccc(-c5cccc(-c6cccc(-c7cccc8ccccc78)c6)c5)c4)c3)n2)cc1 ; c1ccc(-c2cc(-c3cccc(-c4cccc(-c5cccc(-c6cccc(-c7cccc8ccccc78)c6)c5)c4)c3)cc(-c3ccccc3)n2)cc1 ; c1ccc(-c2cc(-c3ccccc3)nc(-c3cccc(-c4cccc(-c5cccc(-c6cccc(-c7cccc8ccccc78)c6)c5)c4)c3)n2)cc1 ; c1ccc(-c2nc(-c3ccccc3)nc(-c3cccc(-c4cccc(-c5cccc(-c6cccc(-c7ccc8ccccc8c7)c6)c5)c4)c3)n2)cc1]

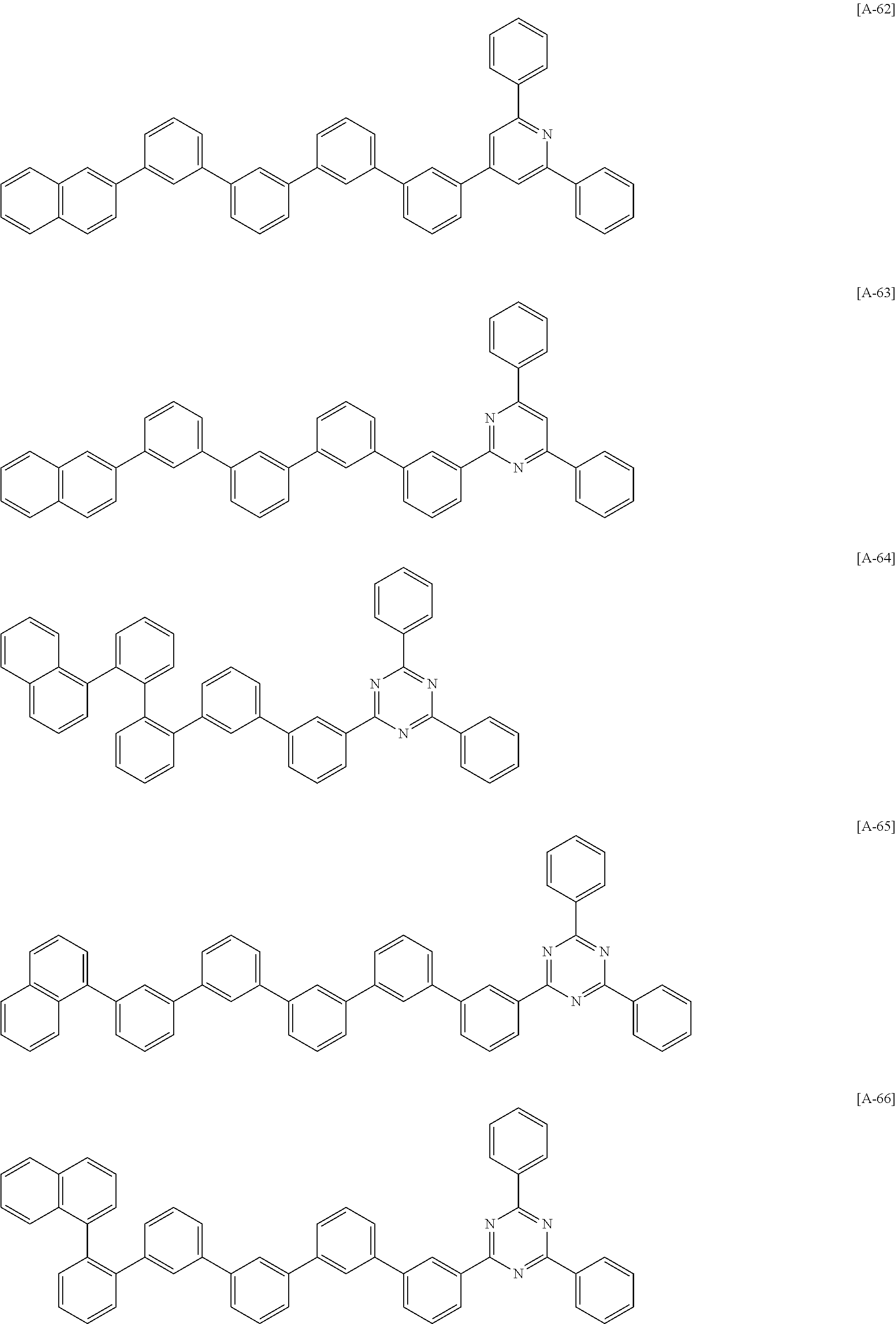 [SMILES: c1ccc(-c2cc(-c3cccc(-c4cccc(-c5cccc(-c6cccc(-c7ccc8ccccc8c7)c6)c5)c4)c3)cc(-c3ccccc3)n2)cc1 ; c1ccc(-c2cc(-c3ccccc3)nc(-c3cccc(-c4cccc(-c5cccc(-c6cccc(-c7ccc8ccccc8c7)c6)c5)c4)c3)n2)cc1 ; c1ccc(-c2nc(-c3ccccc3)nc(-c3cccc(-c4cccc(-c5ccccc5-c5ccccc5-c5cccc6ccccc56)c4)c3)n2)cc1 ; c1ccc(-c2nc(-c3ccccc3)nc(-c3cccc(-c4cccc(-c5cccc(-c6cccc(-c7cccc(-c8cccc9ccccc89)c7)c6)c5)c4)c3)n2)cc1 ; c1ccc(-c2nc(-c3ccccc3)nc(-c3cccc(-c4cccc(-c5cccc(-c6cccc(-c7ccccc7-c7cccc8ccccc78)c6)c5)c4)c3)n2)cc1]

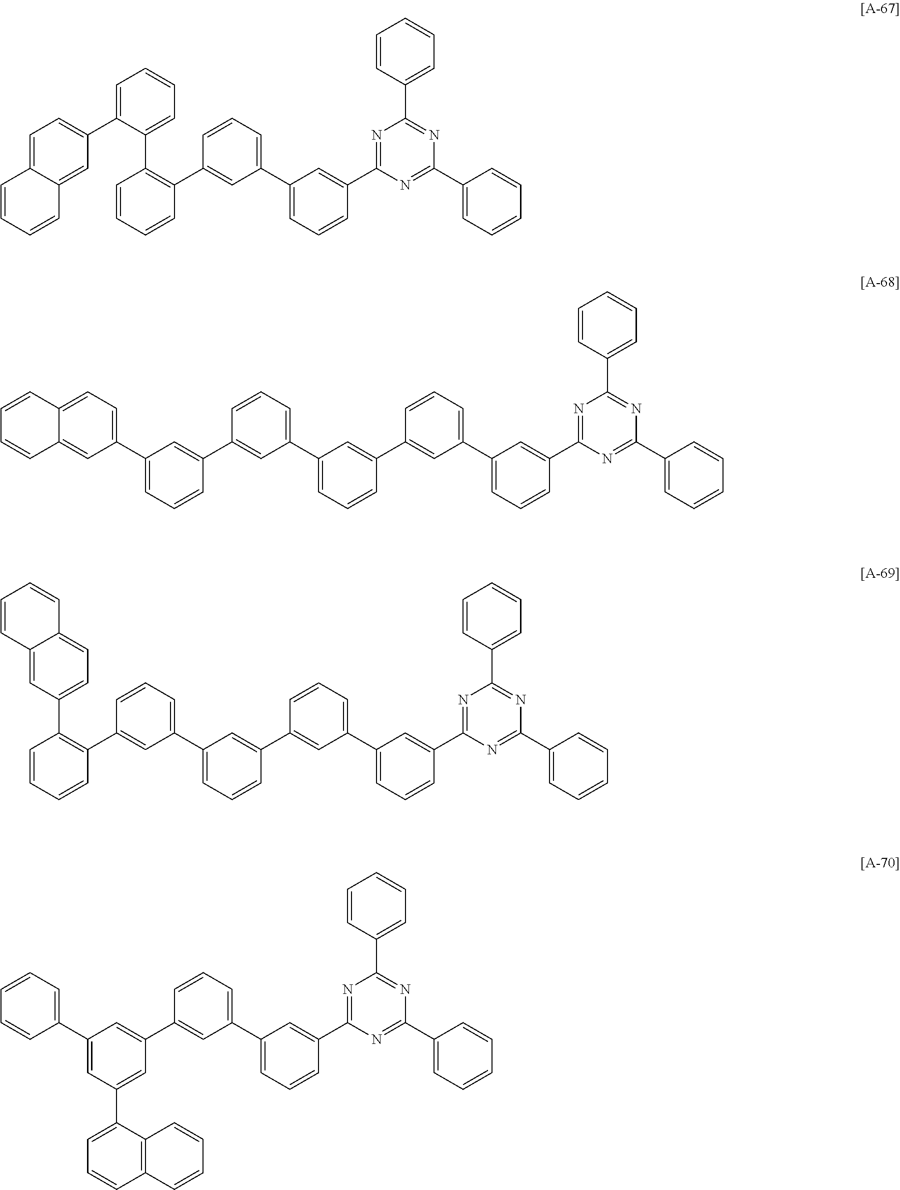 [SMILES: c1ccc(-c2nc(-c3ccccc3)nc(-c3cccc(-c4cccc(-c5ccccc5-c5ccccc5-c5ccc6ccccc6c5)c4)c3)n2)cc1 ; c1ccc(-c2nc(-c3ccccc3)nc(-c3cccc(-c4cccc(-c5cccc(-c6cccc(-c7cccc(-c8ccc9ccccc9c8)c7)c6)c5)c4)c3)n2)cc1 ; c1ccc(-c2nc(-c3ccccc3)nc(-c3cccc(-c4cccc(-c5cccc(-c6cccc(-c7ccccc7-c7ccc8ccccc8c7)c6)c5)c4)c3)n2)cc1 ; c1ccc(-c2cc(-c3cccc(-c4cccc(-c5nc(-c6ccccc6)nc(-c6ccccc6)n5)c4)c3)cc(-c3cccc4ccccc34)c2)cc1]

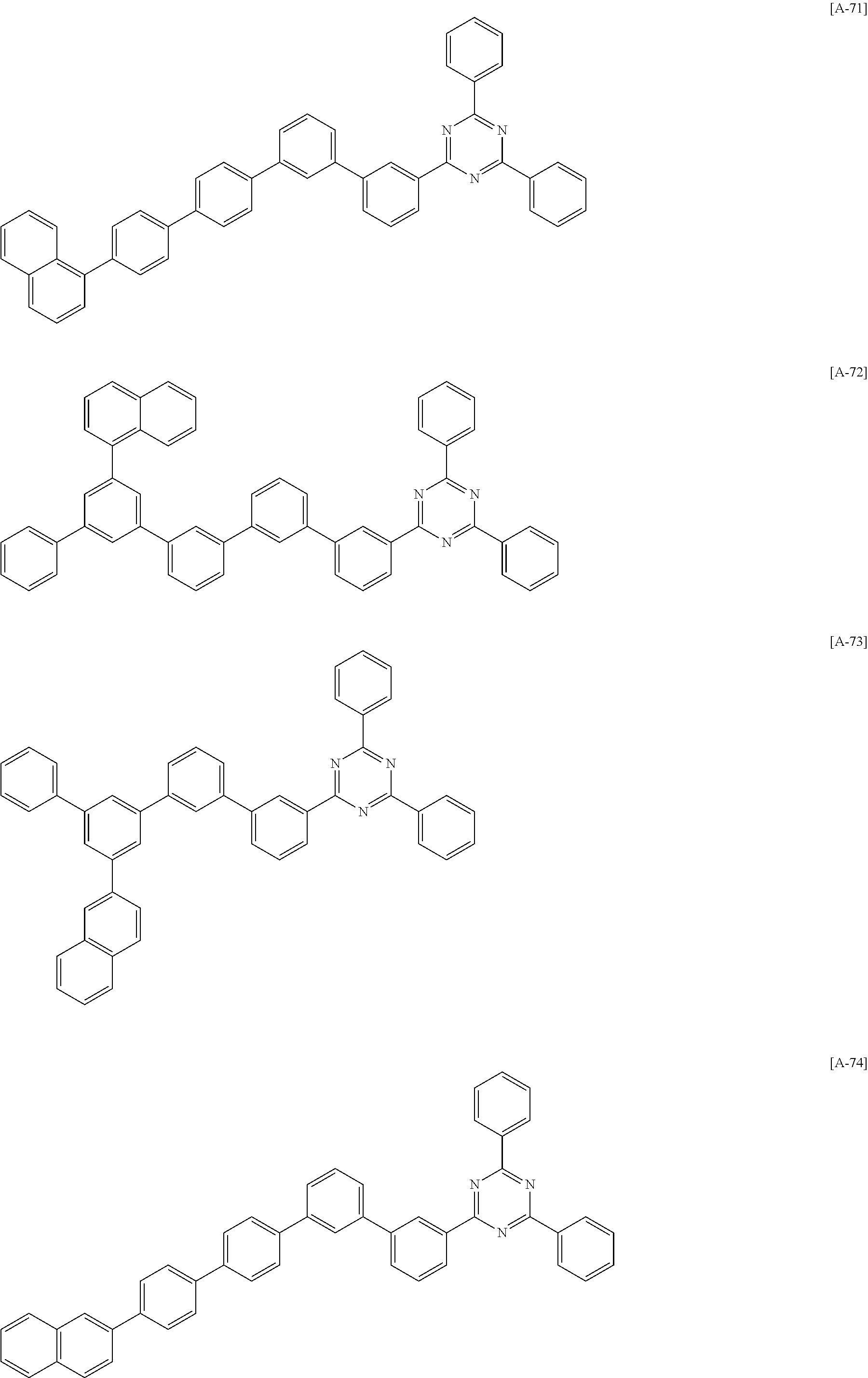 [SMILES: c1ccc(-c2nc(-c3ccccc3)nc(-c3cccc(-c4cccc(-c5ccc(-c6ccc(-c7cccc8ccccc78)cc6)cc5)c4)c3)n2)cc1 ; c1ccc(-c2cc(-c3cccc(-c4cccc(-c5cccc(-c6nc(-c7ccccc7)nc(-c7ccccc7)n6)c5)c4)c3)cc(-c3cccc4ccccc34)c2)cc1 ; c1ccc(-c2cc(-c3cccc(-c4cccc(-c5nc(-c6ccccc6)nc(-c6ccccc6)n5)c4)c3)cc(-c3ccc4ccccc4c3)c2)cc1 ; c1ccc(-c2nc(-c3ccccc3)nc(-c3cccc(-c4cccc(-c5ccc(-c6ccc(-c7ccc8ccccc8c7)cc6)cc5)c4)c3)n2)cc1]

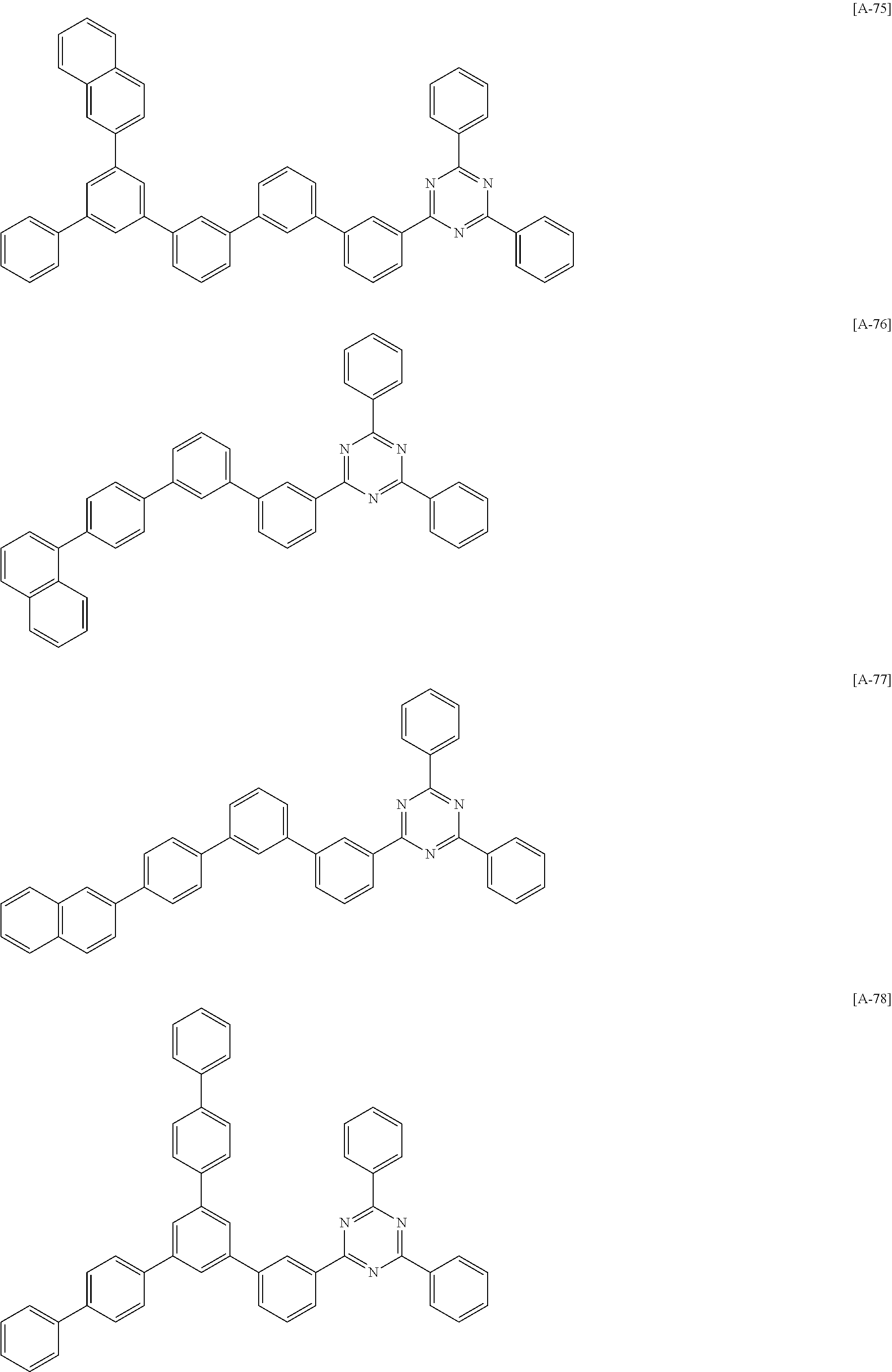 [SMILES: c1ccc(-c2cc(-c3cccc(-c4cccc(-c5cccc(-c6nc(-c7ccccc7)nc(-c7ccccc7)n6)c5)c4)c3)cc(-c3ccc4ccccc4c3)c2)cc1 ; c1ccc(-c2nc(-c3ccccc3)nc(-c3cccc(-c4cccc(-c5ccc(-c6cccc7ccccc67)cc5)c4)c3)n2)cc1 ; c1ccc(-c2nc(-c3ccccc3)nc(-c3cccc(-c4cccc(-c5ccc(-c6ccc7ccccc7c6)cc5)c4)c3)n2)cc1 ; c1ccc(-c2ccc(-c3cc(-c4ccc(-c5ccccc5)cc4)cc(-c4cccc(-c5nc(-c6ccccc6)nc(-c6ccccc6)n5)c4)c3)cc2)cc1]

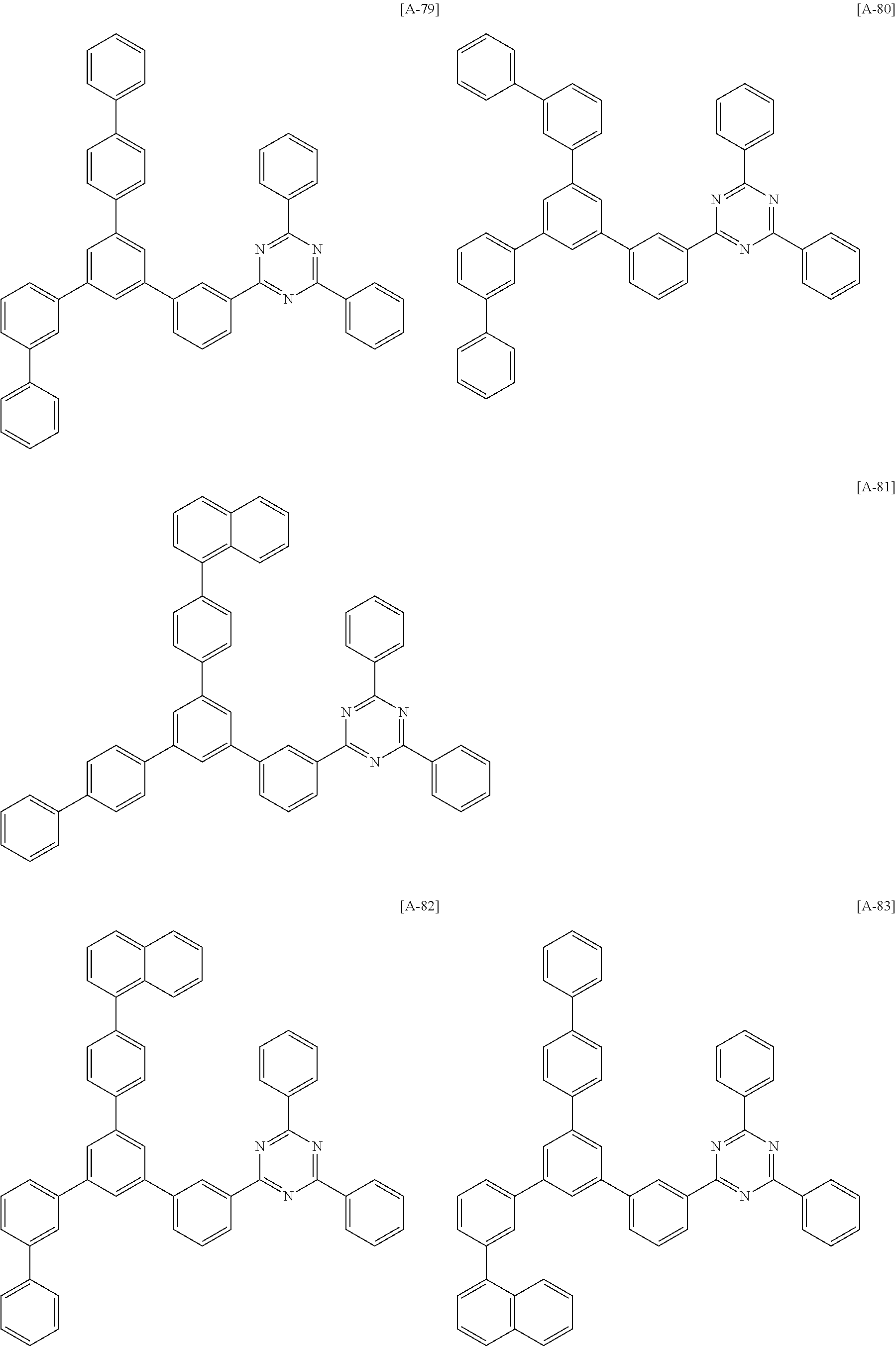

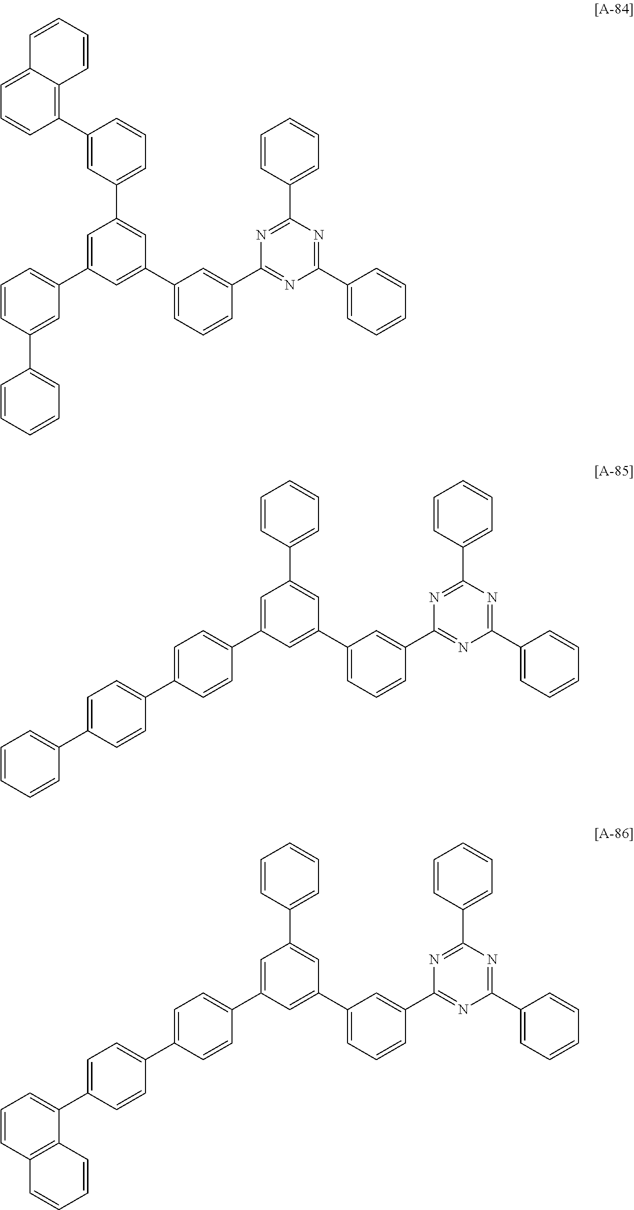 [SMILES: c1ccc(-c2cccc(-c3cc(-c4cccc(-c5nc(-c6ccccc6)nc(-c6ccccc6)n5)c4)cc(-c4cccc(-c5cccc6ccccc56)c4)c3)c2)cc1 ; c1ccc(-c2ccc(-c3ccc(-c4cc(-c5ccccc5)cc(-c5cccc(-c6nc(-c7ccccc7)nc(-c7ccccc7)n6)c5)c4)cc3)cc2)cc1 ; c1ccc(-c2cc(-c3ccc(-c4ccc(-c5cccc6ccccc56)cc4)cc3)cc(-c3cccc(-c4nc(-c5ccccc5)nc(-c5ccccc5)n4)c3)c2)cc1]

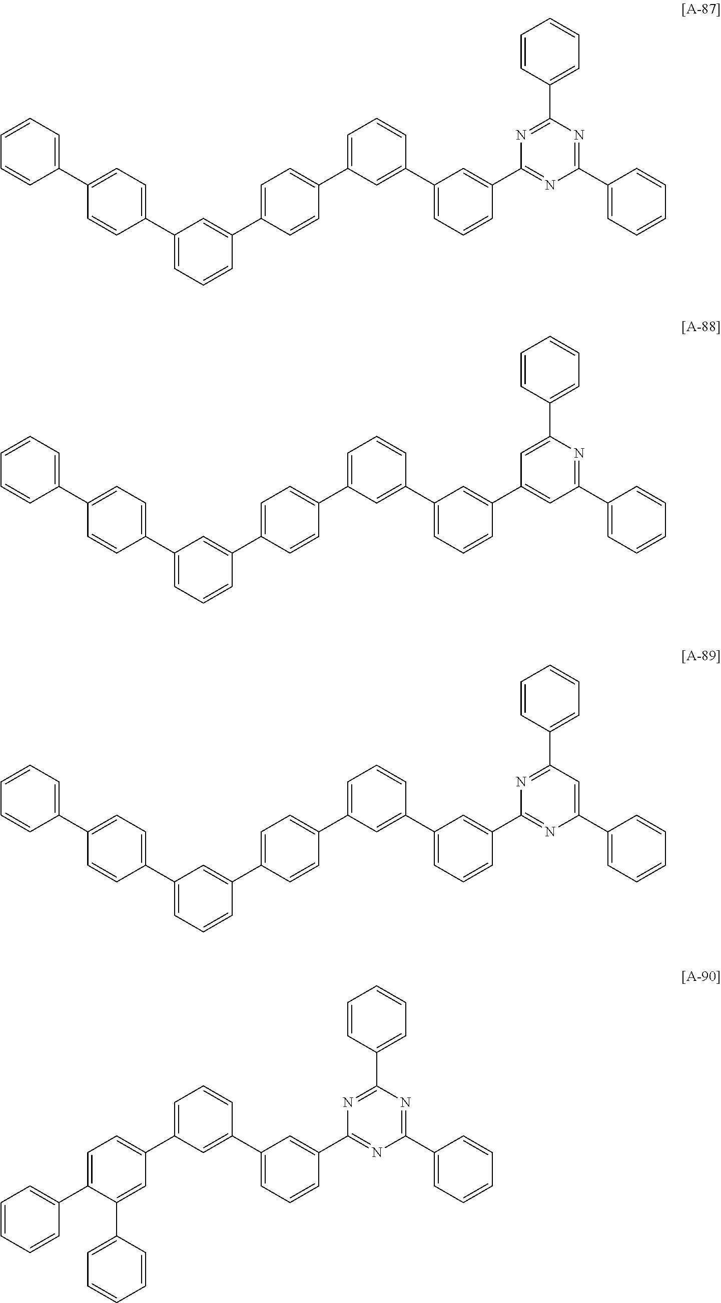 [SMILES: c1ccc(-c2ccc(-c3cccc(-c4ccc(-c5cccc(-c6cccc(-c7nc(-c8ccccc8)nc(-c8ccccc8)n7)c6)c5)cc4)c3)cc2)cc1 ; c1ccc(-c2ccc(-c3cccc(-c4ccc(-c5cccc(-c6cccc(-c7cc(-c8ccccc8)nc(-c8ccccc8)c7)c6)c5)cc4)c3)cc2)cc1 ; c1ccc(-c2ccc(-c3cccc(-c4ccc(-c5cccc(-c6cccc(-c7nc(-c8ccccc8)cc(-c8ccccc8)n7)c6)c5)cc4)c3)cc2)cc1 ; c1ccc(-c2nc(-c3ccccc3)nc(-c3cccc(-c4cccc(-c5ccc(-c6ccccc6)c(-c6ccccc6)c5)c4)c3)n2)cc1]

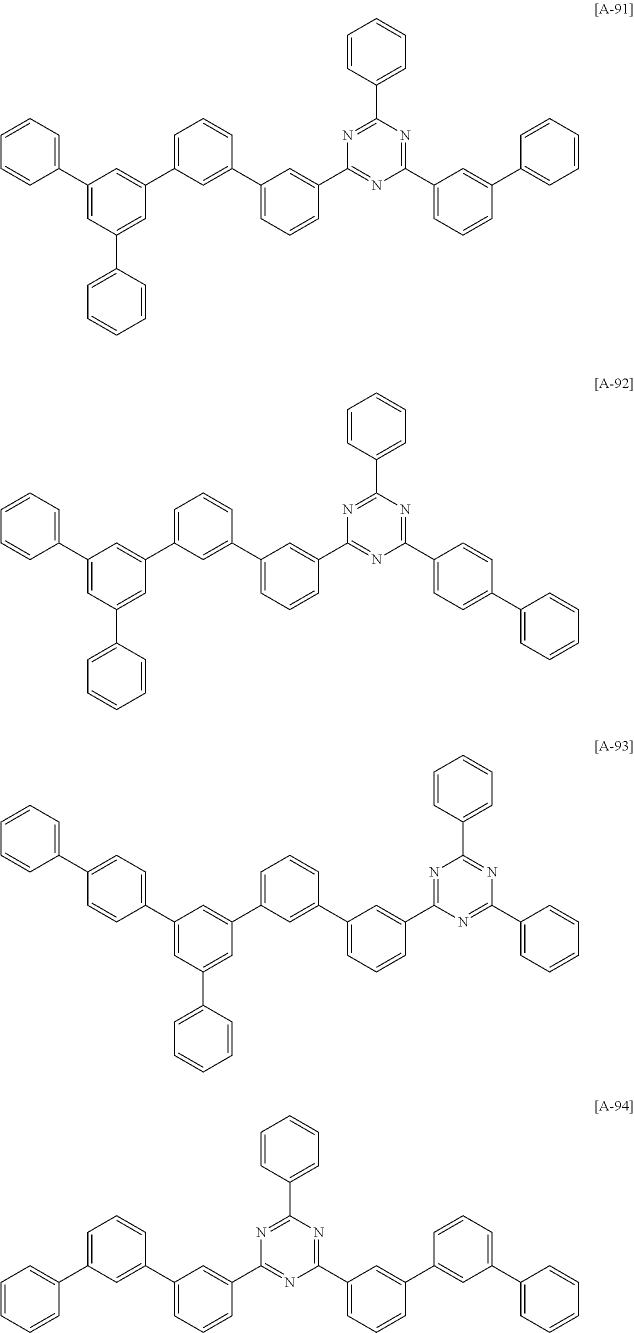 [SMILES: c1ccc(-c2cc(-c3ccccc3)cc(-c3cccc(-c4cccc(-c5nc(-c6ccccc6)nc(-c6cccc(-c7ccccc7)c6)n5)c4)c3)c2)cc1 ; c1ccc(-c2ccc(-c3nc(-c4ccccc4)nc(-c4cccc(-c5cccc(-c6cc(-c7ccccc7)cc(-c7ccccc7)c6)c5)c4)n3)cc2)cc1 ; c1ccc(-c2ccc(-c3cc(-c4ccccc4)cc(-c4cccc(-c5cccc(-c6nc(-c7ccccc7)nc(-c7ccccc7)n6)c5)c4)c3)cc2)cc1 ; c1ccc(-c2cccc(-c3cccc(-c4nc(-c5ccccc5)nc(-c5cccc(-c6cccc(-c7ccccc7)c6)c5)n4)c3)c2)cc1]

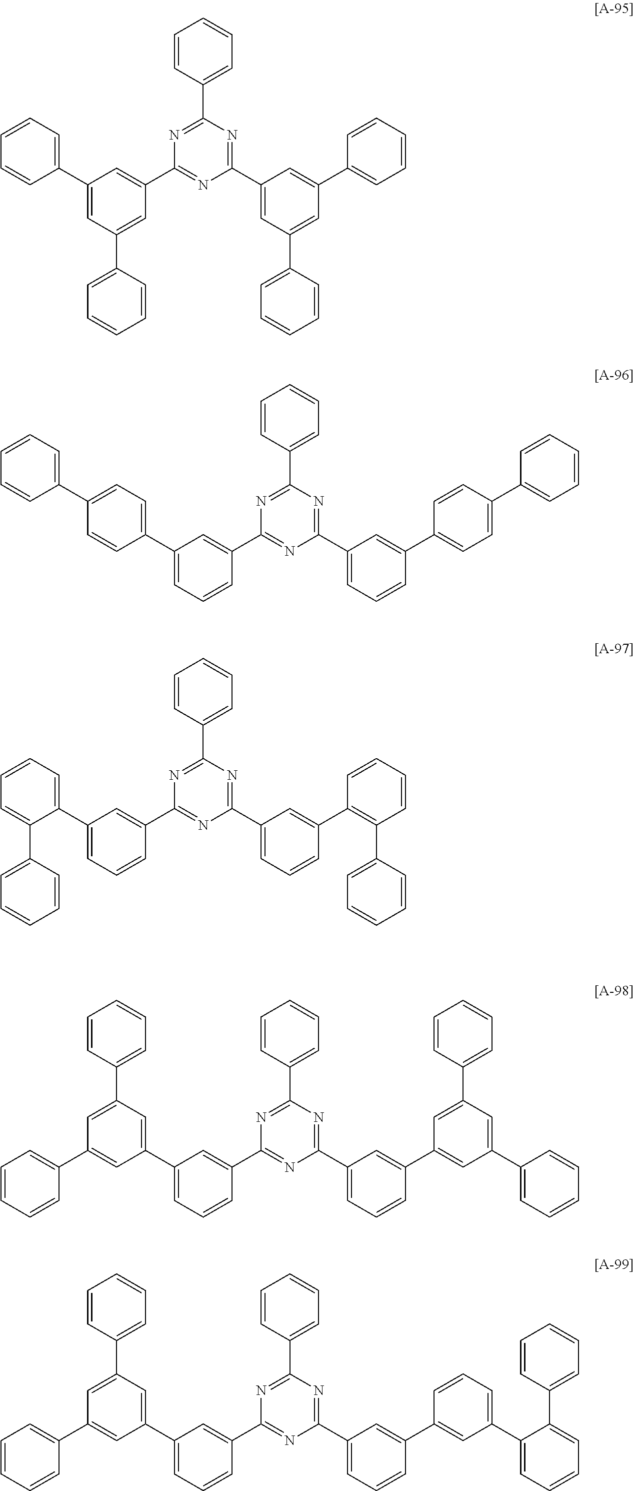 [SMILES: c1ccc(-c2cc(-c3ccccc3)cc(-c3nc(-c4ccccc4)nc(-c4cc(-c5ccccc5)cc(-c5ccccc5)c4)n3)c2)cc1 ; c1ccc(-c2ccc(-c3cccc(-c4nc(-c5ccccc5)nc(-c5cccc(-c6ccc(-c7ccccc7)cc6)c5)n4)c3)cc2)cc1 ; c1ccc(-c2nc(-c3cccc(-c4ccccc4-c4ccccc4)c3)nc(-c3cccc(-c4ccccc4-c4ccccc4)c3)n2)cc1 ; c1ccc(-c2cc(-c3ccccc3)cc(-c3cccc(-c4nc(-c5ccccc5)nc(-c5cccc(-c6cc(-c7ccccc7)cc(-c7ccccc7)c6)c5)n4)c3)c2)cc1 ; c1ccc(-c2cc(-c3ccccc3)cc(-c3cccc(-c4nc(-c5ccccc5)nc(-c5cccc(-c6cccc(-c7ccccc7-c7ccccc7)c6)c5)n4)c3)c2)cc1]

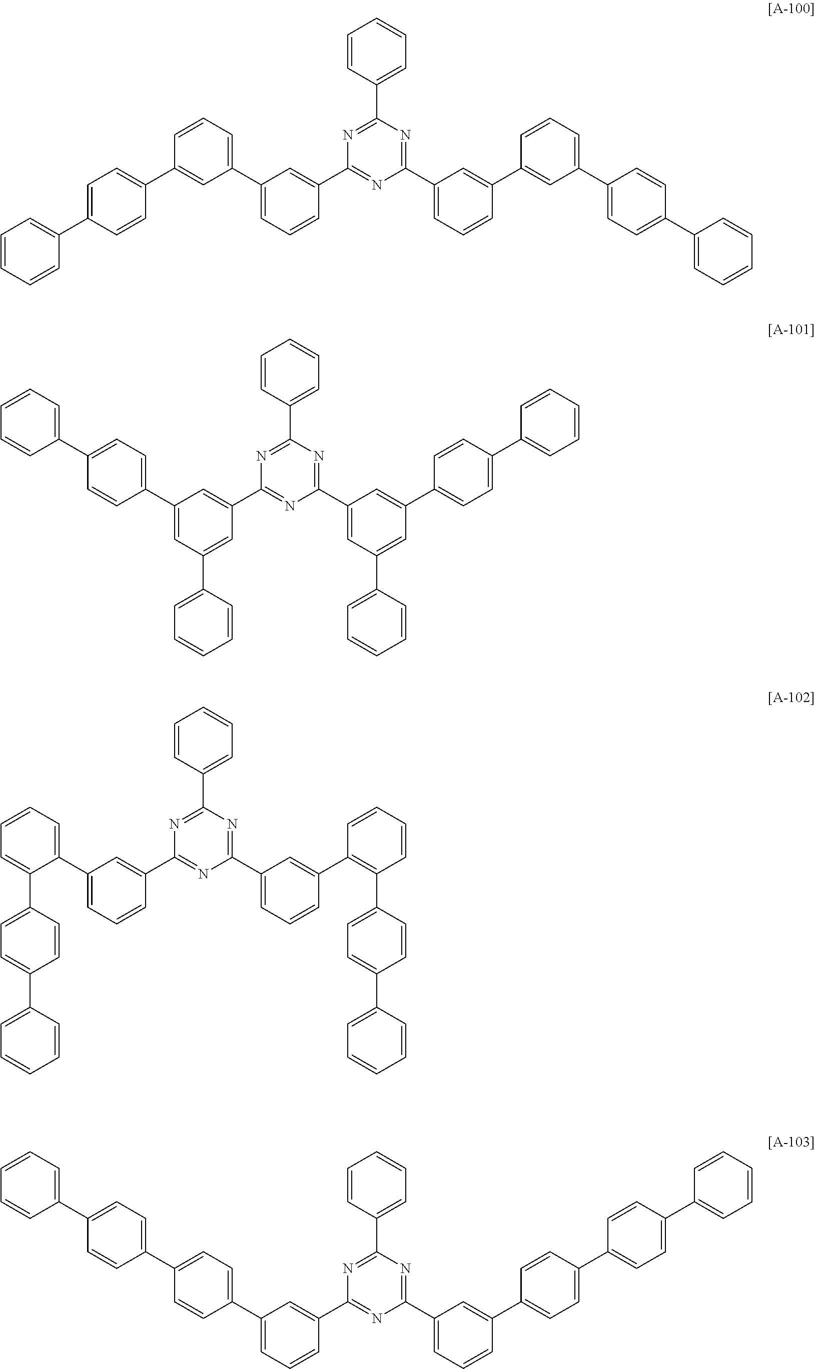 [SMILES: c1ccc(-c2ccc(-c3cccc(-c4cccc(-c5nc(-c6ccccc6)nc(-c6cccc(-c7cccc(-c8ccc(-c9ccccc9)cc8)c7)c6)n5)c4)c3)cc2)cc1 ; c1ccc(-c2ccc(-c3cc(-c4ccccc4)cc(-c4nc(-c5ccccc5)nc(-c5cc(-c6ccccc6)cc(-c6ccc(-c7ccccc7)cc6)c5)n4)c3)cc2)cc1 ; c1ccc(-c2ccc(-c3ccccc3-c3cccc(-c4nc(-c5ccccc5)nc(-c5cccc(-c6ccccc6-c6ccc(-c7ccccc7)cc6)c5)n4)c3)cc2)cc1 ; c1ccc(-c2ccc(-c3ccc(-c4cccc(-c5nc(-c6ccccc6)nc(-c6cccc(-c7ccc(-c8ccc(-c9ccccc9)cc8)cc7)c6)n5)c4)cc3)cc2)cc1]

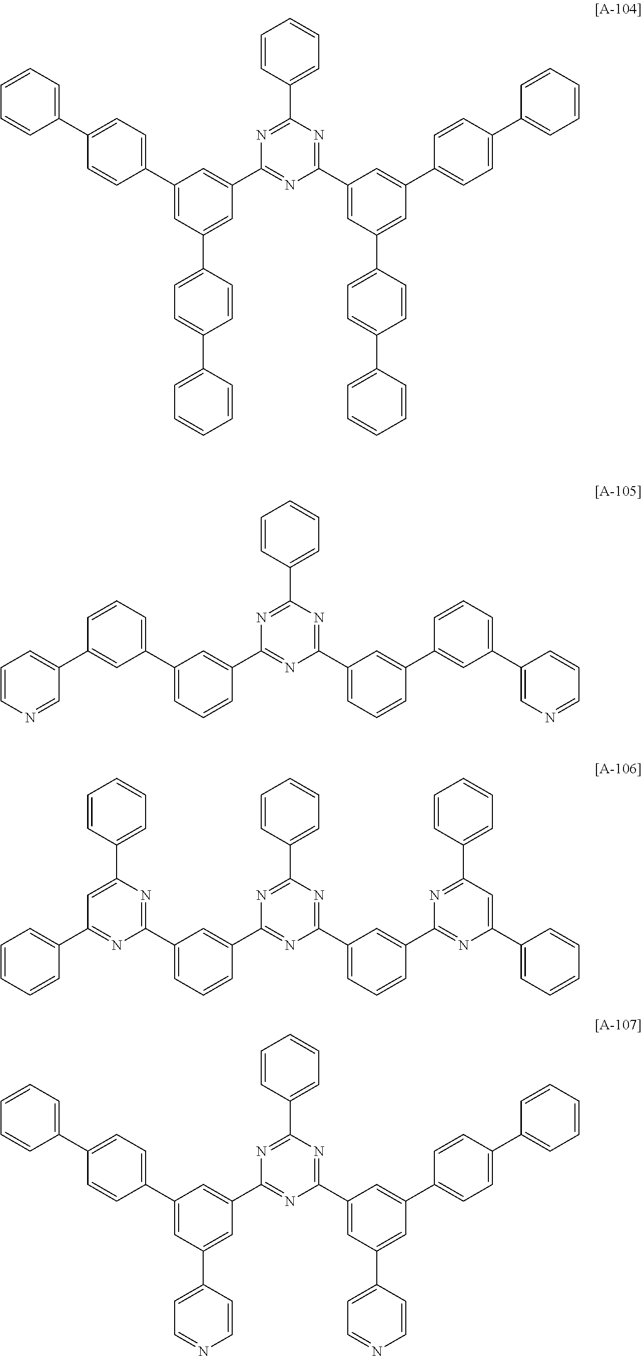 [SMILES: c1ccc(-c2ccc(-c3cc(-c4ccc(-c5ccccc5)cc4)cc(-c4nc(-c5ccccc5)nc(-c5cc(-c6ccc(-c7ccccc7)cc6)cc(-c6ccc(-c7ccccc7)cc6)c5)n4)c3)cc2)cc1 ; c1ccc(-c2nc(-c3cccc(-c4cccc(-c5cccnc5)c4)c3)nc(-c3cccc(-c4cccc(-c5cccnc5)c4)c3)n2)cc1 ; c1ccc(-c2cc(-c3ccccc3)nc(-c3cccc(-c4nc(-c5ccccc5)nc(-c5cccc(-c6nc(-c7ccccc7)cc(-c7ccccc7)n6)c5)n4)c3)n2)cc1 ; c1ccc(-c2ccc(-c3cc(-c4ccncc4)cc(-c4nc(-c5ccccc5)nc(-c5cc(-c6ccncc6)cc(-c6ccc(-c7ccccc7)cc6)c5)n4)c3)cc2)cc1]

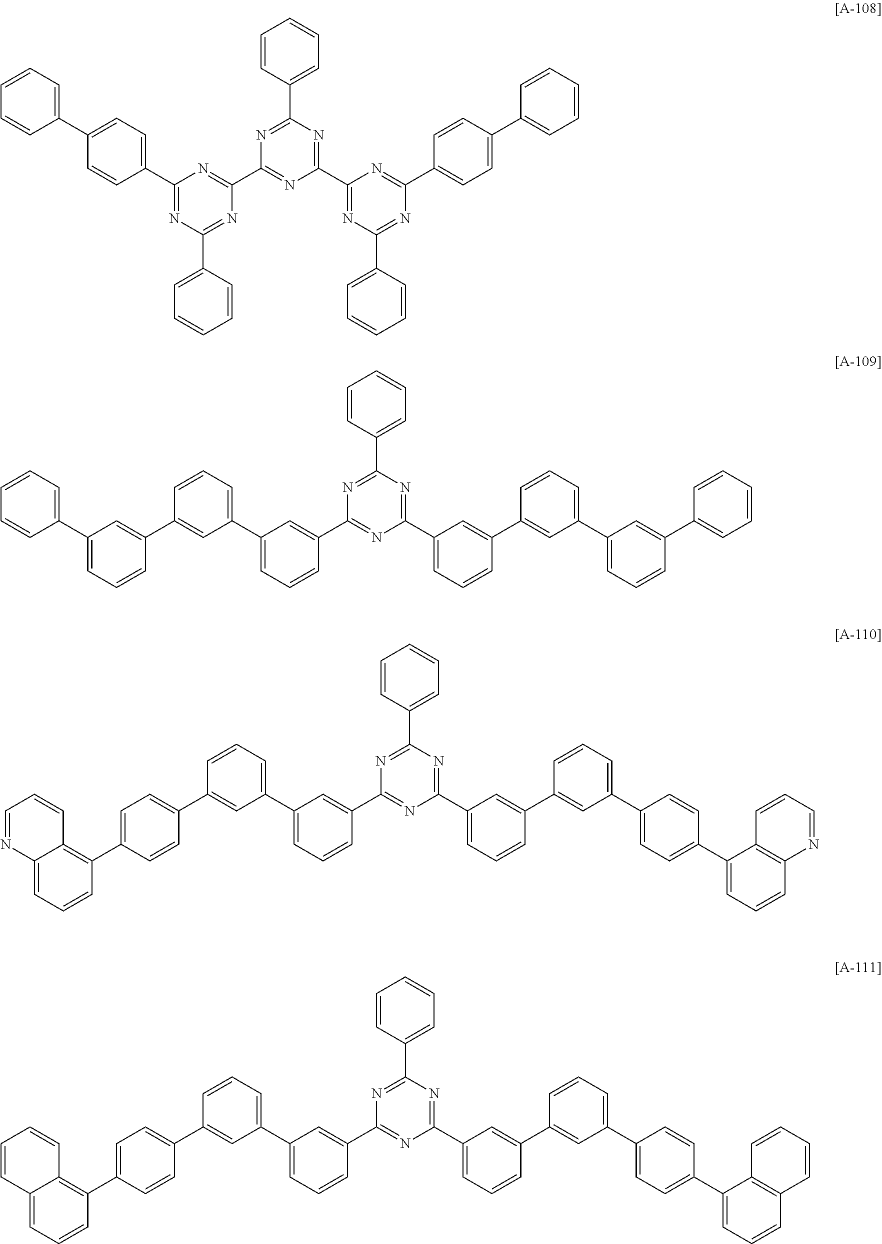

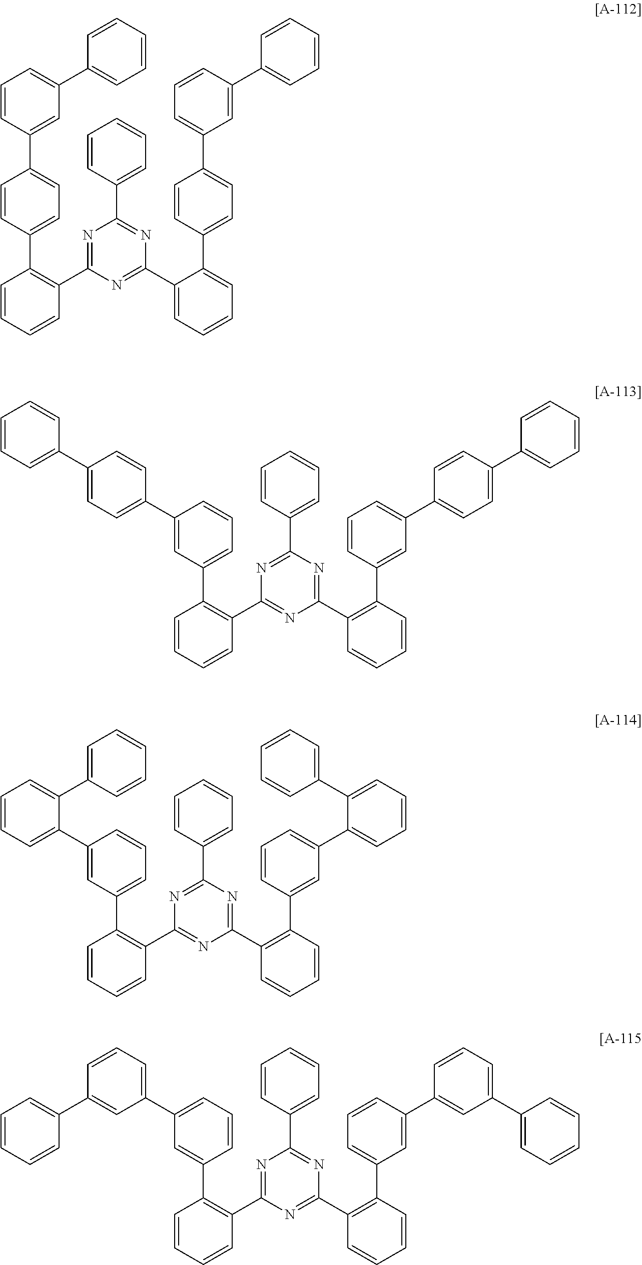 [SMILES: c1ccc(-c2cccc(-c3ccc(-c4ccccc4-c4nc(-c5ccccc5)nc(-c5ccccc5-c5ccc(-c6cccc(-c7ccccc7)c6)cc5)n4)cc3)c2)cc1 ; c1ccc(-c2ccc(-c3cccc(-c4ccccc4-c4nc(-c5ccccc5)nc(-c5ccccc5-c5cccc(-c6ccc(-c7ccccc7)cc6)c5)n4)c3)cc2)cc1 ; c1ccc(-c2nc(-c3ccccc3-c3cccc(-c4ccccc4-c4ccccc4)c3)nc(-c3ccccc3-c3cccc(-c4ccccc4-c4ccccc4)c3)n2)cc1 ; c1ccc(-c2cccc(-c3cccc(-c4ccccc4-c4nc(-c5ccccc5)nc(-c5ccccc5-c5cccc(-c6cccc(-c7ccccc7)c6)c5)n4)c3)c2)cc1]

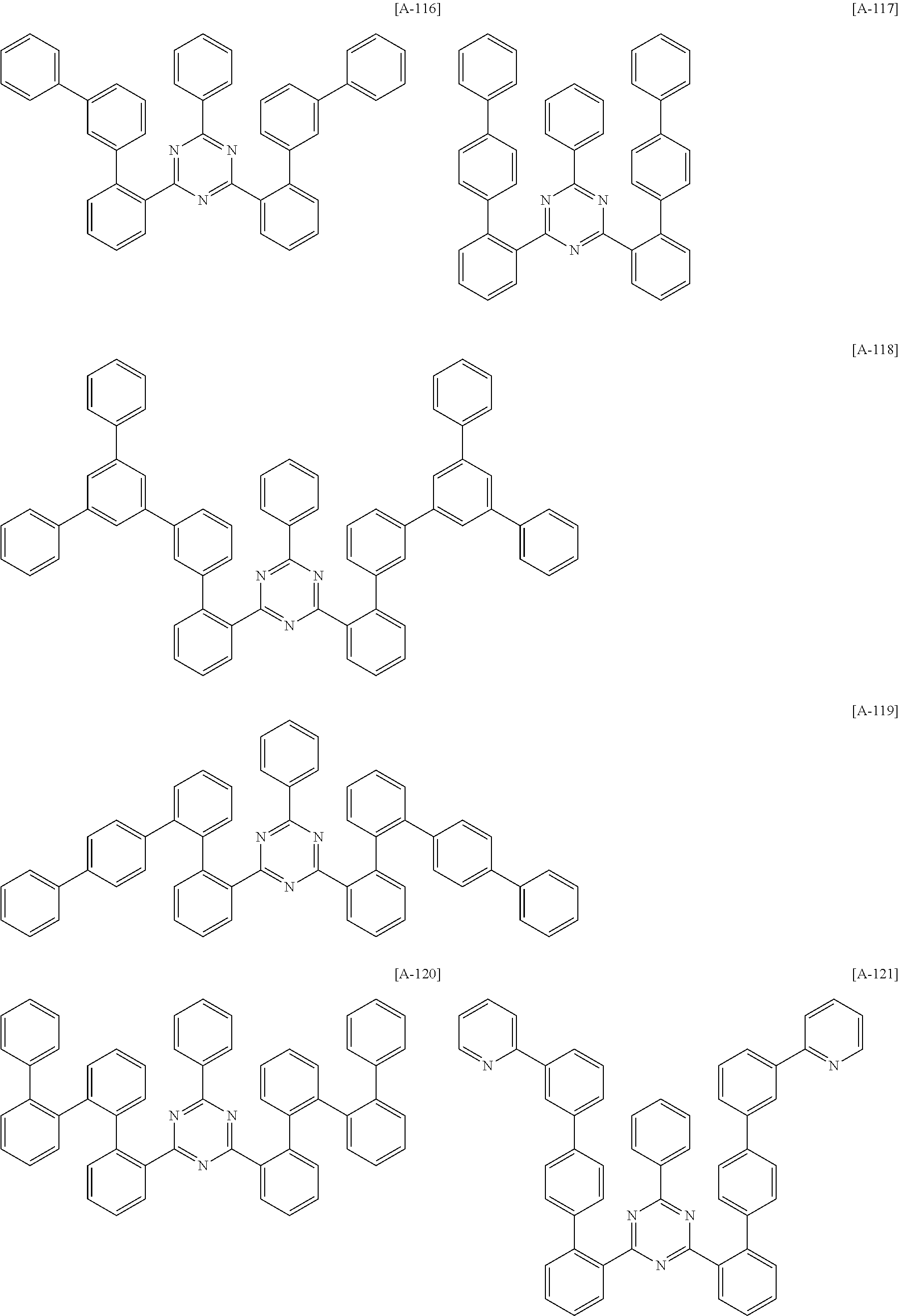

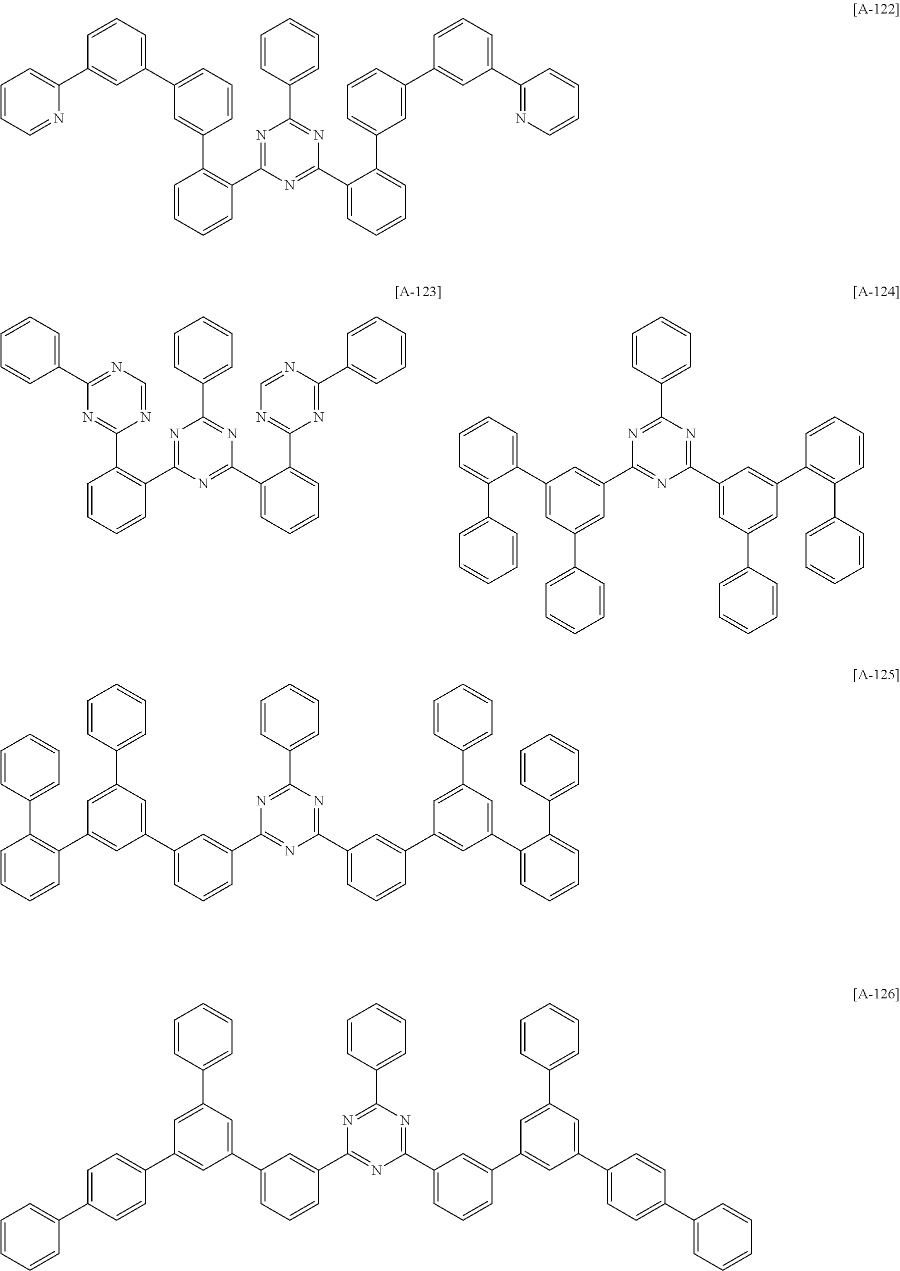

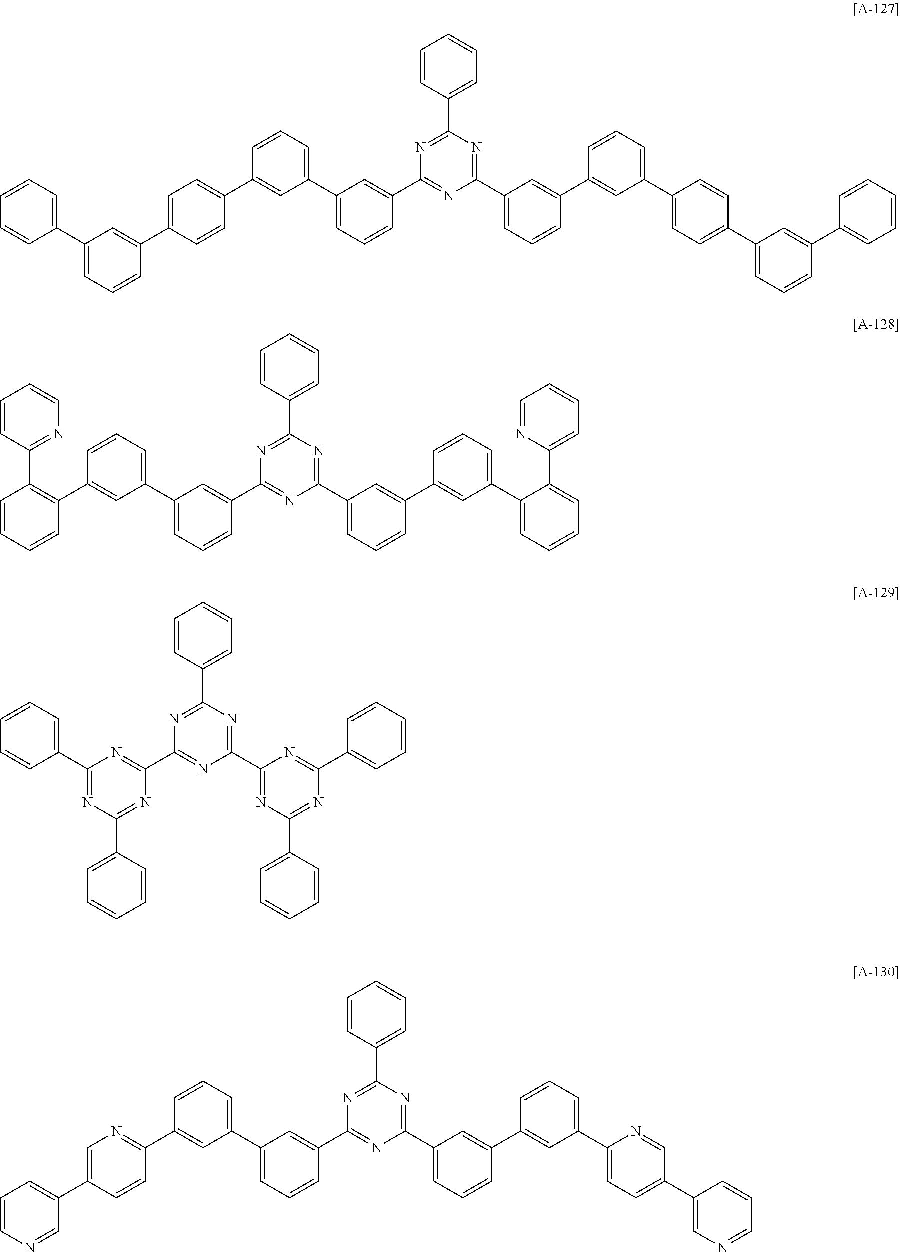 [SMILES: c1ccc(-c2cccc(-c3ccc(-c4cccc(-c5cccc(-c6nc(-c7ccccc7)nc(-c7cccc(-c8cccc(-c9ccc(-c%10cccc(-c%11ccccc%11)c%10)cc9)c8)c7)n6)c5)c4)cc3)c2)cc1 ; c1ccc(-c2nc(-c3cccc(-c4cccc(-c5ccccc5-c5ccccn5)c4)c3)nc(-c3cccc(-c4cccc(-c5ccccc5-c5ccccn5)c4)c3)n2)cc1 ; c1ccc(-c2nc(-c3ccccc3)nc(-c3nc(-c4ccccc4)nc(-c4nc(-c5ccccc5)nc(-c5ccccc5)n4)n3)n2)cc1 ; c1ccc(-c2nc(-c3cccc(-c4cccc(-c5ccc(-c6cccnc6)cn5)c4)c3)nc(-c3cccc(-c4cccc(-c5ccc(-c6cccnc6)cn5)c4)c3)n2)cc1]

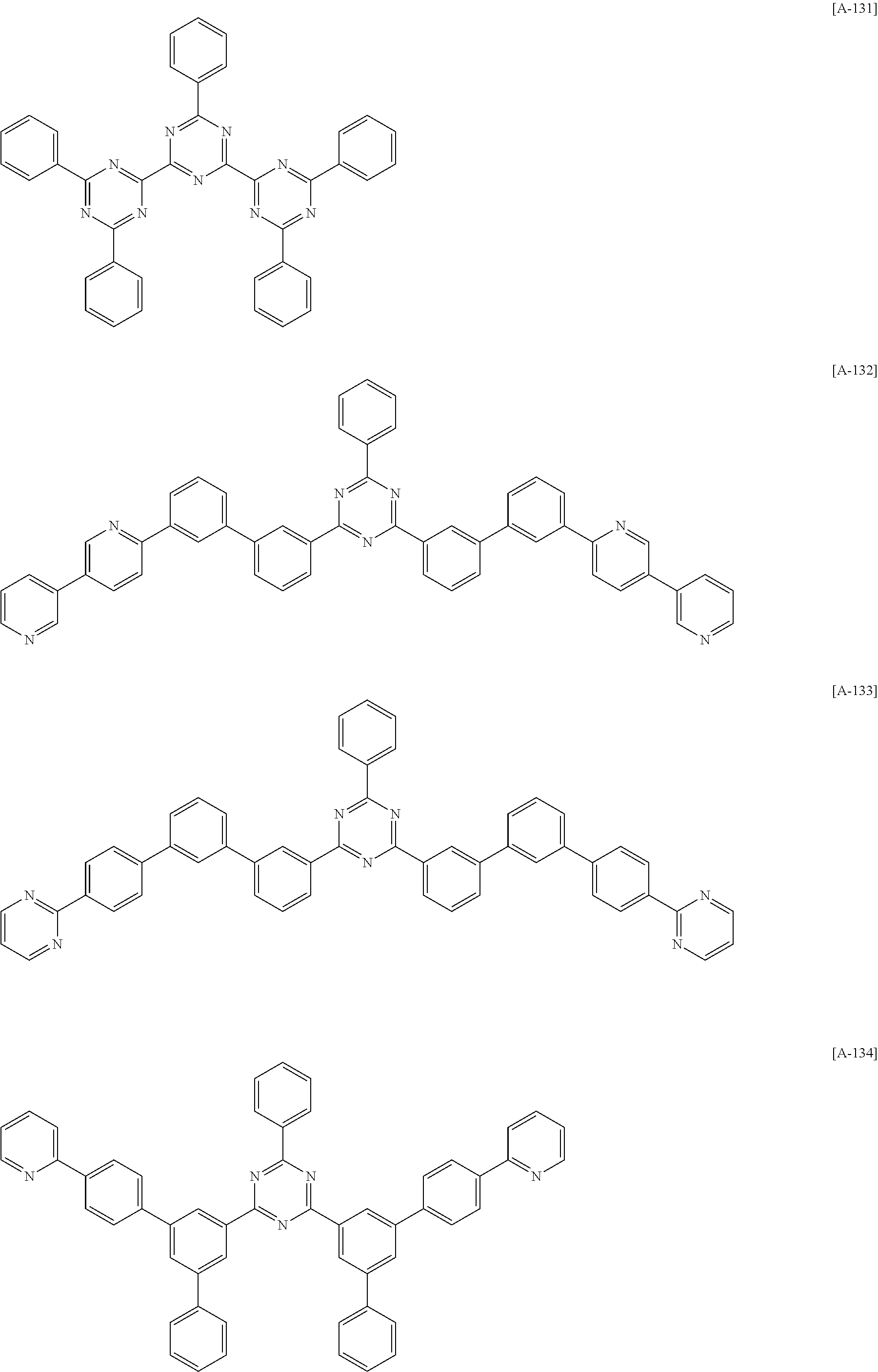 [SMILES: c1ccc(-c2nc(-c3ccccc3)nc(-c3nc(-c4ccccc4)nc(-c4nc(-c5ccccc5)nc(-c5ccccc5)n4)n3)n2)cc1 ; c1ccc(-c2nc(-c3cccc(-c4cccc(-c5ccc(-c6cccnc6)cn5)c4)c3)nc(-c3cccc(-c4cccc(-c5ccc(-c6cccnc6)cn5)c4)c3)n2)cc1 ; c1ccc(-c2nc(-c3cccc(-c4cccc(-c5ccc(-c6ncccn6)cc5)c4)c3)nc(-c3cccc(-c4cccc(-c5ccc(-c6ncccn6)cc5)c4)c3)n2)cc1 ; c1ccc(-c2cc(-c3ccc(-c4ccccn4)cc3)cc(-c3nc(-c4ccccc4)nc(-c4cc(-c5ccccc5)cc(-c5ccc(-c6ccccn6)cc5)c4)n3)c2)cc1]

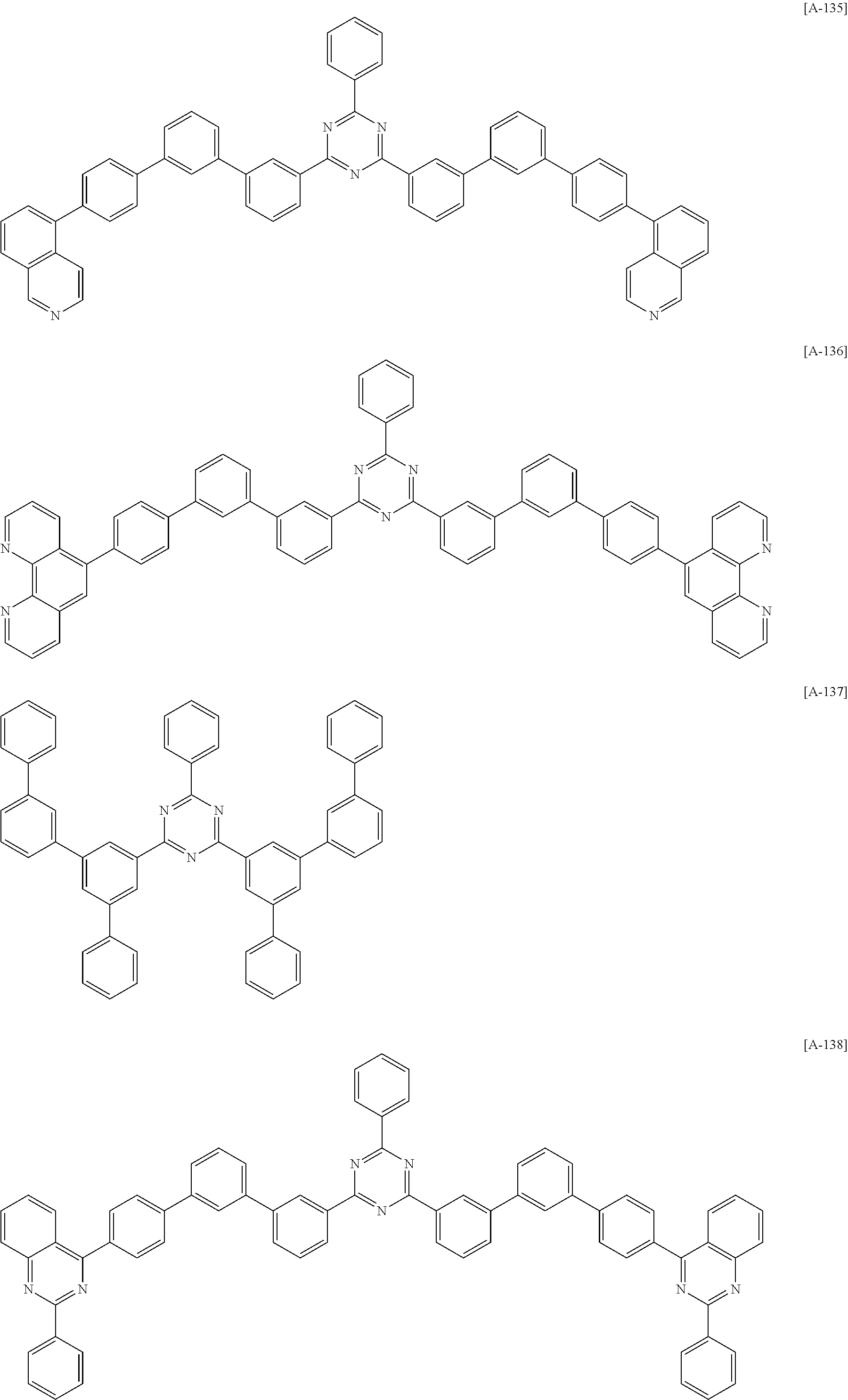

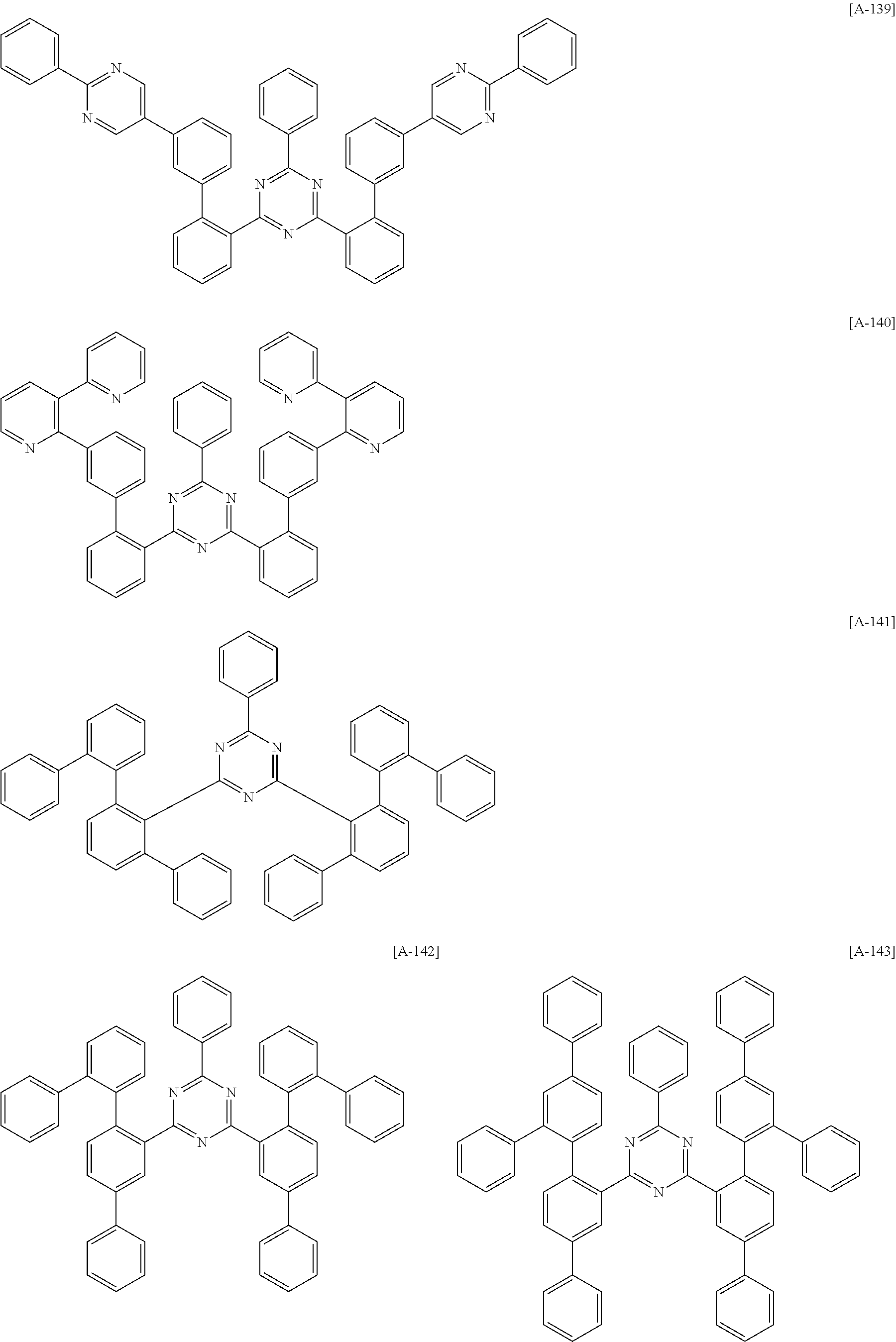

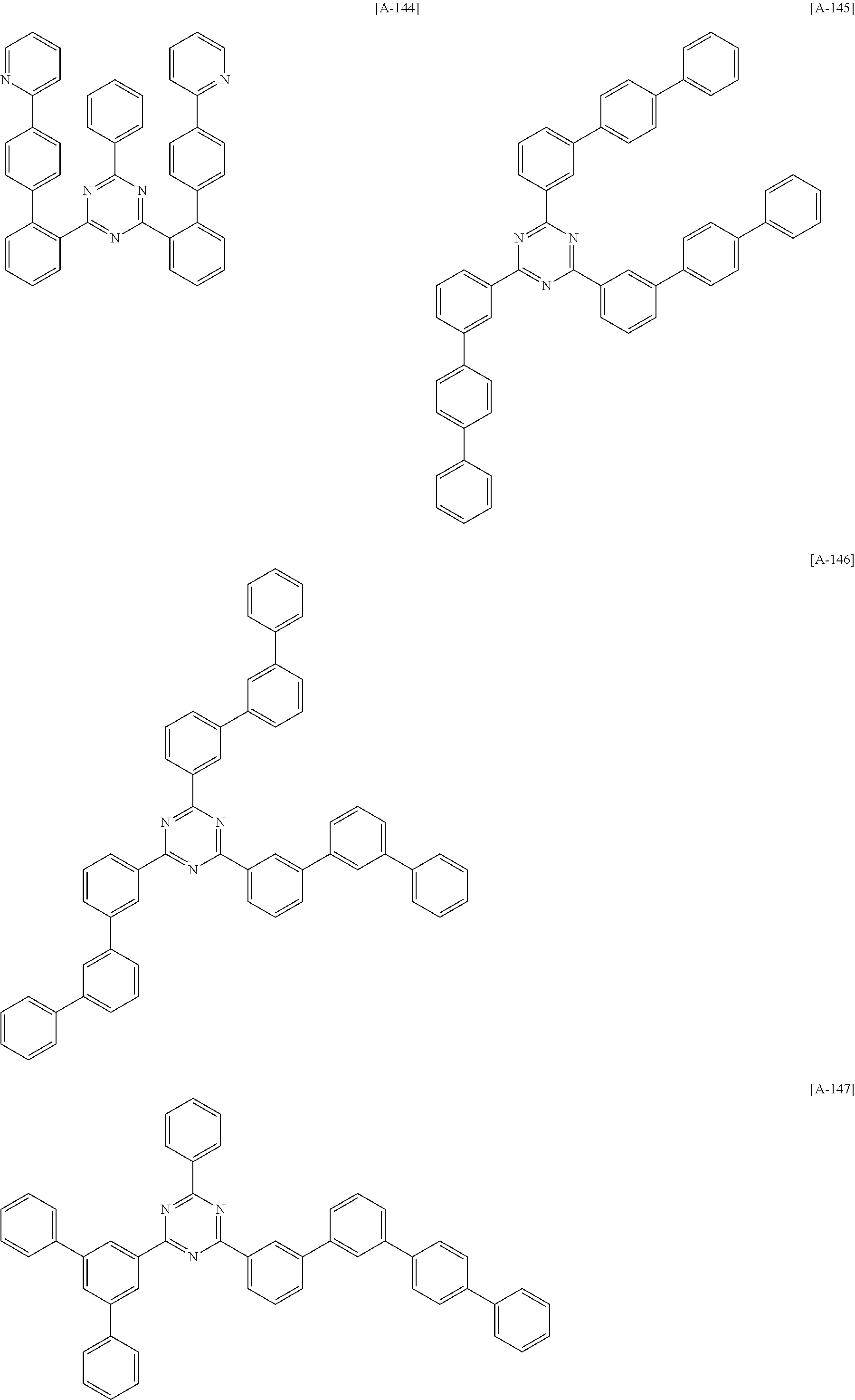 [SMILES: c1ccc(-c2nc(-c3ccccc3-c3ccc(-c4ccccn4)cc3)nc(-c3ccccc3-c3ccc(-c4ccccn4)cc3)n2)cc1 ; c1ccc(-c2ccc(-c3cccc(-c4nc(-c5cccc(-c6ccc(-c7ccccc7)cc6)c5)nc(-c5cccc(-c6ccc(-c7ccccc7)cc6)c5)n4)c3)cc2)cc1 ; c1ccc(-c2cccc(-c3cccc(-c4nc(-c5cccc(-c6cccc(-c7ccccc7)c6)c5)nc(-c5cccc(-c6cccc(-c7ccccc7)c6)c5)n4)c3)c2)cc1 ; c1ccc(-c2ccc(-c3cccc(-c4cccc(-c5nc(-c6ccccc6)nc(-c6cc(-c7ccccc7)cc(-c7ccccc7)c6)n5)c4)c3)cc2)cc1]

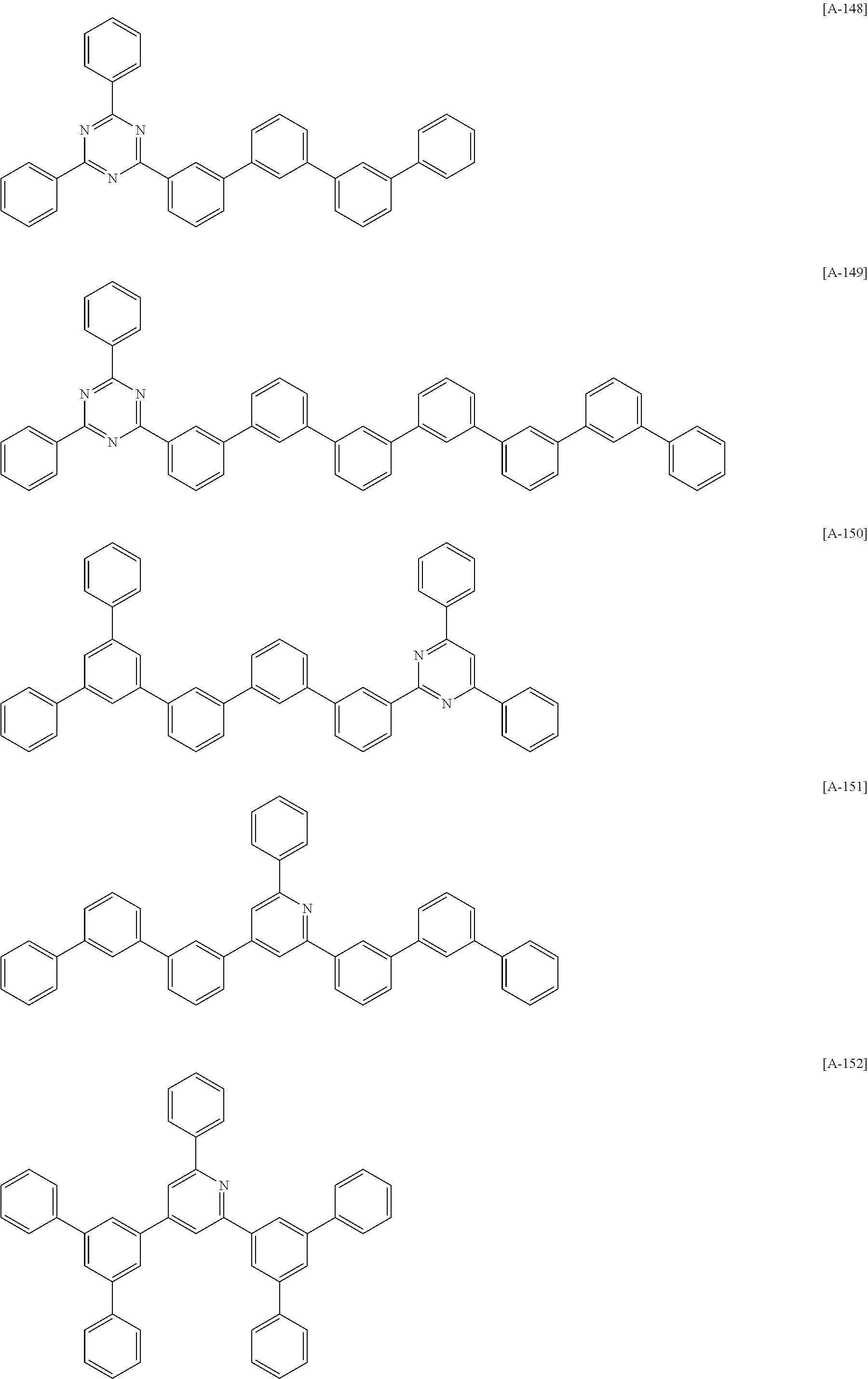 [SMILES: c1ccc(-c2cccc(-c3cccc(-c4cccc(-c5nc(-c6ccccc6)nc(-c6ccccc6)n5)c4)c3)c2)cc1 ; c1ccc(-c2cccc(-c3cccc(-c4cccc(-c5cccc(-c6cccc(-c7cccc(-c8nc(-c9ccccc9)nc(-c9ccccc9)n8)c7)c6)c5)c4)c3)c2)cc1 ; c1ccc(-c2cc(-c3ccccc3)cc(-c3cccc(-c4cccc(-c5cccc(-c6nc(-c7ccccc7)cc(-c7ccccc7)n6)c5)c4)c3)c2)cc1 ; c1ccc(-c2cccc(-c3cccc(-c4cc(-c5ccccc5)nc(-c5cccc(-c6cccc(-c7ccccc7)c6)c5)c4)c3)c2)cc1 ; c1ccc(-c2cc(-c3ccccc3)cc(-c3cc(-c4ccccc4)nc(-c4cc(-c5ccccc5)cc(-c5ccccc5)c4)c3)c2)cc1]

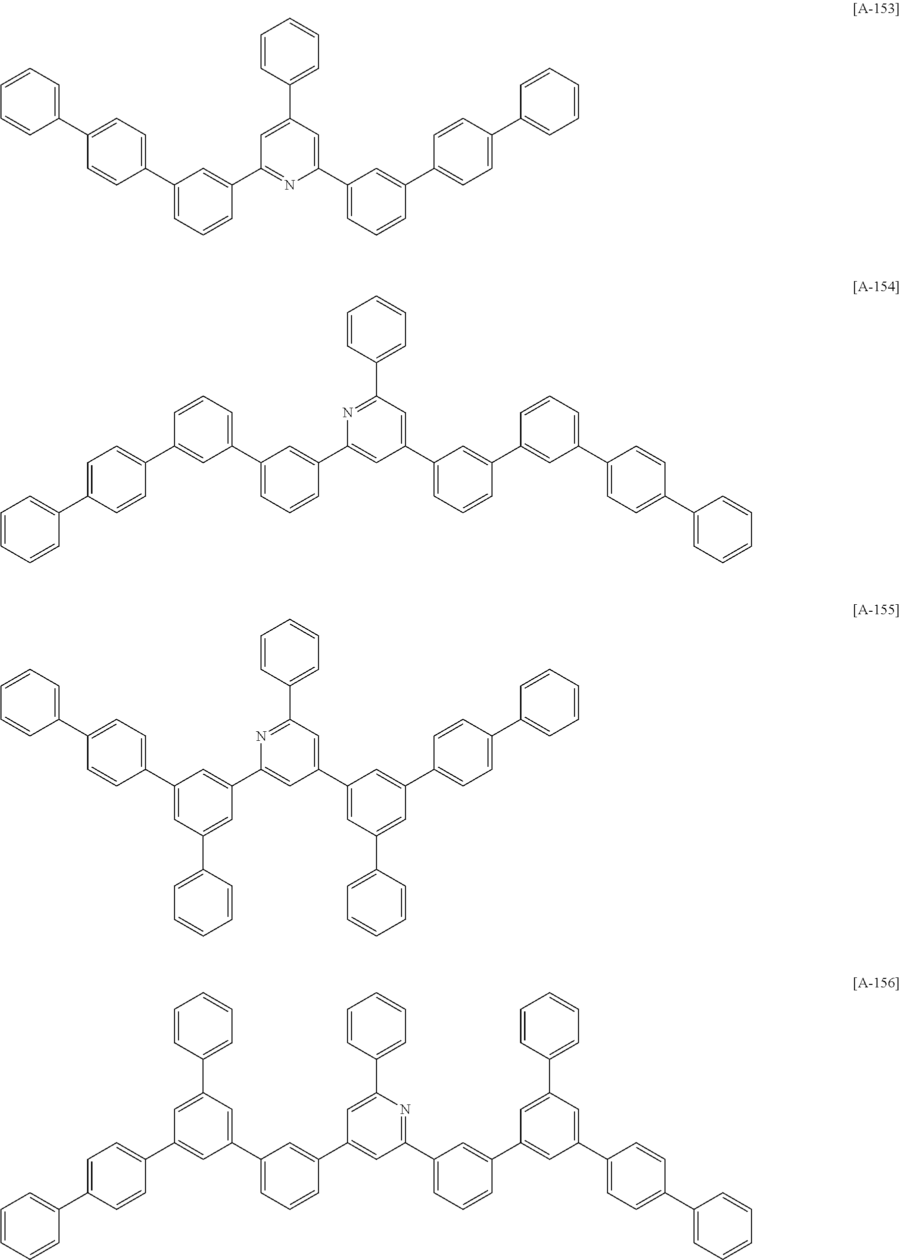 [SMILES: c1ccc(-c2ccc(-c3cccc(-c4cc(-c5ccccc5)cc(-c5cccc(-c6ccc(-c7ccccc7)cc6)c5)n4)c3)cc2)cc1 ; c1ccc(-c2ccc(-c3cccc(-c4cccc(-c5cc(-c6ccccc6)nc(-c6cccc(-c7cccc(-c8ccc(-c9ccccc9)cc8)c7)c6)c5)c4)c3)cc2)cc1 ; c1ccc(-c2ccc(-c3cc(-c4ccccc4)cc(-c4cc(-c5ccccc5)nc(-c5cc(-c6ccccc6)cc(-c6ccc(-c7ccccc7)cc6)c5)c4)c3)cc2)cc1 ; c1ccc(-c2ccc(-c3cc(-c4ccccc4)cc(-c4cccc(-c5cc(-c6ccccc6)nc(-c6cccc(-c7cc(-c8ccccc8)cc(-c8ccc(-c9ccccc9)cc8)c7)c6)c5)c4)c3)cc2)cc1]

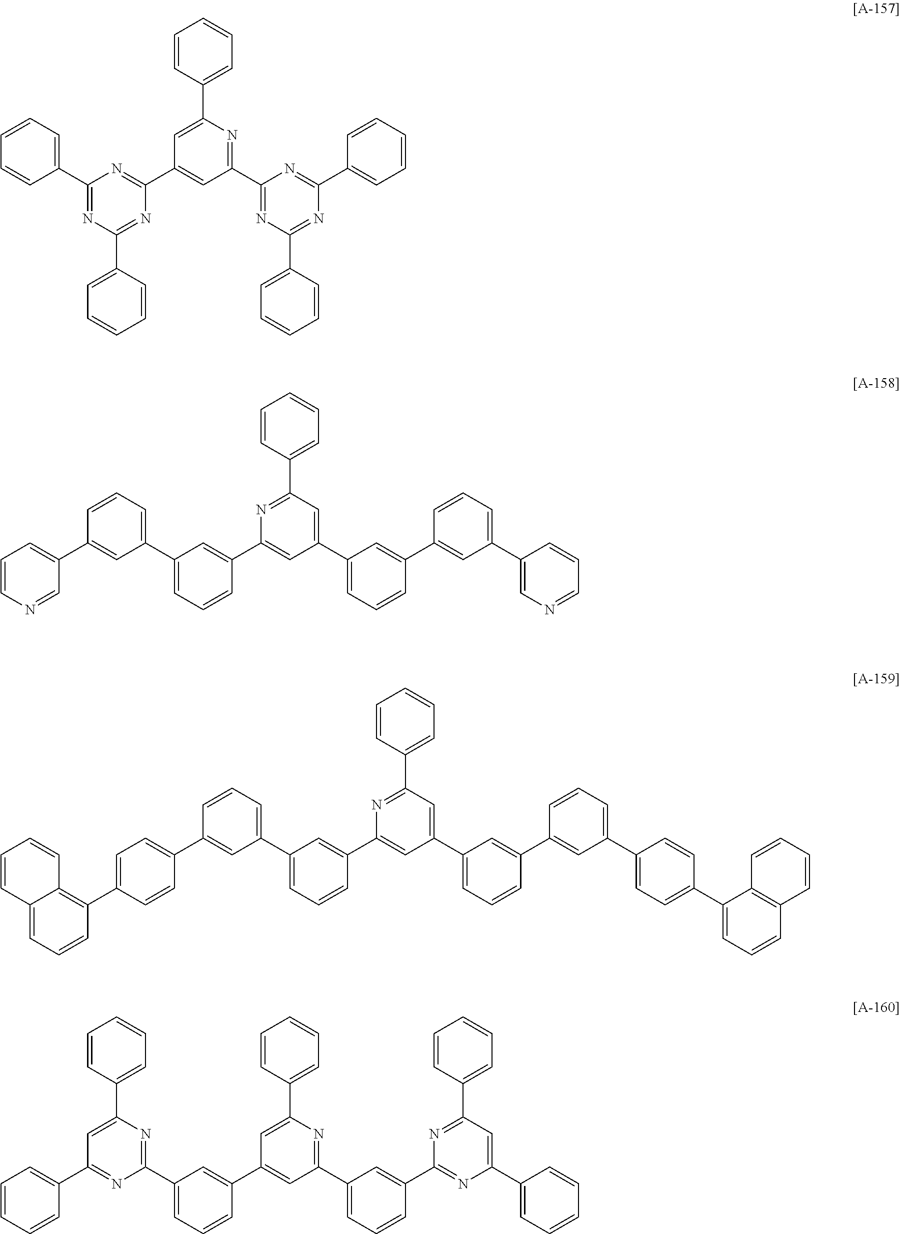 [SMILES: c1ccc(-c2cc(-c3nc(-c4ccccc4)nc(-c4ccccc4)n3)cc(-c3nc(-c4ccccc4)nc(-c4ccccc4)n3)n2)cc1 ; c1ccc(-c2cc(-c3cccc(-c4cccc(-c5cccnc5)c4)c3)cc(-c3cccc(-c4cccc(-c5cccnc5)c4)c3)n2)cc1 ; c1ccc(-c2cc(-c3cccc(-c4cccc(-c5ccc(-c6cccc7ccccc67)cc5)c4)c3)cc(-c3cccc(-c4cccc(-c5ccc(-c6cccc7ccccc67)cc5)c4)c3)n2)cc1 ; c1ccc(-c2cc(-c3cccc(-c4nc(-c5ccccc5)cc(-c5ccccc5)n4)c3)cc(-c3cccc(-c4nc(-c5ccccc5)cc(-c5ccccc5)n4)c3)n2)cc1]

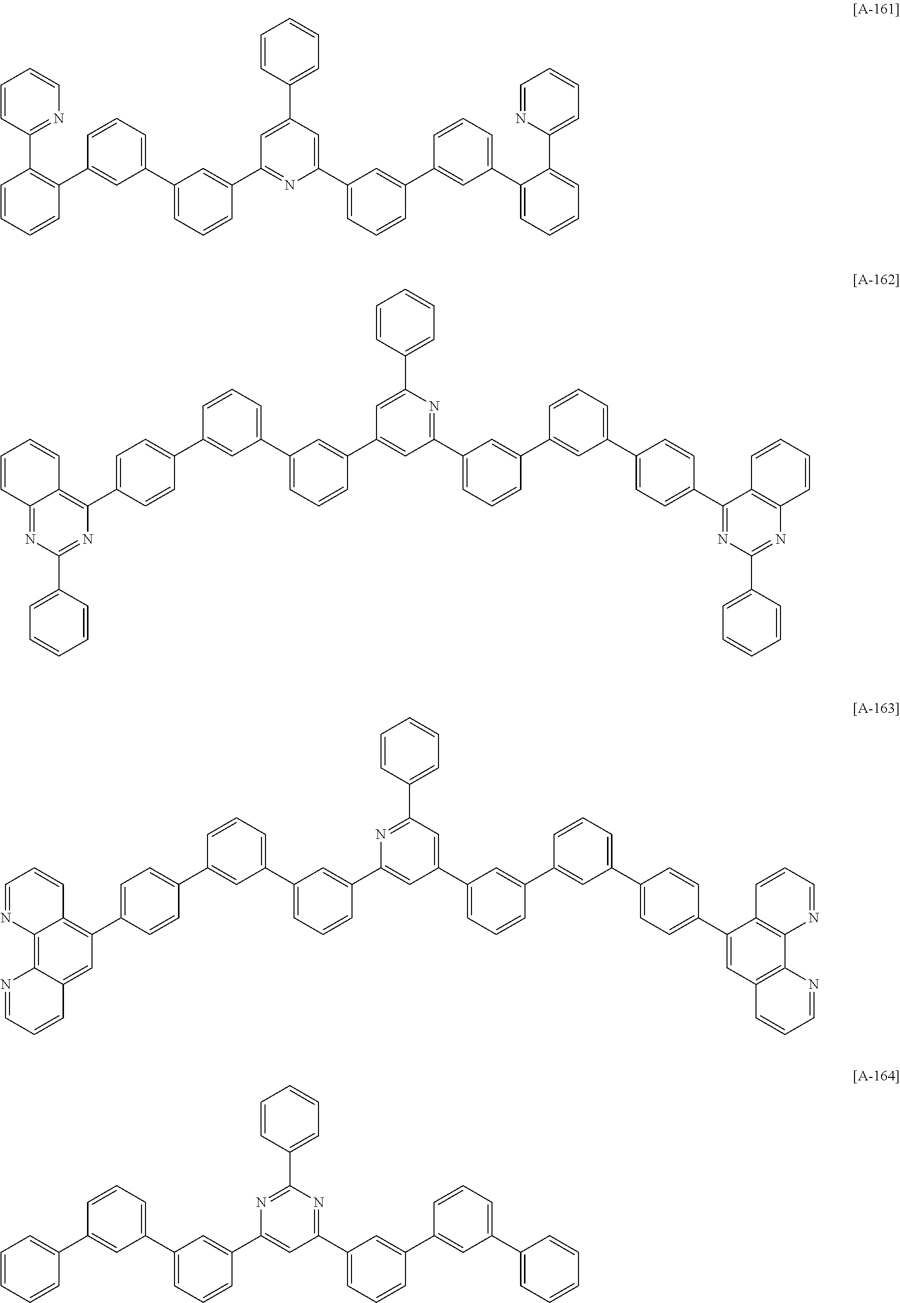 [SMILES: c1ccc(-c2cc(-c3cccc(-c4cccc(-c5ccccc5-c5ccccn5)c4)c3)nc(-c3cccc(-c4cccc(-c5ccccc5-c5ccccn5)c4)c3)c2)cc1 ; c1ccc(-c2cc(-c3cccc(-c4cccc(-c5ccc(-c6nc(-c7ccccc7)nc7ccccc67)cc5)c4)c3)cc(-c3cccc(-c4cccc(-c5ccc(-c6nc(-c7ccccc7)nc7ccccc67)cc5)c4)c3)n2)cc1 ; c1ccc(-c2cc(-c3cccc(-c4cccc(-c5ccc(-c6cc7cccnc7c7ncccc67)cc5)c4)c3)cc(-c3cccc(-c4cccc(-c5ccc(-c6cc7cccnc7c7ncccc67)cc5)c4)c3)n2)cc1 ; c1ccc(-c2cccc(-c3cccc(-c4cc(-c5cccc(-c6cccc(-c7ccccc7)c6)c5)nc(-c5ccccc5)n4)c3)c2)cc1]

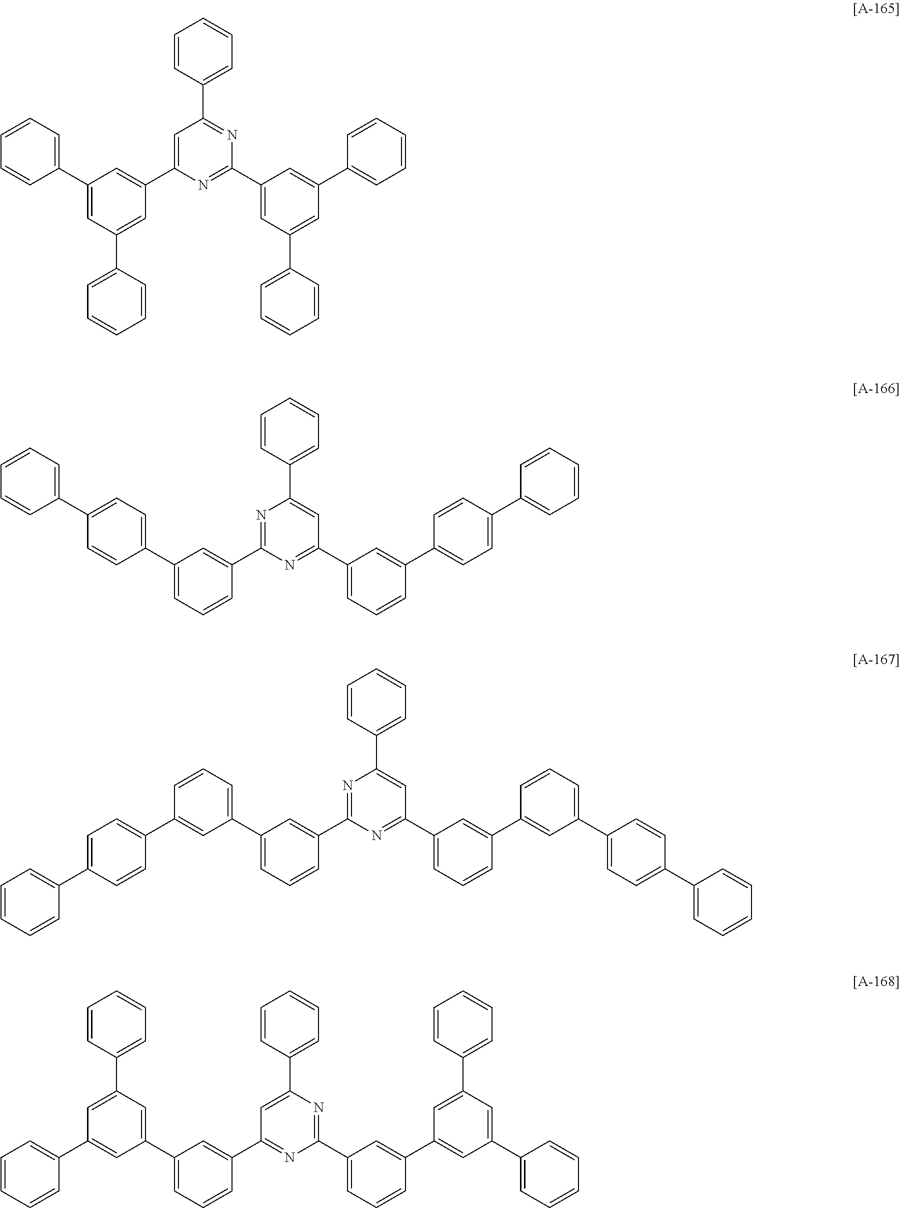 [SMILES: c1ccc(-c2cc(-c3ccccc3)cc(-c3cc(-c4ccccc4)nc(-c4cc(-c5ccccc5)cc(-c5ccccc5)c4)n3)c2)cc1 ; c1ccc(-c2ccc(-c3cccc(-c4cc(-c5ccccc5)nc(-c5cccc(-c6ccc(-c7ccccc7)cc6)c5)n4)c3)cc2)cc1 ; c1ccc(-c2ccc(-c3cccc(-c4cccc(-c5cc(-c6ccccc6)nc(-c6cccc(-c7cccc(-c8ccc(-c9ccccc9)cc8)c7)c6)n5)c4)c3)cc2)cc1 ; c1ccc(-c2cc(-c3ccccc3)cc(-c3cccc(-c4cc(-c5ccccc5)nc(-c5cccc(-c6cc(-c7ccccc7)cc(-c7ccccc7)c6)c5)n4)c3)c2)cc1]

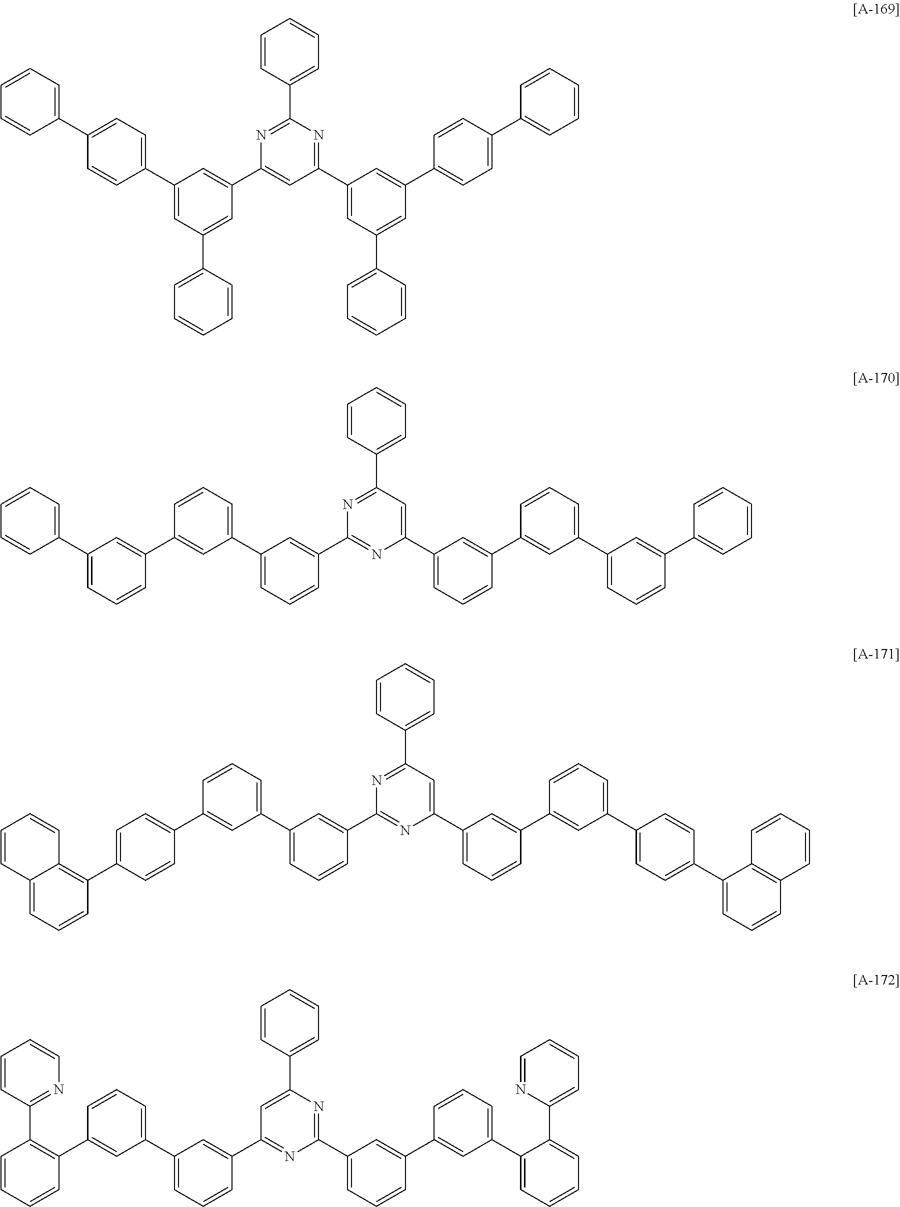 [SMILES: c1ccc(-c2ccc(-c3cc(-c4ccccc4)cc(-c4cc(-c5cc(-c6ccccc6)cc(-c6ccc(-c7ccccc7)cc6)c5)nc(-c5ccccc5)n4)c3)cc2)cc1 ; c1ccc(-c2cccc(-c3cccc(-c4cccc(-c5cc(-c6ccccc6)nc(-c6cccc(-c7cccc(-c8cccc(-c9ccccc9)c8)c7)c6)n5)c4)c3)c2)cc1 ; c1ccc(-c2cc(-c3cccc(-c4cccc(-c5ccc(-c6cccc7ccccc67)cc5)c4)c3)nc(-c3cccc(-c4cccc(-c5ccc(-c6cccc7ccccc67)cc5)c4)c3)n2)cc1 ; c1ccc(-c2cc(-c3cccc(-c4cccc(-c5ccccc5-c5ccccn5)c4)c3)nc(-c3cccc(-c4cccc(-c5ccccc5-c5ccccn5)c4)c3)n2)cc1]

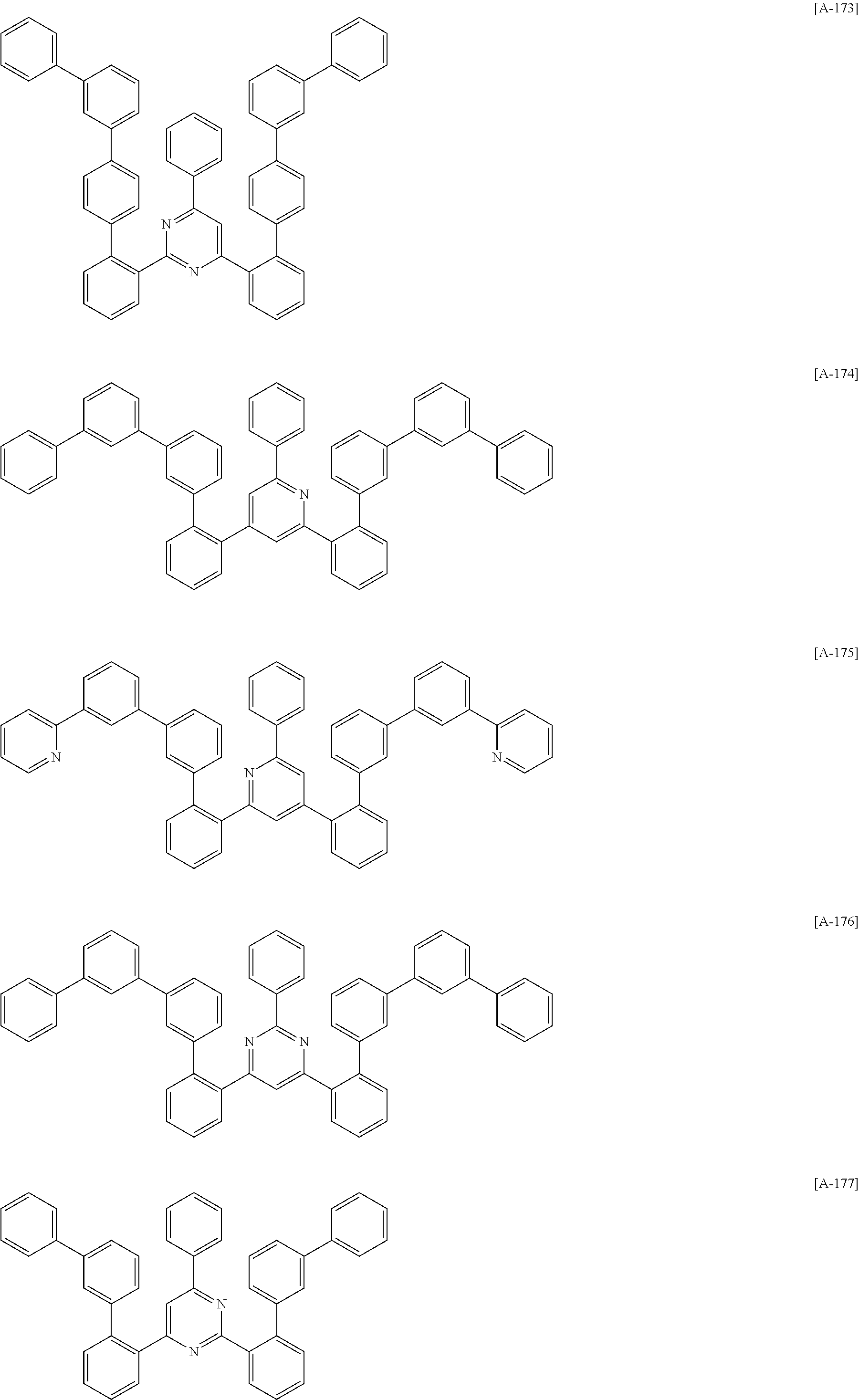

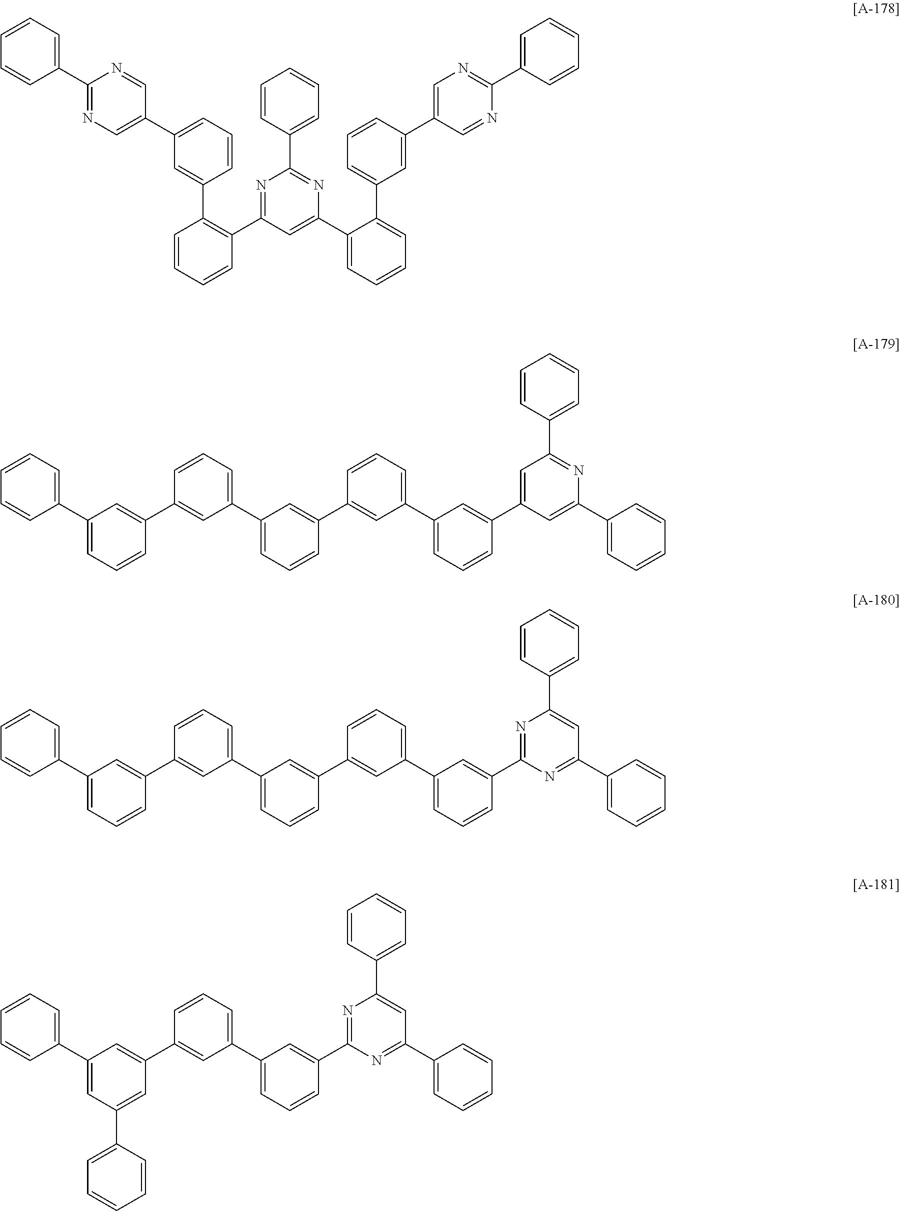 [SMILES: c1ccc(-c2ncc(-c3cccc(-c4ccccc4-c4cc(-c5ccccc5-c5cccc(-c6cnc(-c7ccccc7)nc6)c5)nc(-c5ccccc5)n4)c3)cn2)cc1 ; c1ccc(-c2cccc(-c3cccc(-c4cccc(-c5cccc(-c6cccc(-c7cc(-c8ccccc8)nc(-c8ccccc8)c7)c6)c5)c4)c3)c2)cc1 ; c1ccc(-c2cccc(-c3cccc(-c4cccc(-c5cccc(-c6cccc(-c7nc(-c8ccccc8)cc(-c8ccccc8)n7)c6)c5)c4)c3)c2)cc1 ; c1ccc(-c2cc(-c3ccccc3)cc(-c3cccc(-c4cccc(-c5nc(-c6ccccc6)cc(-c6ccccc6)n5)c4)c3)c2)cc1]

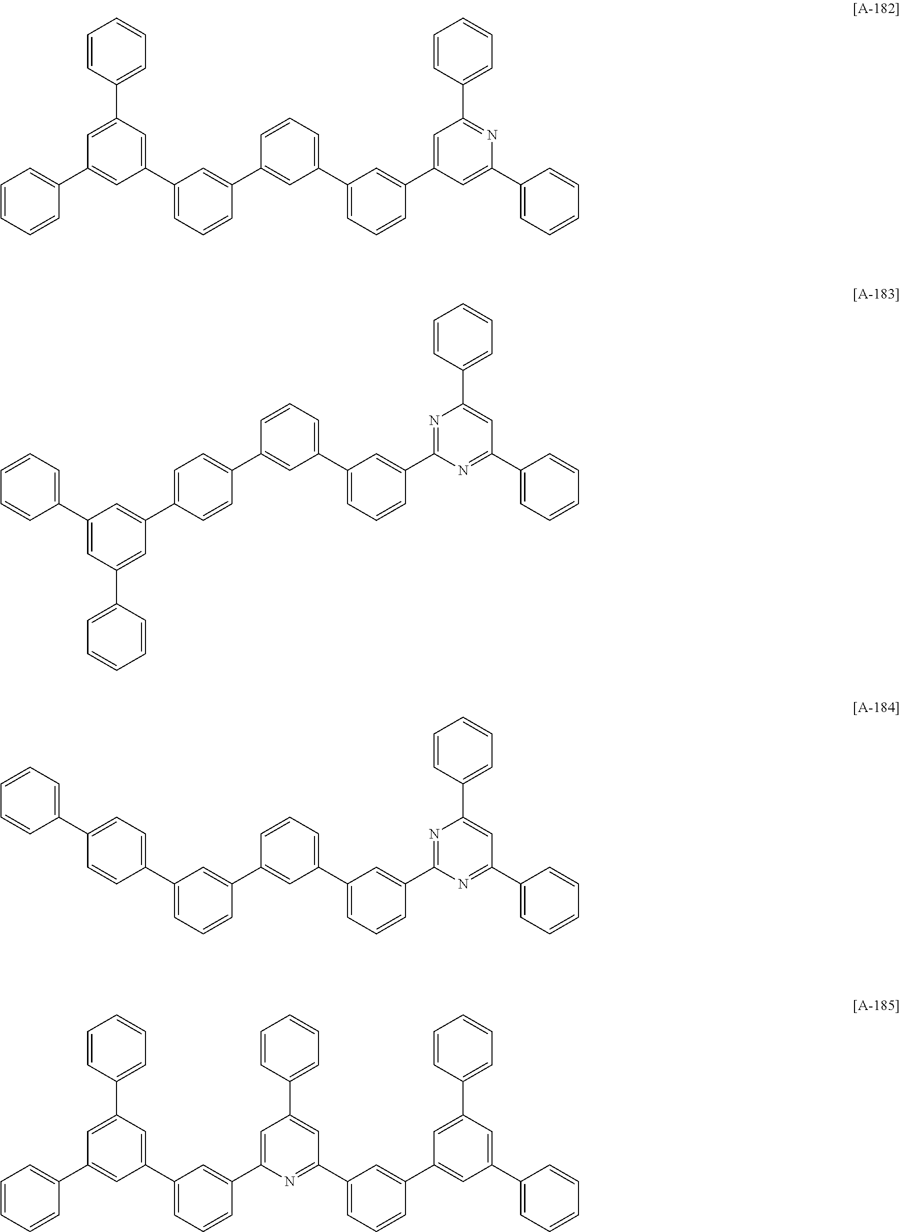 [SMILES: c1ccc(-c2cc(-c3ccccc3)cc(-c3cccc(-c4cccc(-c5cccc(-c6cc(-c7ccccc7)nc(-c7ccccc7)c6)c5)c4)c3)c2)cc1 ; c1ccc(-c2cc(-c3ccccc3)cc(-c3ccc(-c4cccc(-c5cccc(-c6nc(-c7ccccc7)cc(-c7ccccc7)n6)c5)c4)cc3)c2)cc1 ; c1ccc(-c2ccc(-c3cccc(-c4cccc(-c5cccc(-c6nc(-c7ccccc7)cc(-c7ccccc7)n6)c5)c4)c3)cc2)cc1 ; c1ccc(-c2cc(-c3ccccc3)cc(-c3cccc(-c4cc(-c5ccccc5)cc(-c5cccc(-c6cc(-c7ccccc7)cc(-c7ccccc7)c6)c5)n4)c3)c2)cc1]

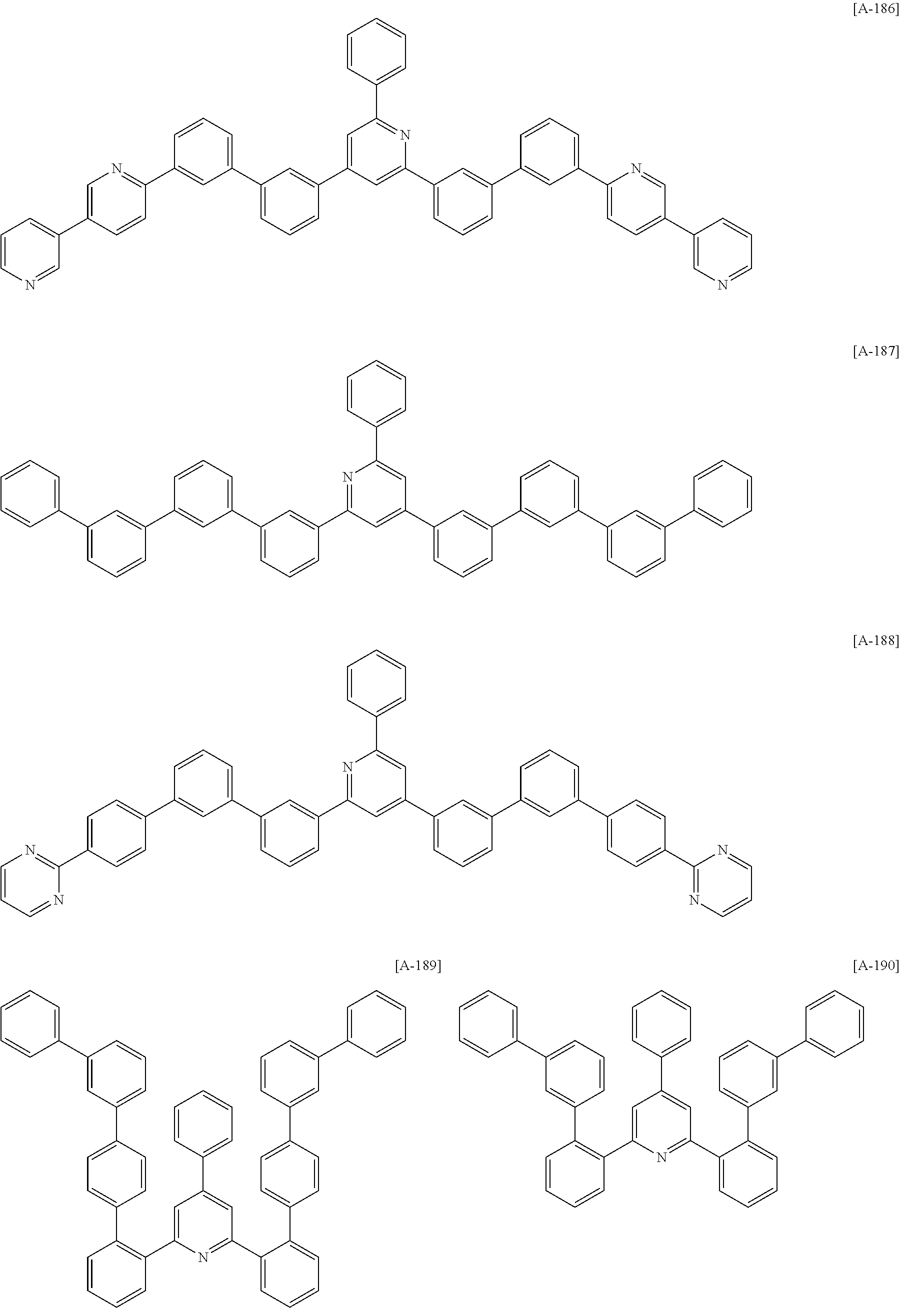

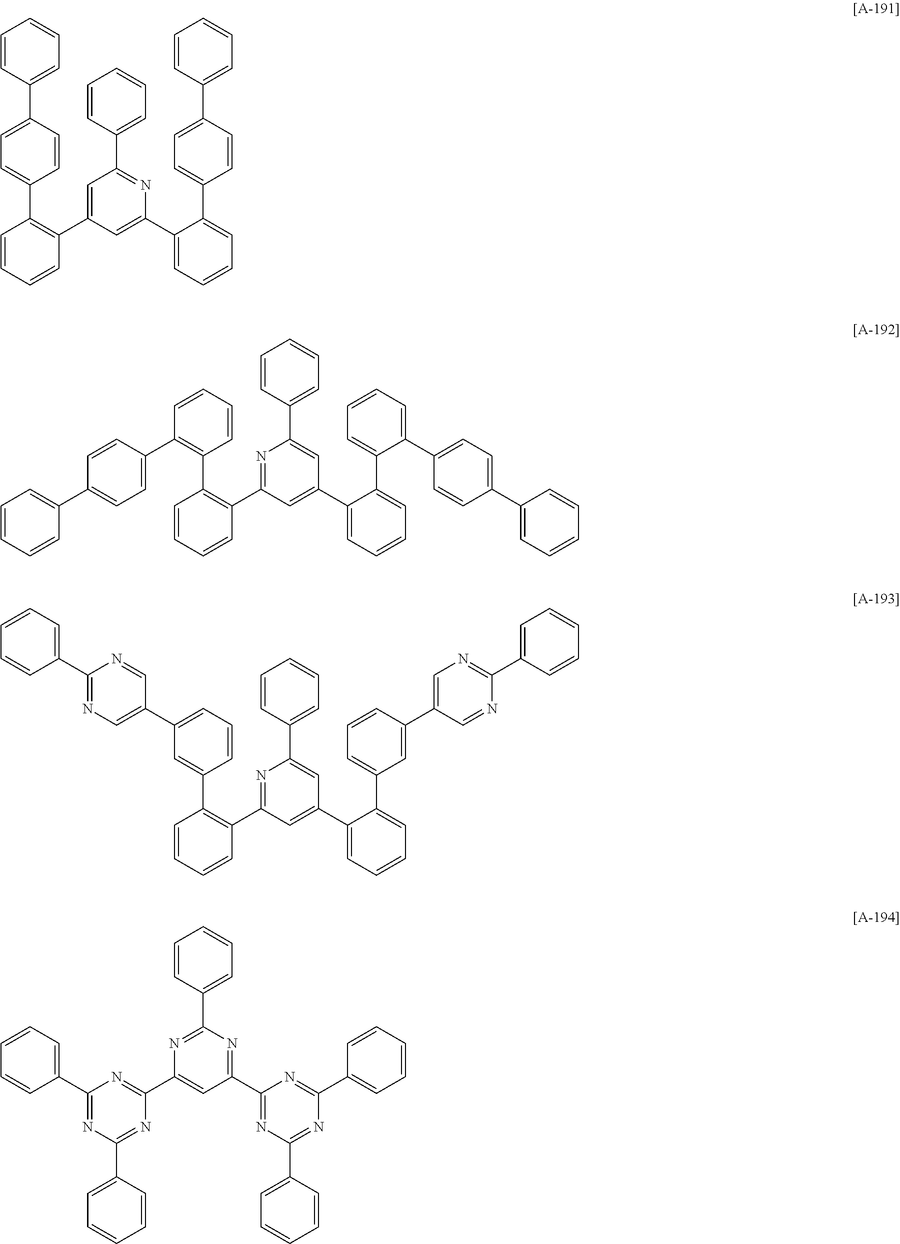 [SMILES: c1ccc(-c2ccc(-c3ccccc3-c3cc(-c4ccccc4)nc(-c4ccccc4-c4ccc(-c5ccccc5)cc4)c3)cc2)cc1 ; c1ccc(-c2ccc(-c3ccccc3-c3ccccc3-c3cc(-c4ccccc4)nc(-c4ccccc4-c4ccccc4-c4ccc(-c5ccccc5)cc4)c3)cc2)cc1 ; c1ccc(-c2cc(-c3ccccc3-c3cccc(-c4cnc(-c5ccccc5)nc4)c3)cc(-c3ccccc3-c3cccc(-c4cnc(-c5ccccc5)nc4)c3)n2)cc1 ; c1ccc(-c2nc(-c3nc(-c4ccccc4)nc(-c4ccccc4)n3)cc(-c3nc(-c4ccccc4)nc(-c4ccccc4)n3)n2)cc1]

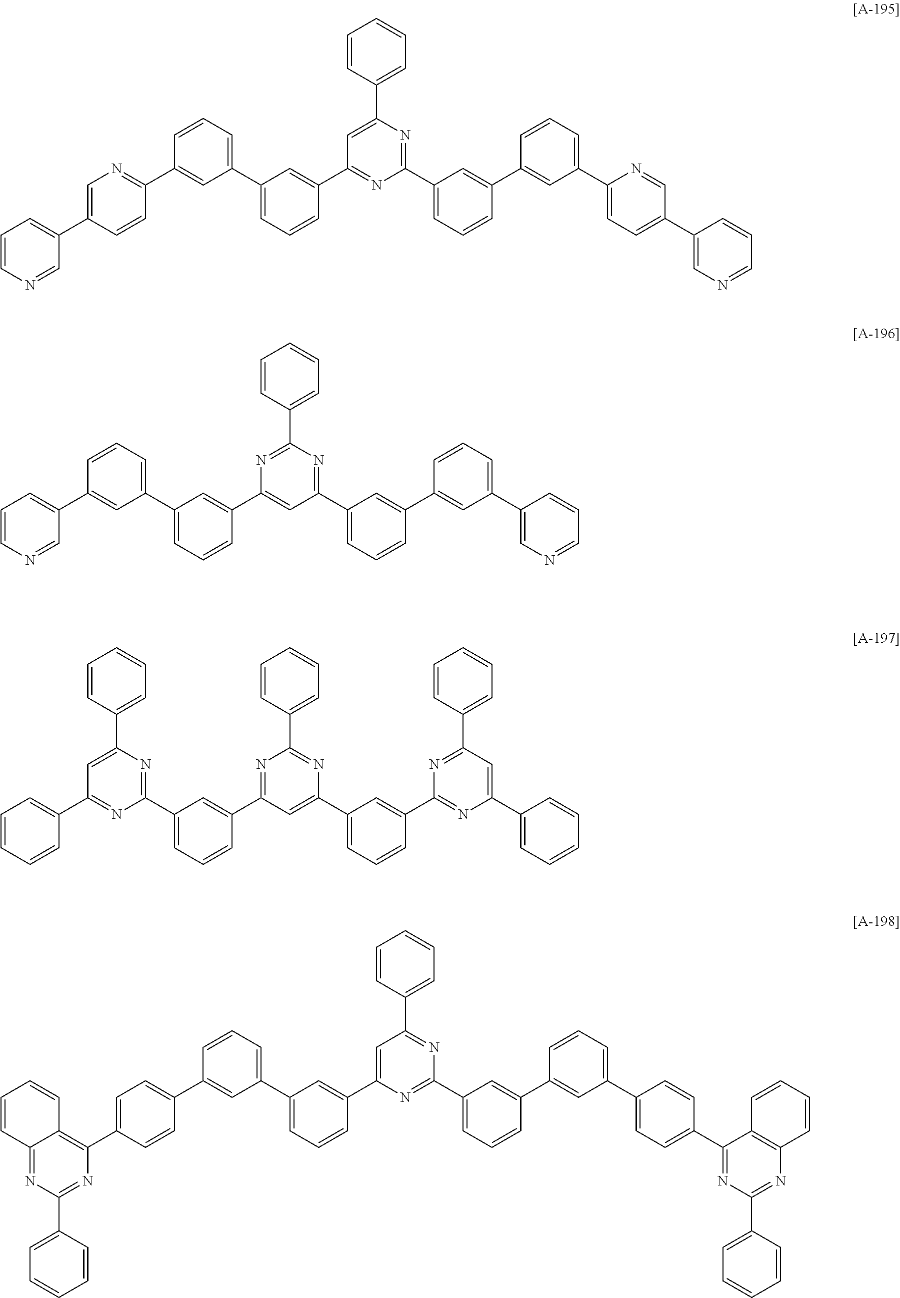 [SMILES: c1ccc(-c2cc(-c3cccc(-c4cccc(-c5ccc(-c6cccnc6)cn5)c4)c3)nc(-c3cccc(-c4cccc(-c5ccc(-c6cccnc6)cn5)c4)c3)n2)cc1 ; c1ccc(-c2nc(-c3cccc(-c4cccc(-c5cccnc5)c4)c3)cc(-c3cccc(-c4cccc(-c5cccnc5)c4)c3)n2)cc1 ; c1ccc(-c2cc(-c3ccccc3)nc(-c3cccc(-c4cc(-c5cccc(-c6nc(-c7ccccc7)cc(-c7ccccc7)n6)c5)nc(-c5ccccc5)n4)c3)n2)cc1 ; c1ccc(-c2cc(-c3cccc(-c4cccc(-c5ccc(-c6nc(-c7ccccc7)nc7ccccc67)cc5)c4)c3)nc(-c3cccc(-c4cccc(-c5ccc(-c6nc(-c7ccccc7)nc7ccccc67)cc5)c4)c3)n2)cc1]

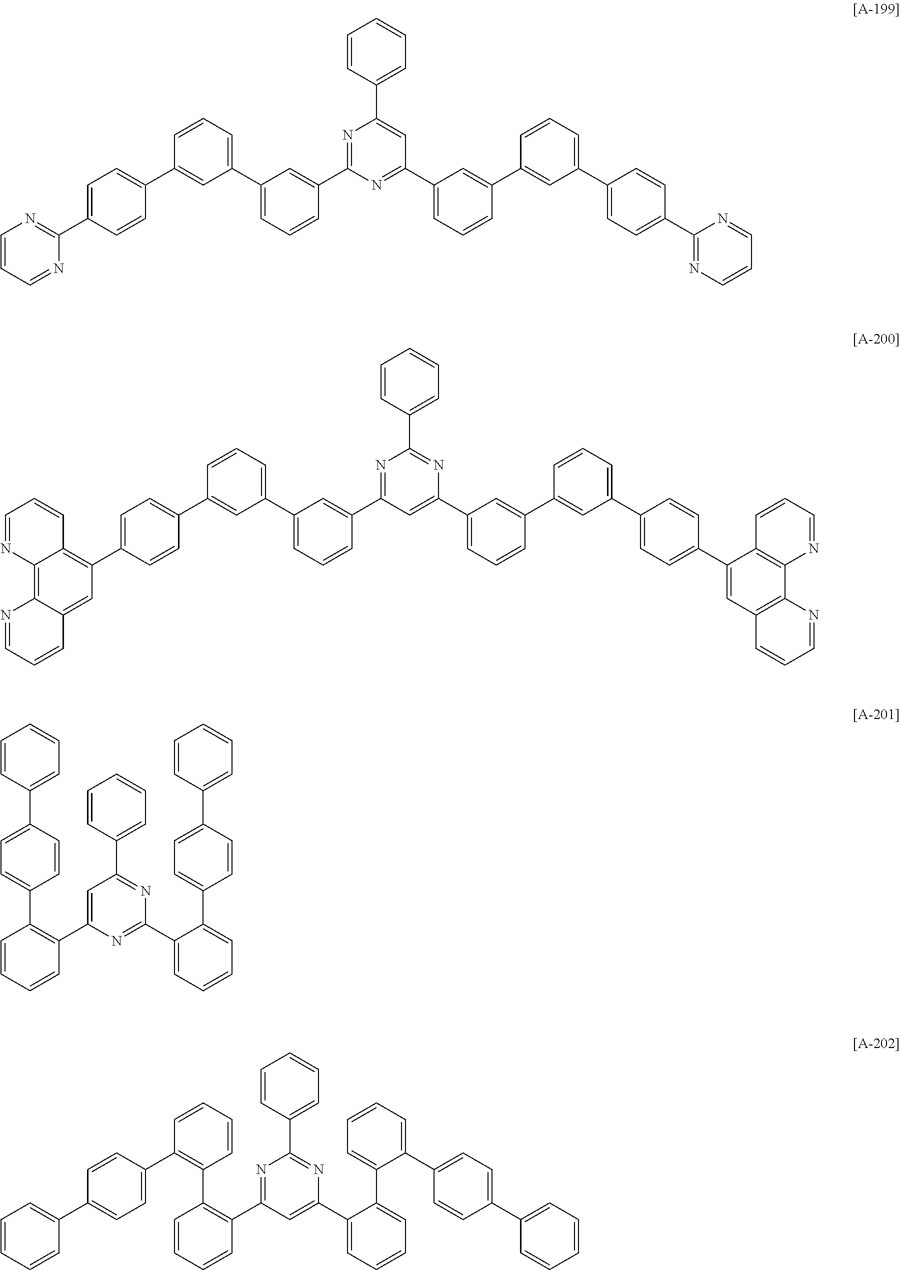 [SMILES: c1ccc(-c2cc(-c3cccc(-c4cccc(-c5ccc(-c6ncccn6)cc5)c4)c3)nc(-c3cccc(-c4cccc(-c5ccc(-c6ncccn6)cc5)c4)c3)n2)cc1 ; c1ccc(-c2nc(-c3cccc(-c4cccc(-c5ccc(-c6cc7cccnc7c7ncccc67)cc5)c4)c3)cc(-c3cccc(-c4cccc(-c5ccc(-c6cc7cccnc7c7ncccc67)cc5)c4)c3)n2)cc1 ; c1ccc(-c2ccc(-c3ccccc3-c3cc(-c4ccccc4)nc(-c4ccccc4-c4ccc(-c5ccccc5)cc4)n3)cc2)cc1 ; c1ccc(-c2ccc(-c3ccccc3-c3ccccc3-c3cc(-c4ccccc4-c4ccccc4-c4ccc(-c5ccccc5)cc4)nc(-c4ccccc4)n3)cc2)cc1]

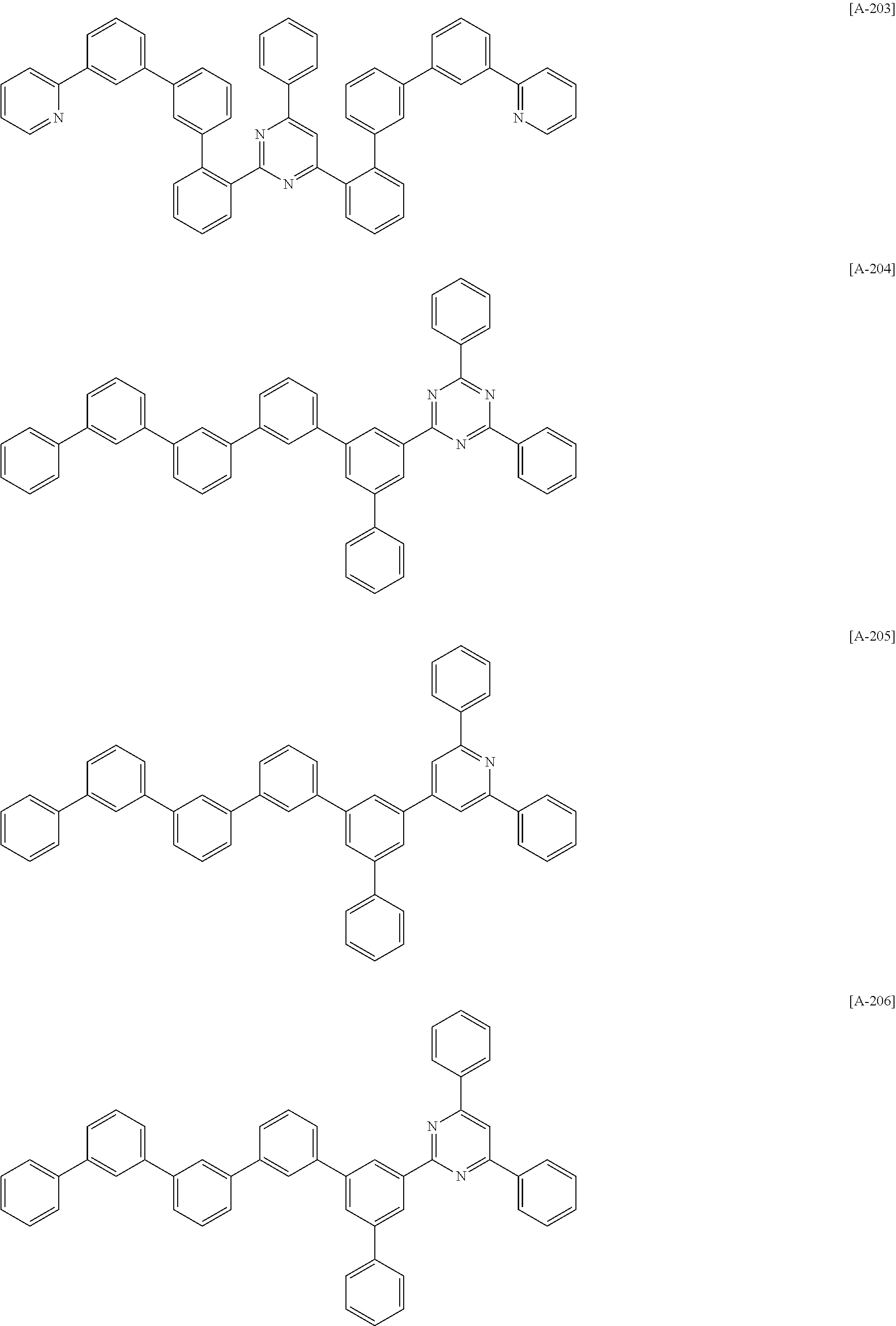 [SMILES: c1ccc(-c2cc(-c3ccccc3-c3cccc(-c4cccc(-c5ccccn5)c4)c3)nc(-c3ccccc3-c3cccc(-c4cccc(-c5ccccn5)c4)c3)n2)cc1 ; c1ccc(-c2cccc(-c3cccc(-c4cccc(-c5cc(-c6ccccc6)cc(-c6nc(-c7ccccc7)nc(-c7ccccc7)n6)c5)c4)c3)c2)cc1 ; c1ccc(-c2cccc(-c3cccc(-c4cccc(-c5cc(-c6ccccc6)cc(-c6cc(-c7ccccc7)nc(-c7ccccc7)c6)c5)c4)c3)c2)cc1 ; c1ccc(-c2cccc(-c3cccc(-c4cccc(-c5cc(-c6ccccc6)cc(-c6nc(-c7ccccc7)cc(-c7ccccc7)n6)c5)c4)c3)c2)cc1]

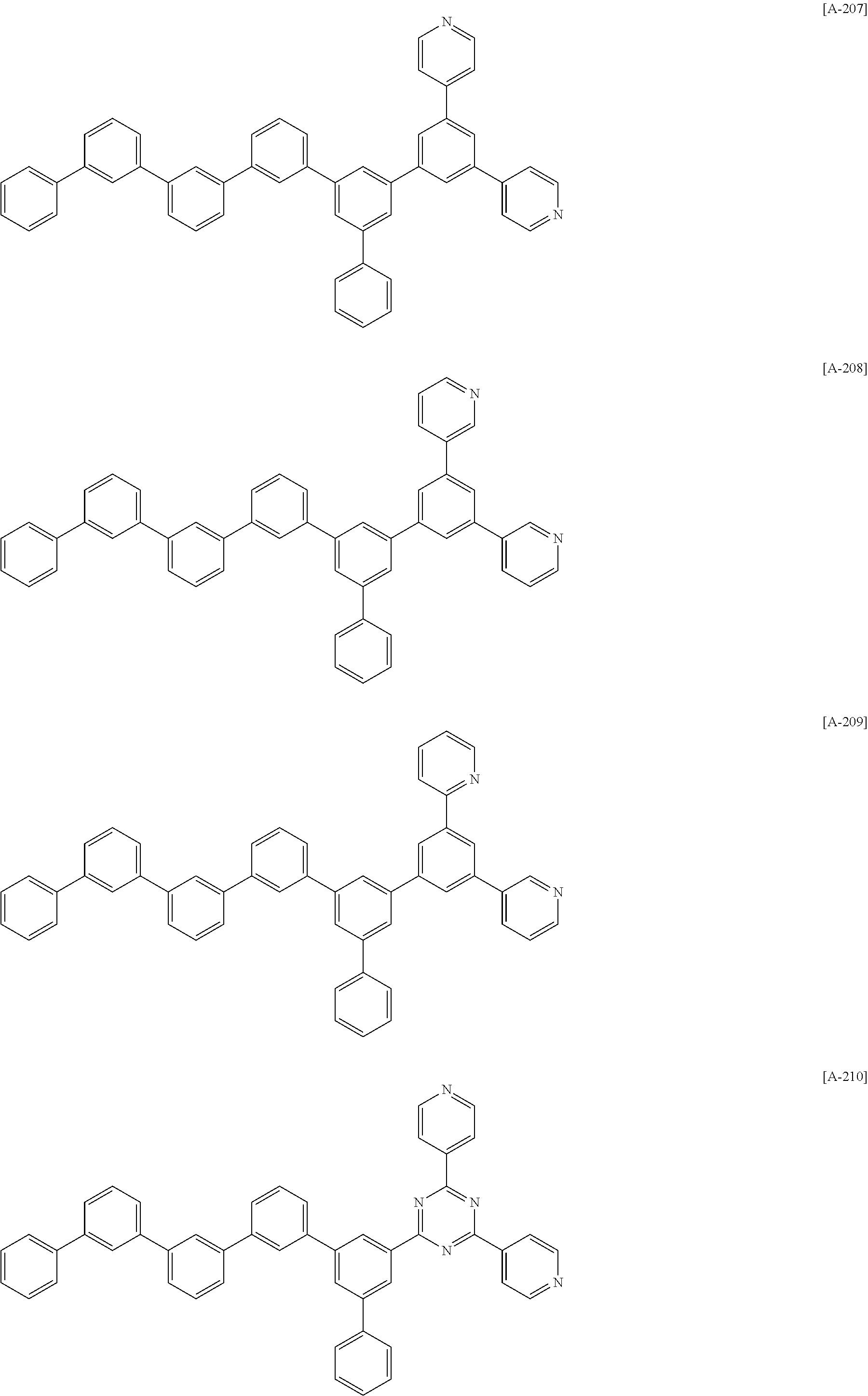 [SMILES: c1ccc(-c2cccc(-c3cccc(-c4cccc(-c5cc(-c6ccccc6)cc(-c6cc(-c7ccncc7)cc(-c7ccncc7)c6)c5)c4)c3)c2)cc1 ; c1ccc(-c2cccc(-c3cccc(-c4cccc(-c5cc(-c6ccccc6)cc(-c6cc(-c7cccnc7)cc(-c7cccnc7)c6)c5)c4)c3)c2)cc1 ; c1ccc(-c2cccc(-c3cccc(-c4cccc(-c5cc(-c6ccccc6)cc(-c6cc(-c7cccnc7)cc(-c7ccccn7)c6)c5)c4)c3)c2)cc1 ; c1ccc(-c2cccc(-c3cccc(-c4cccc(-c5cc(-c6ccccc6)cc(-c6nc(-c7ccncc7)nc(-c7ccncc7)n6)c5)c4)c3)c2)cc1]

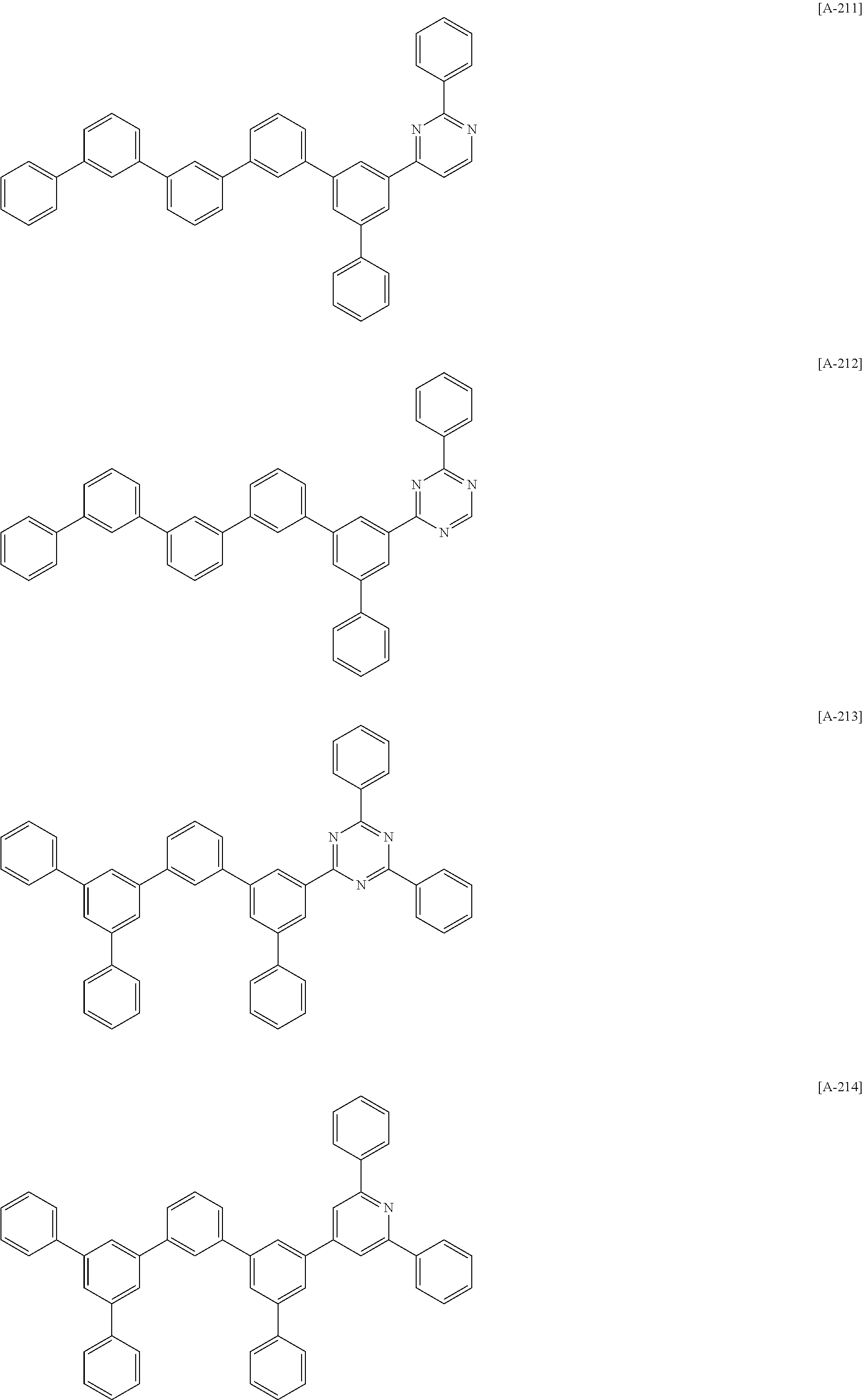 [SMILES: c1ccc(-c2cccc(-c3cccc(-c4cccc(-c5cc(-c6ccccc6)cc(-c6ccnc(-c7ccccc7)n6)c5)c4)c3)c2)cc1 ; c1ccc(-c2cccc(-c3cccc(-c4cccc(-c5cc(-c6ccccc6)cc(-c6ncnc(-c7ccccc7)n6)c5)c4)c3)c2)cc1 ; c1ccc(-c2cc(-c3ccccc3)cc(-c3cccc(-c4cc(-c5ccccc5)cc(-c5nc(-c6ccccc6)nc(-c6ccccc6)n5)c4)c3)c2)cc1 ; c1ccc(-c2cc(-c3ccccc3)cc(-c3cccc(-c4cc(-c5ccccc5)cc(-c5cc(-c6ccccc6)nc(-c6ccccc6)c5)c4)c3)c2)cc1]

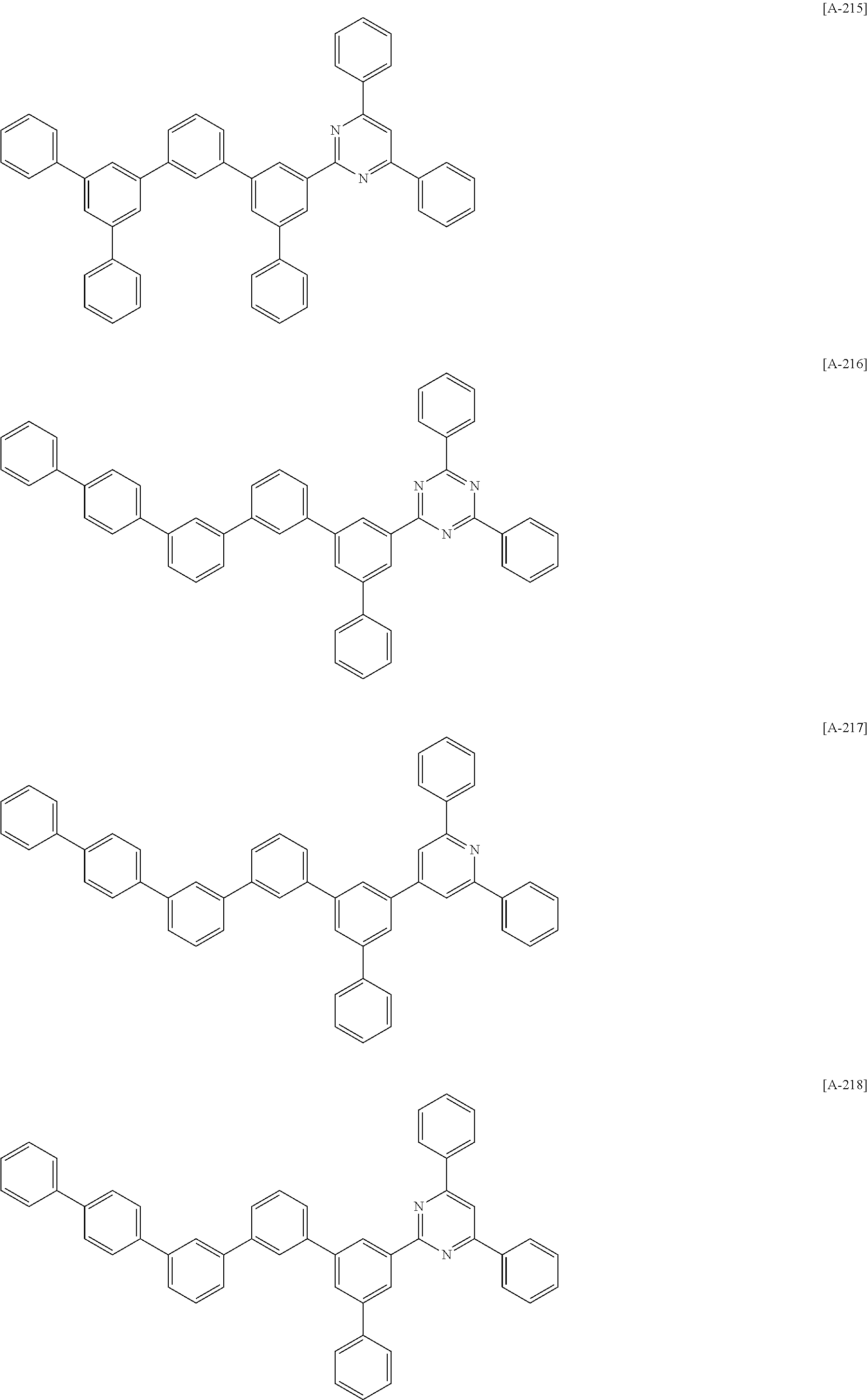 [SMILES: c1ccc(-c2cc(-c3ccccc3)cc(-c3cccc(-c4cc(-c5ccccc5)cc(-c5nc(-c6ccccc6)cc(-c6ccccc6)n5)c4)c3)c2)cc1 ; c1ccc(-c2ccc(-c3cccc(-c4cccc(-c5cc(-c6ccccc6)cc(-c6nc(-c7ccccc7)nc(-c7ccccc7)n6)c5)c4)c3)cc2)cc1 ; c1ccc(-c2ccc(-c3cccc(-c4cccc(-c5cc(-c6ccccc6)cc(-c6cc(-c7ccccc7)nc(-c7ccccc7)c6)c5)c4)c3)cc2)cc1 ; c1ccc(-c2ccc(-c3cccc(-c4cccc(-c5cc(-c6ccccc6)cc(-c6nc(-c7ccccc7)cc(-c7ccccc7)n6)c5)c4)c3)cc2)cc1]

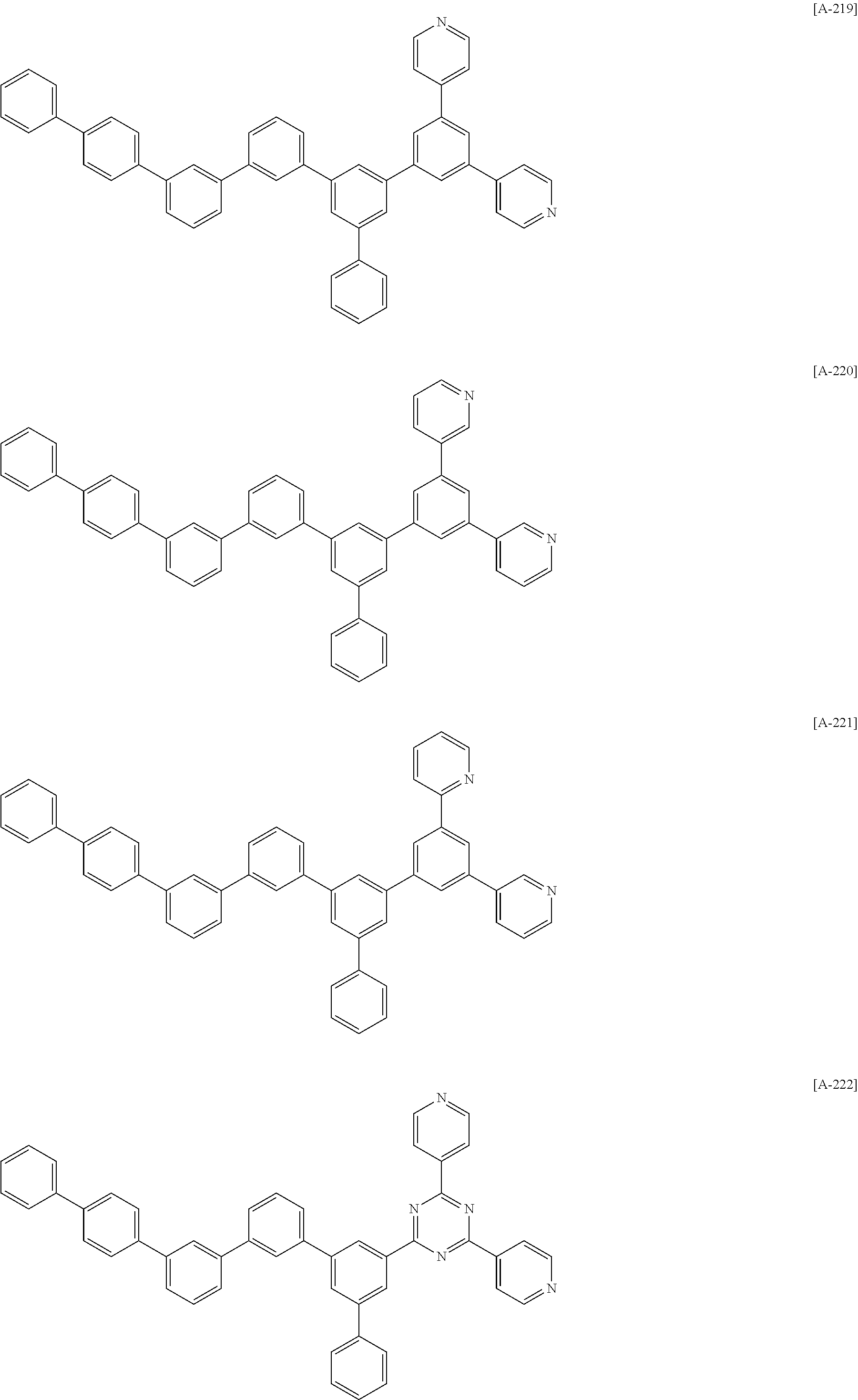 [SMILES: c1ccc(-c2ccc(-c3cccc(-c4cccc(-c5cc(-c6ccccc6)cc(-c6cc(-c7ccncc7)cc(-c7ccncc7)c6)c5)c4)c3)cc2)cc1 ; c1ccc(-c2ccc(-c3cccc(-c4cccc(-c5cc(-c6ccccc6)cc(-c6cc(-c7cccnc7)cc(-c7cccnc7)c6)c5)c4)c3)cc2)cc1 ; c1ccc(-c2ccc(-c3cccc(-c4cccc(-c5cc(-c6ccccc6)cc(-c6cc(-c7cccnc7)cc(-c7ccccn7)c6)c5)c4)c3)cc2)cc1 ; c1ccc(-c2ccc(-c3cccc(-c4cccc(-c5cc(-c6ccccc6)cc(-c6nc(-c7ccncc7)nc(-c7ccncc7)n6)c5)c4)c3)cc2)cc1]

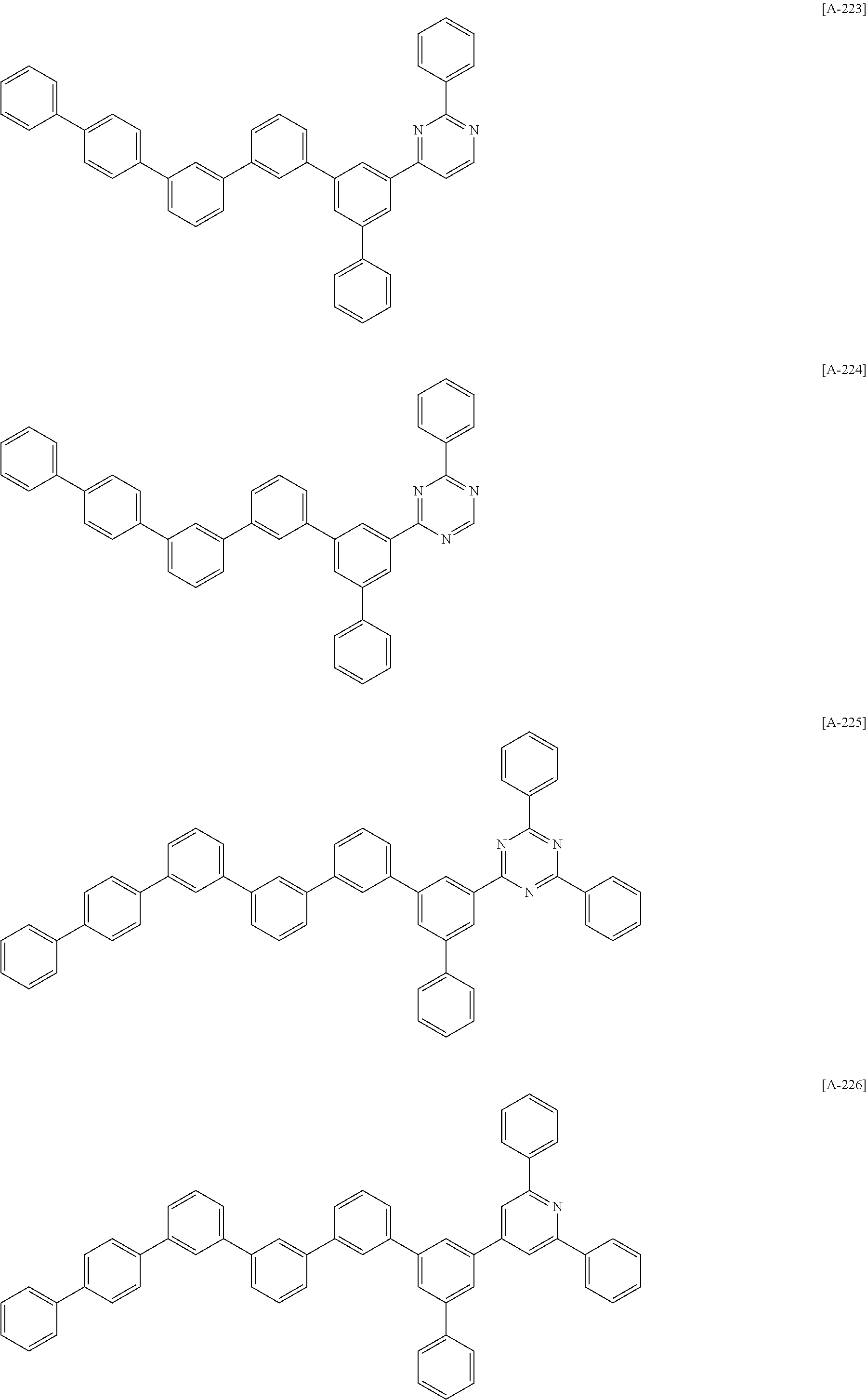 [SMILES: c1ccc(-c2ccc(-c3cccc(-c4cccc(-c5cc(-c6ccccc6)cc(-c6ccnc(-c7ccccc7)n6)c5)c4)c3)cc2)cc1 ; c1ccc(-c2ccc(-c3cccc(-c4cccc(-c5cc(-c6ccccc6)cc(-c6ncnc(-c7ccccc7)n6)c5)c4)c3)cc2)cc1 ; c1ccc(-c2ccc(-c3cccc(-c4cccc(-c5cccc(-c6cc(-c7ccccc7)cc(-c7nc(-c8ccccc8)nc(-c8ccccc8)n7)c6)c5)c4)c3)cc2)cc1 ; c1ccc(-c2ccc(-c3cccc(-c4cccc(-c5cccc(-c6cc(-c7ccccc7)cc(-c7cc(-c8ccccc8)nc(-c8ccccc8)c7)c6)c5)c4)c3)cc2)cc1]

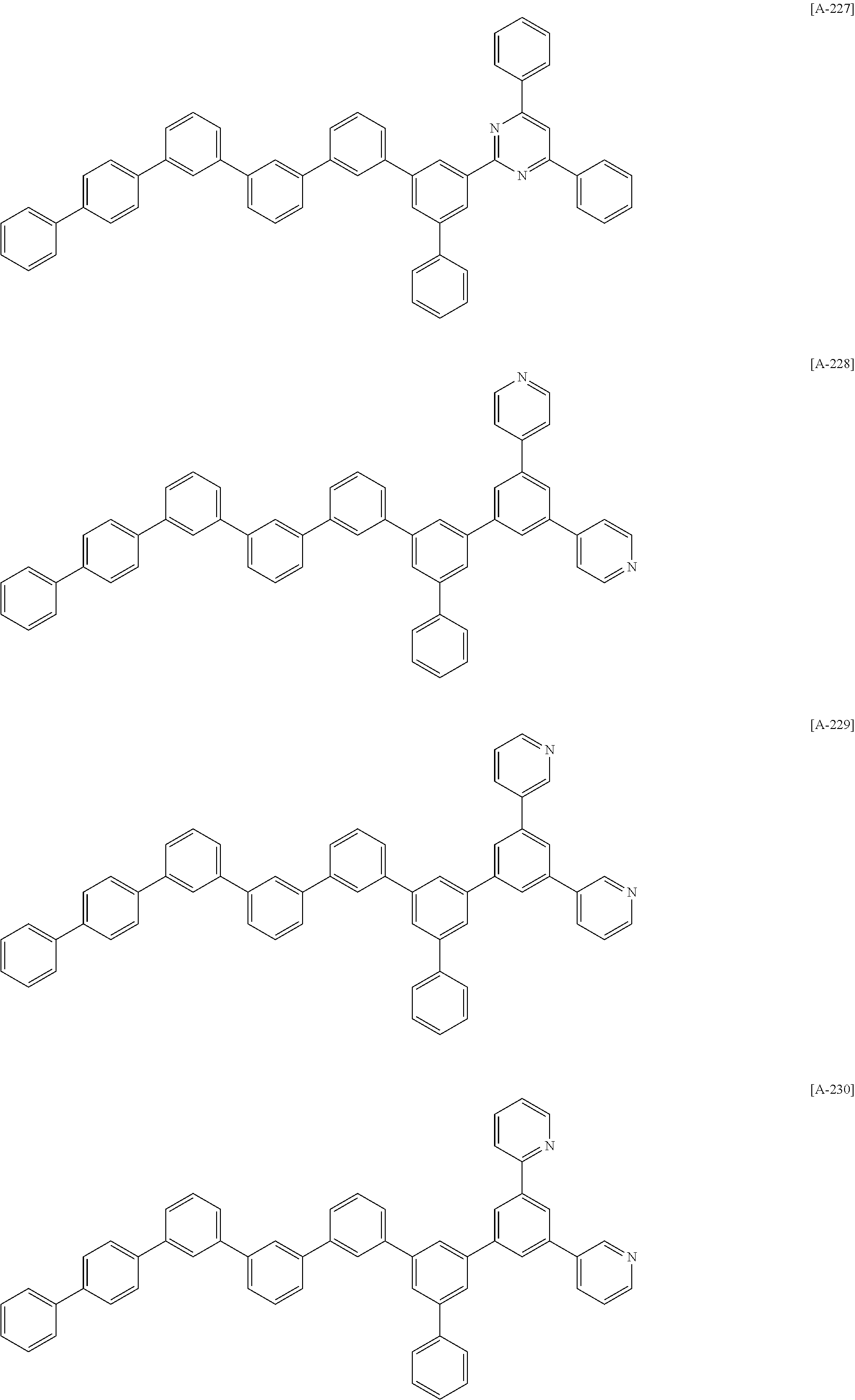 [SMILES: c1ccc(-c2ccc(-c3cccc(-c4cccc(-c5cccc(-c6cc(-c7ccccc7)cc(-c7nc(-c8ccccc8)cc(-c8ccccc8)n7)c6)c5)c4)c3)cc2)cc1 ; c1ccc(-c2ccc(-c3cccc(-c4cccc(-c5cccc(-c6cc(-c7ccccc7)cc(-c7cc(-c8ccncc8)cc(-c8ccncc8)c7)c6)c5)c4)c3)cc2)cc1 ; c1ccc(-c2ccc(-c3cccc(-c4cccc(-c5cccc(-c6cc(-c7ccccc7)cc(-c7cc(-c8cccnc8)cc(-c8cccnc8)c7)c6)c5)c4)c3)cc2)cc1 ; c1ccc(-c2ccc(-c3cccc(-c4cccc(-c5cccc(-c6cc(-c7ccccc7)cc(-c7cc(-c8cccnc8)cc(-c8ccccn8)c7)c6)c5)c4)c3)cc2)cc1]

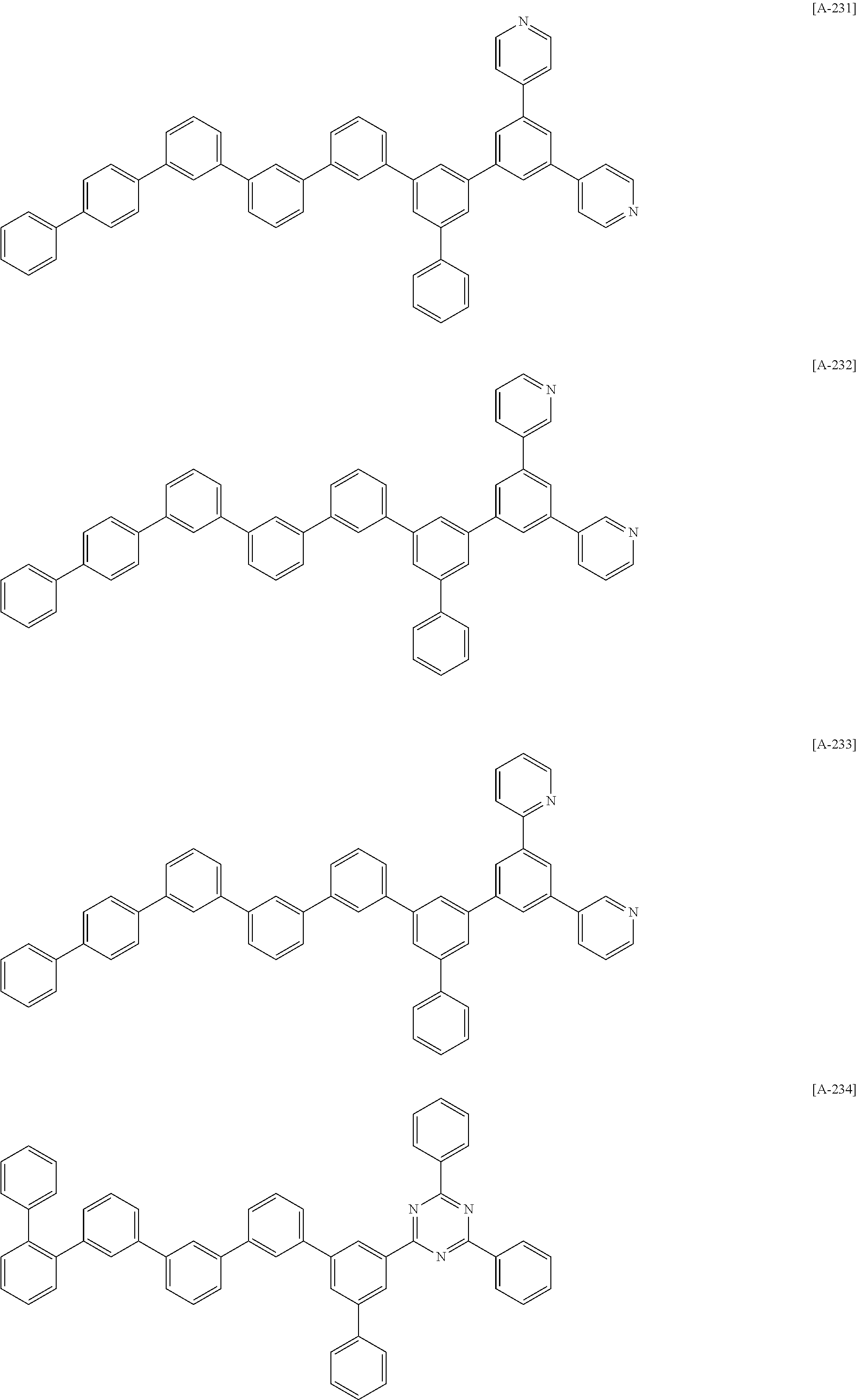

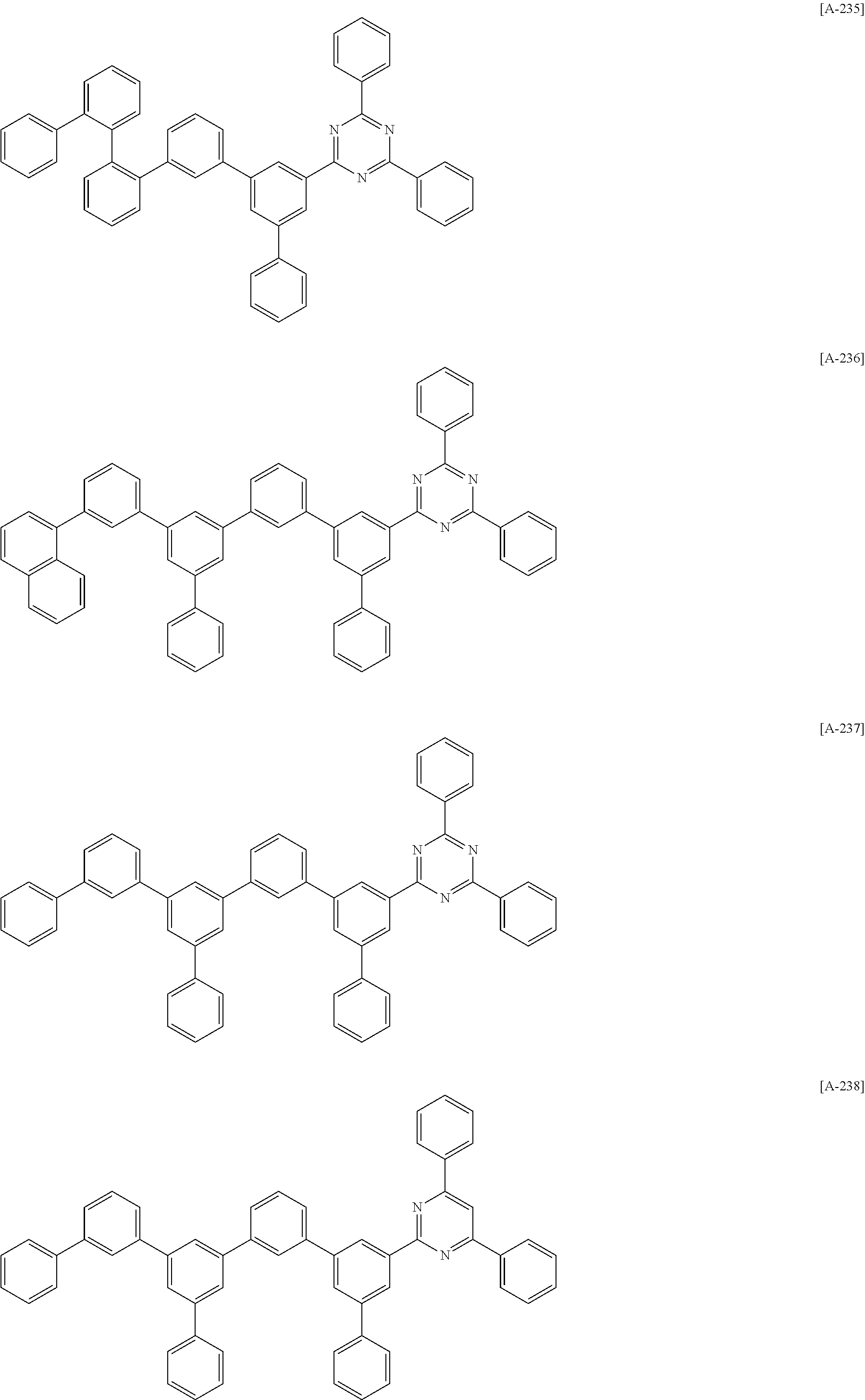 [SMILES: c1ccc(-c2cc(-c3cccc(-c4ccccc4-c4ccccc4-c4ccccc4)c3)cc(-c3nc(-c4ccccc4)nc(-c4ccccc4)n3)c2)cc1 ; c1ccc(-c2cc(-c3cccc(-c4cc(-c5ccccc5)cc(-c5nc(-c6ccccc6)nc(-c6ccccc6)n5)c4)c3)cc(-c3cccc(-c4cccc5ccccc45)c3)c2)cc1 ; c1ccc(-c2cccc(-c3cc(-c4ccccc4)cc(-c4cccc(-c5cc(-c6ccccc6)cc(-c6nc(-c7ccccc7)nc(-c7ccccc7)n6)c5)c4)c3)c2)cc1 ; c1ccc(-c2cccc(-c3cc(-c4ccccc4)cc(-c4cccc(-c5cc(-c6ccccc6)cc(-c6nc(-c7ccccc7)cc(-c7ccccc7)n6)c5)c4)c3)c2)cc1]

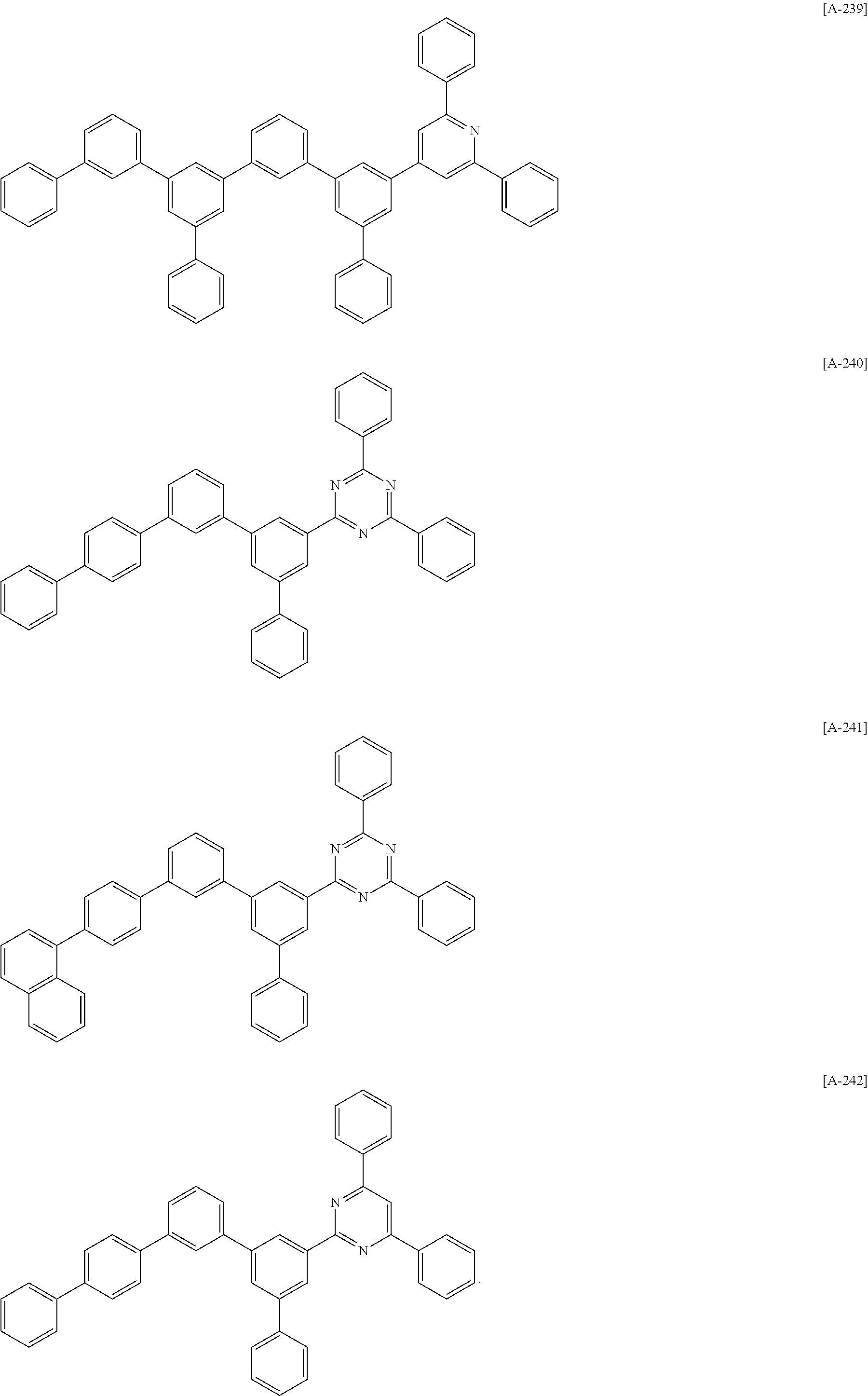 [SMILES: c1ccc(-c2cccc(-c3cc(-c4ccccc4)cc(-c4cccc(-c5cc(-c6ccccc6)cc(-c6cc(-c7ccccc7)nc(-c7ccccc7)c6)c5)c4)c3)c2)cc1 ; c1ccc(-c2ccc(-c3cccc(-c4cc(-c5ccccc5)cc(-c5nc(-c6ccccc6)nc(-c6ccccc6)n5)c4)c3)cc2)cc1 ; c1ccc(-c2cc(-c3cccc(-c4ccc(-c5cccc6ccccc56)cc4)c3)cc(-c3nc(-c4ccccc4)nc(-c4ccccc4)n3)c2)cc1 ; c1ccc(-c2ccc(-c3cccc(-c4cc(-c5ccccc5)cc(-c5nc(-c6ccccc6)cc(-c6ccccc6)n5)c4)c3)cc2)cc1]

The first compound may be used with at least one second compound having a carbazole moiety in an emission layer.

The second compound may be represented by Chemical Formula 2.

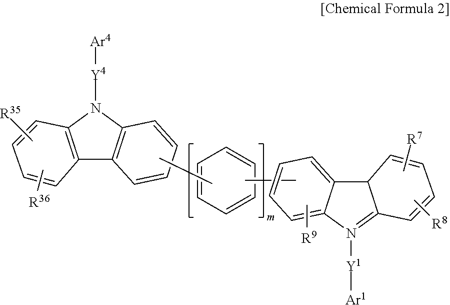

In Chemical Formula 2,

Y¹ and Y⁴ are independently a single bond, a substituted or unsubstituted C6 to C30 arylene group, a substituted or unsubstituted C2 to C30 heteroarylene group, or a combination thereof,

Ar¹ and Ar⁴ are independently a substituted or unsubstituted C6 to C30 aryl group, a substituted or unsubstituted C2 to C30 heterocyclic group, or a combination thereof,

R⁷ to R⁹, R³⁵, and R³⁶ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, and

m is one of integers of 0 to 4.

The compound represented by Chemical Formula 2 has bipolar characteristics in which hole characteristics are relatively strong and may increase charge mobility and stability when used with the first compound for an emission layer and simultaneously used for a hole transport auxiliary layer neighboring the emission layer and thus prevent accumulation of holes and/or electrons on the interface of the hole transport layer and the emission layer and increase a charge balance. Accordingly, luminous efficiency and life-span characteristics of an organic optoelectronic device may be significantly improved.

The Ar¹ and Ar⁴ of Chemical Formula 2 may be, for example, a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted benzofuranyl group, a substituted or unsubstituted benzothiophenyl group, a substituted or unsubstituted fluorenyl group, a substituted or unsubstituted dibenzothiophenyl group, a substituted or unsubstituted dibenzofuranyl group, a substituted or unsubstituted pyridinyl group, a substituted or unsubstituted pyrimidinyl group, or a combination thereof.

Specifically, Chemical Formula 2 may be one of structures of Group 3 and the .—Y¹—Ar¹ and *—Y⁴—Ar⁴ may independently be one of substituents of Group 4.

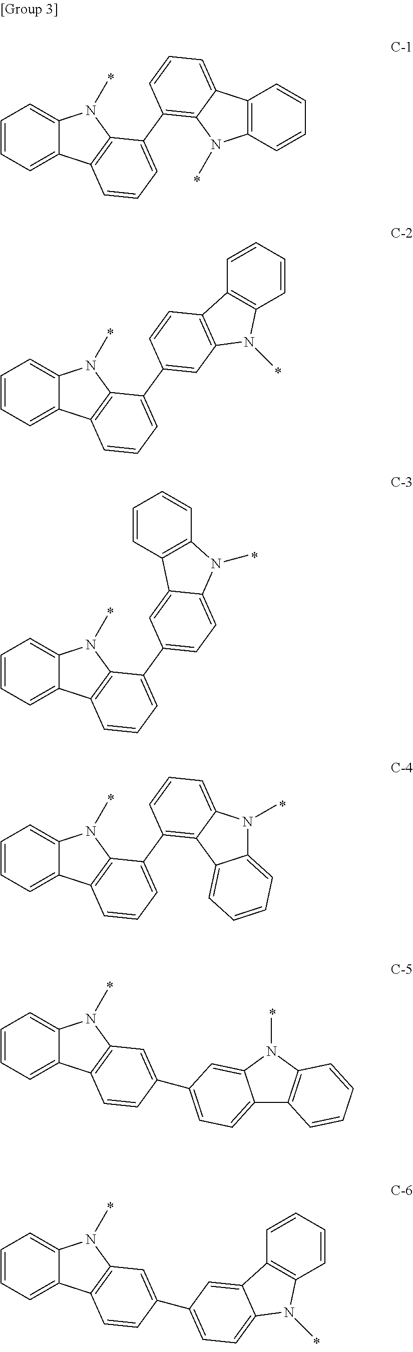

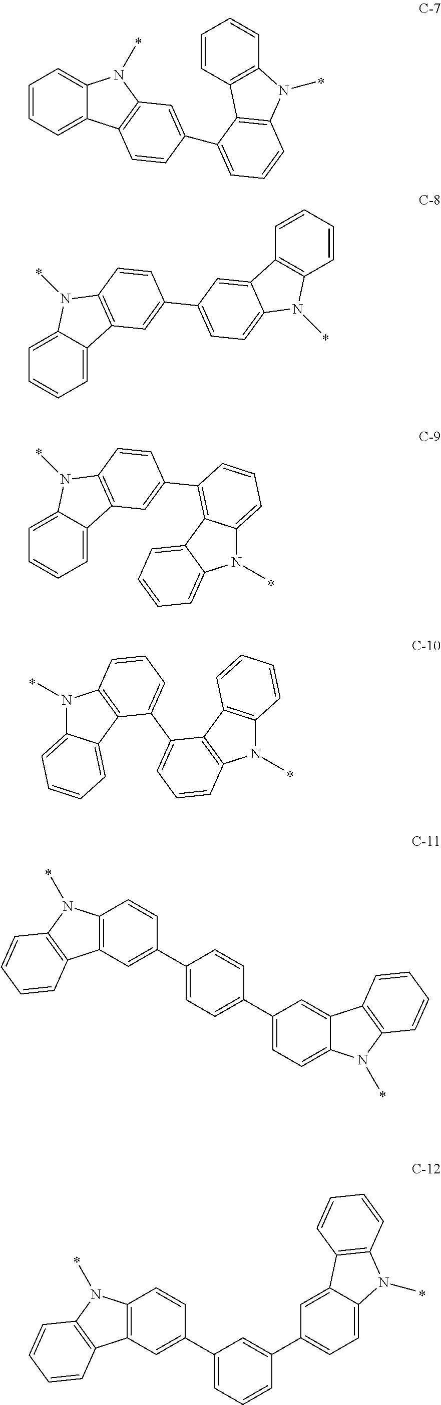

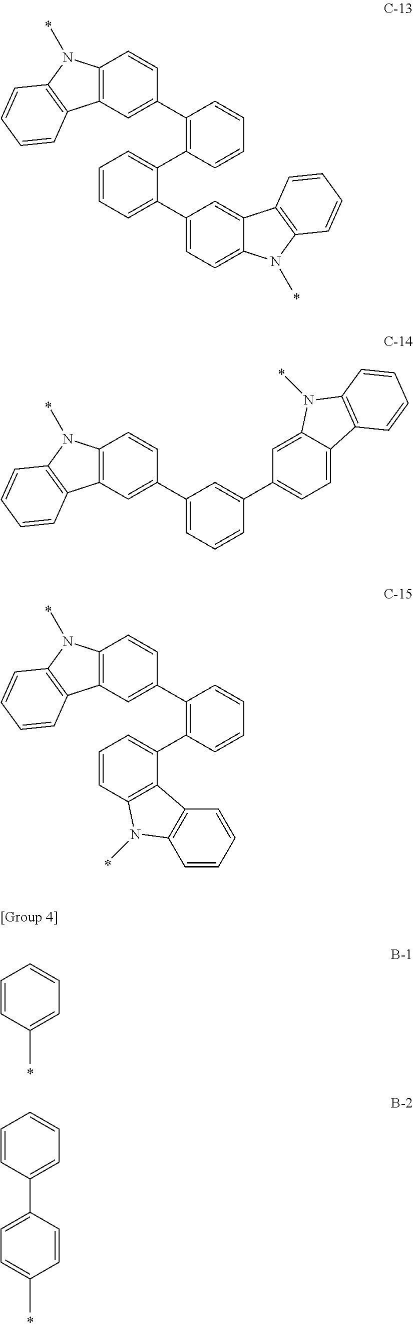

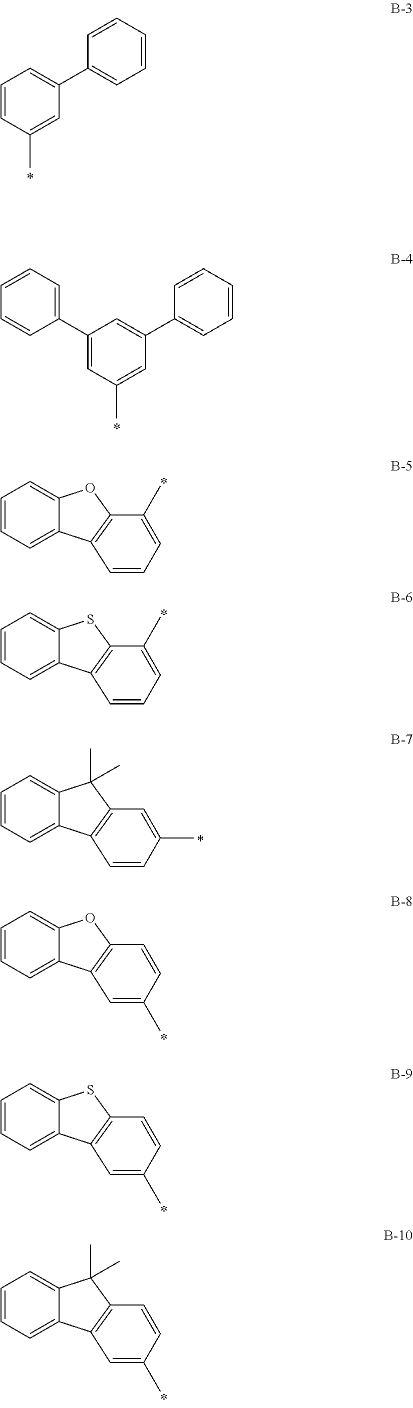

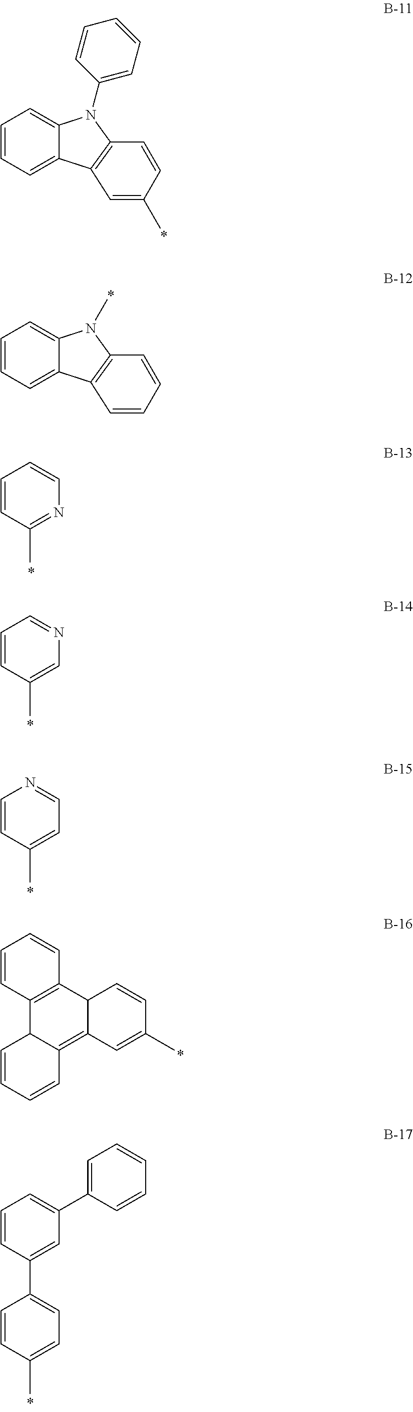

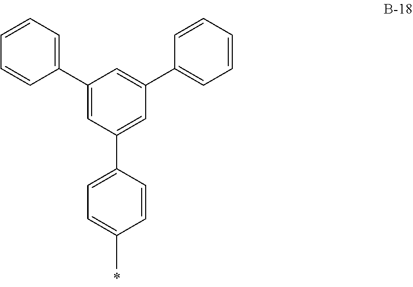 [SMILES: Cc1ccc(-c2cc(-c3ccccc3)cc(-c3ccccc3)c2)cc1]

In the groups 3 and 4, * indicates a linking point.

In addition, Ar¹ and Ar⁴ of Chemical Formula 2 may be selected from substituted or unsubstituted groups in Group 5 but are not limited thereto.

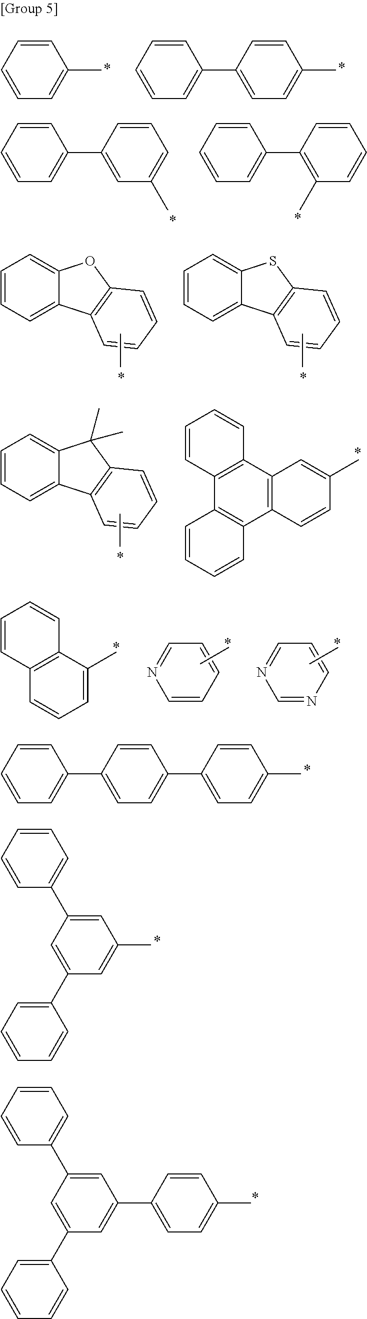

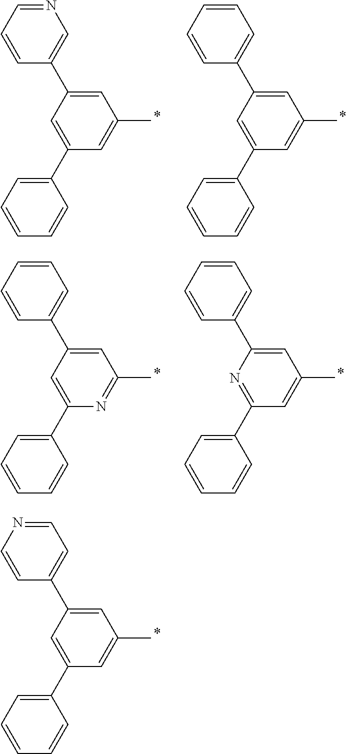

In Group 5, * indicates a linking point.

The second compound represented by Chemical Formula 2 may be for example compounds of Group B but is not limited thereto.

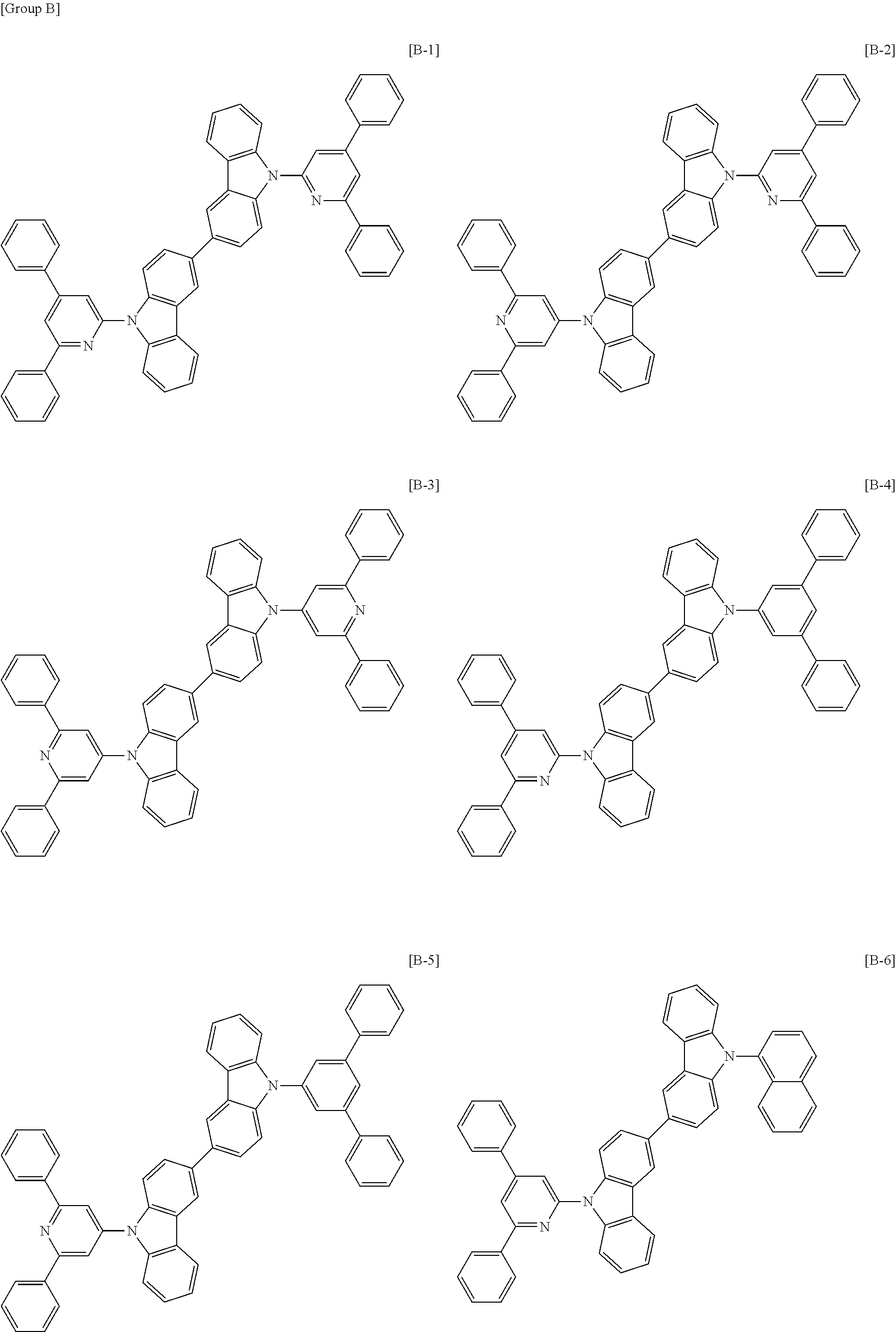

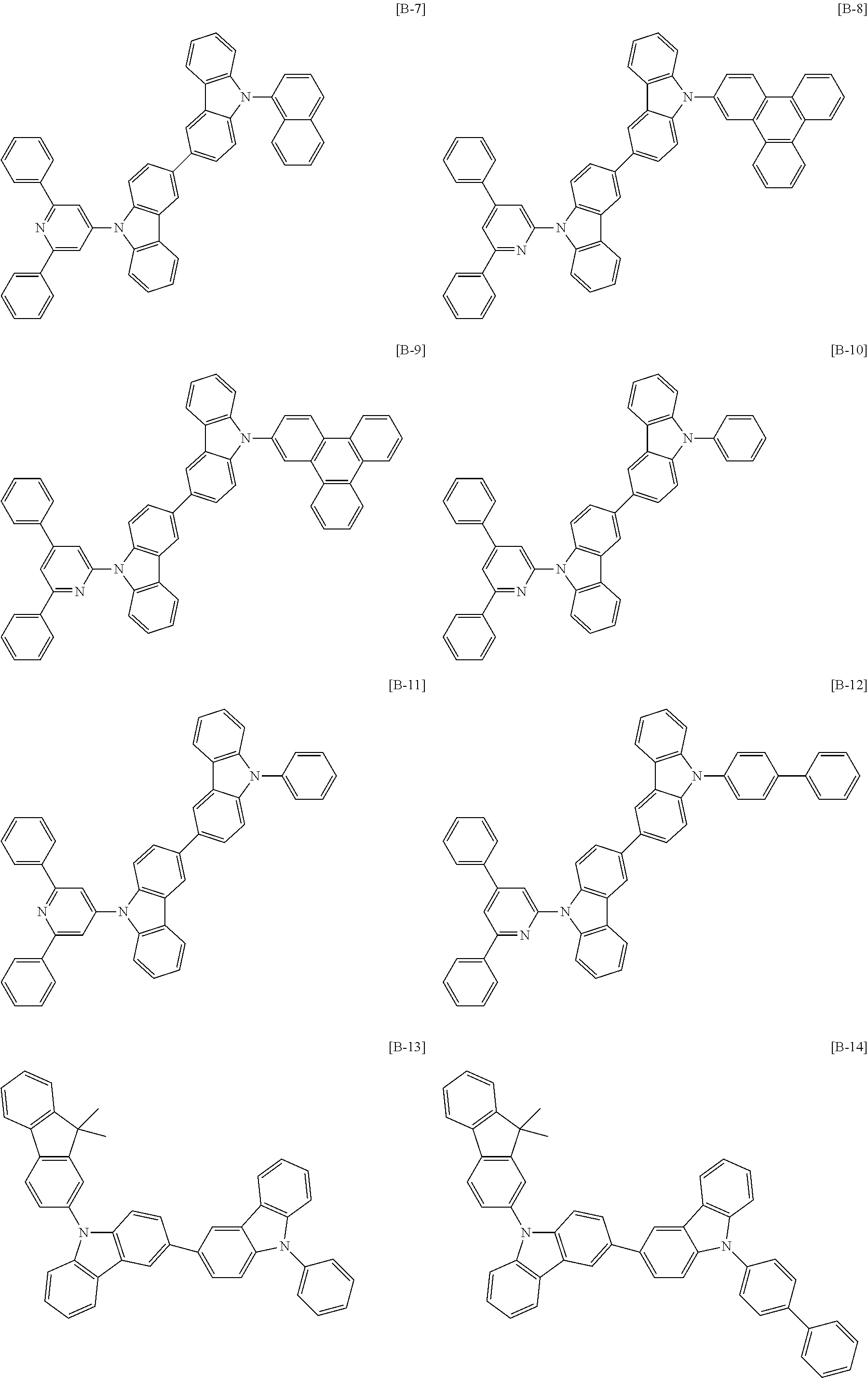

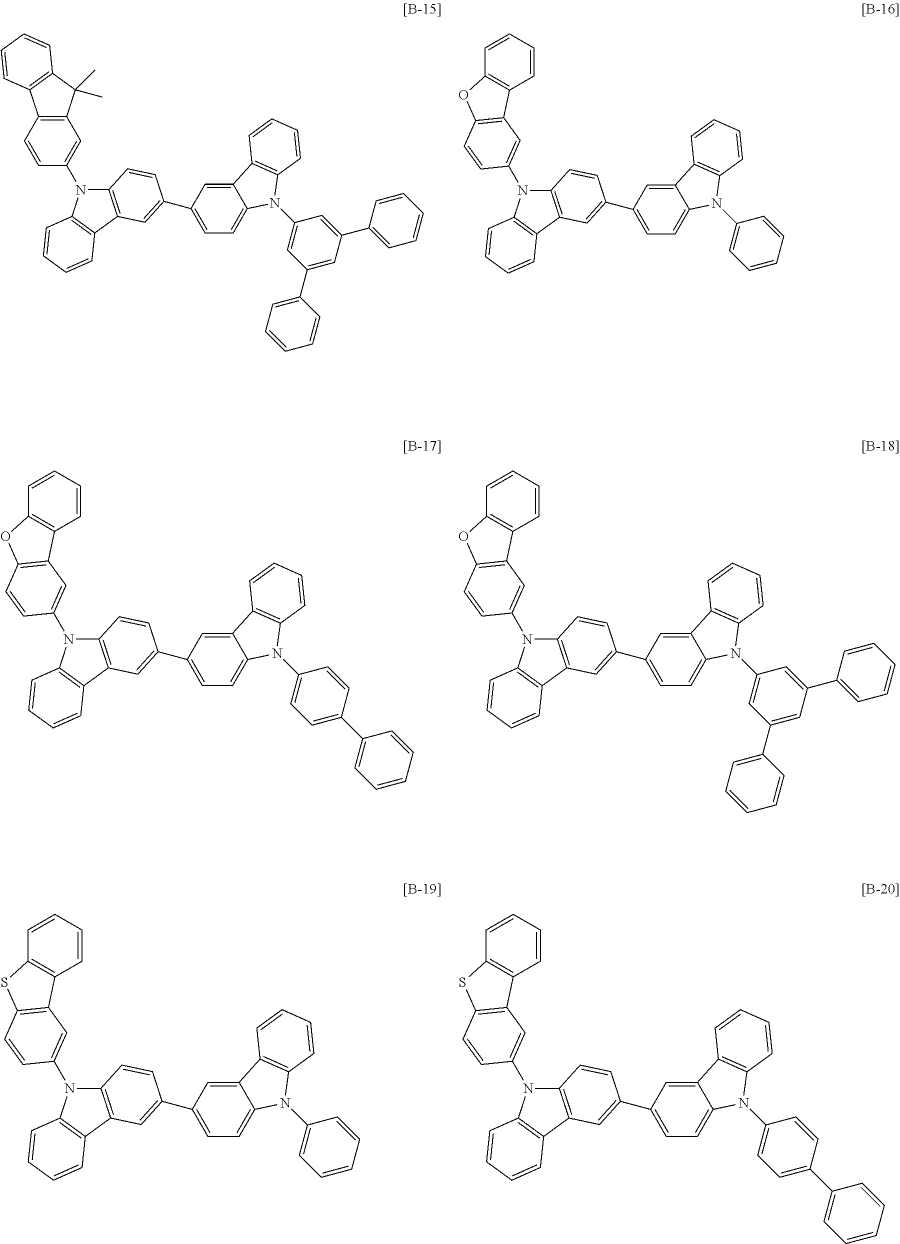

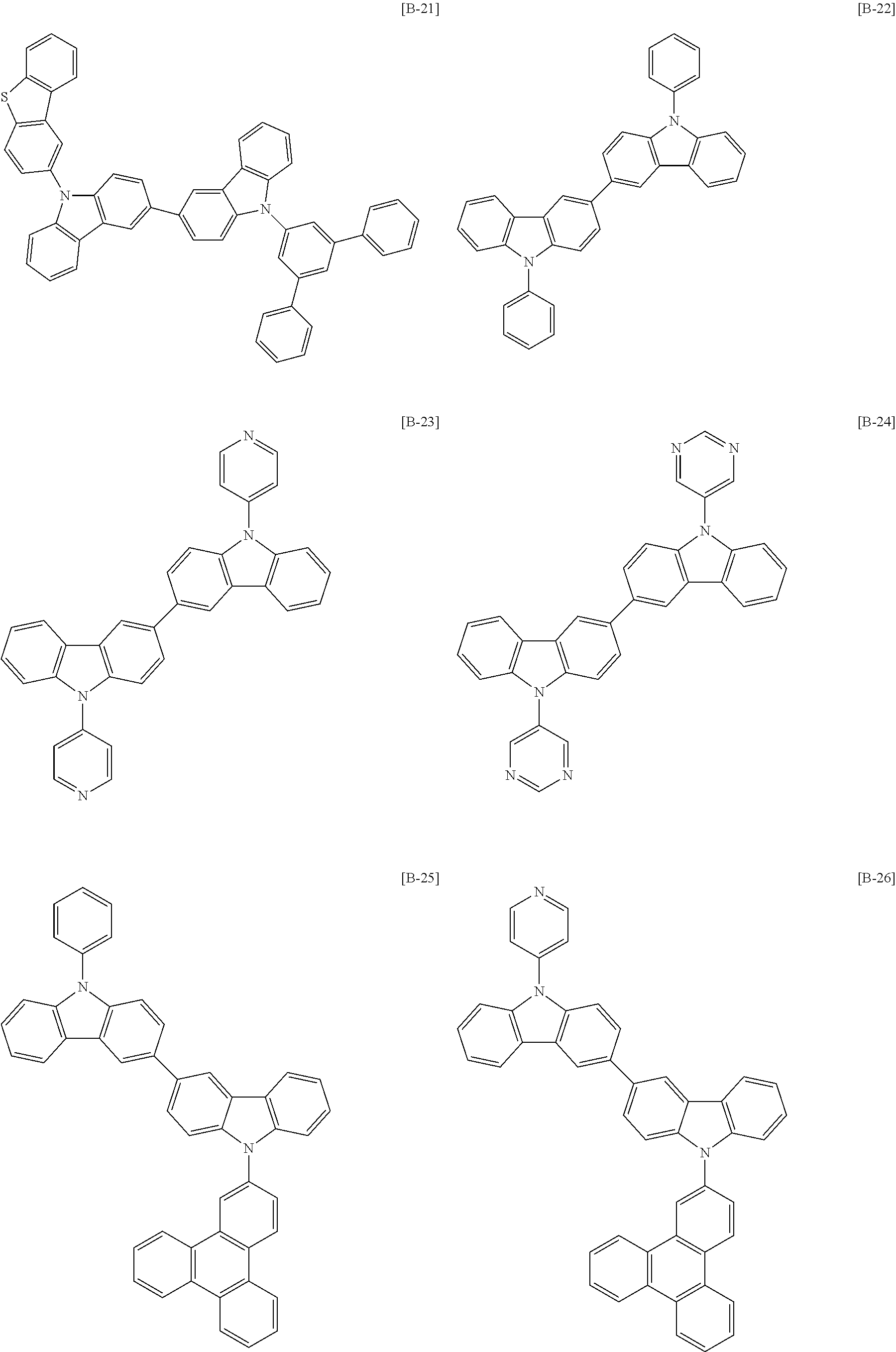

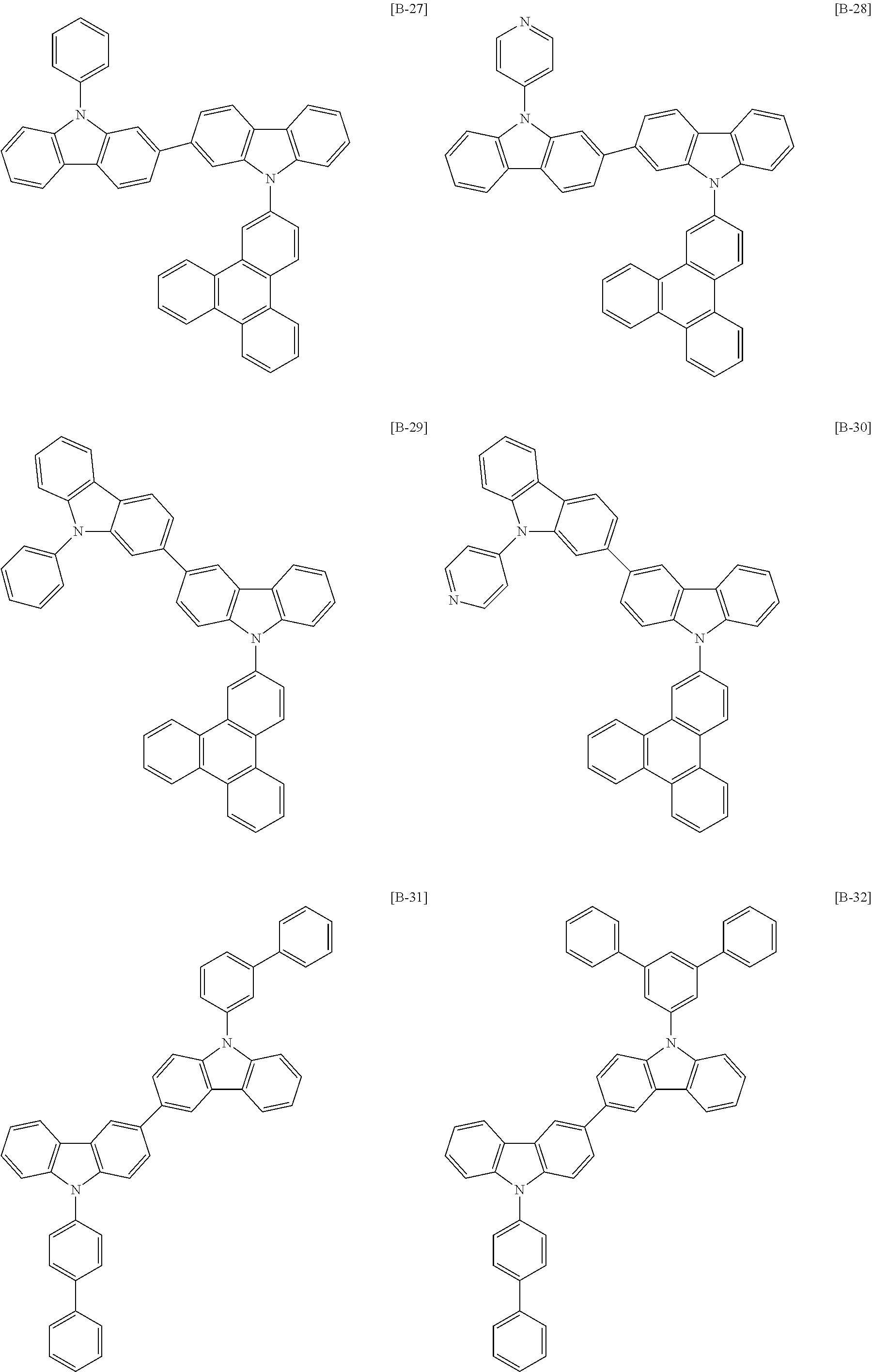

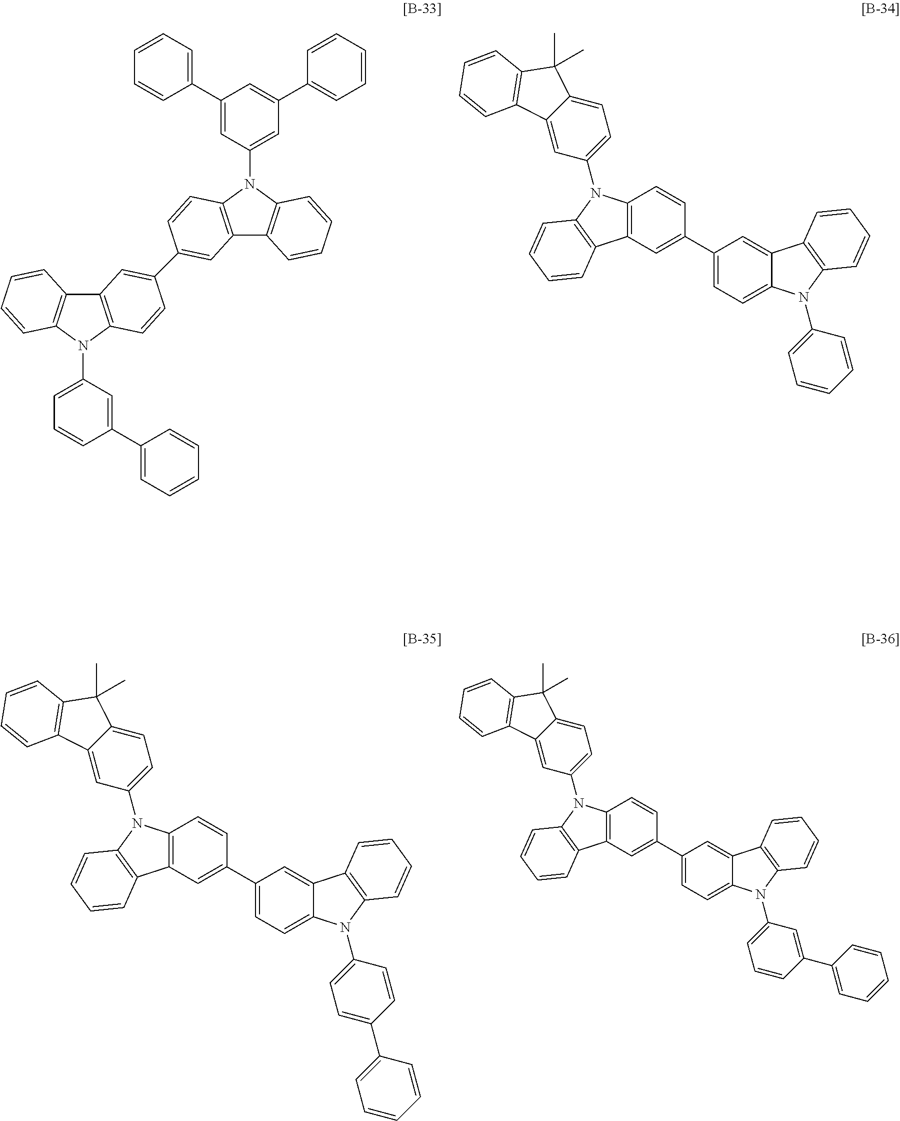 [SMILES: c1ccc(-c2cccc(-n3c4ccccc4c4cc(-c5ccc6c(c5)c5ccccc5n6-c5cc(-c6ccccc6)cc(-c6ccccc6)c5)ccc43)c2)cc1 ; CC1(C)c2ccccc2-c2cc(-n3c4ccccc4c4cc(-c5ccc6c(c5)c5ccccc5n6-c5ccccc5)ccc43)ccc21 ; CC1(C)c2ccccc2-c2cc(-n3c4ccccc4c4cc(-c5ccc6c(c5)c5ccccc5n6-c5ccc(-c6ccccc6)cc5)ccc43)ccc21 ; CC1(C)c2ccccc2-c2cc(-n3c4ccccc4c4cc(-c5ccc6c(c5)c5ccccc5n6-c5cccc(-c6ccccc6)c5)ccc43)ccc21]

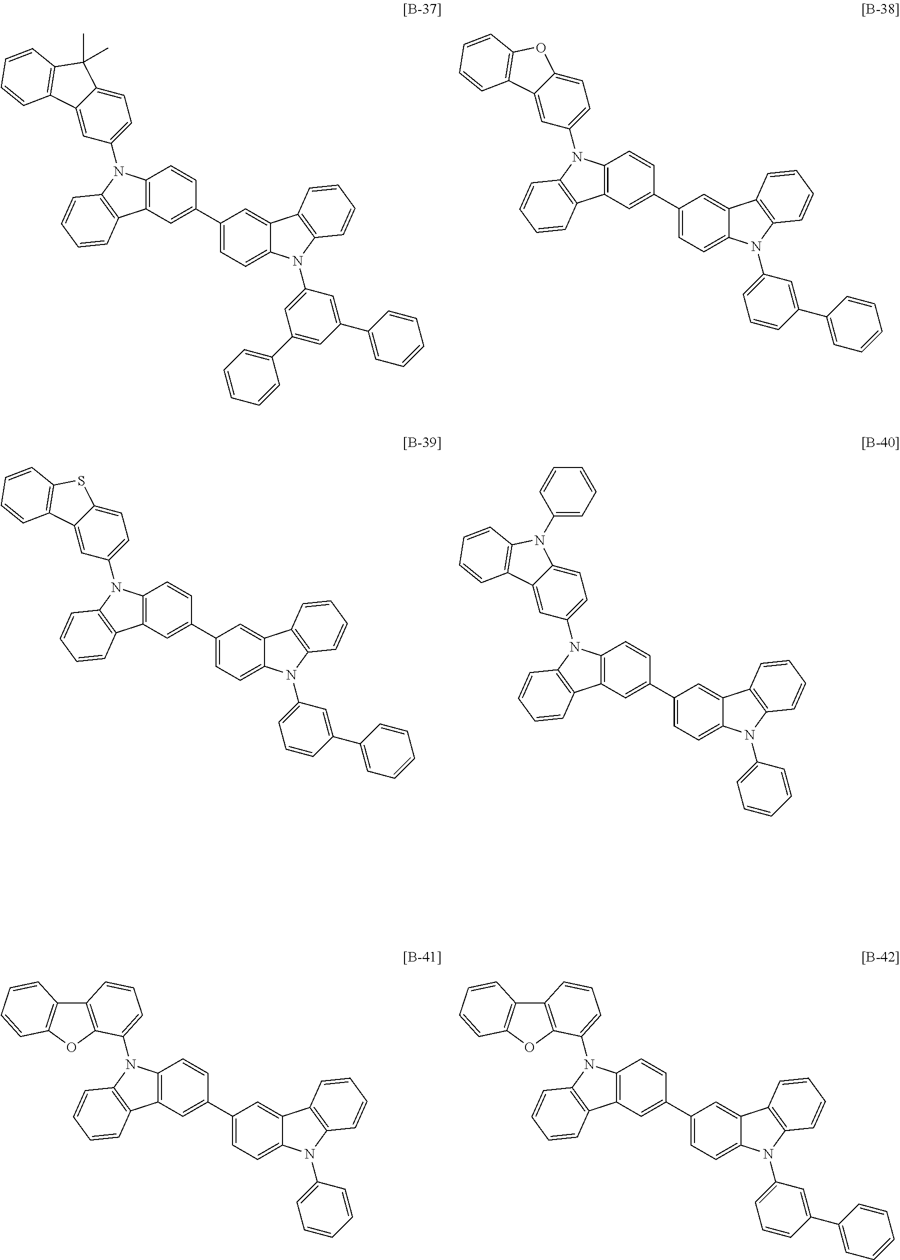

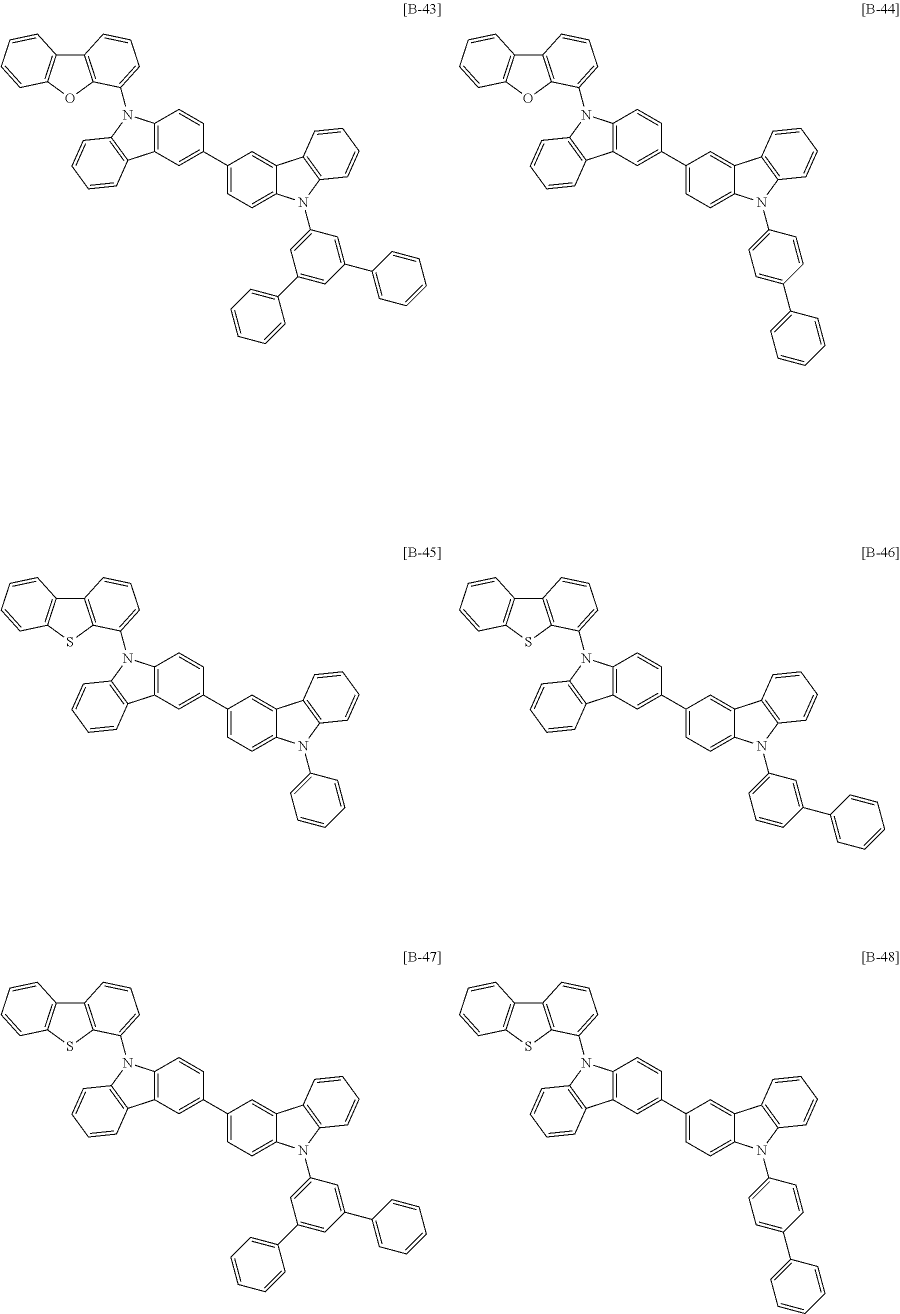

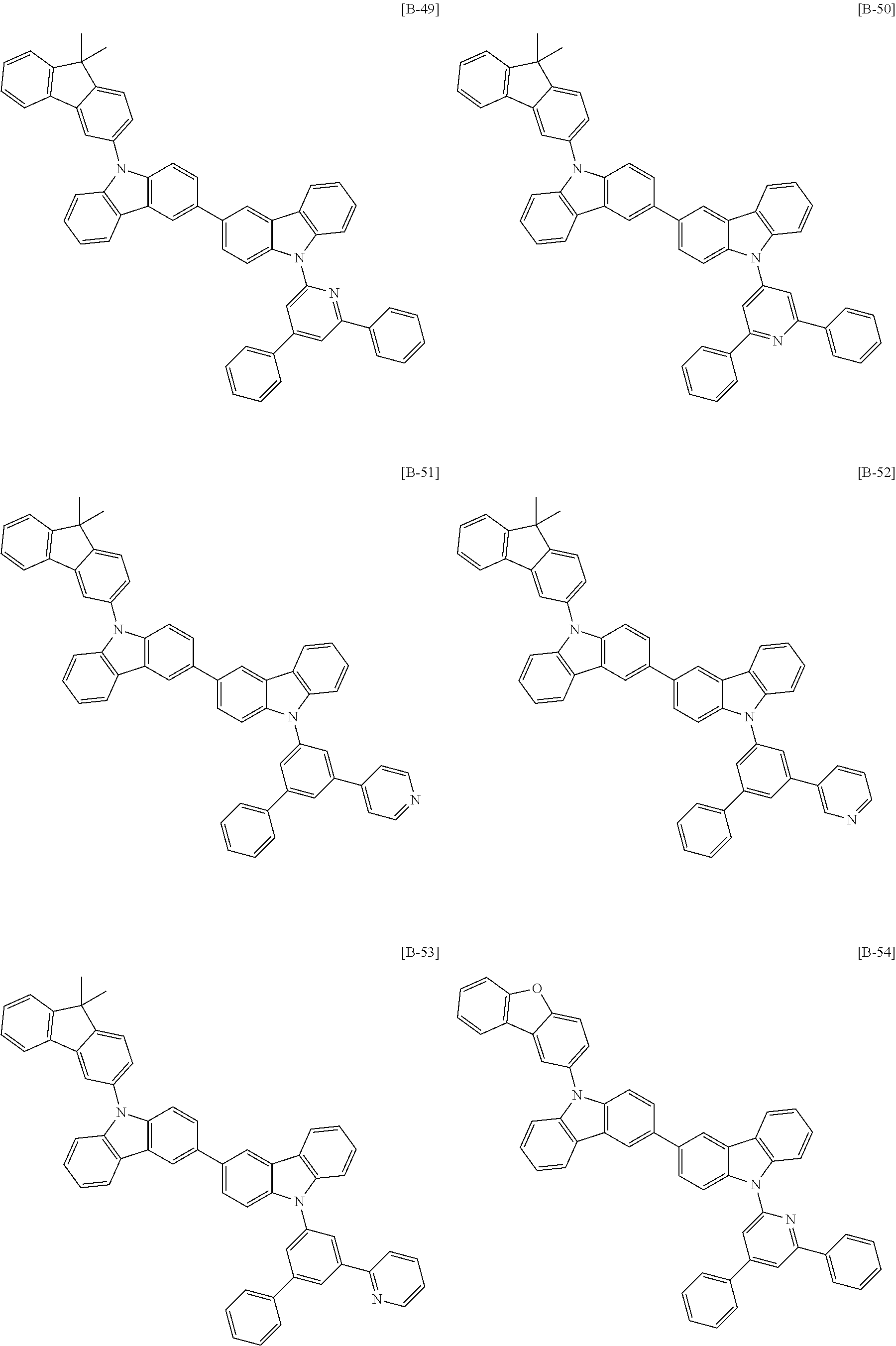

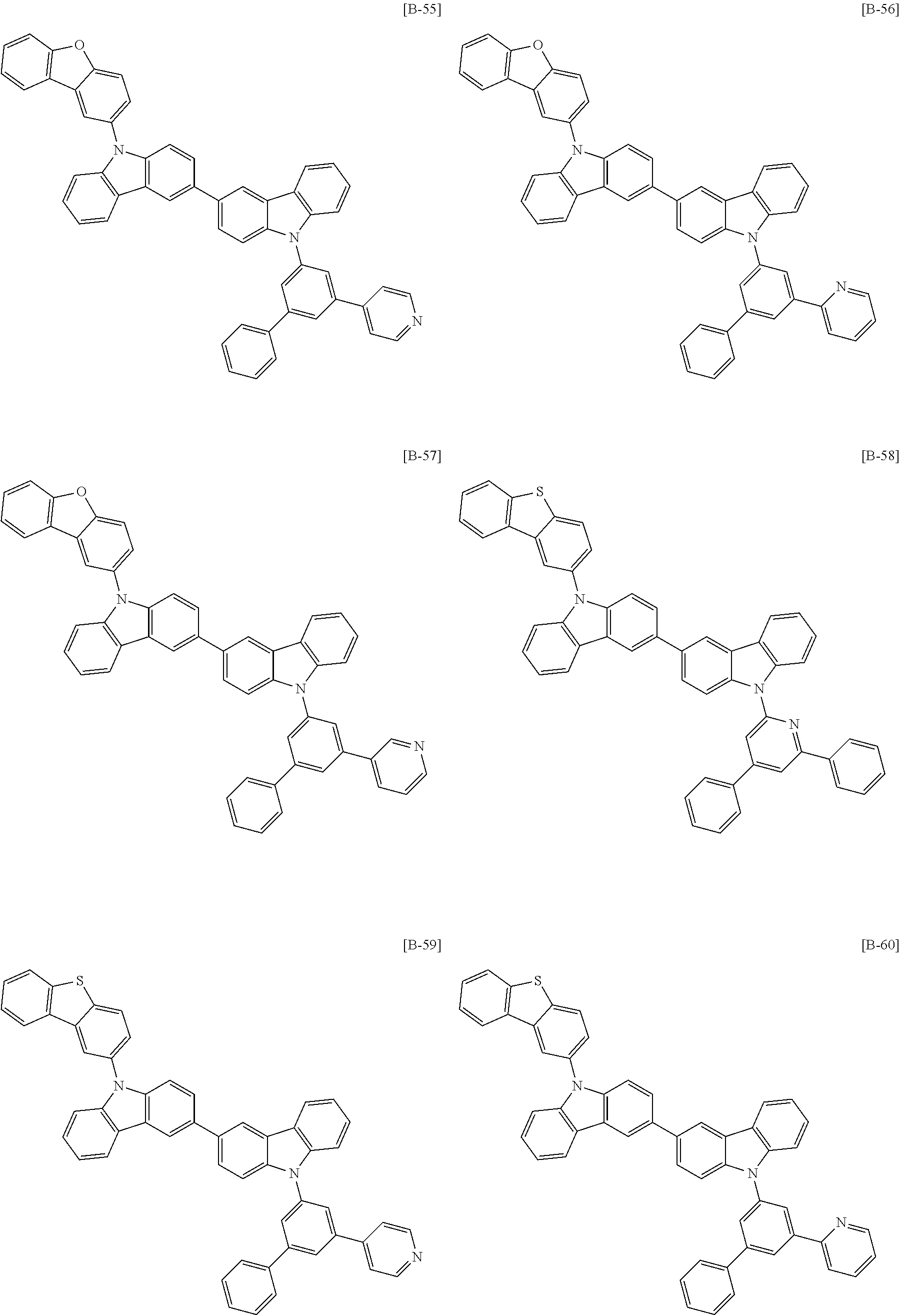

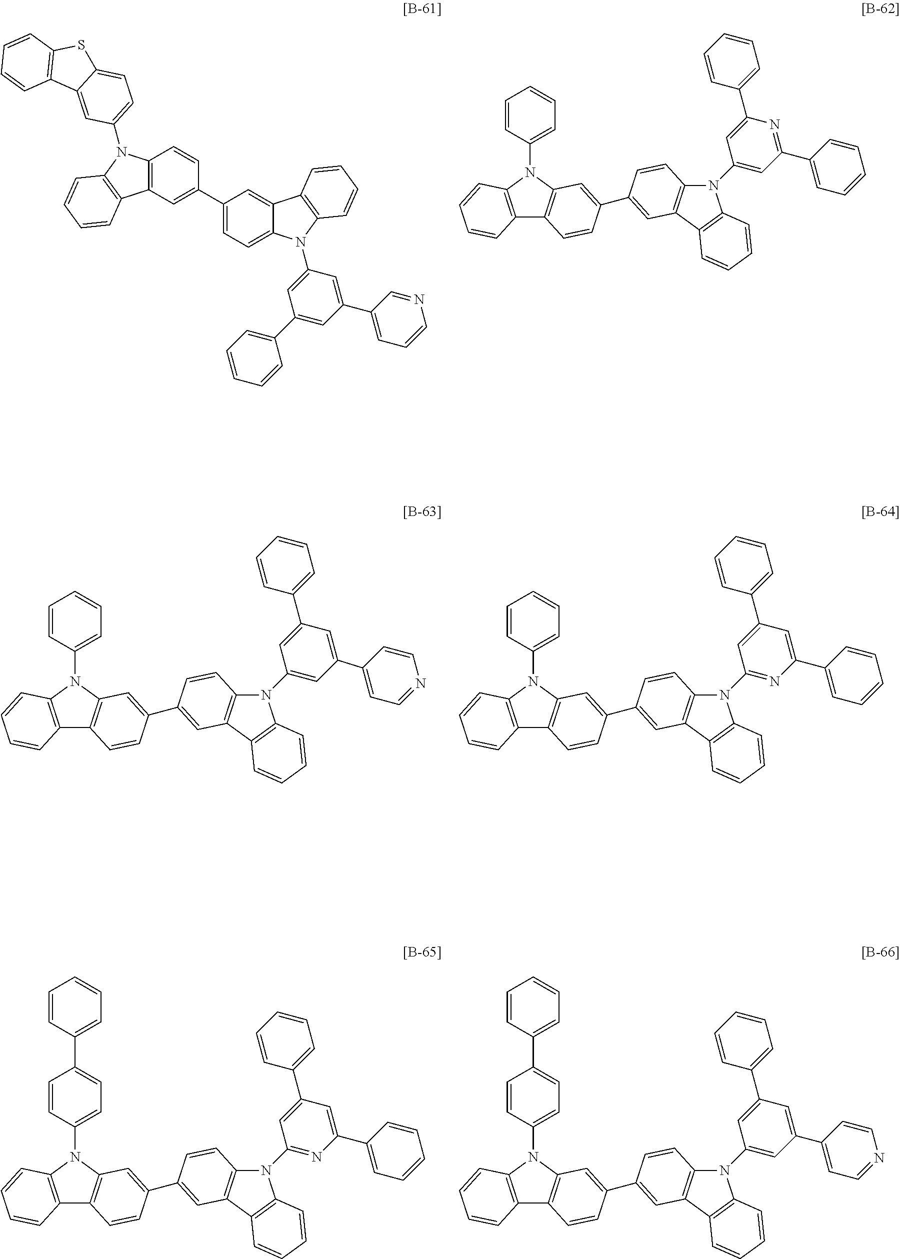

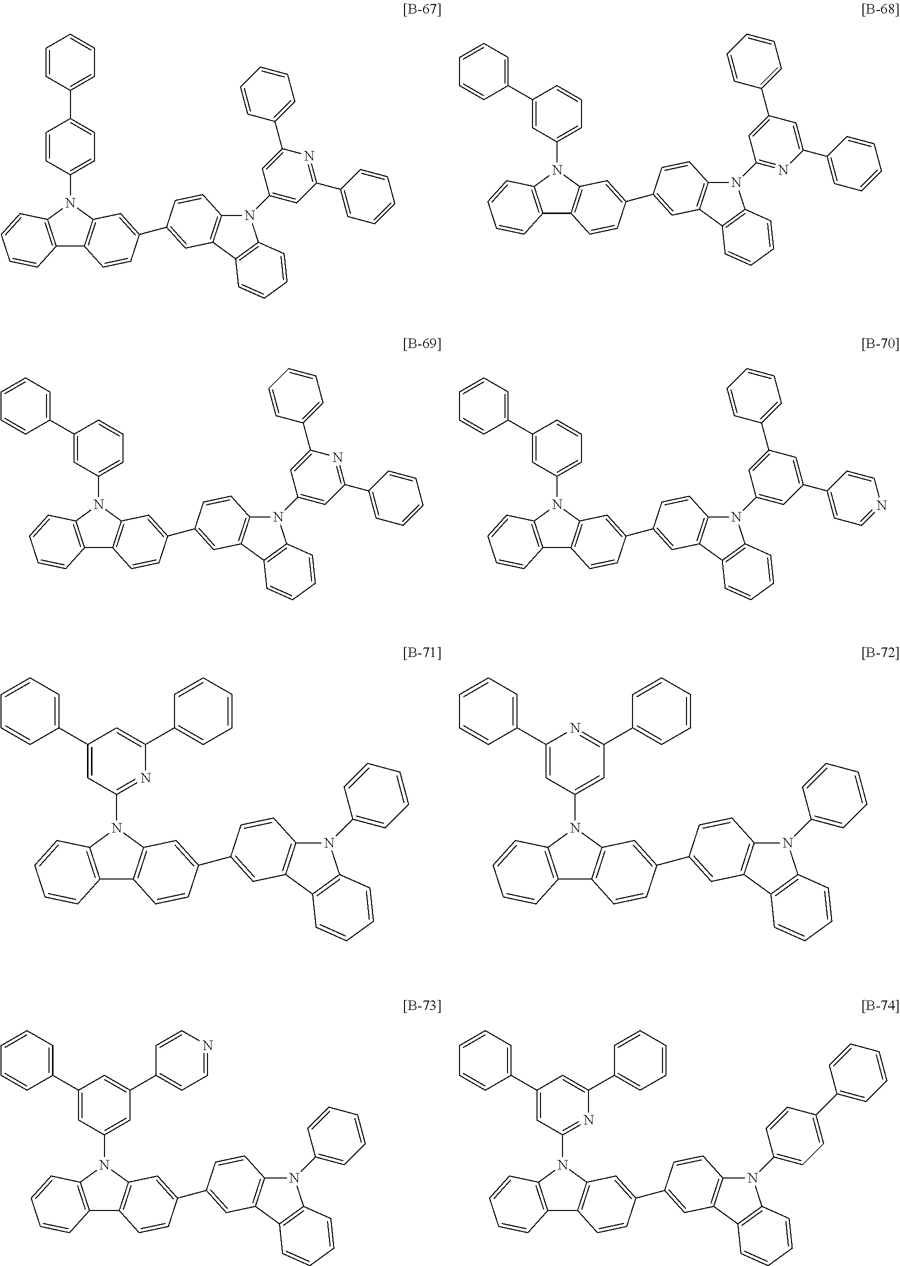

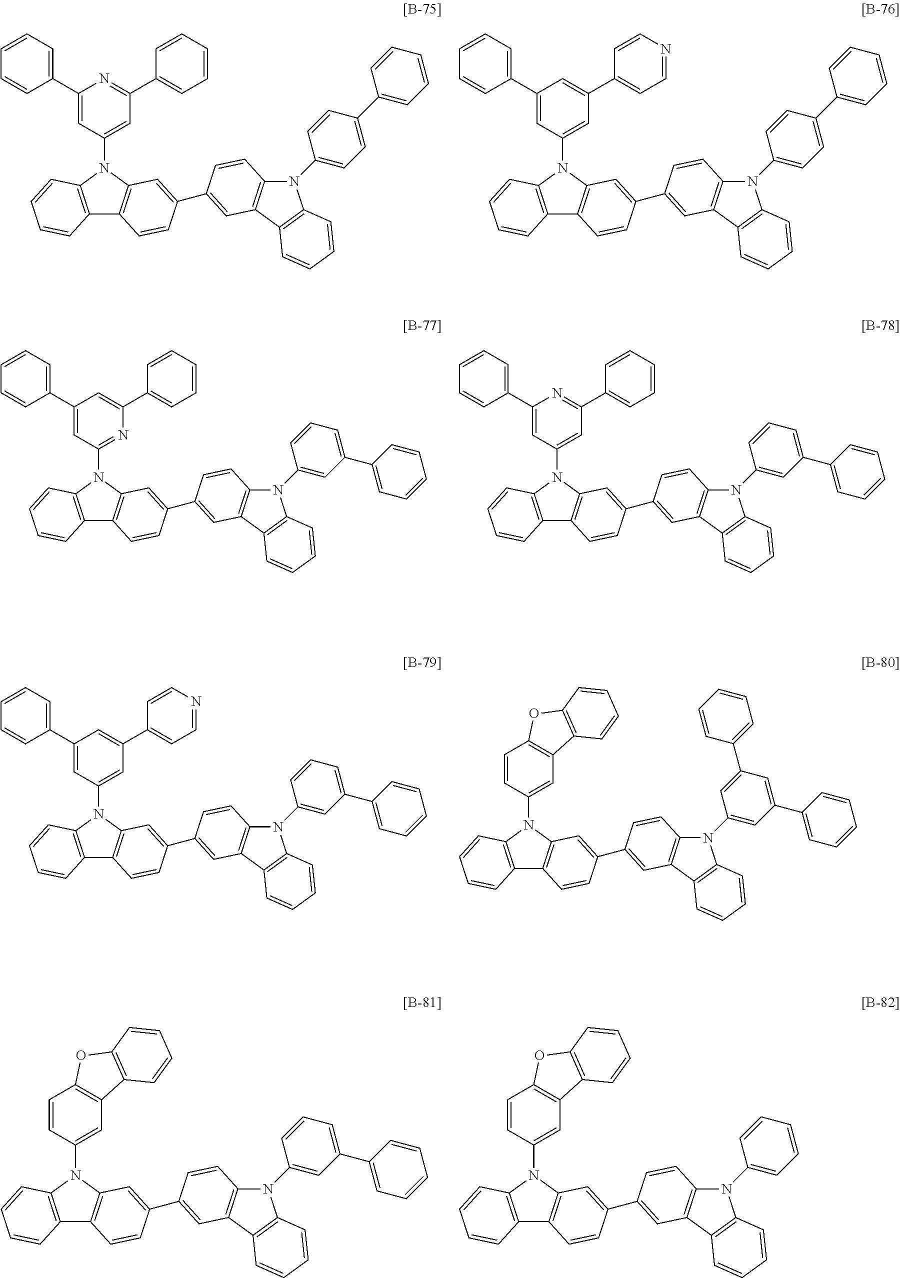

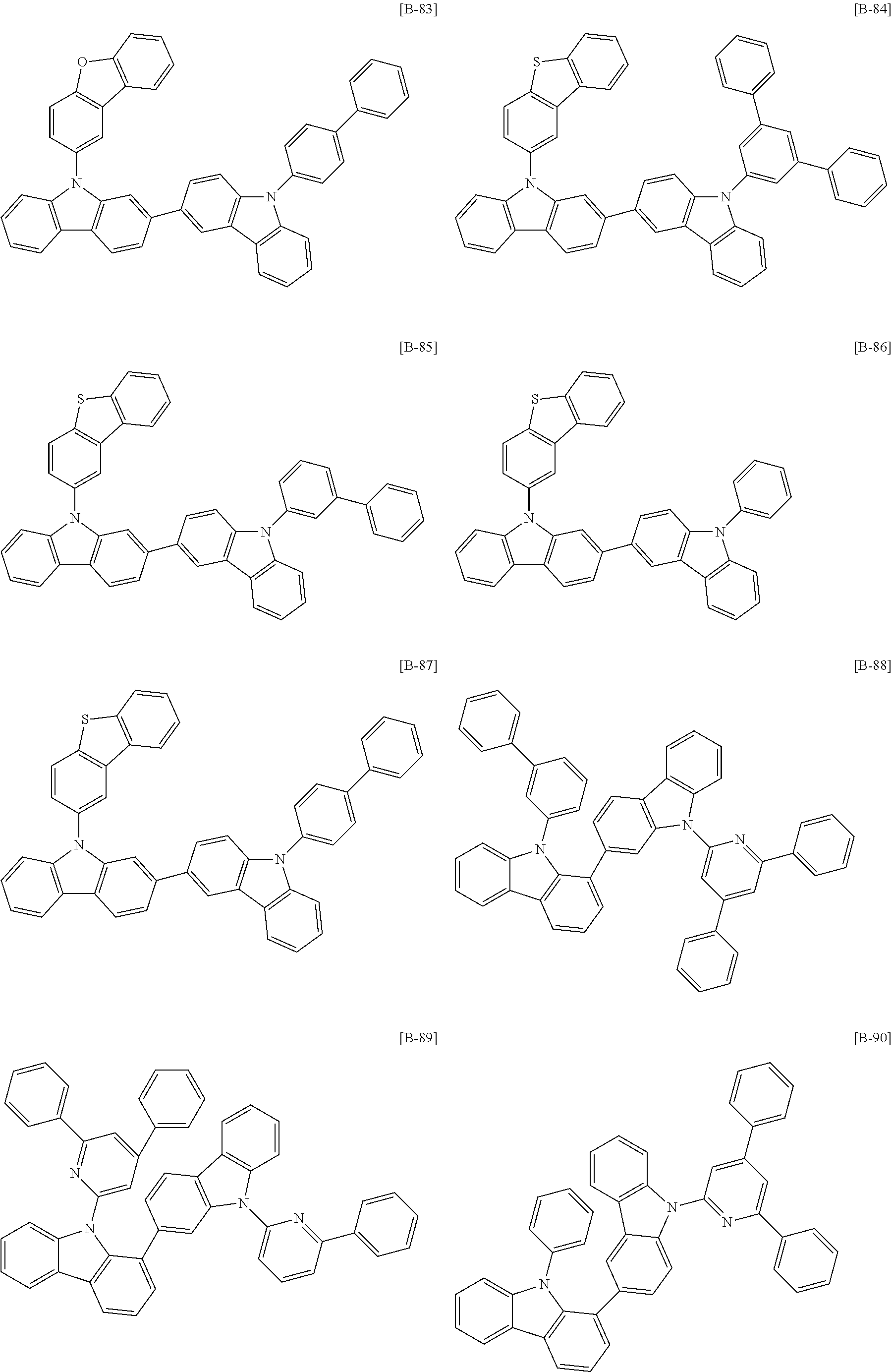

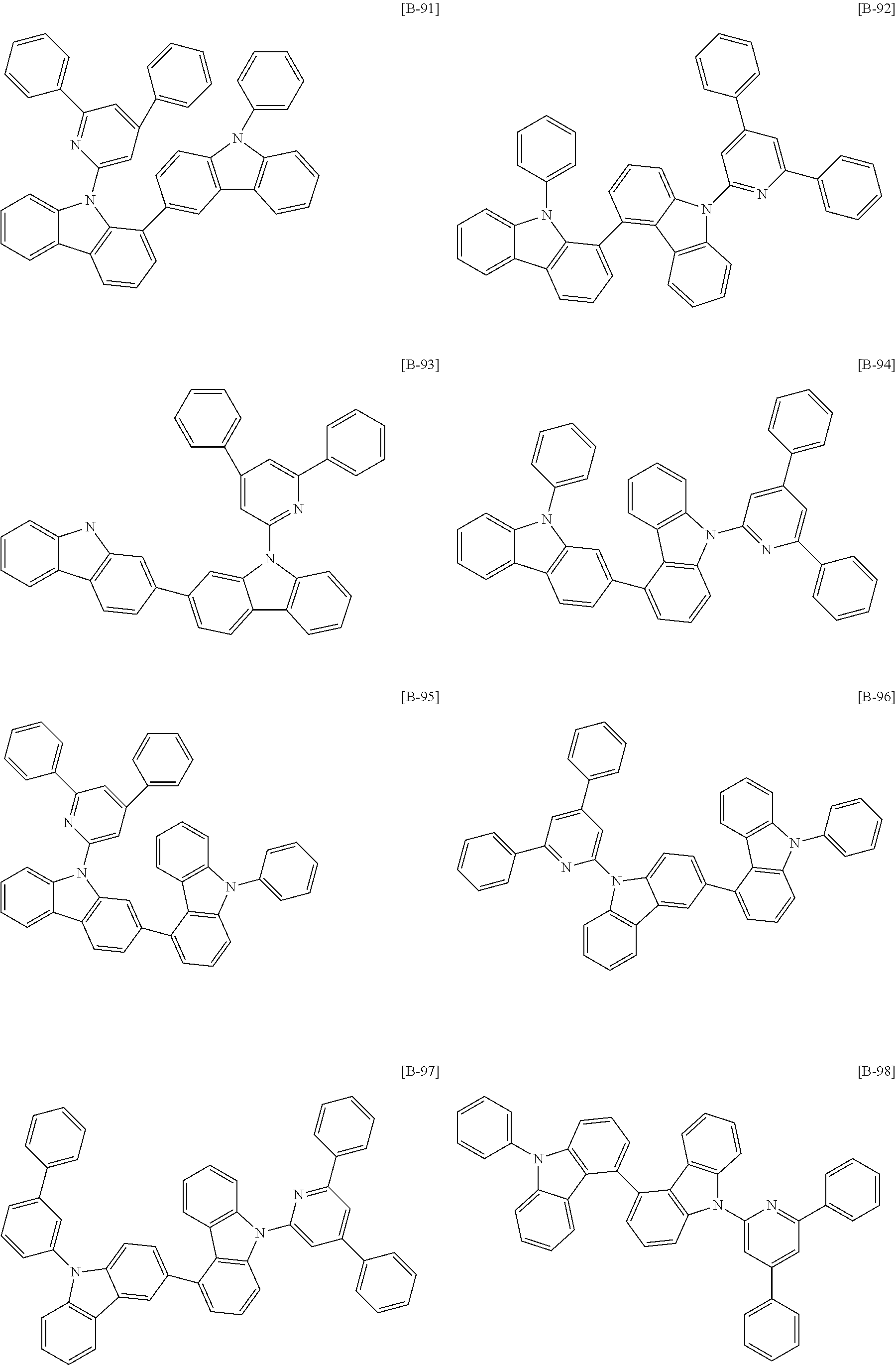

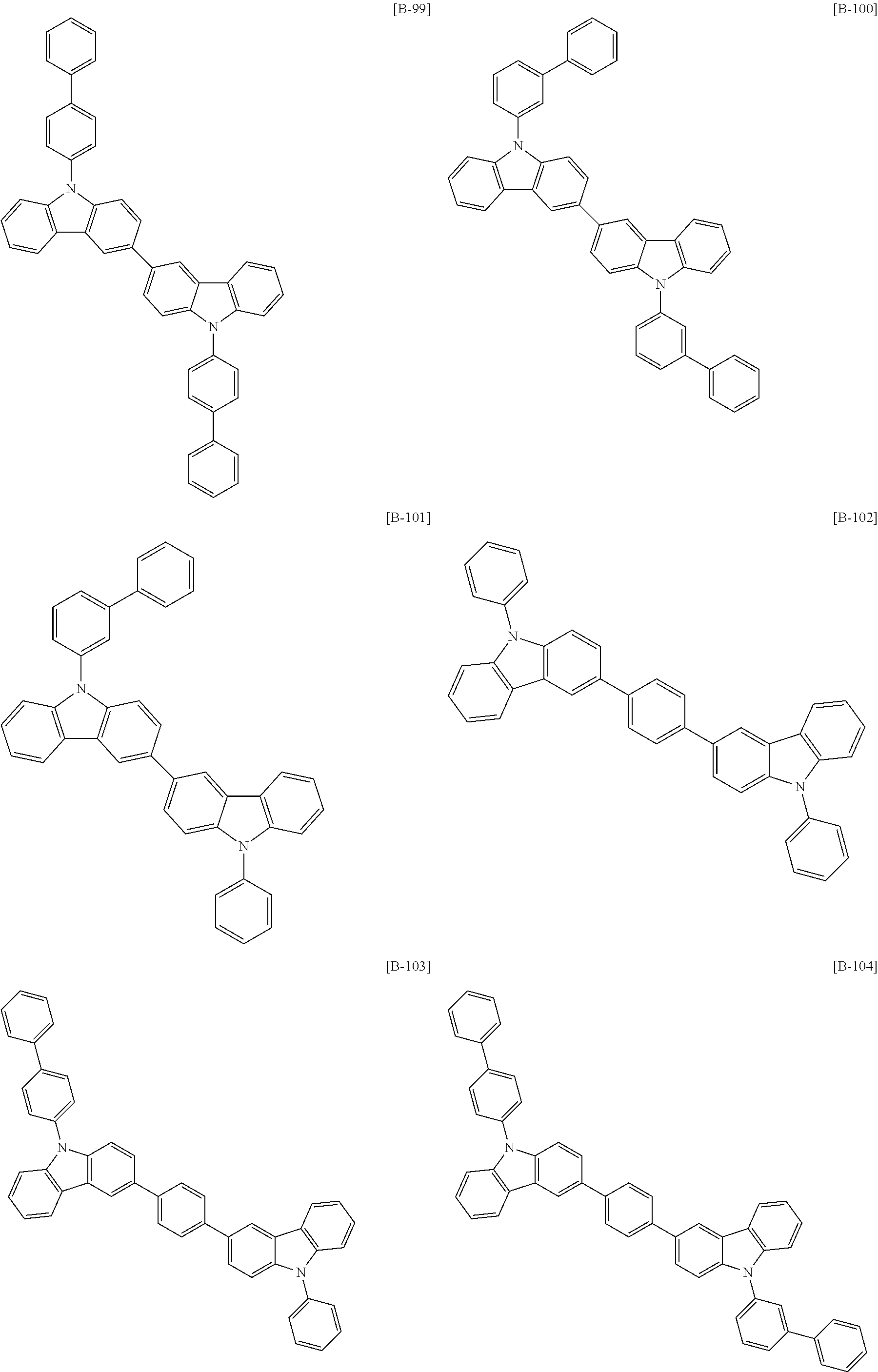

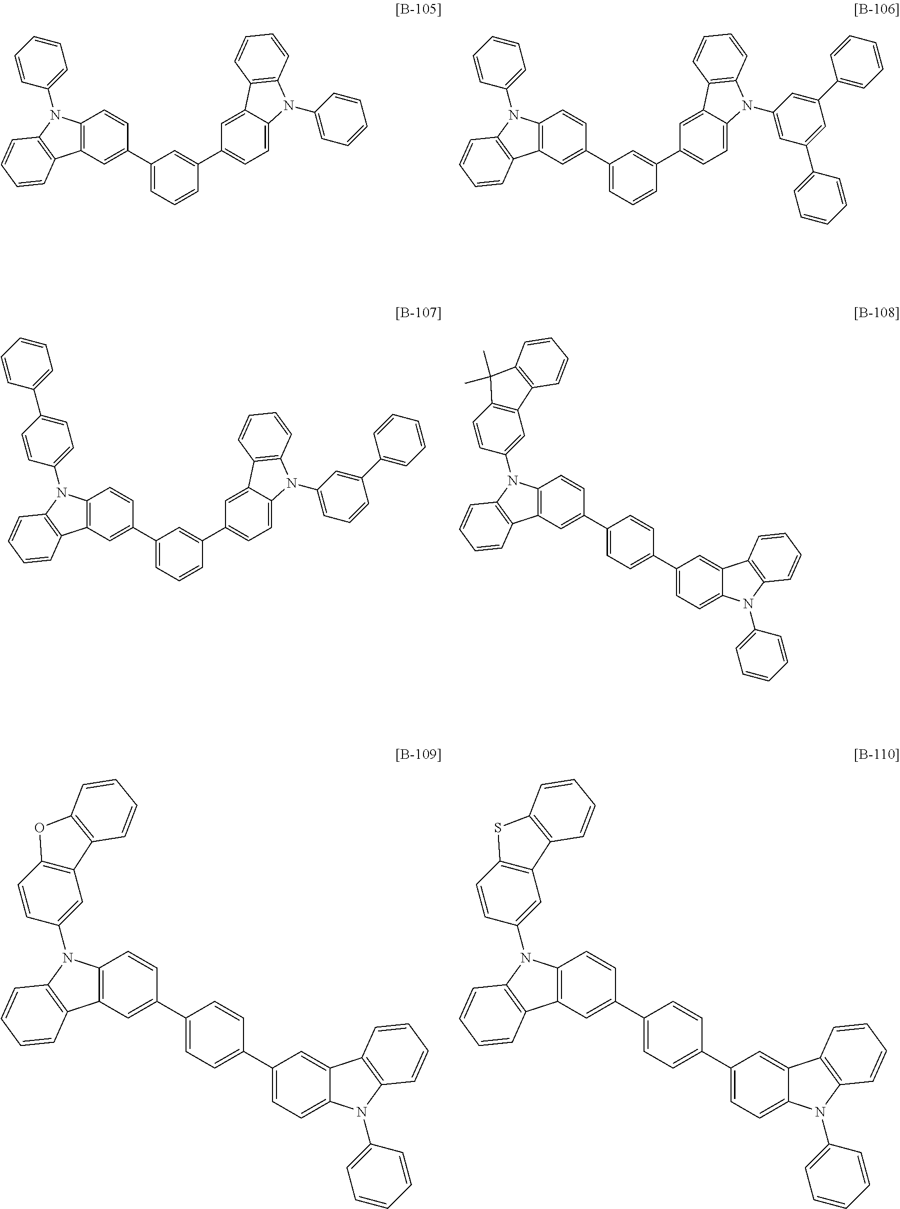

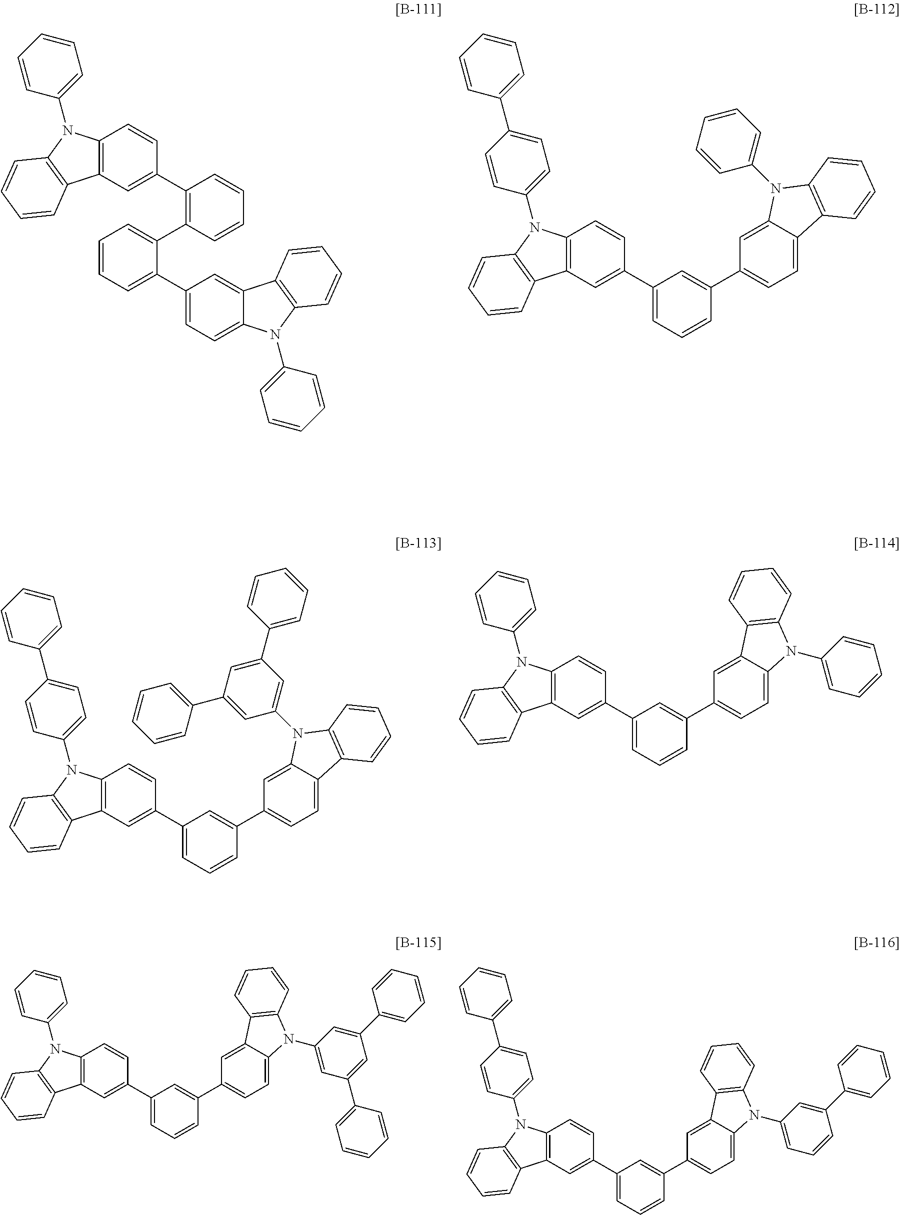

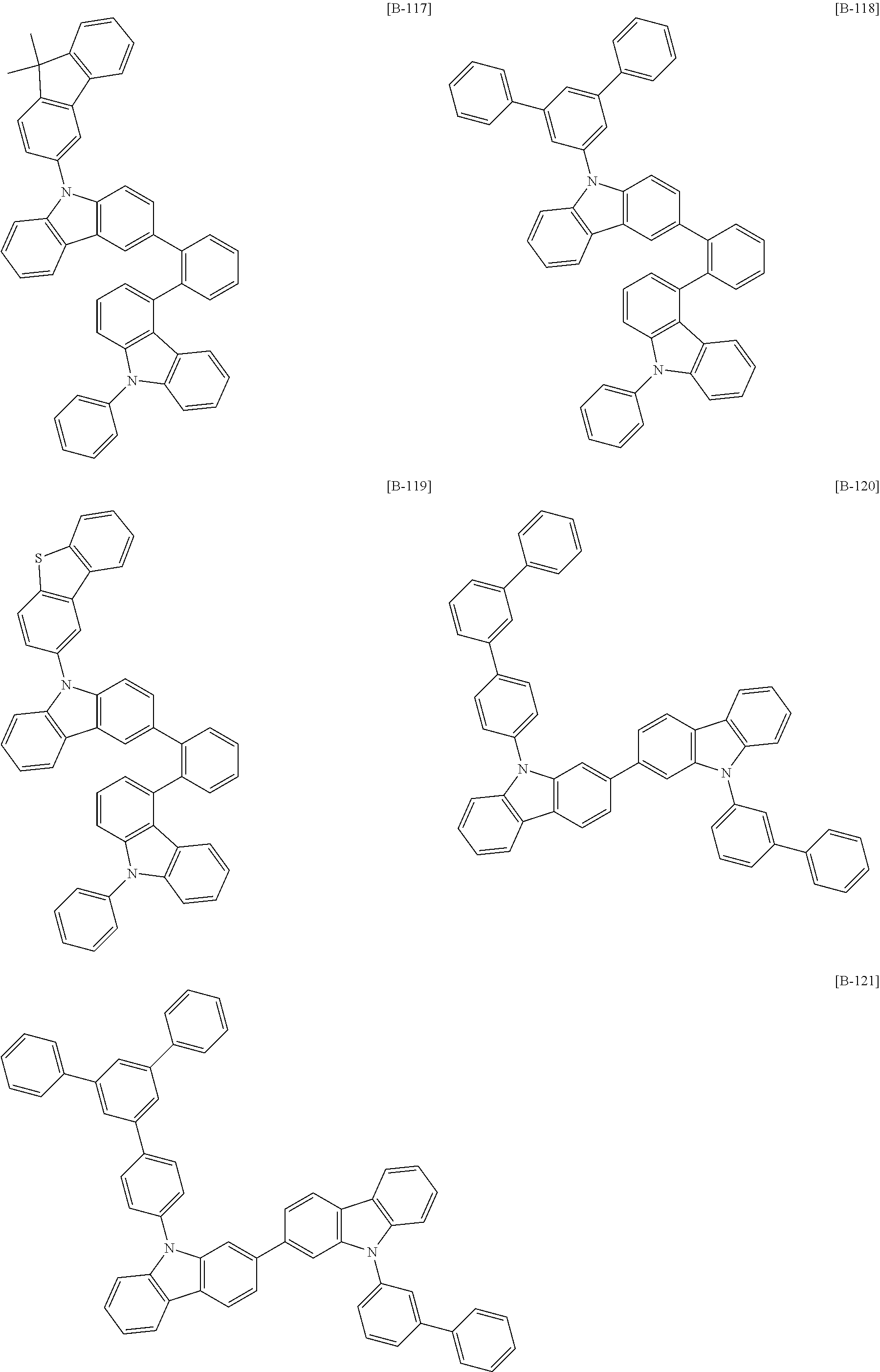 [SMILES: CC1(C)c2ccccc2-c2cc(-n3c4ccccc4c4cc(-c5ccccc5-c5cccc6c5c5ccccc5n6-c5ccccc5)ccc43)ccc21 ; c1ccc(-c2cc(-c3ccccc3)cc(-n3c4ccccc4c4cc(-c5ccccc5-c5cccc6c5c5ccccc5n6-c5ccccc5)ccc43)c2)cc1 ; c1ccc(-n2c3ccccc3c3c(-c4ccccc4-c4ccc5c(c4)c4ccccc4n5-c4ccc5sc6ccccc6c5c4)cccc32)cc1 ; c1ccc(-c2cccc(-c3ccc(-n4c5ccccc5c5ccc(-c6ccc7c8ccccc8n(-c8cccc(-c9ccccc9)c8)c7c6)cc54)cc3)c2)cc1 ; c1ccc(-c2cc(-c3ccccc3)cc(-c3ccc(-n4c5ccccc5c5ccc(-c6ccc7c8ccccc8n(-c8cccc(-c9ccccc9)c8)c7c6)cc54)cc3)c2)cc1]

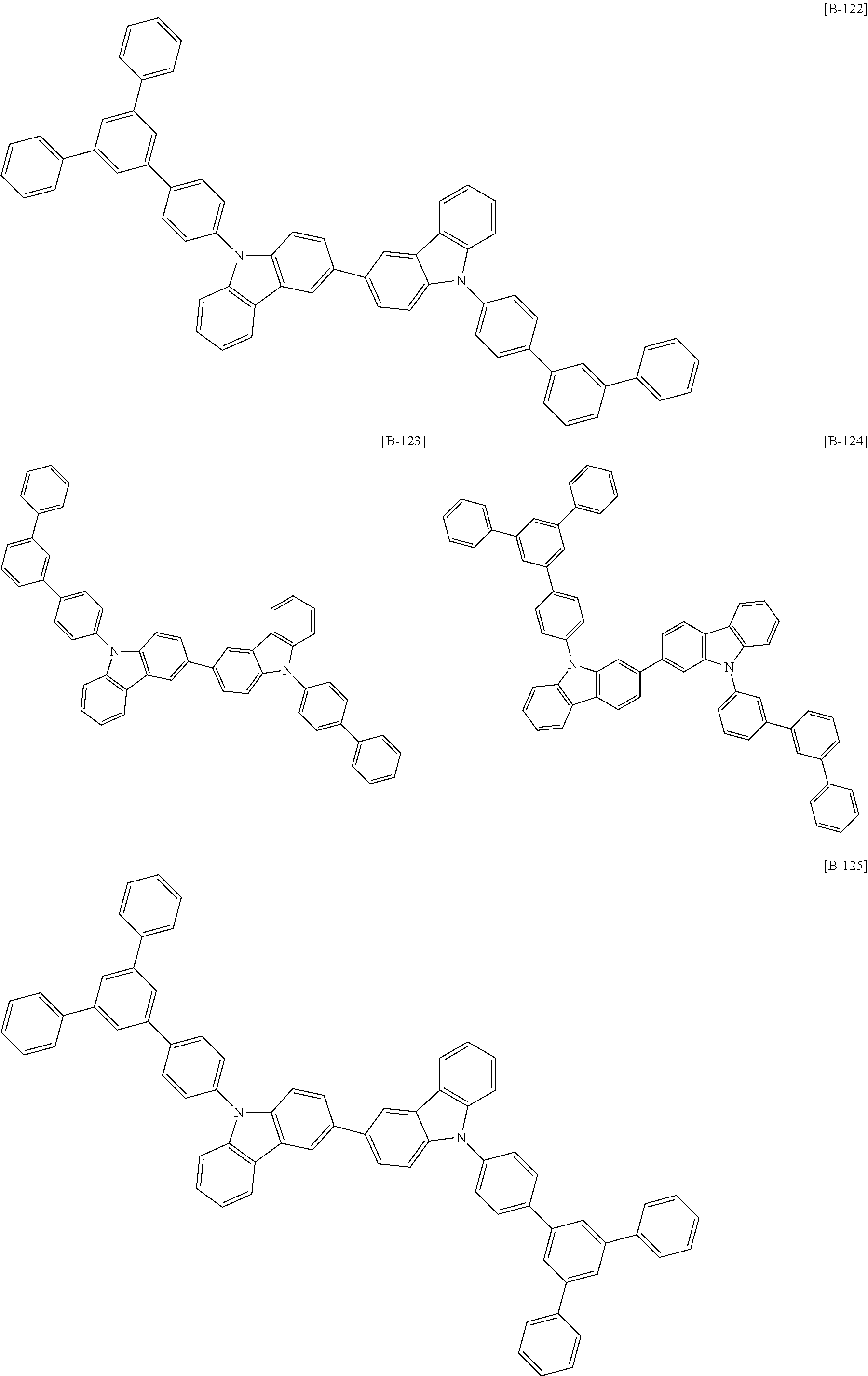

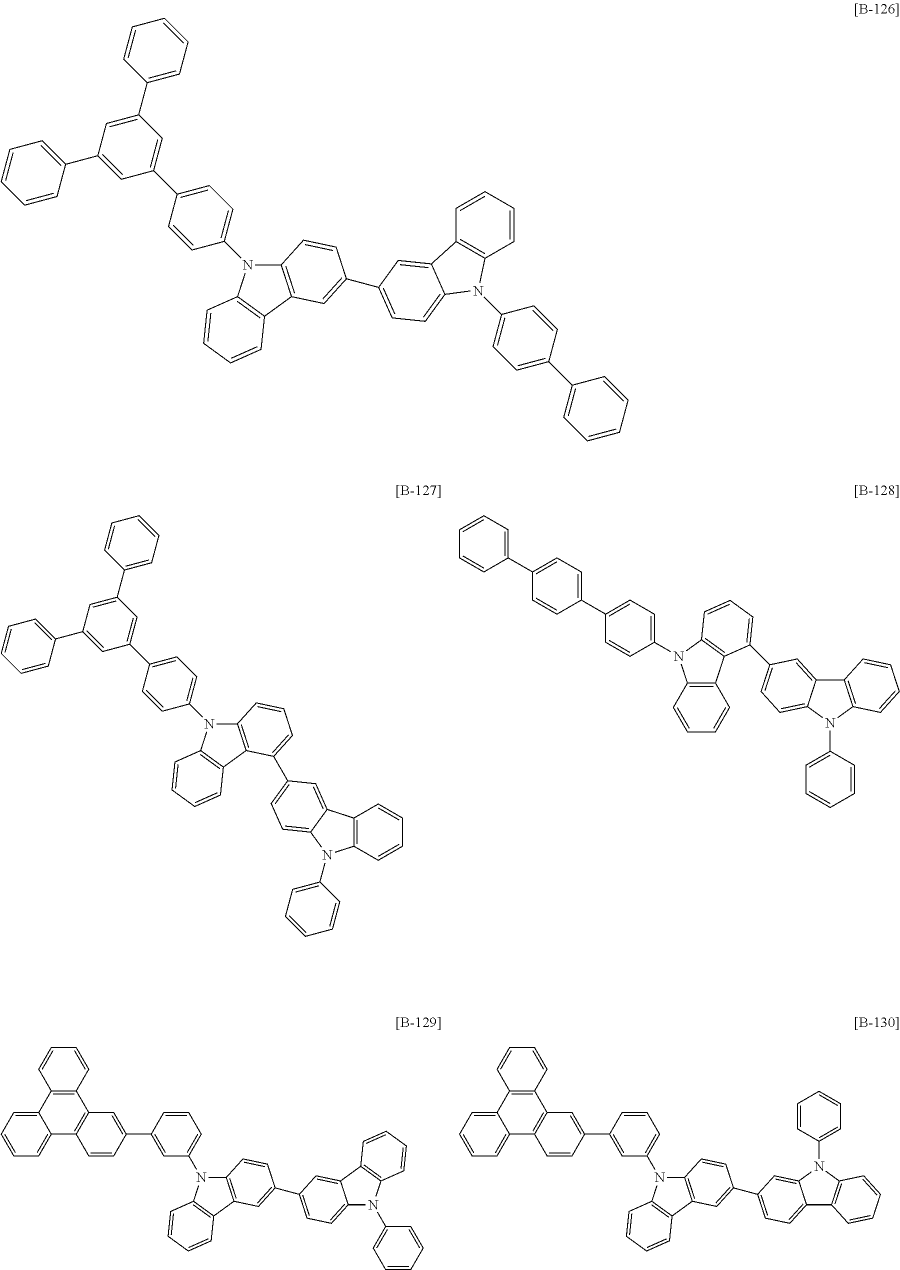 [SMILES: c1ccc(-c2ccc(-n3c4ccccc4c4cc(-c5ccc6c(c5)c5ccccc5n6-c5ccc(-c6cc(-c7ccccc7)cc(-c7ccccc7)c6)cc5)ccc43)cc2)cc1 ; c1ccc(-c2cc(-c3ccccc3)cc(-c3ccc(-n4c5ccccc5c5c(-c6ccc7c(c6)c6ccccc6n7-c6ccccc6)cccc54)cc3)c2)cc1 ; c1ccc(-c2ccc(-c3ccc(-n4c5ccccc5c5c(-c6ccc7c(c6)c6ccccc6n7-c6ccccc6)cccc54)cc3)cc2)cc1 ; c1ccc(-n2c3ccccc3c3cc(-c4ccc5c(c4)c4ccccc4n5-c4cccc(-c5ccc6c7ccccc7c7ccccc7c6c5)c4)ccc32)cc1 ; c1ccc(-n2c3ccccc3c3ccc(-c4ccc5c(c4)c4ccccc4n5-c4cccc(-c5ccc6c7ccccc7c7ccccc7c6c5)c4)cc32)cc1]

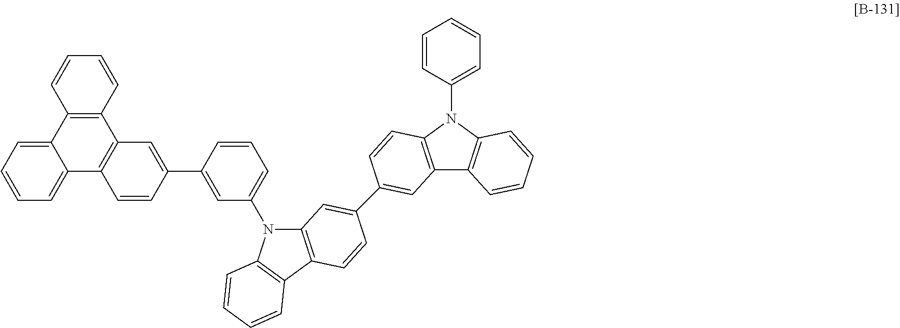 [SMILES: c1ccc(-n2c3ccccc3c3cc(-c4ccc5c6ccccc6n(-c6cccc(-c7ccc8c9ccccc9c9ccccc9c8c7)c6)c5c4)ccc32)cc1]

Since the hole characteristics of the second compound are relatively determined related with the first compound, at least either one of Ar¹ and Ar⁴ of Chemical Formula 2 may include a substituent having weak electron characteristics such as a substituted or unsubstituted pyridinyl group.

In this case, a LUMO energy level of the second compound may be about −1.7 eV.

Specifically, the LUMO energy level of the second compound may be about −1.7 eV to about −2.1 eV.

Specifically, in the emission layer 32, the first compound and the second compound are simultaneously included as a host, and may include, for example at least one first compound represented by Chemical Formula 1-I; and at least one second compound of Group B.

As described above, the emission layer 32 includes the first compound having bipolar characteristics in which electron characteristics are relatively strong and the second compound having relatively strong hole characteristics and thereby increases mobility of electrons and holes and remarkably improves luminous efficiency compared with the compounds alone.

When a material having biased electron or hole characteristics is used to form an emission layer, excitons in a device including the emission layer are relatively more generated due to recombination of carriers on the interface between the emission layer and the electron or hole transport layer. As a result, the molecular excitons in the emission layer interact with charges on the interface of the hole transport layer and thus, cause a roll-off of sharply deteriorating efficiency and also, sharply deteriorate light emitting life-span characteristics.

In order to solve the problems, the first and second compounds are simultaneously included in the emission layer to make a light emitting region not be biased to either of the electron transport layer or the hole transport layer, and additionally, the hole transport auxiliary layer including the third compound is disposed between the hole transport layer and the emission layer, and thereby charges are prevented from being accumulated at the interface between the hole transport layer and the emission layer and a device capable of adjusting carrier balance in the emission layer may be provided. Accordingly, roll-off characteristics of an organic optoelectronic device may be improved and simultaneously life-span characteristics may be remarkably improved.

In the emission layer 32, the first compound and the second compound may be included as a host, and may be included in a weight ratio of. for example about 1:10 to about 10:1, specifically about 2:8 to about 8:2, about 3:7 to about 7:3, about 4:6 to about 6:4, or about 5:5.

And in the embodiment of the invention, the first compound and the second compound may be included in a weight ratio of about 9:1 to about 1:1, about 4:1 to about 1:1.

Most specifically, the first compound and the second compound may be included in a weight ratio of about 1:1 or about 4:1, but are not limited thereto.

Within the ranges, bipolar characteristics may be effectively realized to improve efficiency and life-span simultaneously.

The emission layer 32 may further include at least one compound in addition to the first compound and the second compound.

The emission layer 32 may further include a dopant. The dopant is mixed with a host in a small amount to cause light emission, and may be generally a material such as a metal complex that emits light by multiple excitation into a triplet or more. The dopant may be, for example an inorganic, organic, or organic/inorganic compound, and one or more kinds thereof may be used.

The dopant may be a red, green, or blue dopant, for example a phosphorescent dopant. The phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example a compound represented by Chemical Formula Z, but is not limited thereto.

L₂MX [Chemical Formula Z]

In Chemical Formula Z, M is a metal, and L and X are the same or different, and are a ligand to form a complex compound with M.

The M may be, for example, Ir. Pt, Os, Ti, Zr, Hf, Eu, Tb, Tm, Fe, Co, Ni, Ru, Rh, Pd, or a combination thereof, and the L and X may be, for example a bidendate ligand.

The hole transport auxiliary layer 33 includes the third compound having relatively good hole characteristics.

The hole transport auxiliary layer 33 includes the third compound and thus may reduce a HOMO energy level difference between the hole transport layer 31 and the emission layer 32, adjust hole injection characteristics, and reduce accumulation of holes on the interface of the hole transport auxiliary layer 33 and the emission layer 32 and thus a quenching phenomenon that excitons are quenched by polaron on the interface. Accordingly, a device may be less deteriorated but stabilized, and thus efficiency and life-span of the device may be improved.

The third compound may be represented by Chemical Formula 3.

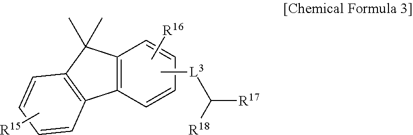

In Chemical Formula 3,

R¹⁵ to R¹⁸ are independently, hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof, and

L³ is a single bond, a substituted or unsubstituted C6 to C30 arylene group, a substituted or unsubstituted C2 to C30 heteroarylene group, or a combination thereof.

According to an embodiment, R¹⁵ and R¹⁶ of Chemical Formula 5 may independently be hydrogen,

the R¹⁷ and R¹⁸ may independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylenyl group, and

L³ may be a single bond, a substituted or unsubstituted phenylene group, a substituted or unsubstituted biphenylene group, a substituted or unsubstituted terphenylenyl group, or a substituted or unsubstituted naphthylenyl group.

R¹⁷ and R¹⁸ of Chemical Formula 3 may be selected from, for example substituted or unsubstituted groups of Group 6, but are not limited thereto.

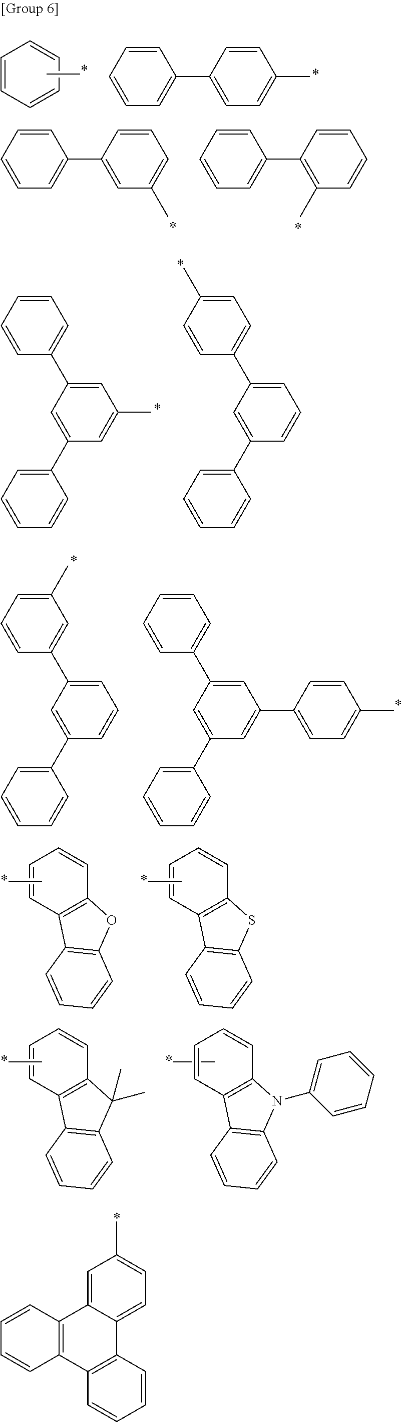

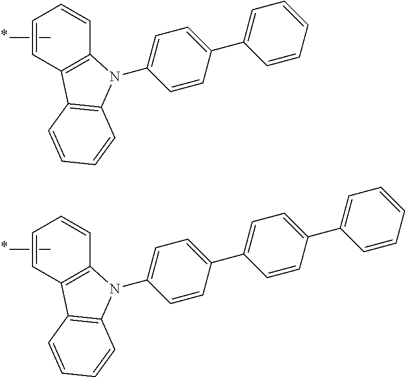

In Group 6, * is a linking point.

The third compound may be selected from, for example compounds of Group C, but is not limited thereto.

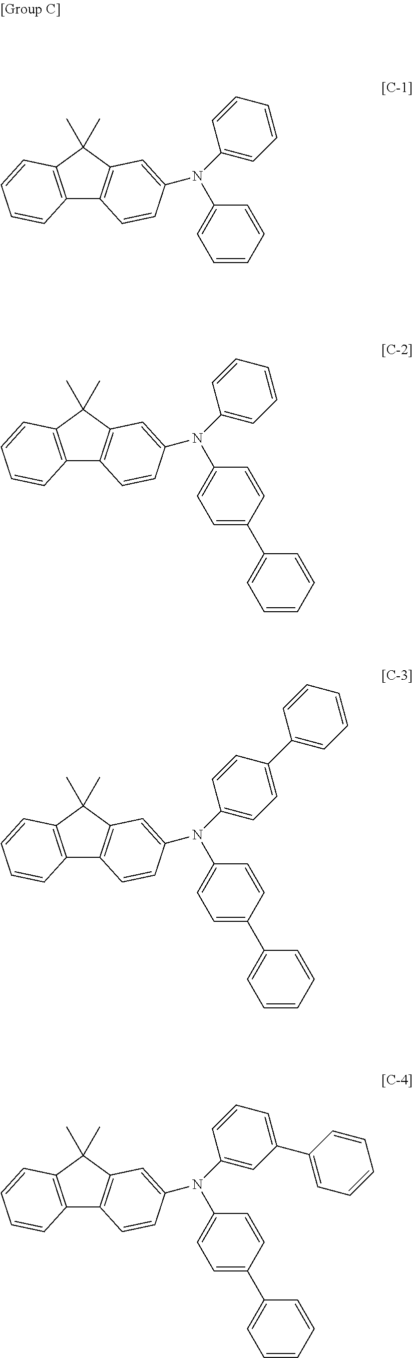 [SMILES: CC1(C)c2ccccc2-c2ccc(N(c3ccccc3)c3ccccc3)cc21 ; CC1(C)c2ccccc2-c2ccc(N(c3ccccc3)c3ccc(-c4ccccc4)cc3)cc21 ; CC1(C)c2ccccc2-c2ccc(N(c3ccc(-c4ccccc4)cc3)c3ccc(-c4ccccc4)cc3)cc21 ; CC1(C)c2ccccc2-c2ccc(N(c3ccc(-c4ccccc4)cc3)c3cccc(-c4ccccc4)c3)cc21]

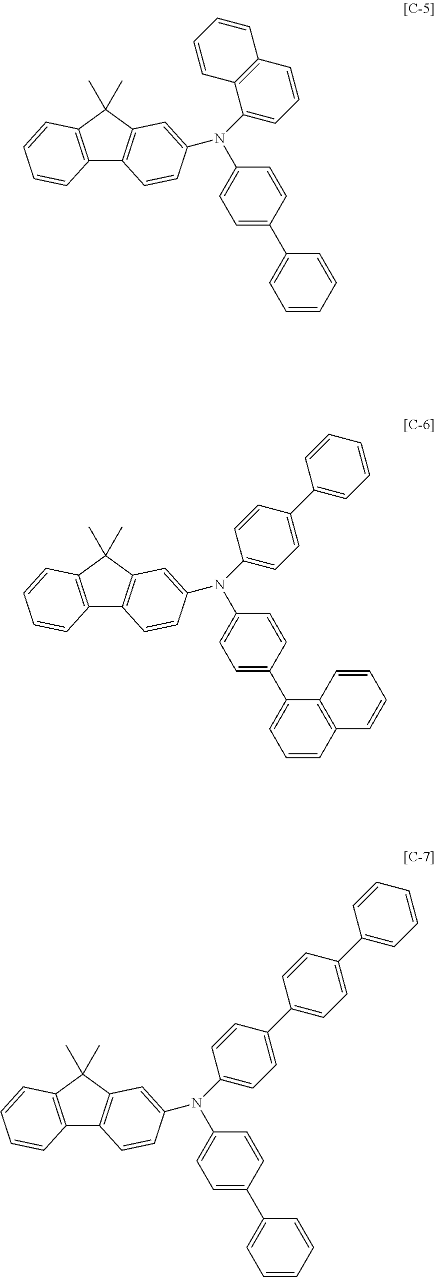 [SMILES: CC1(C)c2ccccc2-c2ccc(N(c3ccc(-c4ccccc4)cc3)c3cccc4ccccc34)cc21 ; CC1(C)c2ccccc2-c2ccc(N(c3ccc(-c4ccccc4)cc3)c3ccc(-c4cccc5ccccc45)cc3)cc21 ; CC1(C)c2ccccc2-c2ccc(N(c3ccc(-c4ccccc4)cc3)c3ccc(-c4ccc(-c5ccccc5)cc4)cc3)cc21]

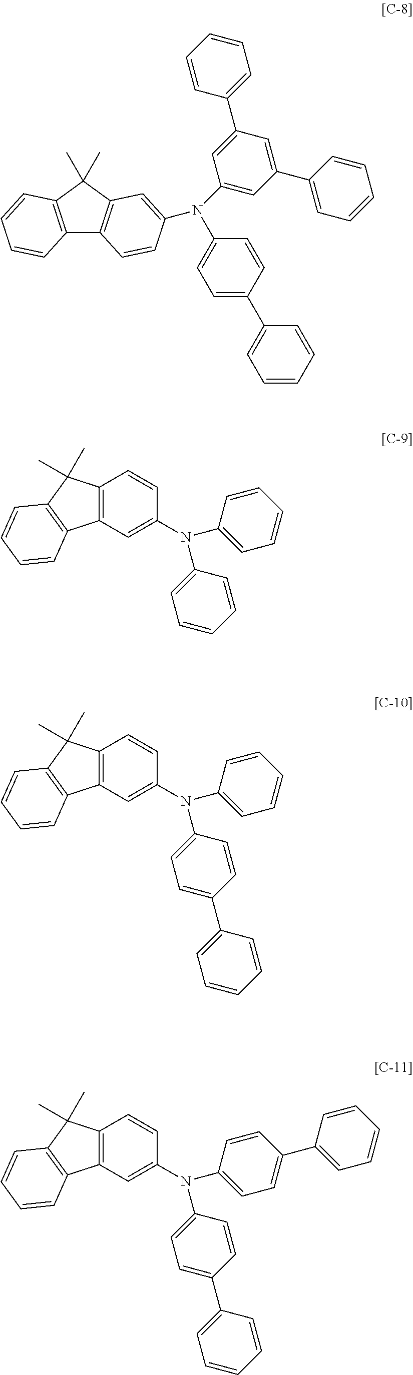 [SMILES: CC1(C)c2ccccc2-c2ccc(N(c3ccc(-c4ccccc4)cc3)c3cc(-c4ccccc4)cc(-c4ccccc4)c3)cc21 ; CC1(C)c2ccccc2-c2cc(N(c3ccccc3)c3ccccc3)ccc21 ; CC1(C)c2ccccc2-c2cc(N(c3ccccc3)c3ccc(-c4ccccc4)cc3)ccc21 ; CC1(C)c2ccccc2-c2cc(N(c3ccc(-c4ccccc4)cc3)c3ccc(-c4ccccc4)cc3)ccc21]

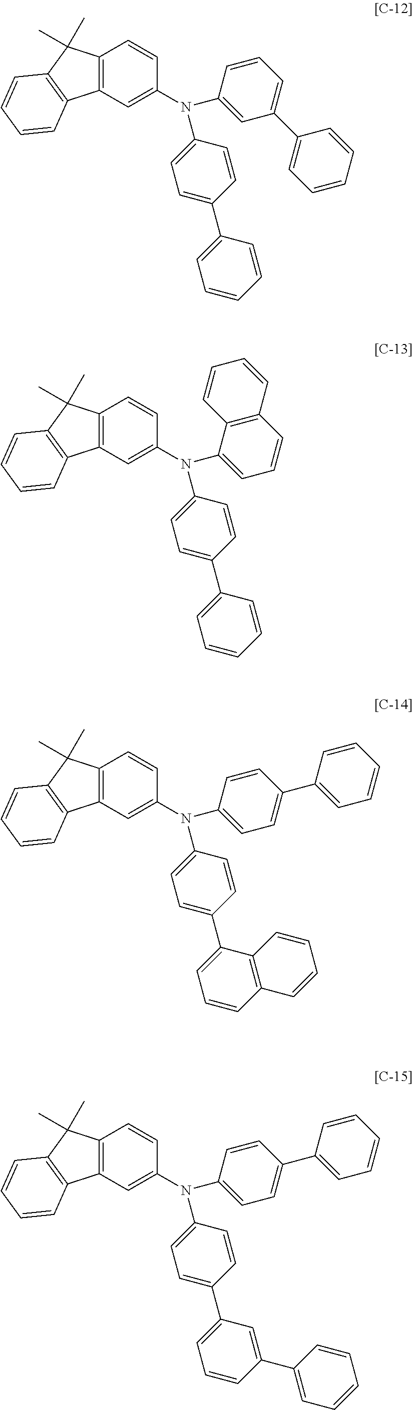 [SMILES: CC1(C)c2ccccc2-c2cc(N(c3ccc(-c4ccccc4)cc3)c3cccc(-c4ccccc4)c3)ccc21 ; CC1(C)c2ccccc2-c2cc(N(c3ccc(-c4ccccc4)cc3)c3cccc4ccccc34)ccc21 ; CC1(C)c2ccccc2-c2cc(N(c3ccc(-c4ccccc4)cc3)c3ccc(-c4cccc5ccccc45)cc3)ccc21 ; CC1(C)c2ccccc2-c2cc(N(c3ccc(-c4ccccc4)cc3)c3ccc(-c4cccc(-c5ccccc5)c4)cc3)ccc21]

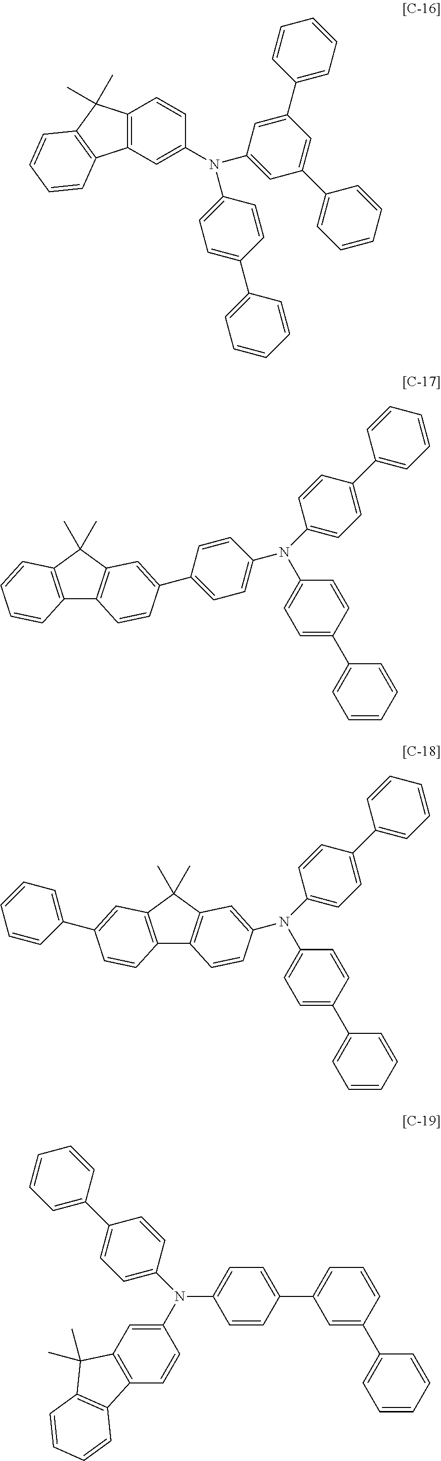 [SMILES: CC1(C)c2ccccc2-c2cc(N(c3ccc(-c4ccccc4)cc3)c3cc(-c4ccccc4)cc(-c4ccccc4)c3)ccc21 ; CC1(C)c2ccccc2-c2ccc(-c3ccc(N(c4ccc(-c5ccccc5)cc4)c4ccc(-c5ccccc5)cc4)cc3)cc21 ; CC1(C)c2cc(-c3ccccc3)ccc2-c2ccc(N(c3ccc(-c4ccccc4)cc3)c3ccc(-c4ccccc4)cc3)cc21 ; CC1(C)c2ccccc2-c2ccc(N(c3ccc(-c4ccccc4)cc3)c3ccc(-c4cccc(-c5ccccc5)c4)cc3)cc21]

According to an embodiment, an organic optoelectronic device may simultaneously include an emission layer simultaneously including the first compound having electron characteristics and the second compound having strong hole characteristics and

a hole transport auxiliary layer including the third compound adjusting hole injection characteristics and thus having sufficient hole transport characteristics by decreasing a HOMO energy level between the hole transport layer 31 and the emission layer 32.

The first compound, the second compound, and the third compound are used together and thus may reduce a quenching phenomenon that excitons are quenched by polaron on the interface between the hole transport auxiliary layer 33 and the emission layer 32. Accordingly, a device may be less deteriorated but stabilized, and efficiency and life-span of the device may be improved.

In addition, the hole transport auxiliary layer is disposed between the emission layer and the hole transport layer and thus may gradually adjust HOMO energy levels of the anode 10, the hole transport layer 31, and the hole transport auxiliary layer 33 and efficiently transport holes and resultantly, improve efficiency and contribute to a long life-span.

Specifically, the emission layer may include, for example at least one first compound represented by Chemical Formula 1-I and at least one second compound of Group B.

The hole transport auxiliary layer 35 may be applied on a hole transport layer by a deposition or inkjet process with a thickness of about 0.1 nm to about 20.0 nm, for example about 0.2 nm to about 10.0 nm, about 0.3 nm to about 5 nm. about 0.3 nm to about 2 nm, or about 0.4 nm to about 1.0 nm.

The organic layer 30 may further include an electron transport layer 34. The electron transport layer 34 makes electron transfer from the cathode 20 to the emission layer 32 easy, and may be omitted as needed.

The organic layer 30 may optionally further include a hole injection layer 37 between the anode 10 and the hole transport layer 31 and/or an electron injection layer 36 between the cathode 20 and the electron transport layer 34.

In an example of the present invention, the hole transport auxiliary layer may contact the hole transport layer and the emission layer, respectively in an organic optoelectronic device.

In an example of the present invention, the emission layer may further include a dopant, for example, a phosphorescent dopant, a fluorescent dopant, and the like.

In an embodiment, the organic optoelectronic device may be selected from an organic light emitting diode, an organic photoelectric device, an organic solar cell, an organic transistor, an organic photo conductor drum, and an organic memory device.

The organic light emitting diode may be applied to an organic light emitting diode (OLED) display.

Hereinafter, the embodiments are illustrated in more detail with reference to examples. These examples, however, are not in any sense to be interpreted as limiting the scope of the invention.

Hereinafter, a starting material and a reactant used in Synthesis Examples and Examples were purchased from Sigma-Aldrich Corporation or TCI Inc. unless there was particularly mentioned.

Synthesis of First Compound

(Representative Synthesis)

A representative synthesis is expressed by Reaction Scheme.

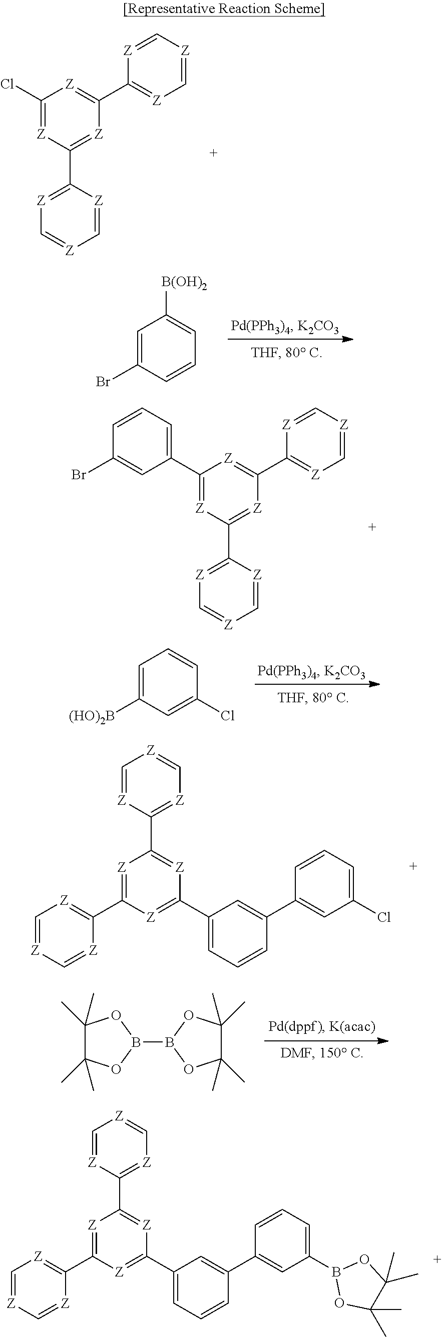

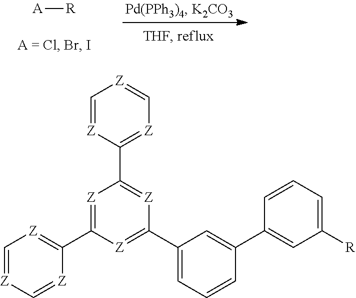

(Synthesis of Intermediate)

### SYNTHESIS EXAMPLE 1

### Synthesis of Intermediate I-1

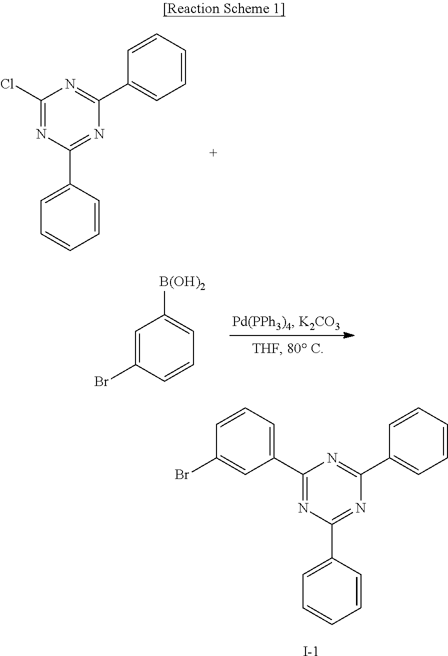 [SMILES: Clc1nc(-c2ccccc2)nc(-c2ccccc2)n1 ; OB(O)c1cccc(Br)c1 ; Brc1cccc(-c2nc(-c3ccccc3)nc(-c3ccccc3)n2)c1 ; [I-]]

The compound, 2-chloro-4,6-diphenyl-1,3,5-triazine (50 g, 187 mmol) was dissolved in 1 L of THF (tetrahydrofuran) in a nitrogen environment, (3-bromophenyl)boronic acid (45 g, 224.12 mmol) and tetrakis(triphenylphosphine)palladium (2.1 g, 1.87 mmol) were added thereto, and the mixture was stirred. Potassium carbonate saturated in water (64 g, 467 mmol) was added thereto, and the resulting mixture was heated and refluxed at 80° C. for 12 hours. When the reaction was complete, water was added to the reaction solution. dichloromethane (DCM) was used for an extraction, and an extract therefrom was filtered after removing moisture with anhydrous MgSO₄ and then, concentrated under a reduced pressure. This obtained residue was separated and purified through flash column chromatography to obtain the intermediate I-1 (69 g and 95%).

HRMS (70 eV, EI+): m/z calcd for C21H14BrN3:387.0371, found: 387.

Elemental Analysis: C, 65%; H, 4%

### SYNTHESIS EXAMPLE 2

### Synthesis of Intermediate I-2

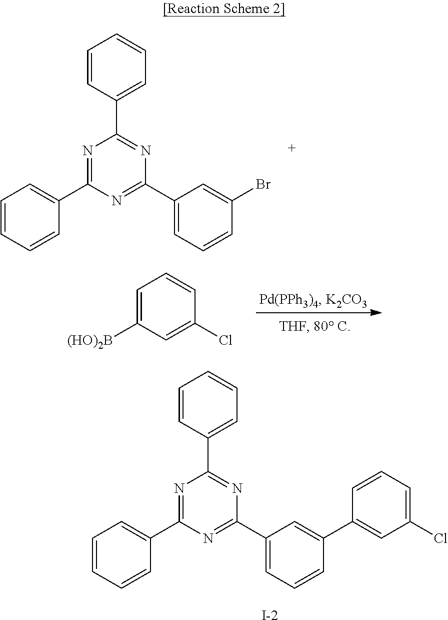

The intermediate I-1 (50 g, 128 mmol) was dissolved in 1 L of THF in a nitrogen environment, (3-chlorophenyl)boronic acid (24 g, 155 mmol) and tetrakis(triphenylphosphine)palladium (1.5 g, 1.3 mmol) were added thereto, and the mixture was stirred. Potassium carbonate saturated in water (44 g, 320 mmol) was added thereto, and the resulting mixture was heated and refluxed at 80° C. for 12 hours. When the reaction was complete, water was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain the intermediate I-2 (51 g and 95%).

HRMS (70 eV, EI+): m/z calcd for C27H18ClN3:419.1189, found: 419.

Elemental Analysis: C, 77%; H, 4%

### SYNTHESIS EXAMPLE 3

### Synthesis of Intermediate I-3

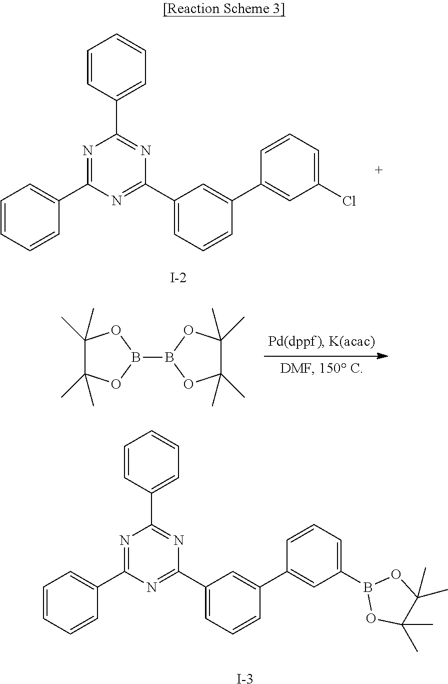

The intermediate I-2 (100 g, 238 mmol) was dissolved in 1 L of dimethylforamide (DMF) in a nitrogen environment, bis(pinacolato)diboron (72.5 g, 285 mmol), (1,1′-bis(diphenylphosphine)ferrocene)dichloropalladium (II) (2 g, 2.38 mmol), and potassium acetate (58 g, 595 mmol) were added thereto, and the mixture was heated and refluxed at 150° C. for 48 hours. When the reaction was complete, water was added to the reaction solution, and the resulting mixture was filtered and dried in a vacuum oven. The obtained residue was separated and purified through flash column chromatography to obtain the intermediate I-3 (107 g, 88%).

HRMS (70 eV, EI+): m/z calcd for C33H30BN302:511.2431, found: 511

Elemental Analysis: C, 77%; H, 6%

### SYNTHESIS EXAMPLE 4

### Synthesis of Intermediate I-4

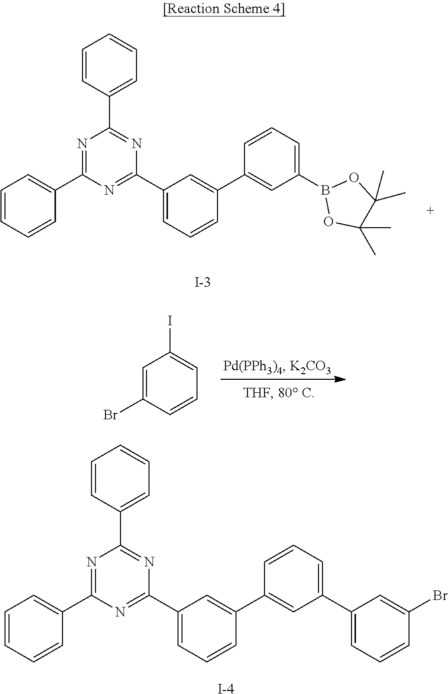

The intermediate I-3 (50 g, 98 mmol) was dissolved in 1 L of THF in a nitrogen environment, 1-bromo-3-iodobenzene (33 g, 117 mmol) and tetrakis(triphenylphosphine)palladium (1 g, 0.98 mmol) were added thereto, and the mixture was stirred. Potassium carbonate saturated in water (34 g, 245 mmol) was added thereto, and the mixture was heated and refluxed at 80° C. for 12 hours. When the reaction was complete, water was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain the intermediate I-4 (50 g and 95%).

HRMS (70 eV, EI+): m/z calcd for C30H27BO2:539.0997, found: 539.

Elemental Analysis: C, 73.34; H, 4.10

### SYNTHESIS EXAMPLE 5

### Synthesis of Intermediate I-5

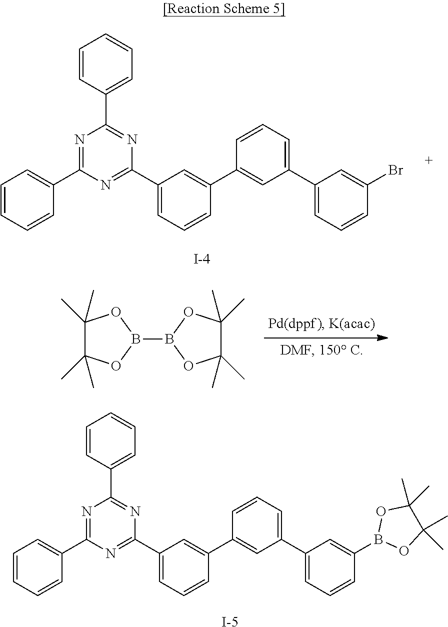

The intermediate I-4 (100 g, 185 mmol) was dissolved in 1 L of dimethylforamide (DMF) in a nitrogen environment, bis(pinacolato)diboron (56 g, 222 mmol), (1,1′-bis(diphenylphosphine)ferrocene)dichloropalladium (II) (1.5 g, 1.85 mmol), and potassium acetate (45 g, 595 mmol) were added thereto, and the mixture was heated and refluxed at 150° C. for 5 hours. When the reaction was complete, water was added to the reaction solution, and the mixture was filtered and dried in a vacuum oven. The obtained residue was separated and purified through flash column chromatography to obtain an intermediate I-5 (95 g and 88%).

HRMS (70 eV, EI+): m/z calcd for C39H34BN3O2:587.2744, found: 587

Elemental Analysis: C, 80%; H, 6%

Synthesis of Final Compound

### SYNTHESIS EXAMPLE 6

### Synthesis of Compound A-1

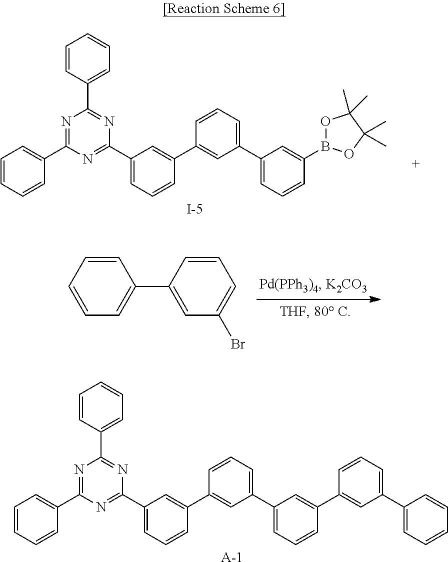

The intermediate I-5 (20 g, 34 mmol) was dissolved in 0.2 L of tetrahydrofuran (THF) in a nitrogen environment, 3-bromo-1,1′-biphenyl (9.5 g, 40 mmol) and tetrakis(triphenylphosphine)palladium (0.39 g, 0.34 mmol) were added thereto, and the mixture was stirred. Potassium carbonate saturated in water (12 g, 85 mmol) was added thereto, and the resulting mixture was heated and refluxed at 80° C. for 20 hours. When the reaction was complete, water was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain the compound A-1 (24 g and 70%). A molecular weight of the compound A-1 was 613.2518.

HRMS (70 eV, EI+): m/z calcd for C45H31N3:613.2518, found: 613 Elemental Analysis: C, 88%; H, 5%

### SYNTHESIS EXAMPLE 7

### Synthesis of Compound A-19

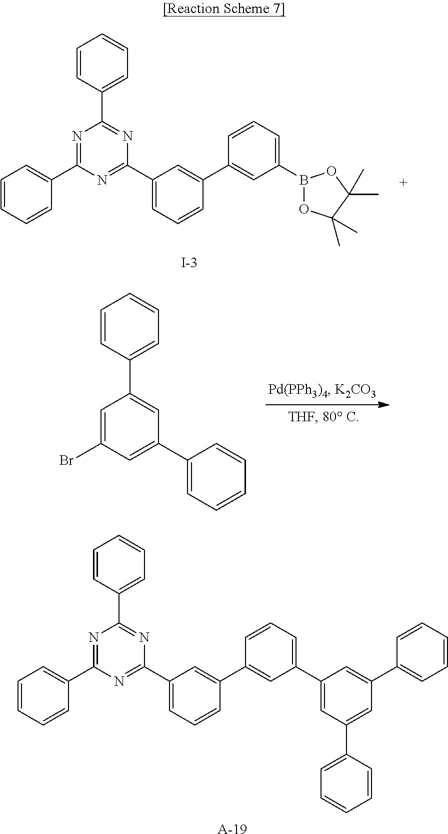

The intermediate I-3 (20 g, 39.1 mmol) was dissolved in 0.2 L of tetrahydrofuran (THF) in a nitrogen environment, 5′-bromo-1,1′:3′,1″-terphenyl (14.5 g, 47 mmol) and tetrakis(triphenylphosphine)palladium (0.45 g, 0.39 mmol) were added thereto, and the mixture was stirred. Potassium carbonate saturated in water (9.7 g, 99 mmol) was added thereto, and the resulting mixture was heated and refluxed at 80° C. for 20 hours. When the reaction was complete, water was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain the compound A-19 (20 g and 83%). A molecular weight of the compound A-19 was 613.2518.

HRMS (70 eV, EI+): m/z calcd for C45H31N3:613.2518, found: 613

Elemental Analysis: C, 88%; H, 5%

### SYNTHESIS EXAMPLE 8

### Synthesis of Compound A-46

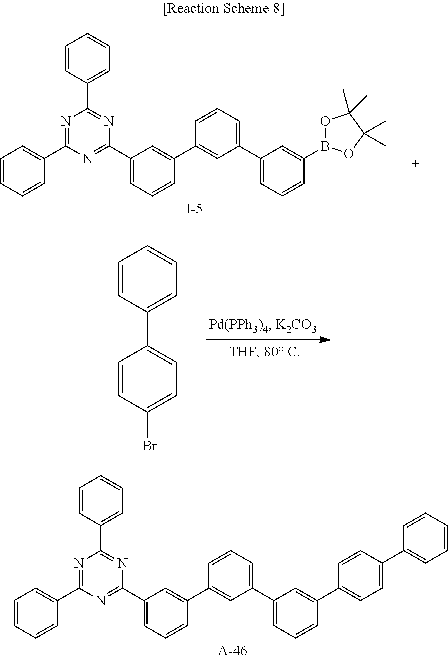

The intermediate I-5 (23.9 g, 40.73 mmol) was dissolved in 0.2 L of tetrahydrofuran (THF) in a nitrogen environment, 4-bromo-biphenyl (11.4 g, 48.88 mmol) and tetrakis(triphenylphosphine)palladium (0.94 g, 0.81 mmol) were added thereto, and the mixture was stirred. Potassium carbonate saturated in water (11.3 g, 81.5 mmol) was added thereto, and the resulting mixture was heated and refluxed at 80° C. for 20 hours. When the reaction was complete, water was added to the reaction solution, dichloromethane (DCM) was used for an extraction, an extract therefrom was filtered after removing moisture with anhydrous MgSO₄ and concentrated under a reduced pressure. The obtained residue was separated and purified through flash column chromatography to obtain the compound A-46 (20.4 g, 82%). A molecular weight of the compound A-46 was 613.75.

Synthesis of Second Compound

### SYNTHESIS EXAMPLE 9

### Synthesis of Compound B-10

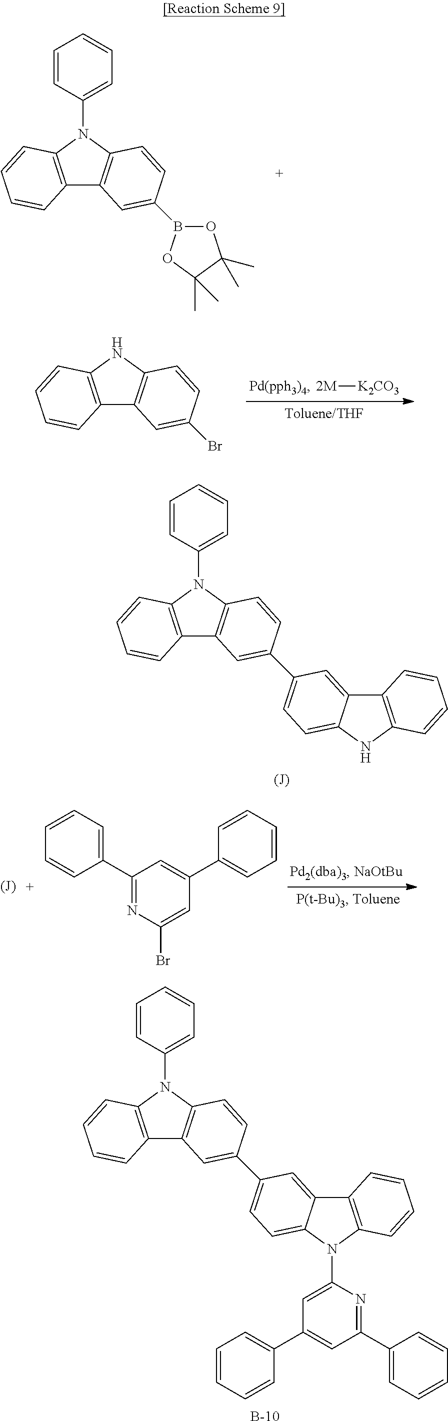

First Step: Synthesis of Compound J

The compound, 9-phenyl-3-(4,4,5,5-tetramethyl-1,3,2-dioxaborolan-2-yl)-9H-carbazole (26.96 g, 81.4 mmol) was dissolved in 0.2 L of toluene/THF in a nitrogen environment, 3-bromo-9H-carbazole (23.96 g, 97.36 mmol) and tetrakis(triphenylphosphine)palladium (0.90 g, 0.8 mmmol) were added thereto, and the mixture was stirred. Potassium carbonate saturated in water (28 g, 203.49 mmol) was added thereto, and the resulting mixture was heated and refluxed at 120° C. for 12 hours. When the reaction was complete, water was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain the compound J (22.6 g, 68%).

HRMS (70 eV, EI+): m/z calcd for C30H20N2:408.16, found: 408

Elemental Analysis: C, 88%; H, 5%

Second Step: Synthesis of Compound B-10

The compound J (22.42 g, 54.88 mmol) was dissolved in 0.2 L of toluene in a nitrogen environment, 2-bromo-4,6-diphenylpyridine (20.43 g, 65.85 mmol), NaOtBu (7.92 g, 82.32 mmol), tris(dibenzylideneacetone)dipalladium (0) (1.65 g, 1.65 mmol), and tri-tert-butylphosphine (1.78 g, 4.39 mmol) were added thereto, and the mixture was heated and refluxed at 120° C. for 12 hours. When the reaction was complete, water was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain the compound B-10 (28.10 g, 80%).

HRMS (70 eV, EI+): m/z calcd for C47H31N3:637.25, found: 637

Elemental Analysis: C, 89%; H, 5%

### SYNTHESIS EXAMPLE 10

### Synthesis of Compound B-30

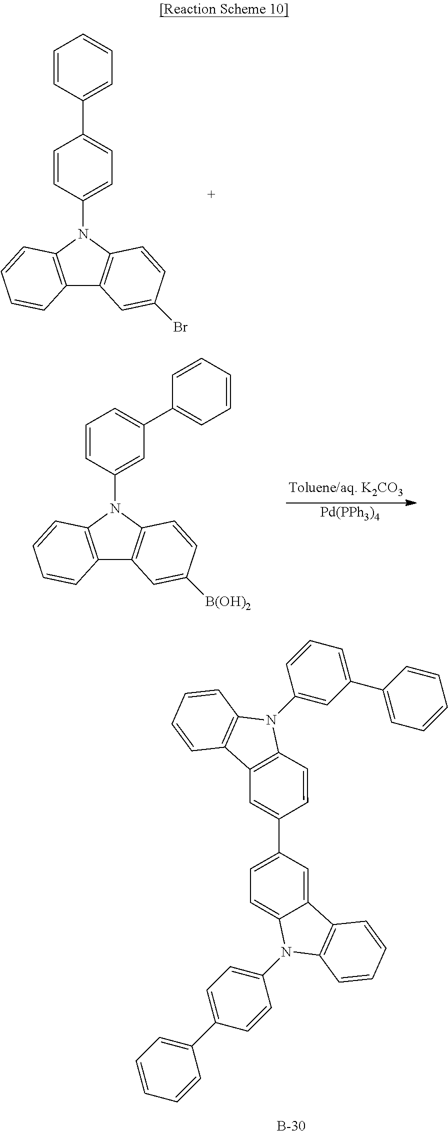

The compound, biphenylcarbazolylbromide (12.33 g, 30.95 mmol) was dissolved in 0.2 L of toluene in a nitrogen environment, 9-([1,1′-biphenyl]-3-yl)-9H-carbazole-3-boronic acid (12.37 g, 34.05 mmol) and tetrakis(triphenylphosphine)palladium (1.07 g, 0.93 mmmol) were added thereto, and the mixture was stirred. Potassium carbonate saturated in water (12.83 g, 92.86 mmol) was added thereto, and the resulting mixture was heated and refluxed at 120° C. for 12 hours. When the reaction was complete, water was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain a compound B-30 (18.7 g, 92%).

HRMS (70 eV, EI+): m/z calcd for C48H32N2:636.26, found: 636

Elemental Analysis: C, 91%; H, 5%

### SYNTHESIS EXAMPLE 11

### Synthesis of Compound D-128

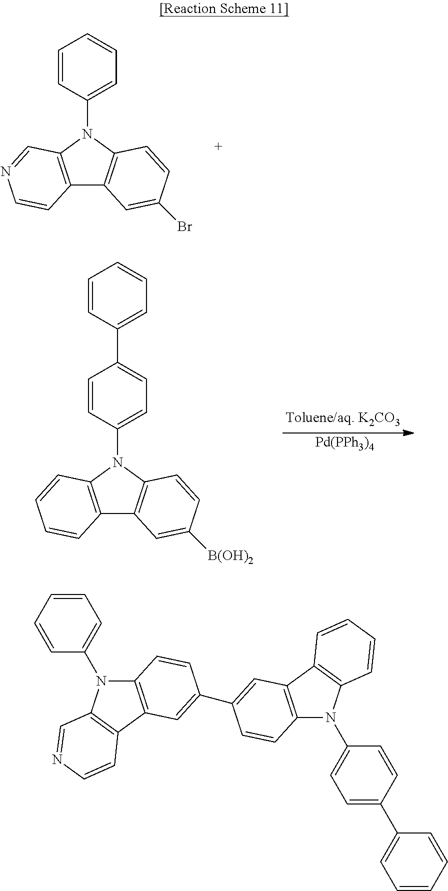 [SMILES: Brc1ccc2c(c1)c1ccncc1n2-c1ccccc1 ; OB(O)c1ccc2c(c1)c1ccccc1n2-c1ccc(-c2ccccc2)cc1 ; c1ccc(-c2ccc(-n3c4ccccc4c4cc(-c5ccc6c(c5)c5ccncc5n6-c5ccccc5)ccc43)cc2)cc1 ; "Pd(PPh3)4"]

The compound, 6-bromo-9-phenyl-9H-b-carboline (14.4 g, 44.51 mmol) was dissolved in 0.2 L of toluene in a nitrogen environment, 9-(biphenyl-4-yl)-3-boronic acid-9H-carbazole (19.4 g, 53.41 mmol) and tetrakis(triphenylphosphine)palladium (1.03 g, 0.89 mmmol) were added thereto, and the mixture was stirred. Potassium carbonate saturated in water (12.3 g, 89.02 mmol) was added thereto, and the resulting mixture was heated and refluxed at 120° C. for 28 hours. When the reaction was complete, water was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain the compound D-128 (17.5 g, 70%).

HRMS (70 eV, EI+): m/z calcd for C41H27N3: 561.67, found: 562

Elemental Analysis: C, 88%; H, 5% N: 7%

### SYNTHESIS EXAMPLE 12

### Synthesis of Compound D-129

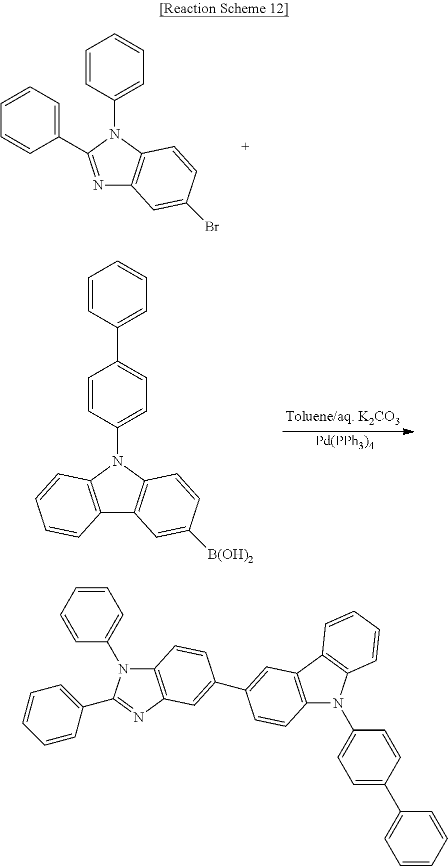 [SMILES: Brc1ccc2c(c1)nc(-c1ccccc1)n2-c1ccccc1 ; OB(O)c1ccc2c(c1)c1ccccc1n2-c1ccc(-c2ccccc2)cc1 ; c1ccc(-c2ccc(-n3c4ccccc4c4cc(-c5ccc6c(c5)nc(-c5ccccc5)n6-c5ccccc5)ccc43)cc2)cc1 ; "Pd(PPh3)4"]

The compound, 5-bromo-1,2-diphenyl-1H-benzoimidazole (14.9 g, 42.54 mmol) was dissolved in 0.2 L of toluene in a nitrogen environment, 9-(biphenyl-4-yl)-3-boronic acid-9H-carbazole (15.5 g, 42.54 mmol) and tetrakis(triphenylphosphine)palladium (0.98 g, 0.85 mmmol) were added thereto, and the mixture was stirred. Potassium carbonate saturated in water (11.8 g, 85.01 mmol) was added thereto, and the resulting mixture was heated and refluxed at 120° C. for 20 hours. When the reaction was complete, was added to the reaction solution, dichloromethane (DCM) was used for an extraction, and an extract therefrom was filtered after removing moisture with anhydrous MgSO₄ and concentrated under a reduced pressure. The obtained residue was separated and purified through flash column chromatography to obtain the compound D-129 (19.8 g, 79%).

HRMS (70 eV, EI+): m/z calcd for C43H29N3: 587.71, found: 588

Elemental Analysis: C, 88%; H. 5% N: 7%

Synthesis of Third Compound

### SYNTHESIS EXAMPLE 13

### Synthesis of Compound C-3

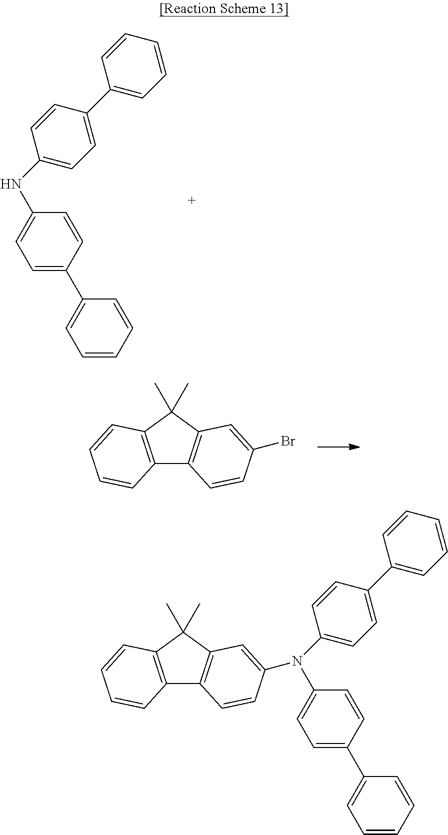 [SMILES: c1ccc(-c2ccc(Nc3ccc(-c4ccccc4)cc3)cc2)cc1 ; CC1(C)c2ccccc2-c2ccc(Br)cc21 ; CC1(C)c2ccccc2-c2ccc(N(c3ccc(-c4ccccc4)cc3)c3ccc(-c4ccccc4)cc3)cc21]

2-bromofluorene (11.7 g, 42.83 mmol), bis-biphenyl-4-yl-amine (12.51 g, 38.94 mmol), and sodium t-butoxide (7.86 g, 81.76 mmol) were put, and toluene (155 ml) was added thereto to dissolve them. Then, Pd(dba)₂ (0.357 g,0.39 mmol) and tri-tertiary-butylphosphine (0.236 g, 1.17 mmol) were sequentially added thereto, and the mixture was refluxed and stirred under a nitrogen atmosphere for 4 hours. When the reaction was complete, toluene and distilled water were used for an extraction, an organic layer therefrom was dried with magnesium sulfate and filtered, and the filtered solution was concentrated under a reduced pressure. A product therefrom was purified with n-hexane/dichloromethane (8:2 of a volume ratio) through silica gel column chromatography to obtain a desired compound, a compound C-3 (17.5 g, 88%).

### SYNTHESIS EXAMPLE 14

### Synthesis of Compound C-6

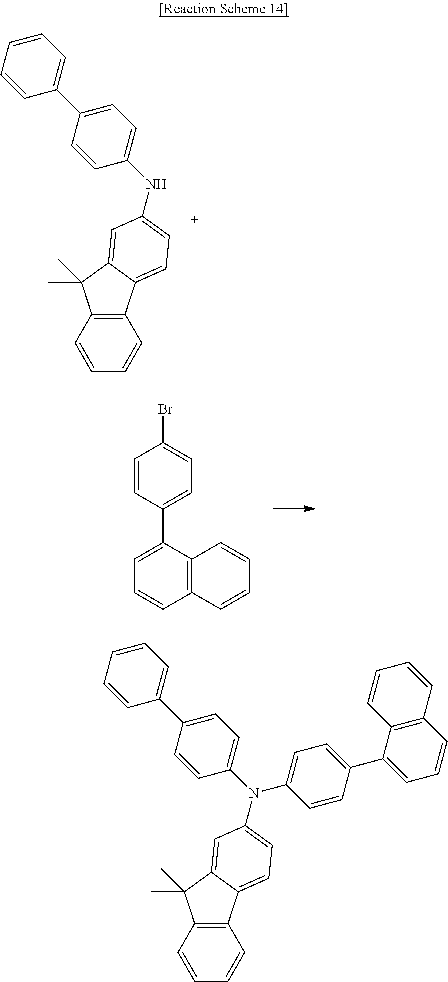

1-(4-bromo-phenyl)-naphthalene (12.6 g, 44.35 mmol), biphenyl-4-yl-(9,9-dimethyl-9H-fluoren-2-yl)-amine (16.0 g, 44.35 mmol), and sodium t-butoxide (8.5 g, 88.7 mmol) were put, and toluene (200 ml) was added thereto to dissolve them. Pd(dba)₂ (0.82 g, 0.89 mmol) and tri-tertiary-butylphosphine (0.54 g, 2.66 mmol) were sequentially added thereto, and the mixture was refluxed and stirred under a nitrogen atmosphere for 6 hours. When the reaction was complete, toluene and distilled water were used for an extraction, an organic layer therefrom was dried with magnesium sulfate and filtered, and filtered, and the filtered solution was concentrated under a reduced pressure. Then, a product therefrom was purified with n-hexane/dichloromethane (8:2 of a volume ratio) through silica gel column chromatography to obtain a desired compound, a compound C-6 (22.0 g, 88%).

### SYNTHESIS EXAMPLE 15

### Synthesis of Compound C-19

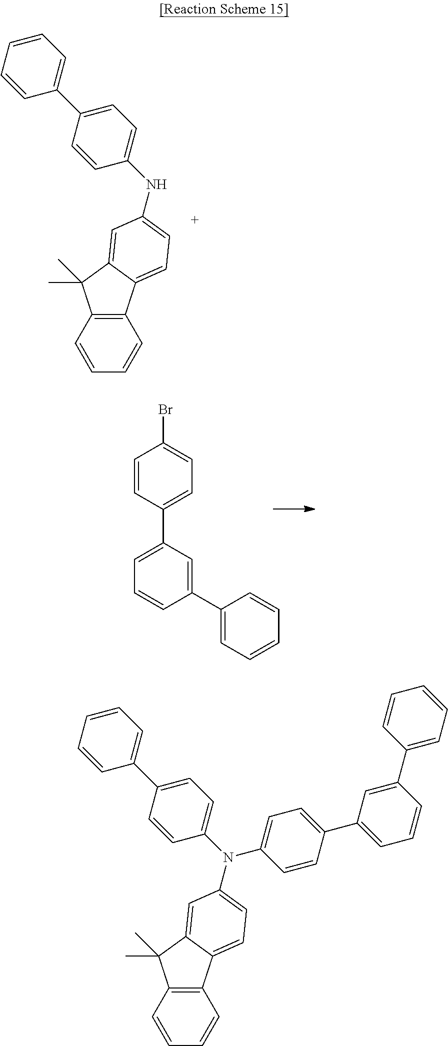 [SMILES: CC1(C)c2ccccc2-c2ccc(Nc3ccc(-c4ccccc4)cc3)cc21 ; Brc1ccc(-c2cccc(-c3ccccc3)c2)cc1 ; CC1(C)c2ccccc2-c2ccc(N(c3ccc(-c4ccccc4)cc3)c3ccc(-c4cccc(-c5ccccc5)c4)cc3)cc21]

4-bromo-[1,1′;3′,1″]terphenyl (13.1 g, 42.39 mmol), biphenyl-4-yl-(9,9-dimethyl-9H-fluoren-2-yl)-amine (15.3 g, 42.39 mmol), and sodium t-butoxide (8.2 g, 84.78 mmol) are put, and toluene (200 ml) was added thereto to dissolve them. Pd(dba)₂ (0.78 g, 0.89 mmol) and tri-tertiary-butylphosphine (0.52 g, 2.54 mmol) were sequentially added thereto, and the mixture was refluxed and stirred under a nitrogen atmosphere for 6 hours. When the reaction was complete, toluene and distilled water were used for an extraction, an organic layer therefrom was dried with magnesium sulfate and filtered, and the filtered solution was concentrated under a reduced pressure. Then, a product therefrom was purified with n-hexane/dichloromethane (8:2 of a volume ratio) through silica gel column chromatography to obtain a desired compound, a compound C-19 (20.4 g, 82%).

### SYNTHESIS EXAMPLE 16

### Synthesis of Compound C-11

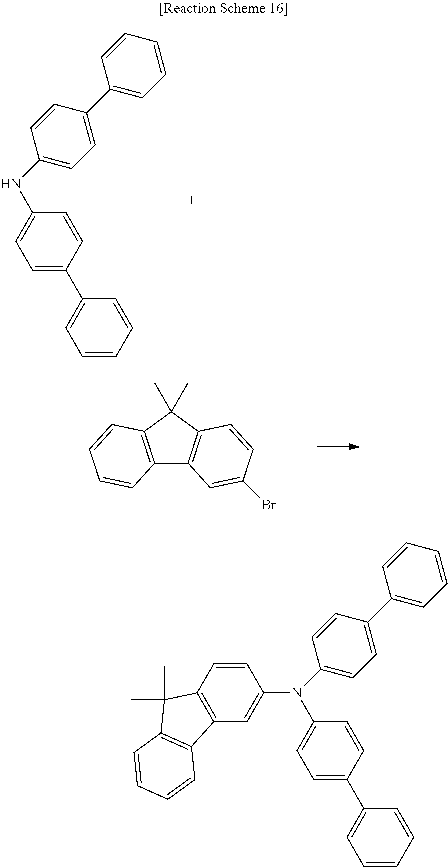 [SMILES: c1ccc(-c2ccc(Nc3ccc(-c4ccccc4)cc3)cc2)cc1 ; CC1(C)c2ccccc2-c2cc(Br)ccc21 ; CC1(C)c2ccccc2-c2cc(N(c3ccc(-c4ccccc4)cc3)c3ccc(-c4ccccc4)cc3)ccc21]

3-bromofluorene (11.7 g, 42.83 mmol), bis-biphenyl-4-yl-amine (12.51 g, 38.94 mmol), and sodium t-butoxide (7.86 g, 81.76 mmol) were put, and toluene (155 ml) was added thereto to dissolve them. Pd(dba)₂ (0.357 g, 0.39 mmol) and tri-tertiary-butylphosphine (0.236 g, 1.17 mmol) were sequentially added thereto, and the resulting mixture was refluxed and stirred under a nitrogen atmosphere for 4 hours. When the reaction was complete, toluene and distilled water were used for an extraction, an organic layer therefrom was dried with magnesium sulfate and filtered, and the filter solution was concentrated under a reduced pressure. Then, a product therefrom was purified with n-hexane/dichloromethane (8:2 of a volume ratio) through silica gel column chromatography to obtain a desired compound C-11 (17.2 g, 86%).

### SYNTHESIS EXAMPLE 17

### Synthesis of Compound C-8

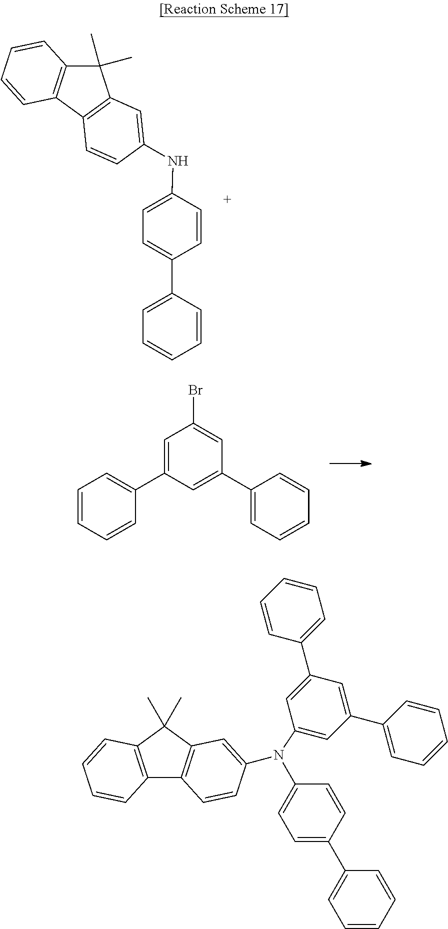

1-bromo-3,5-terphenyl (11.53 g, 37.3 mmol), biphenyl-4-yl-(9,9-dimethyl-9H-fluorene-2-yl)-amine (12.25 g, 33.9 mmol), and sodium t-butoxide (6.84 g, 71.21 mmol) were dissolved in toluene (135 ml). Pd(dba)₂ (0.31 g, 0.34 mmol) and tri-tertiary-butylphosphine (0.21 g, 0.10 mmol) were sequentially added thereto, and the resulting mixture was refluxed and stirred under a nitrogen atmosphere for 4 hours. When the reaction was complete, toluene and distilled water were used for an extraction, an organic layer therefrom was dried with magnesium sulfate and filtered, and the filtered solution was concentrated under a reduced pressure. Then, a product therefrom was purified with n-hexane/dichloromethane (8:2 of a volume ratio) through silica gel column chromatography to obtain a desired compound C-8 (18.1 g, 91%).

### SYNTHESIS EXAMPLE 18

### Synthesis of Compound C-15

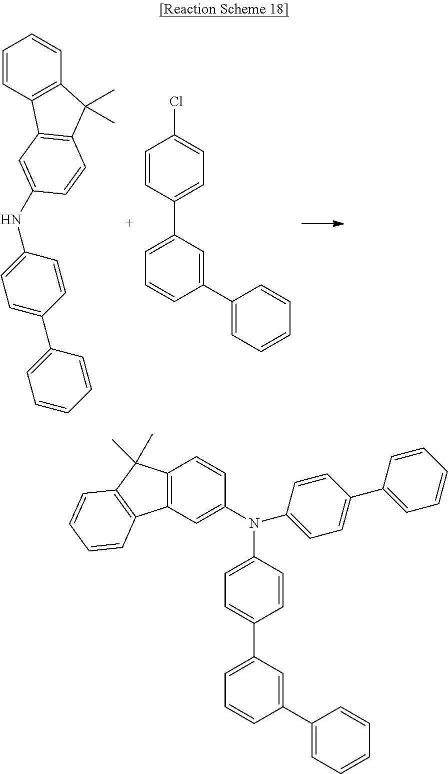

4-chloro-(1,3-terphenyl) (9.88 g, 37.3 mmol), biphenyl-4-yl-(9,9-dimethyl-9H-fluorene-2-yl)-amine (12.25 g, 33.9 mmol), and sodium t-butoxide (6.84 g, 71.21 mmol) were dissolved in toluene (135 ml). Pd(dba)₂ (0.31 g, 0.34 mmol) and tri-tertiary-butylphosphine (0.21 g, 0.10 mmol) were sequentially added thereto, and the resulting mixture was refluxed and stirred under a nitrogen atmosphere for 4 hours. When the reaction was complete, toluene and distilled water were used for an extraction, an organic layer therefrom was dried with magnesium sulfate and filtered, and the filtered solution was concentrated under a reduced pressure. Then, a product therefrom was purified with n-hexane/dichloromethane (8:2 of a volume ratio) through silica gel column chromatography to obtain a desired compound C-15 (17.7 g, 89%).

Manufacture of Organic Light Emitting Diode

### EXAMPLE 1

A glass substrate coated with ITO (indium tin oxide) to be 1500 Å thick was ultrasonic wave-washed with a distilled water. The glass substrate was ultrasonic wave-washed with a solvent such as isopropyl alcohol, acetone, methanol, and the like. moved to a plasma cleaner, cleaned by using oxygen plasma for 10 minutes, and then, moved to a vacuum depositor. This ITO transparent electrode was used as a positive electrode, a 700 Å-thick hole injection layer was formed thereon by vacuum-depositing N4,N4′-diphenyl-N4,N4′-bis(9-phenyl-9H-carbazol-3-yl)biphenyl-4,4′-diamine) (the compound A), and a hole transport layer was formed on the hole injection layer by depositing 1,4,5,8,9,11-hexaazatriphenylene-hexacarbonitrile (HAT-CN) (the compound B) to be 50 Å thick and then, N-(biphenyl-4-yl)-9,9-dimethyl-N-(4-(9-phenyl-9H-carbazol-3-yl)phenyl)-9H-fluoren-2-amine) (the compound C) to be 700 Å thick. On the hole transport layer, a 50 Å-thick hole transport auxiliary layer was formed by vacuum-depositing the compound C-3 according to Synthesis Example 13. Subsequently, on the hole transport auxiliary layer, the compound A-1 of Synthesis Example 6 and the compound B-10 of Synthesis Example 9 were simultaneously used as a host and 10 wt % of tris(4-methyl-2,5-diphenylpyridine)iridium(III) (compound D) was doped as dopant by a vacuum deposition to form a 400 Å-thick emission layer. Herein, the compound A-1 and the compound B-10 were used in a weight ratio of 1:1.

Then, on the emission layer, a 310 Å-thick electron transport layer was formed by simultaneously vacuum-depositing (8-(4-(4-(naphthalen-2-yl)-6-(naphthalen-3-yl)-1,3,5-triazin-2-yl)phenyl)quinoline) (the compound E) and Liq in a ratio of 1:1, and a cathode was formed by sequentially vacuum-depositing Liq to be 15 Å thick and Al to be 1200 Å thick on the electron transport layer to manufacture an organic light emitting diode.

The organic light emitting diode had a six-layered organic thin film structure and specifically,

ITO/A (700 Å)/B (50 Å)/C (720 Å)/hole transport auxiliary layer [compound C-3 (320 Å)]/EML[compound A-1: compound B-10: D=X:X:7 wt %] (400 Å)/E:Liq 300 Å/Liq (15 Å)/Al (1200 Å).

(X=a weight ratio)

### EXAMPLE 2

An organic light emitting diode was manufactured according to the same method as Example 1 except for using the compound B-30 according to Synthesis Example 10 instead of the compound B-10 for the emission layer.

### EXAMPLE 3

An organic light emitting diode was manufactured according to the same method as Example 1 except for using the compound A-1 and the compound B-10 in a weight ratio of 4:1 instead of 1:1.

### EXAMPLE 4

An organic light emitting diode was manufactured according to the same method as Example 2 except for using the compound A-19 according to Synthesis Example 7 instead of the compound A-1 for the emission layer and

the compound C-6 according to Synthesis Example 14 instead of the compound C-3 for the hole transport auxiliary layer.

### EXAMPLE 5

An organic light emitting diode was manufactured according to the same method as Example 2 except for using the compound A-46 according to Synthesis Example 8 instead of the compound A-1 for the emission layer and

the compound C-19 according to Synthesis Example 15 instead of the compound C-3 for the hole transport auxiliary layer.

### EXAMPLE 6

An organic light emitting diode was manufactured according to the same method as Example 5 except for using the compound A-46 and the compound B-30 in a weight ratio of 4:1 instead of 1:1.

### REFERENCE EXAMPLE 1

An organic light emitting diode was manufactured according to the same method as Example 1 except for using the compound D-128 according to Synthesis Example 11 instead of the compound B-10 for the emission layer.

### REFERENCE EXAMPLE 2

An organic light emitting diode was manufactured according to the same method as Example 1 except for using the compound D-129 according to Synthesis Example 12 instead of the compound B-10 for the emission layer.

### COMPARATIVE EXAMPLE 1

An organic light emitting diode was manufactured according to the same method as Example 1 except for using the compound A-1 as a SINGLE host instead of the compound A-1 and the compound B-10 as a MIXED host for the emission layer.

### COMPARATIVE EXAMPLE 2

An organic light emitting diode was manufactured according to the same method as Example 1 except for using the compound B-10 as a SINGLE host instead of the compound A-1 and the compound B-10 as a MIXED host for the emission layer.

### COMPARATIVE EXAMPLE 3

An organic light emitting diode was manufactured according to the same method as Example 1 except for using the compound D-128 as a SINGLE host instead of the compound A-1 and the compound B-10 as a MIXED host for the emission layer.

### COMPARATIVE EXAMPLE 4

An organic light emitting diode was manufactured according to the same method as Example 1 except for using the compound D-129 as a SINGLE host instead of the compound A-1 and the compound B-10 as a MIXED host for the emission layer.

Evaluation

Luminous efficiency and roll-off characteristics of each organic light emitting diode according to Examples 1 to 6, Reference Example 1, Reference Example 2 and Comparative Examples 1 to 4 were measured.

Specific measurement methods were as follows, and the results were provided in Table 1.

(1) Measurement of Current Density Change Depending on Voltage Change

The obtained organic light emitting diodes were measured for current value flowing in the unit device while increasing the voltage from 0 V to 10 V using a current-voltage meter (Keithley 2400), the measured current value was divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

Current efficiency (cd/A) at the same current density (10 mA/cm²) were calculated by using the luminance, current density, and voltages (V) from the items (1) and (2).

(4) Measurement of Life-Span

Life-span was obtained by measuring a time taken until current efficiency (cd/A) decreased down to 90% while luminance (cd/m²) was maintained at 5000 cd/m².

(5) Roll-Off Characteristics

Efficiency roll-off was calculated as a percentage through (Max measurement−Measurement at 6000 cd/m²/Max measurement) among the measurements of (3).

TABLE 1

| | Hole | Emission layer | | | | | |
|---|---|---|---|---|---|---|---|
| | transport | First | Second | | Luminous | Life- | Roll- |
| | auxiliary | compound | compound | H1:H2 | efficiency | span | off |
| | layer | (H1) | (H2) | (wt:wt) | (cd/A) | (T90) | (%) |
| Ex. 1 | C-3 | A-1 | B-10 | 1:1 | 57.2 | 1200 | 11.5 |
| Ex. 2 | C-3 | A-1 | B-30 | 1:1 | 58.4 | 1800 | 10.8 |
| Ex. 3 | C-3 | A-1 | B-10 | 4:1 | 63.5 | 1700 | 9.3 |
| Ex. 4 | C-6 | A-19 | B-30 | 1:1 | 59.3 | 1500 | 12.5 |
| Ex. 5 | C-19 | A-46 | B-30 | 1:1 | 57.6 | 2000 | 11.5 |
| Ex. 6 | C-19 | A-46 | B-30 | 4:1 | 57.6 | 1900 | 10.1 |
| Ref. Ex. 1 | C-3 | A-1 | D-128 | 1:1 | 55.4 | 800 | 15.3 |
| Ref. Ex. 2 | C-3 | A-1 | D-129 | 1:1 | 56.7 | 900 | 17.2 |
| Comp. Ex. 1 | C-3 | A-1 | | — | 58.0 | 1200 | 17.5 |
| Comp. Ex. 2 | C-3 | | B-10 | — | 45.4 | 500 | 21.2 |
| Comp. Ex. 3 | C-3 | | D-128 | — | 44.2 | — | 23.5 |
| Comp. Ex. 4 | C-3 | | D-129 | — | 43.7 | — | 18.4 |

Referring to Table 1, when a material having biased electron or hole characteristics is used to form an emission layer, excitons in a device including the emission layer are relatively more generated due to recombination of carriers on the interface between the emission layer and the electron or hole transport layer. As a result, the molecular excitons in the emission layer interact with charges on the interface of the hole transport layer and thus, cause a roll-off of sharply deteriorating efficiency and also, sharply deteriorate light emitting life-span characteristics. In order to solve the problems, the first and second hosts are simultaneously included in the emission layer to make a light emitting region not biased to either of the electron transport layer or the hole transport layer

Particularly, the homo position of the second host is more widely distributed when there is one carbazole than when there are two carbazoles and thus may stably carry more holes and provide a device capable of balancing carriers in the emission layer and thus remarkably improve roll-off characteristics and life-span characteristics.

While this invention has been described in connection with what is presently considered to be practical exemplary embodiments, it is to be understood that the invention is not limited to the disclosed embodiments, but, on the contrary, is intended to cover various modifications and equivalent arrangements included within the spirit and scope of the appended claims. Therefore, the aforementioned embodiments should be understood to be exemplary but not limiting the present invention in any way.

<Description of Symbols>

| | |
|---|---|
| 10: anode | 20: cathode |
| 30: organic layer | 31: hole transport layer |
| 32: emission layer | 33: hole transport auxiliary layer |
| 34: electron transport layer | 35: electron transport auxiliary layer |
| 36: electron injection layer | 37: hole injection layer |

What is claimed is:

1. An organic optoelectronic device, comprising
an anode and a cathode facing each other,
an emission layer between the anode and the cathode, a hole transport layer between the anode and the emission layer, and
a hole transport auxiliary layer between the hole transport layer and the emission layer,
wherein the emission layer includes at least one first compound represented by Chemical Formula 1; and at least one second compound represented by Chemical Formula 2, and
the hole transport auxiliary layer includes at least one third compound represented by Chemical Formula 3: 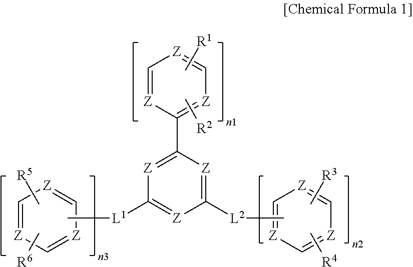
wherein, in Chemical Formula 1,
Z is independently N, C, or CR^{a},
at least one of Z is N,
R¹ to R⁶ and R^{a} are independently hydrogen, deuterium, a substituted or unsubstituted C1 to C 10 alkyl group, a substituted or unsubstituted C6 to C30 aryl group, a substituted or unsubstituted C2 to C30 heterocyclic group, or a combination thereof,
R³ ₜₒ R⁶ are independently present or adjacent groups are linked to each other to provide a ring,
L¹ and L² are independently a single bond, a substituted or unsubstituted C6 to C30 arylene group, a substituted or unsubstituted C2 to C30 heteroarylene group, or a combination thereof,
n1 is one of integers of 0 to 3, and
n2 and n3 are independently one of integers of 1 to 5; 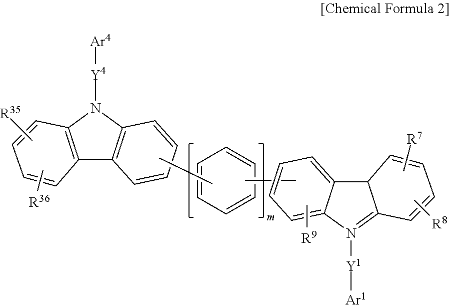
wherein, in Chemical Formula 2,
Y¹ and Y⁴ are independently a single bond, a substituted or unsubstituted C6 to C30 arylene group, a substituted or unsubstituted C2 to C30 heteroarylene group, or a combination thereof,
Ar¹ and Ar⁴ are independently a substituted or unsubstituted C6 to C30 aryl group, a substituted or unsubstituted C2 to C30 heterocyclic group, or a combination thereof,
R⁷ to R⁹, R³⁵, and R³⁶ are independently hydrogen, deuterium, a substituted or unsubstituted C1 to C20 alkyl group, a substituted or unsubstituted C6 to C50 aryl group, a substituted or unsubstituted C2 to C50 heterocyclic group, or a combination thereof, and
m is one of integers of 0 to 4, 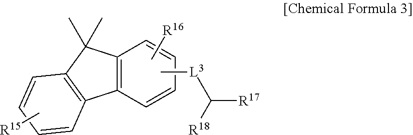
wherein, in Chemical Formula 3,
R¹⁵ to R¹⁸ are independently, hydrogen, deuterium, a substituted or unsubstituted C1 to C30 alkyl group, a substituted or unsubstituted C6 to C30 aryl group, a substituted or unsubstituted C2 to C30 heteroaryl group, or a combination thereof,
L³ is a single bond, a substituted or unsubstituted C6 to C30 arylene group, a substituted or unsubstituted C2 to C30 heteroarylene group, or a combination thereof, and
“substituted” of Chemical Formulae 1 to 3 refers to replacement of at least one hydrogen by a deuterium, a halogen, a hydroxyl group, an amino group, C1 to C30 amine group, a nitro group, a C1 to C40 silyl group, a C1 to C30 alkyl group, a C3 to C30 cycloalkyl group, a C2 to C30 heterocycloalkyl group, a C6 to C30 aryl group, a C2 to C30 heterocyclic group, a C1 to C20 alkoxy group, a fluoro group, a C1 to C 10 trifluoroalkyl group, or a cyano group.

2. The organic optoelectronic device of claim 1, wherein Chemical Formula 1 is represented by Chemical Formula 1-I or 1-II: 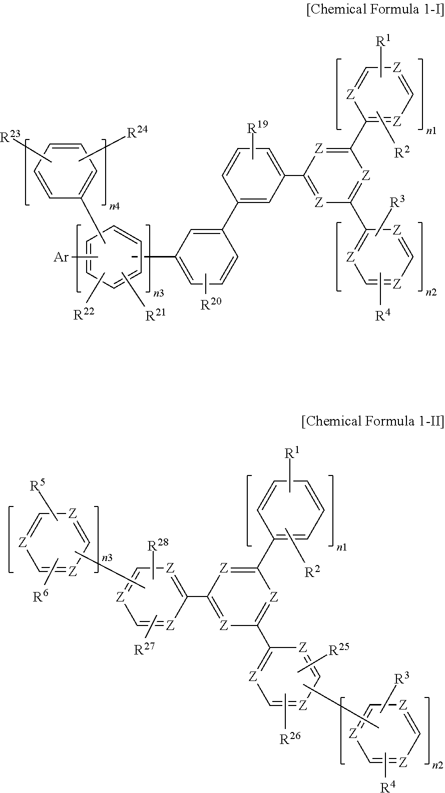
wherein, in Chemical Formulae 1-I and 1-II,
Z is independently N, C, or CR^{a},
at least one of Z is N,
Ar is independently a substituted or unsubstituted C6 to C30 aryl group, a substituted or unsubstituted C2 to C30 heterocyclic group, or a combination thereof,
R¹ to R⁶, R¹⁹ to R²⁸, and R^{a} are independently hydrogen, deuterium, a substituted or unsubstituted C1 to C10 alkyl group, a substituted or unsubstituted C6 to C12 aryl group, a substituted or unsubstituted C2 to C30 heterocyclic group, or a combination thereof,
R³ to R⁶, R²¹ and R²², and R²³ and R²⁴ are independently present or adjacent groups are linked to each other to provide a ring,
n1 is one of integers of 0 to 3,
n2 and n3 are independently one of integers of 1 to 5, and
n4 is an integer ranging from 0 to 2,
wherein, “substituted” is the same as defined in claim 1.

3. The organic optoelectronic device of claim 2, wherein Chemical Formula 1-I is represented by one of Chemical Formulae 1-IA to 1-IC: 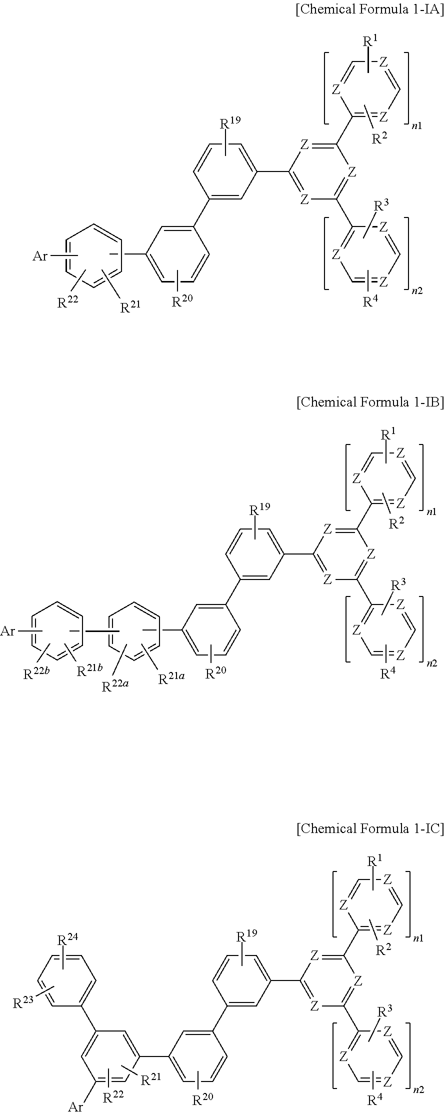
wherein, in Chemical Formulae I -IA to I -IC,
Z is independently N, C, or CR^{a},
at least one of Z is N,
R¹ to R⁴, R¹⁹ to R²⁴, and R^{a} are independently hydrogen, deuterium, a substituted or unsubstituted C1 to C10 alkyl group, a substituted or unsubstituted C6 to C12 aryl group, a substituted or unsubstituted C2 to C30 heteroaryl group, or a combination thereof,
Ar is independently a substituted or unsubstituted C6 to C30 aryl group, a substituted or unsubstituted C2 to C30 heterocyclic group, or a combination thereof,
R³ and R⁴, R²¹ and R²², and R²³ and R²⁴ are independently present or adjacent groups are linked to each other to provide a ring,
R^{21a} and R^{21b} are the same as R²¹,
R^{22a} and R^{22b} are the same as R²²,
n1 is one of integers of 0 to 3, and
n2 is one of integers of 1 to 5,
wherein, “substituted” is the same as defined in claim 1.

4. The organic optoelectronic device of claim 2, wherein Chemical Formula 1-II is represented by Chemical Formulae 1-IIA or 1-IIB: 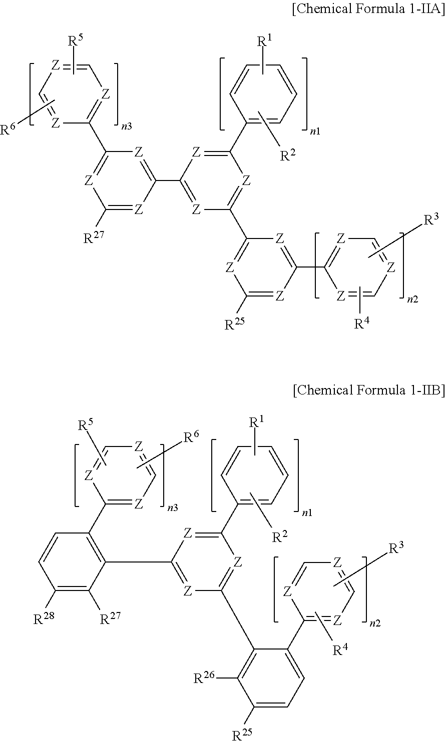
wherein, in Chemical Formulae 1-IIA and 1-IIB,
Z is independently N, C, or CR^{a},
at least one of Z is N,
R¹ to R⁶, R²⁵ to R²⁸, and R^{a} are independently hydrogen, deuterium, a substituted or unsubstituted C1 to C10 alkyl group, a substituted or unsubstituted C6 to C 12 aryl group, a substituted or unsubstituted C2 to C30 heterocyclic group, or a combination thereof,
R³ to R⁶ are independently present or adjacent groups are linked to each other to provide a ring,
n1 is one of integers of 0 to 3, and
n2 and n3 are independently one of integers of 1 to 5,
wherein, “substituted” is the same as defined in claim 1.

5. The organic optoelectronic device of claim 1, wherein Ar¹ and Ar⁴ of Chemical Formula 2 are independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted triphenylenyl group, a substituted or unsubstituted benzofuranyl group, a substituted or unsubstituted benzothiophenyl group, a substituted or unsubstituted fluorenyl group, a substituted or unsubstituted dibenzofuranyl group, a substituted or unsubstituted dibenzothiophenyl group, a substituted or unsubstituted pyridinyl group, a substituted or unsubstituted pyrimidinyl group, or a combination thereof.

6. The organic optoelectronic device of claim 1, wherein Chemical Formula 2 is one of structures of Group 3, and the .—Y¹—Ar¹ and .—Y⁴—Ar⁴ are independently one of substituents of Group 4: 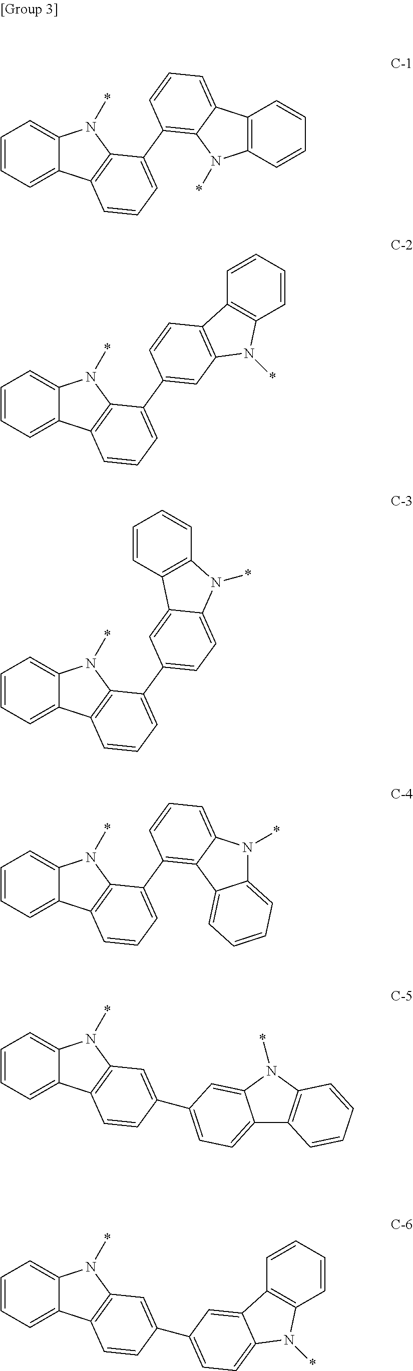 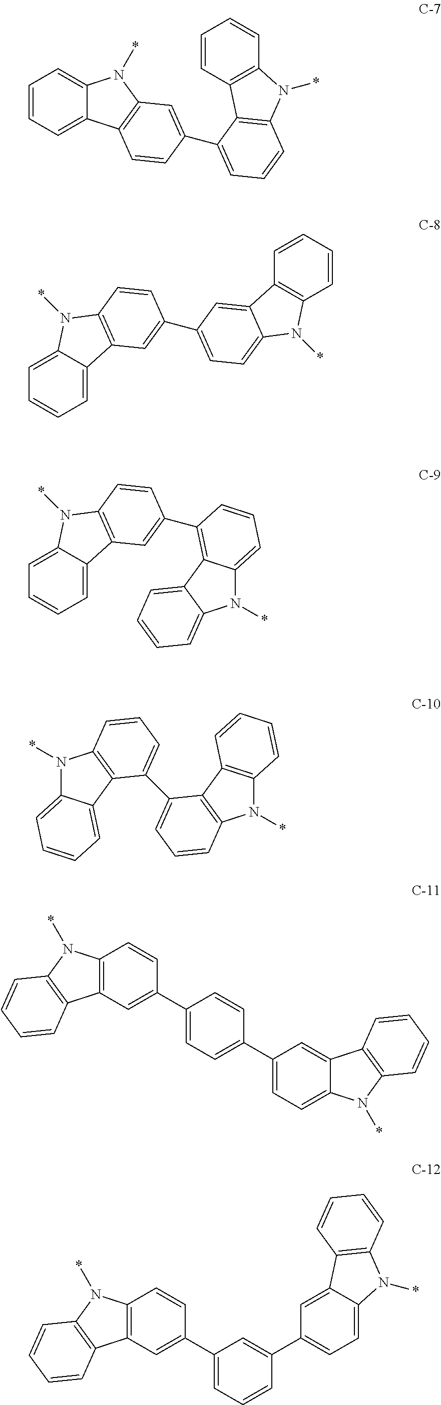 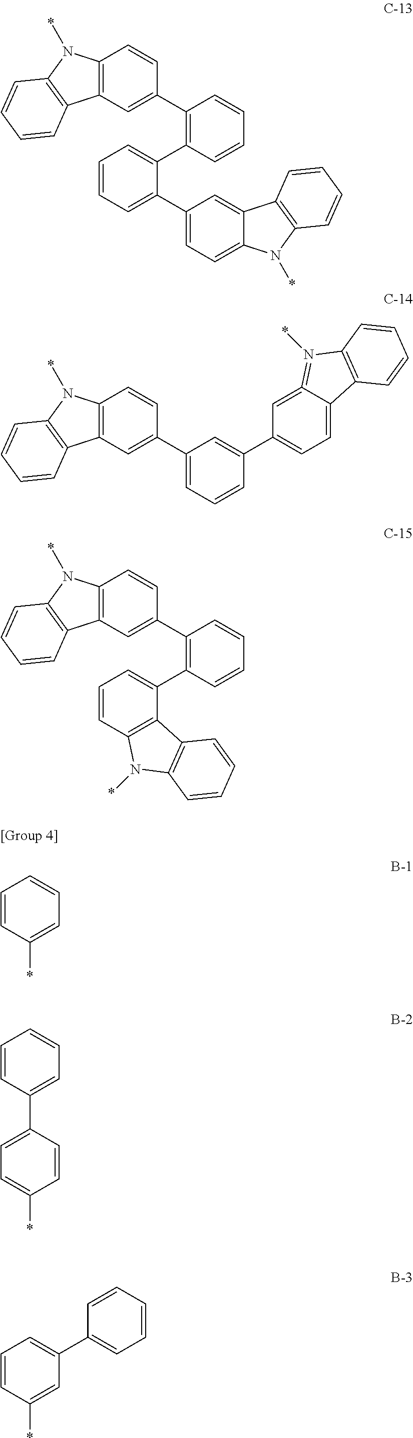 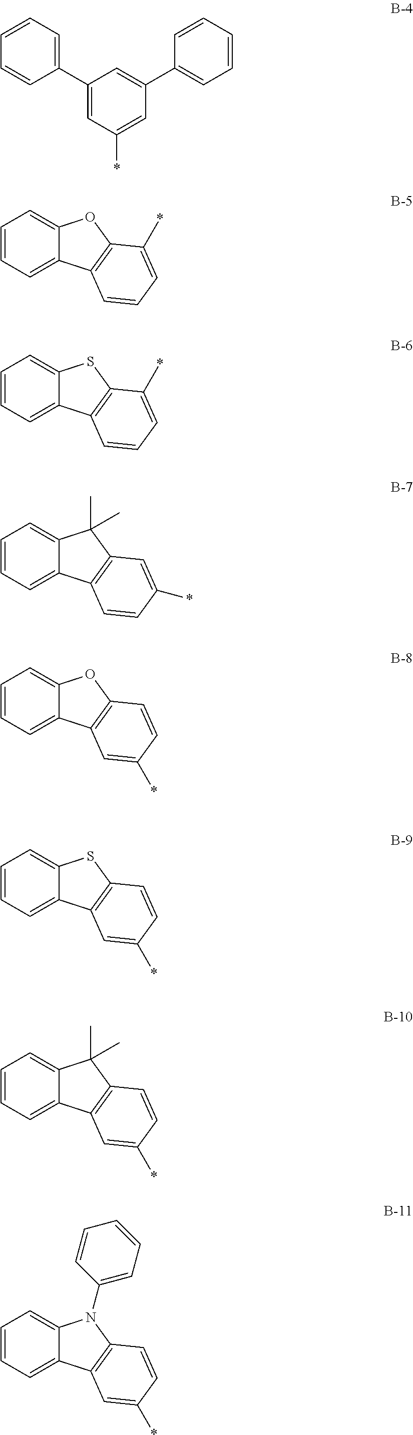 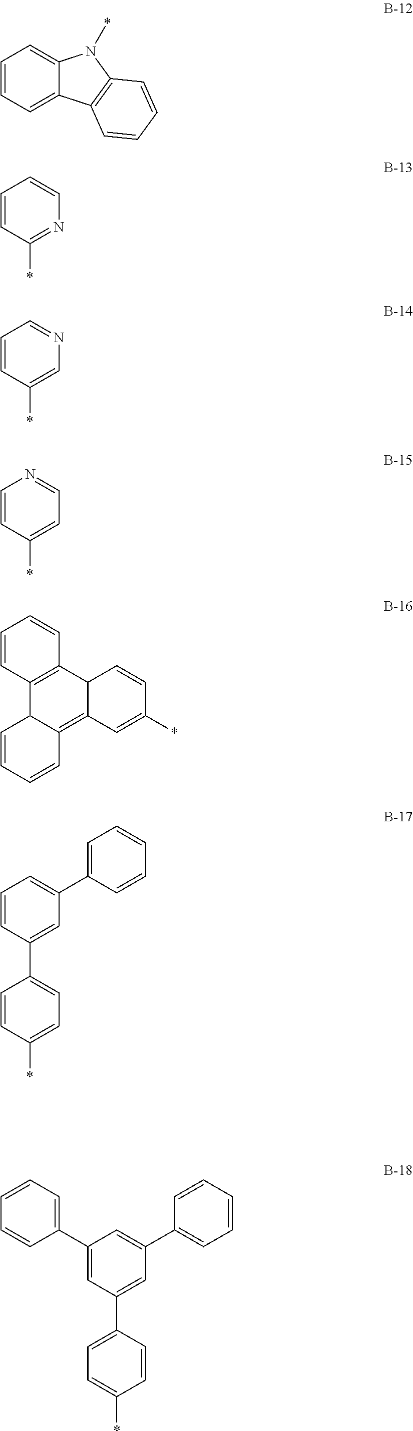
wherein, in Group 3 and Group 4, * is a linking point.

7. The organic optoelectronic device of claim 1, wherein Ar¹ and Ar⁴ of Chemical Formula 2 are independently selected from substituted or unsubstituted groups of Group 5: 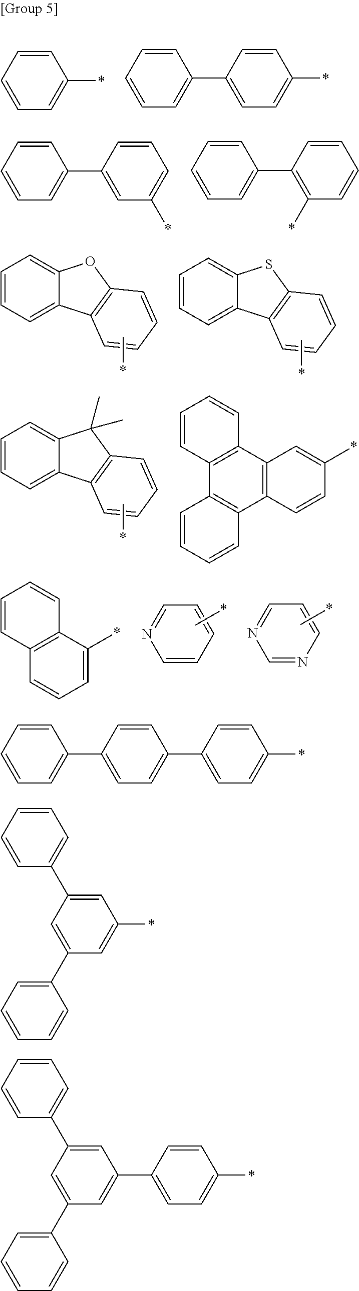 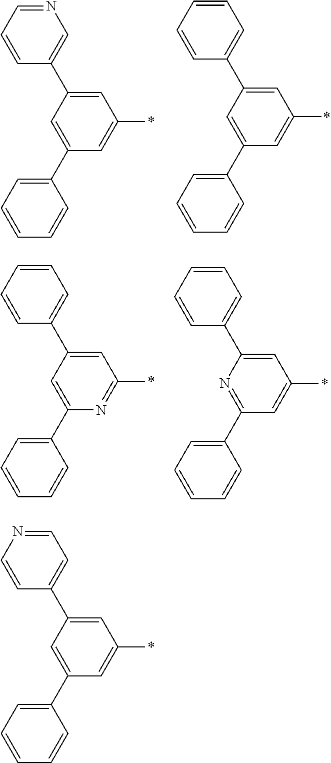
wherein, in Group 5, * is a linking point.

8. The organic optoelectronic device of claim 1, wherein R¹⁵ and R¹⁶ of Chemical Formula 3 are independently hydrogen,
R¹⁷ and R¹⁸ are independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, or a substituted or unsubstituted triphenylenyl group, and L³ is a single bond, a substituted or unsubstituted phenylene group, a substituted or unsubstituted biphenylene group, a substituted or unsubstituted terphenylenyl group, or a substituted or unsubstituted naphthylenyl group.

9. The organic optoelectronic device of claim 1, wherein R¹⁷ and R¹⁸ of Chemical Formula 3 are selected from substituted or unsubstituted groups of Group 6: 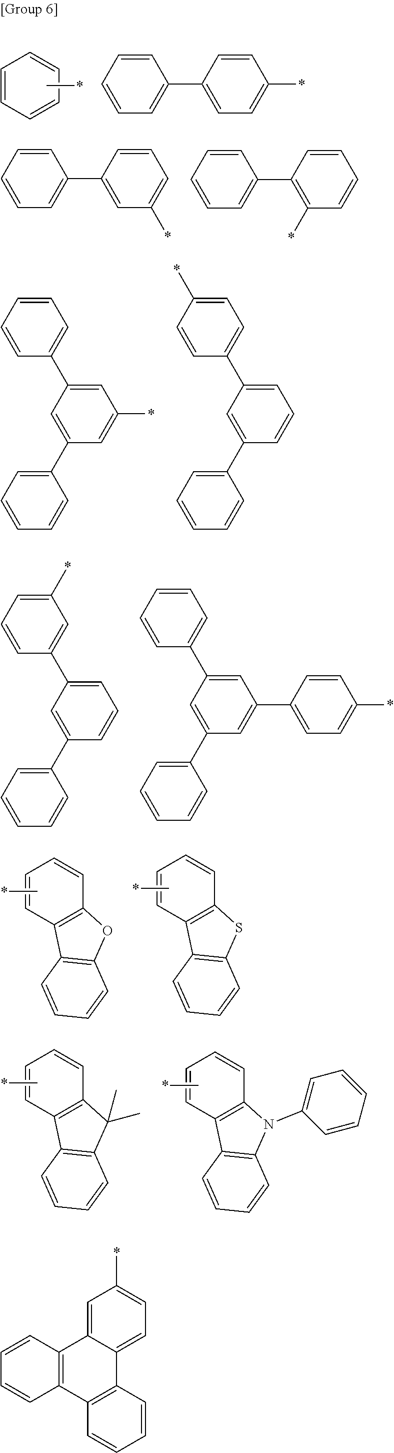 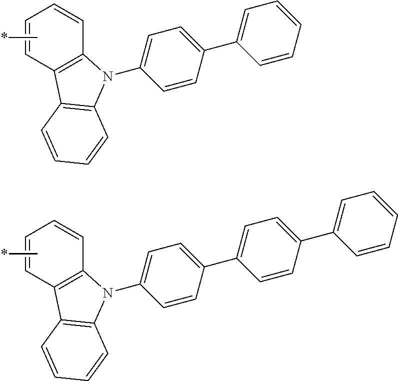
wherein, in Group 6, * is a linking point.

10. The organic optoelectronic device of claim 1, wherein the third compound may be selected from, for example compounds of Group C:

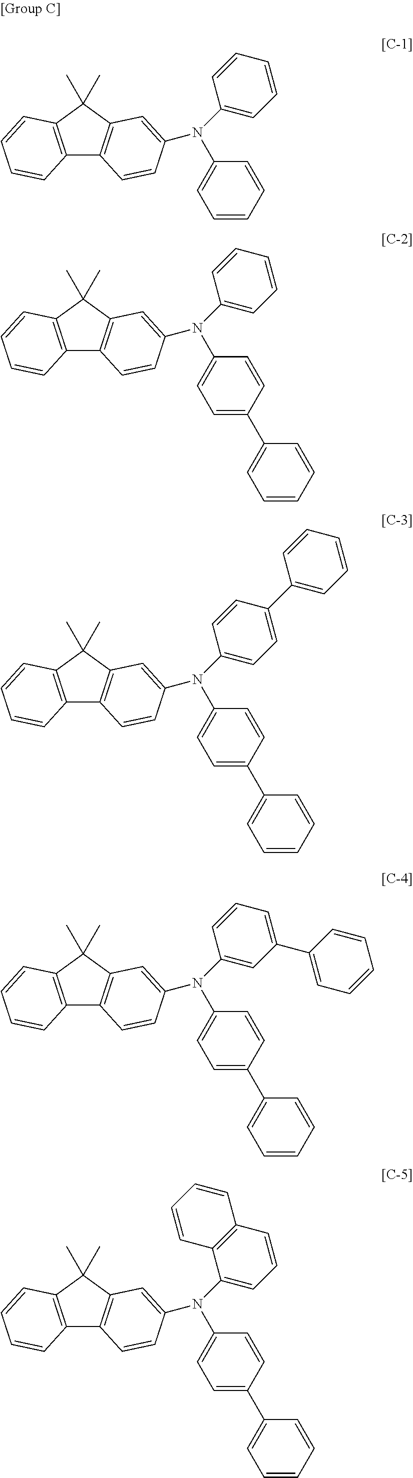

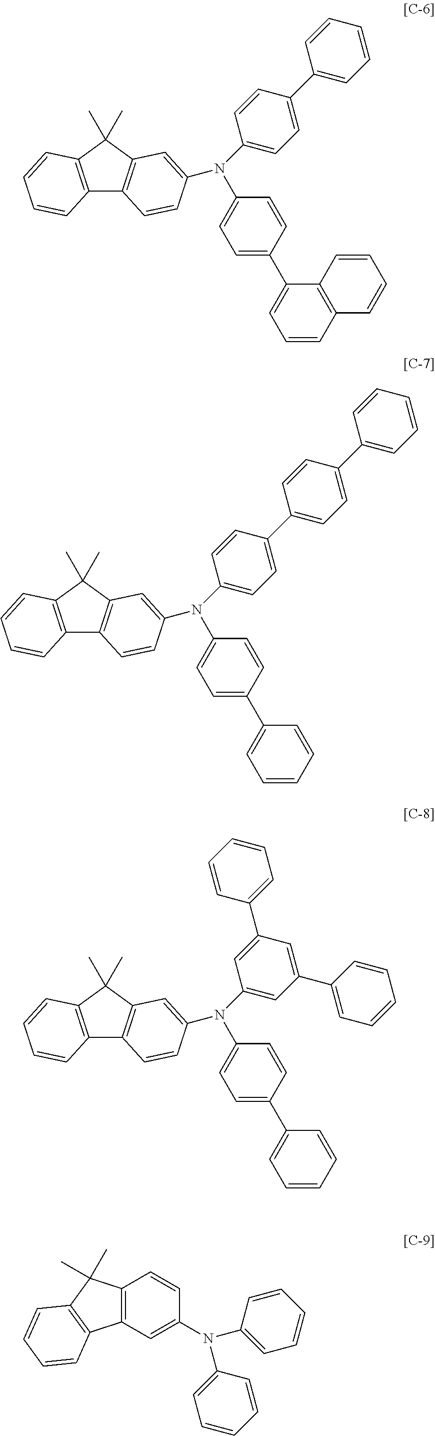 [SMILES: CC1(C)c2ccccc2-c2ccc(N(c3ccc(-c4ccccc4)cc3)c3ccc(-c4cccc5ccccc45)cc3)cc21 ; CC1(C)c2ccccc2-c2ccc(N(c3ccc(-c4ccccc4)cc3)c3ccc(-c4ccc(-c5ccccc5)cc4)cc3)cc21 ; CC1(C)c2ccccc2-c2ccc(N(c3ccc(-c4ccccc4)cc3)c3cc(-c4ccccc4)cc(-c4ccccc4)c3)cc21 ; CC1(C)c2ccccc2-c2cc(N(c3ccccc3)c3ccccc3)ccc21]

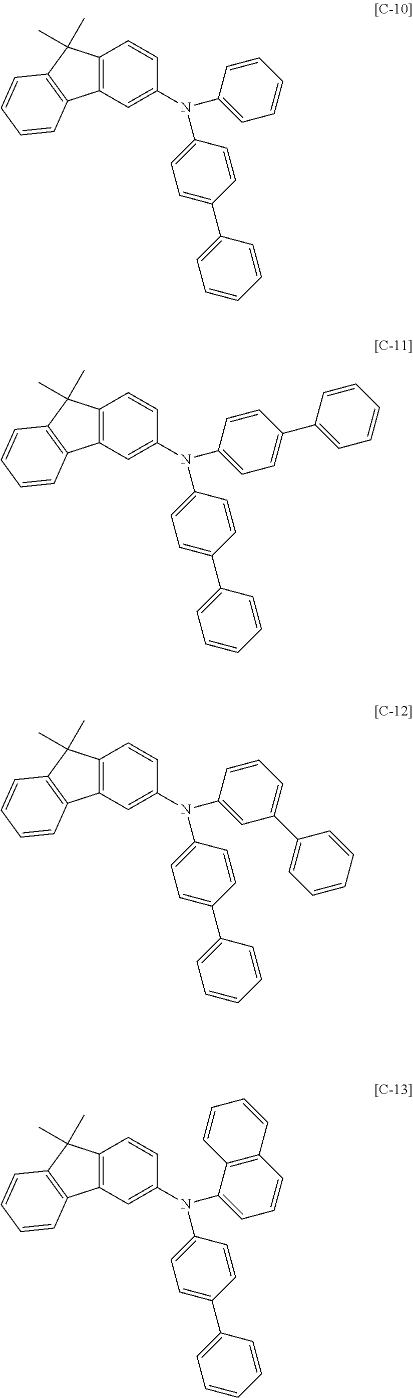 [SMILES: CC1(C)c2ccccc2-c2cc(N(c3ccccc3)c3ccc(-c4ccccc4)cc3)ccc21 ; CC1(C)c2ccccc2-c2cc(N(c3ccc(-c4ccccc4)cc3)c3ccc(-c4ccccc4)cc3)ccc21 ; CC1(C)c2ccccc2-c2cc(N(c3ccc(-c4ccccc4)cc3)c3cccc(-c4ccccc4)c3)ccc21 ; CC1(C)c2ccccc2-c2cc(N(c3ccc(-c4ccccc4)cc3)c3cccc4ccccc34)ccc21]

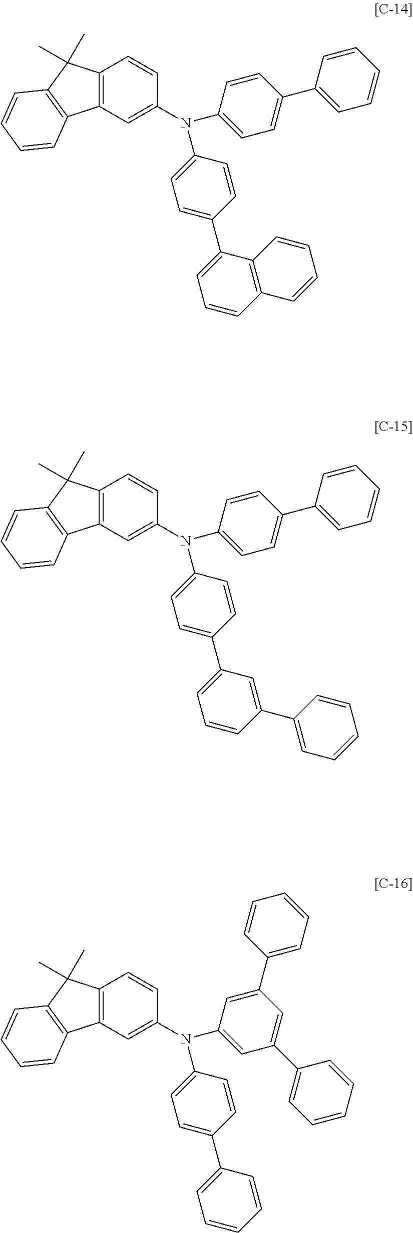 [SMILES: CC1(C)c2ccccc2-c2cc(N(c3ccc(-c4ccccc4)cc3)c3ccc(-c4cccc5ccccc45)cc3)ccc21 ; CC1(C)c2ccccc2-c2cc(N(c3ccc(-c4ccccc4)cc3)c3ccc(-c4cccc(-c5ccccc5)c4)cc3)ccc21 ; CC1(C)c2ccccc2-c2cc(N(c3ccc(-c4ccccc4)cc3)c3cc(-c4ccccc4)cc(-c4ccccc4)c3)ccc21]

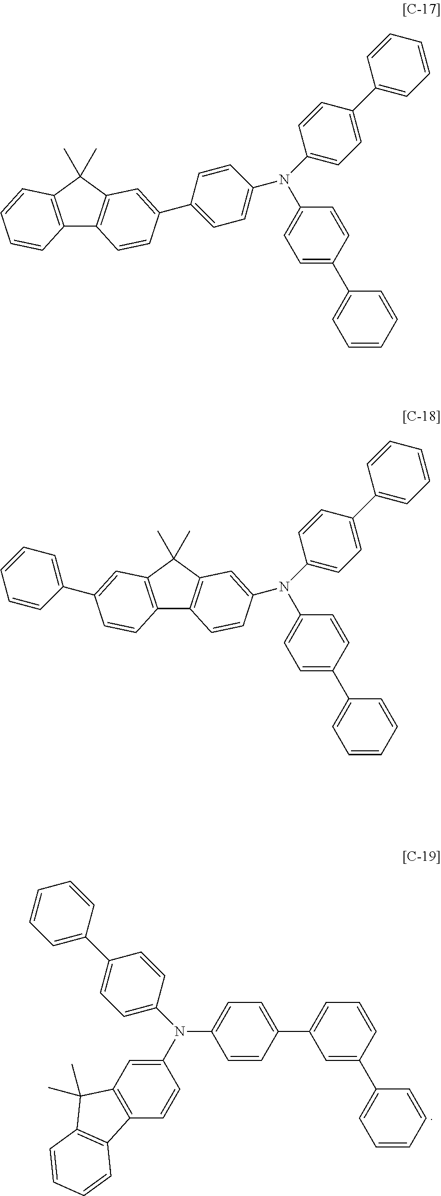 [SMILES: CC1(C)c2ccccc2-c2ccc(-c3ccc(N(c4ccc(-c5ccccc5)cc4)c4ccc(-c5ccccc5)cc4)cc3)cc21 ; CC1(C)c2cc(-c3ccccc3)ccc2-c2ccc(N(c3ccc(-c4ccccc4)cc3)c3ccc(-c4ccccc4)cc3)cc21 ; CC1(C)c2ccccc2-c2ccc(N(c3ccc(-c4ccccc4)cc3)c3ccc(-c4cccc(-c5ccccc5)c4)cc3)cc21]

11. The organic optoelectronic device of claim 1, wherein the emission layer comprises at least one first compound represented by Chemical Formula 1-I, and
at least one second compound of Group B: 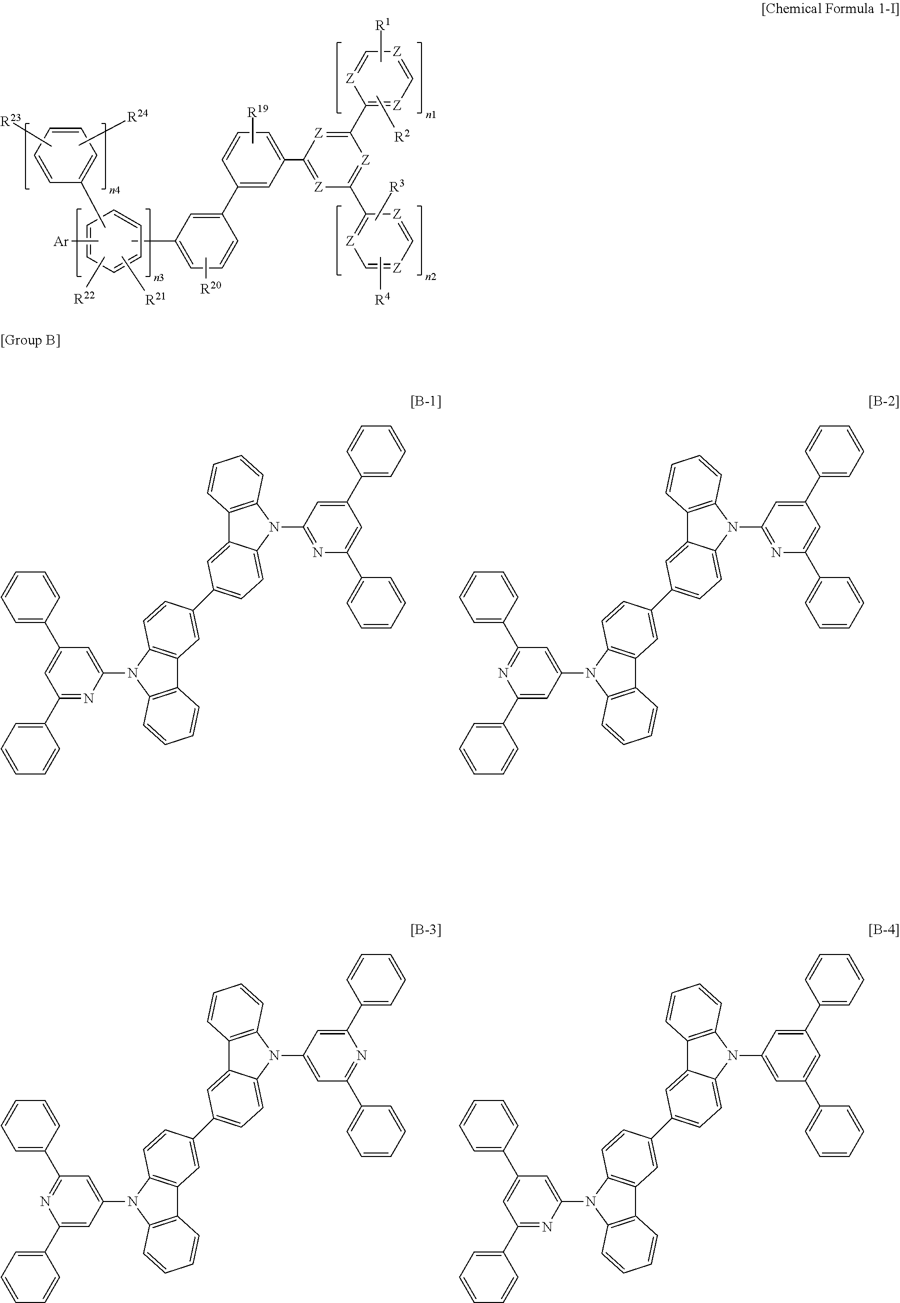 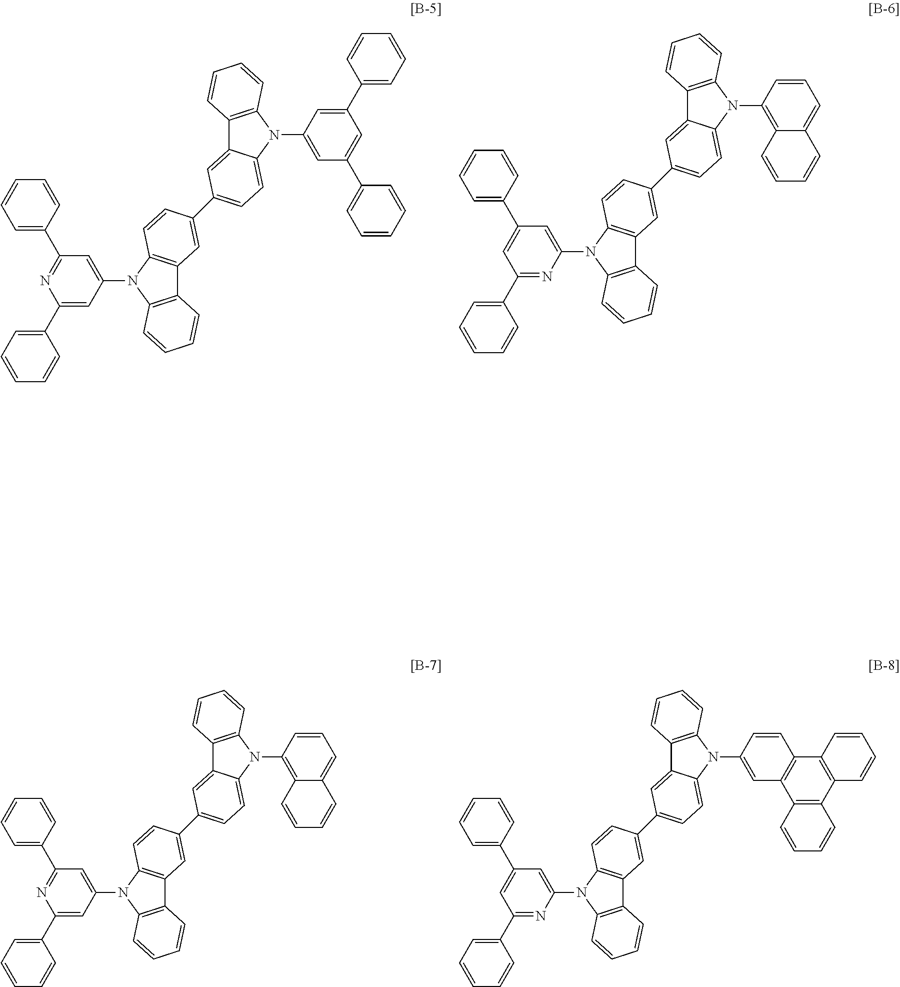 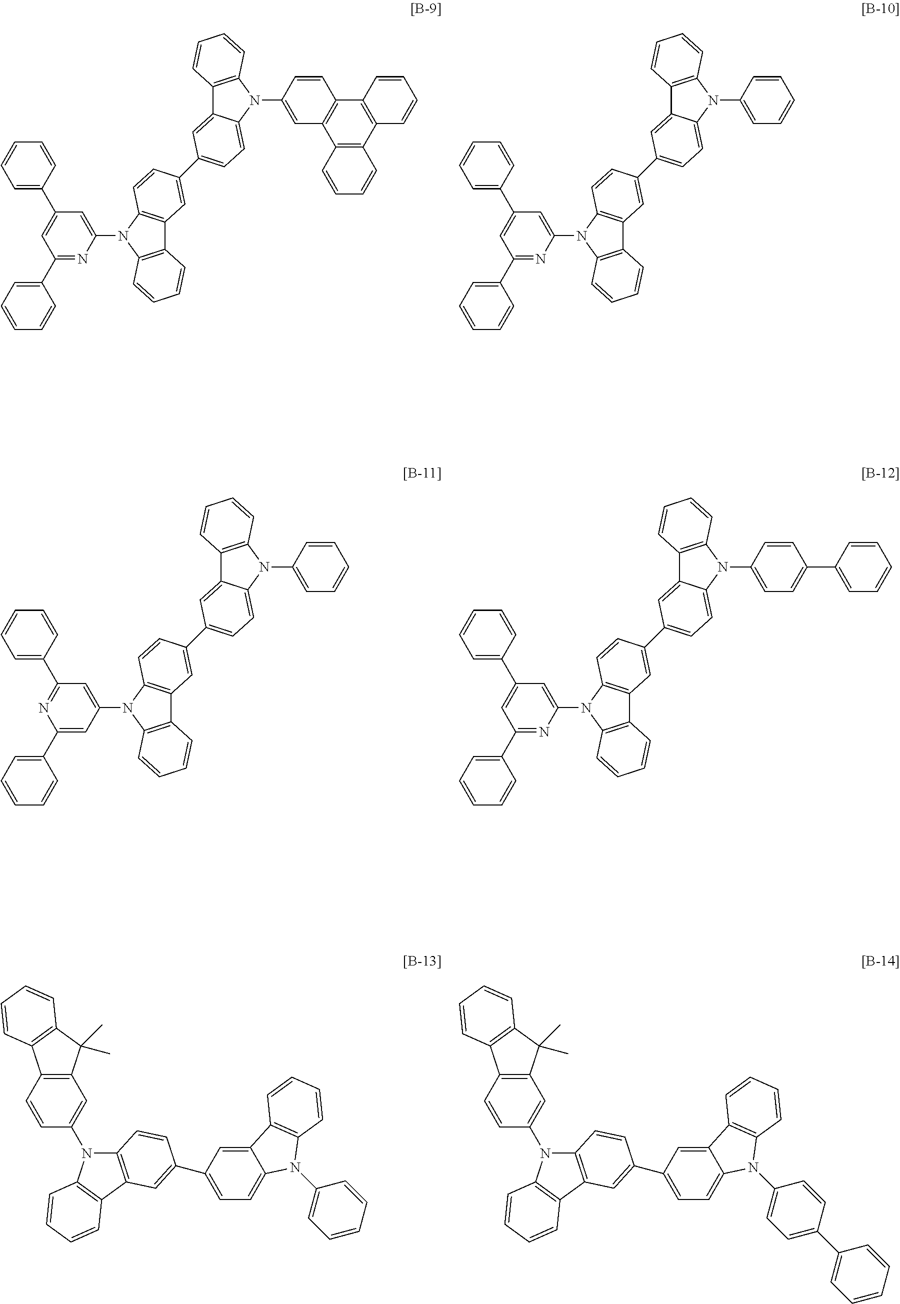 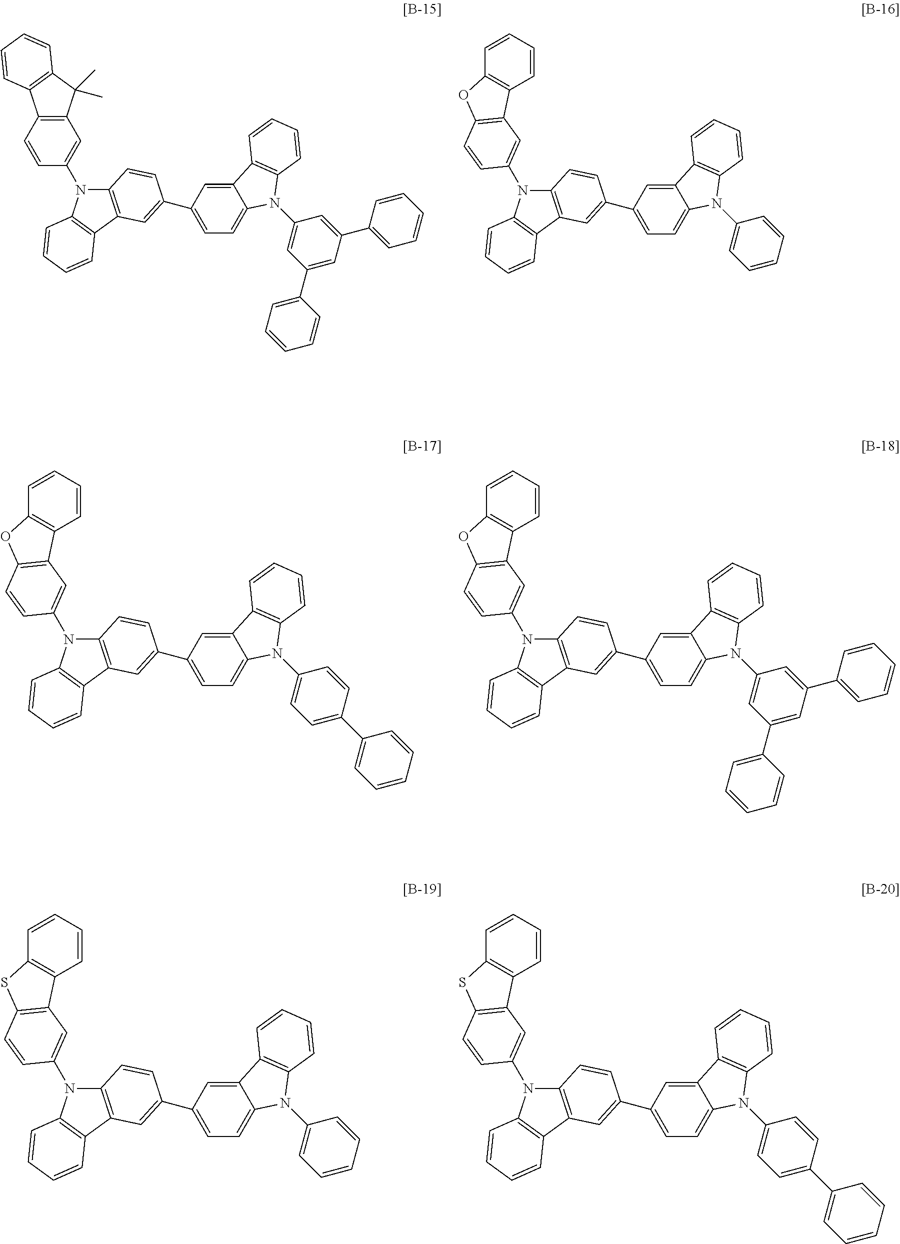 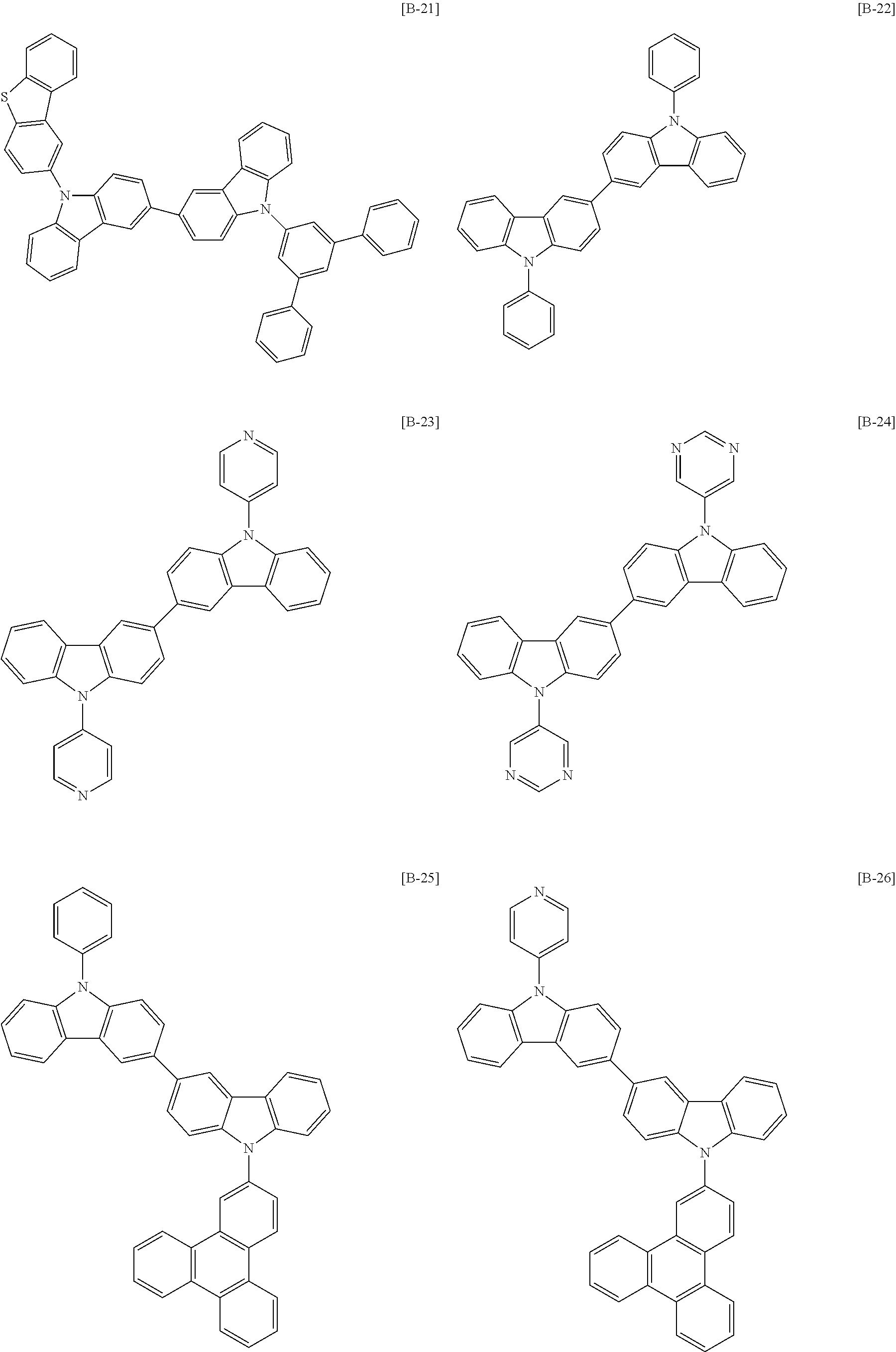 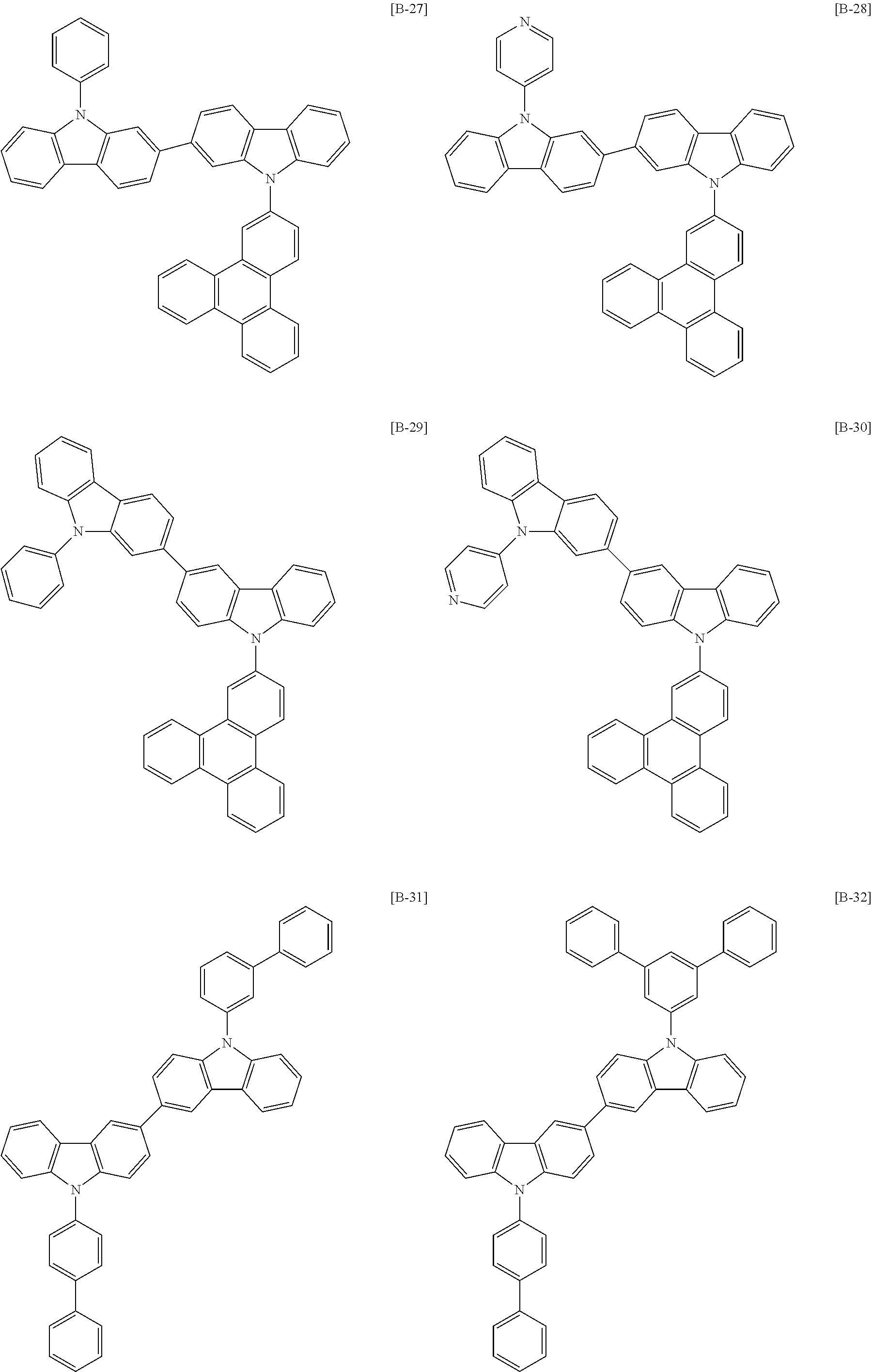 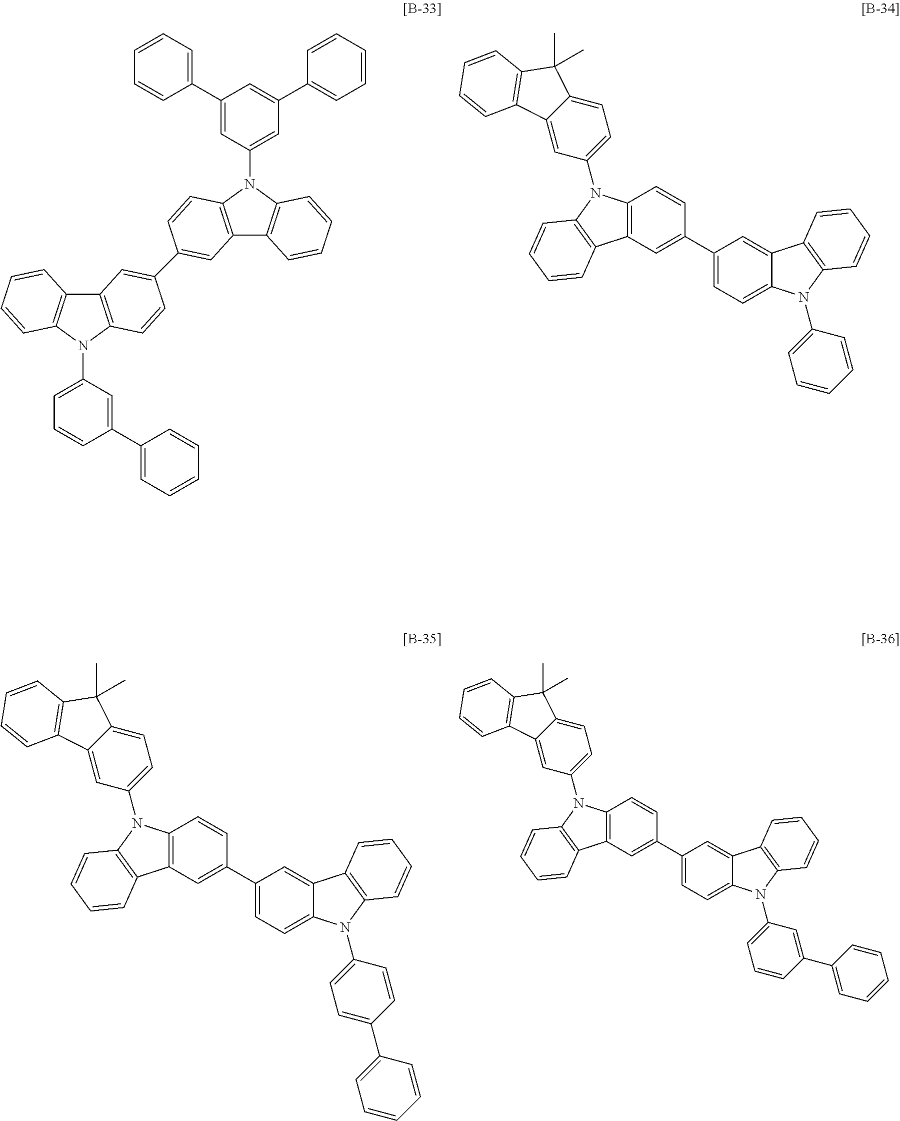 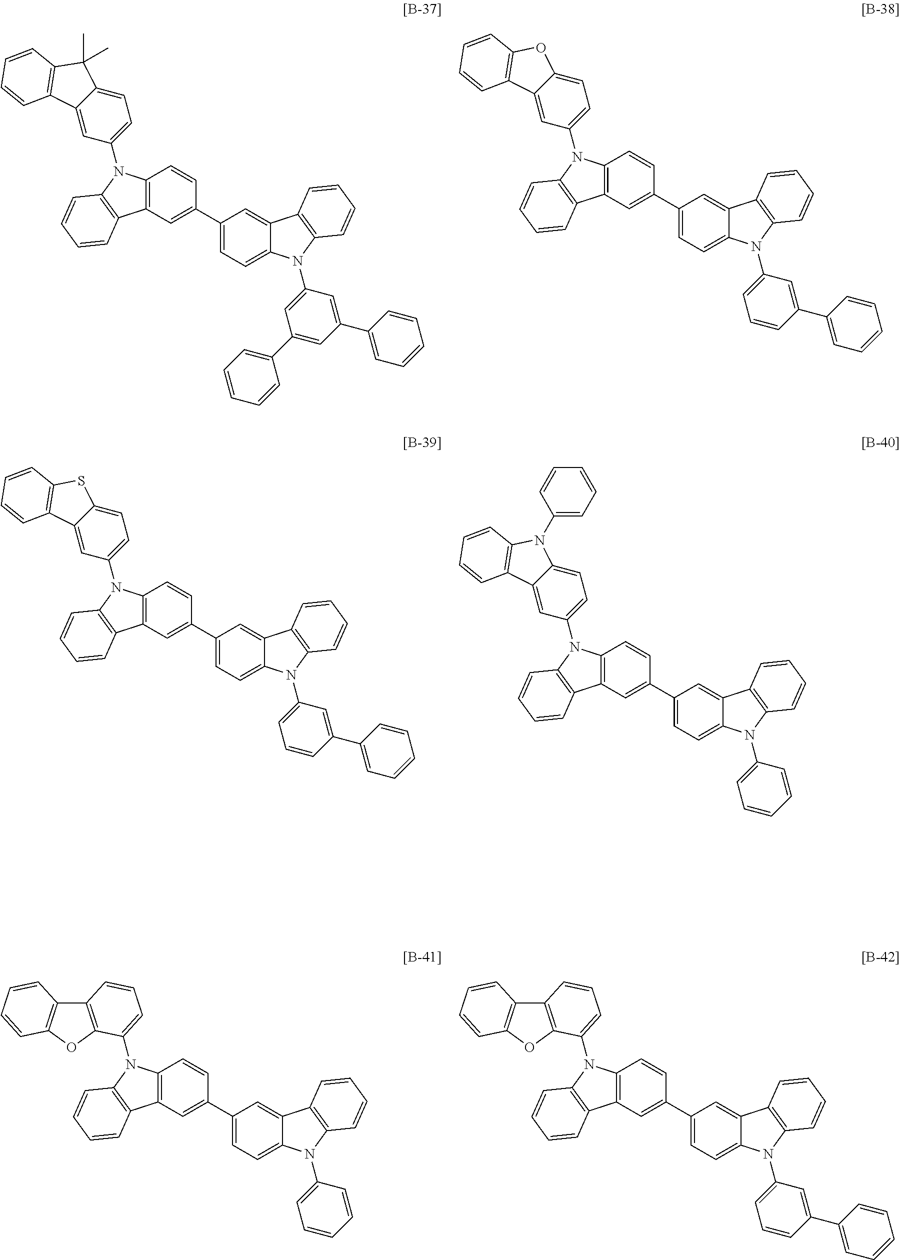 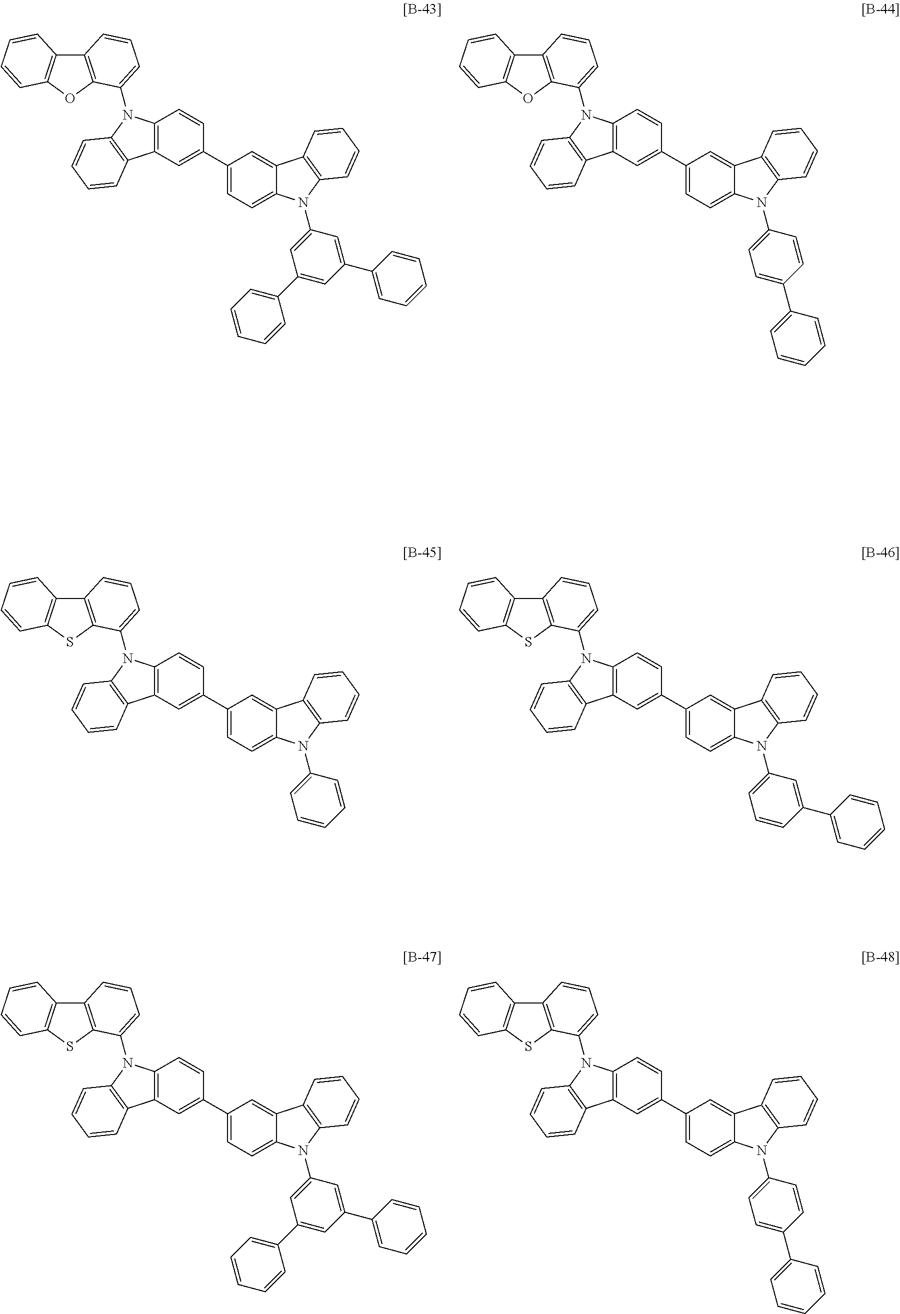 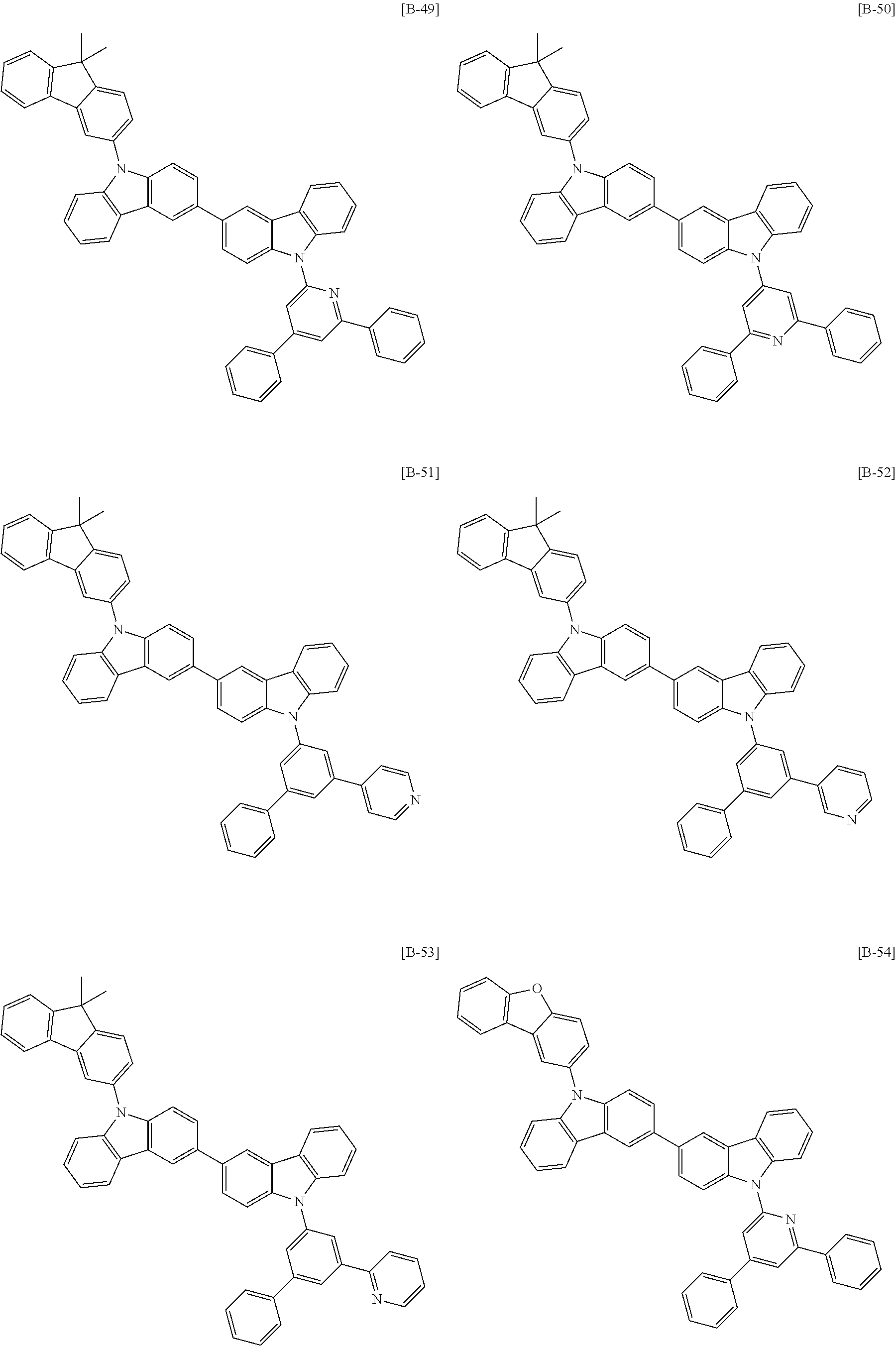 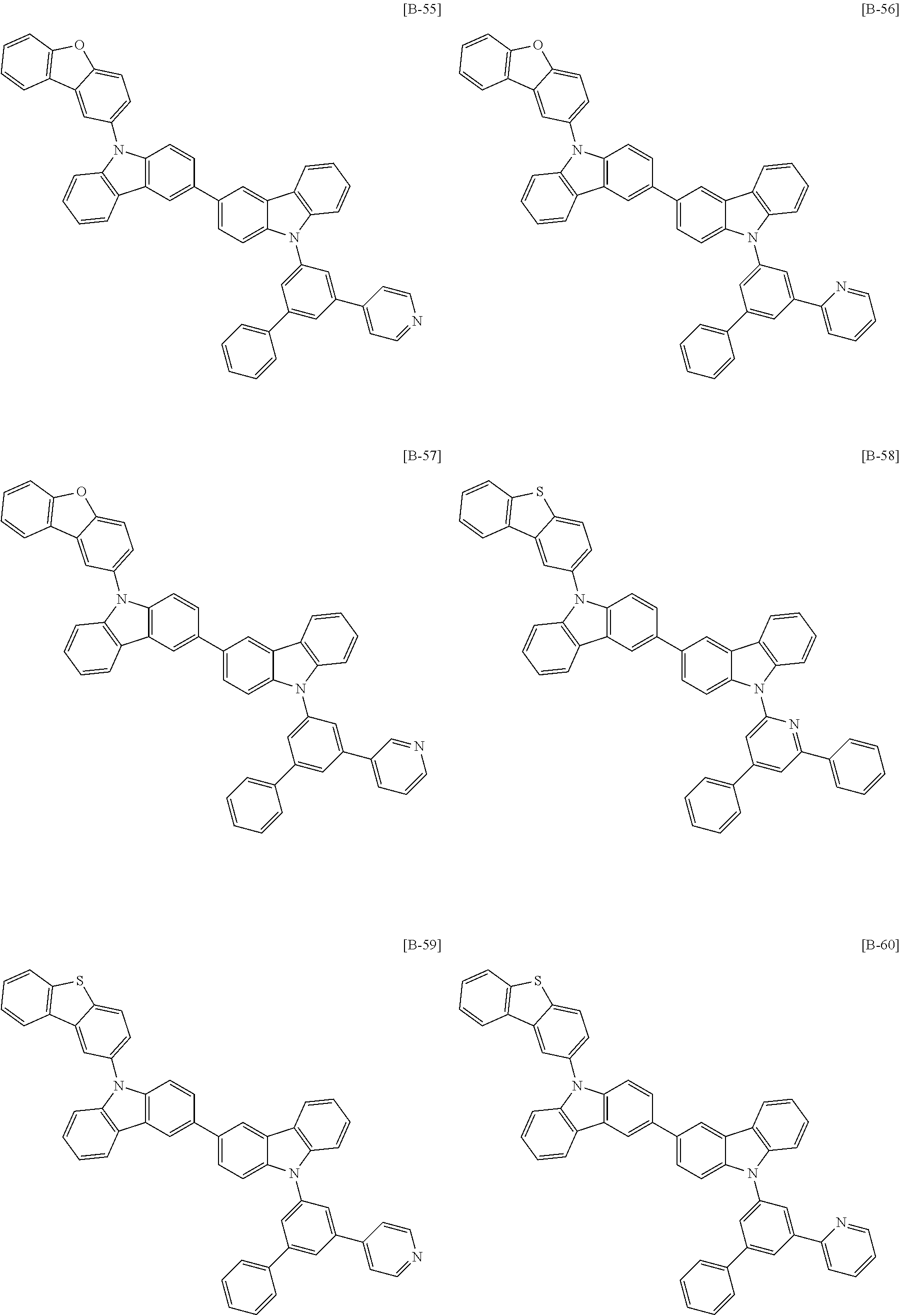 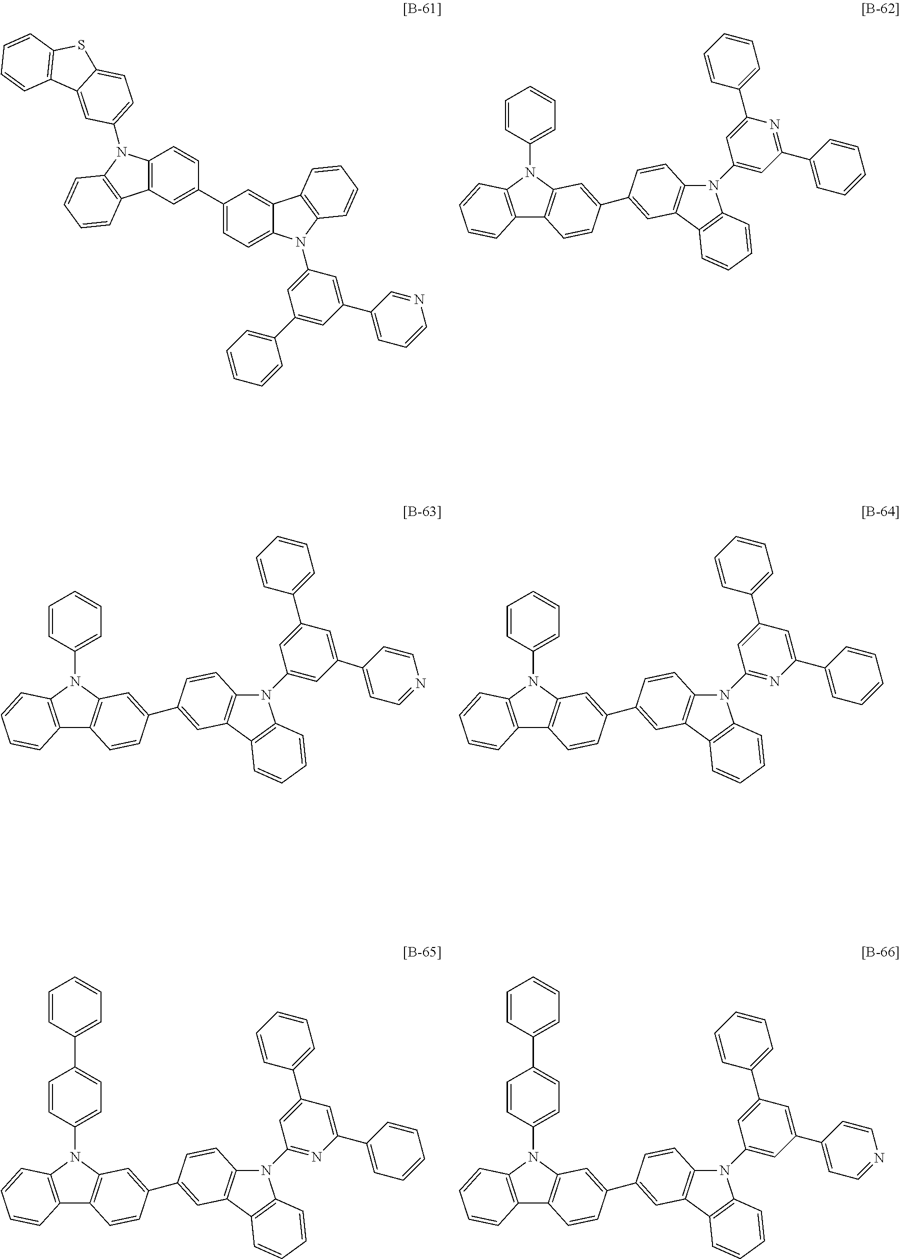 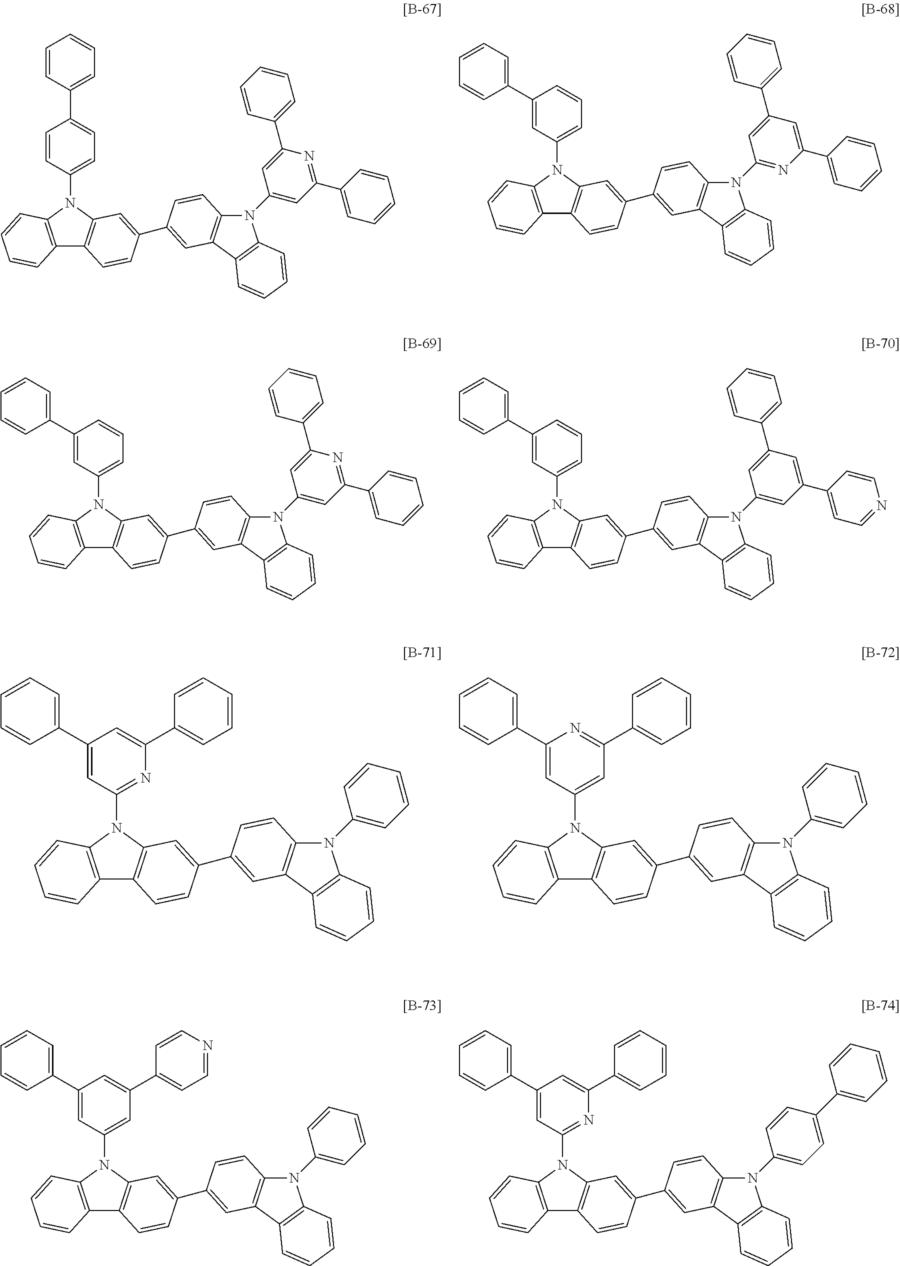 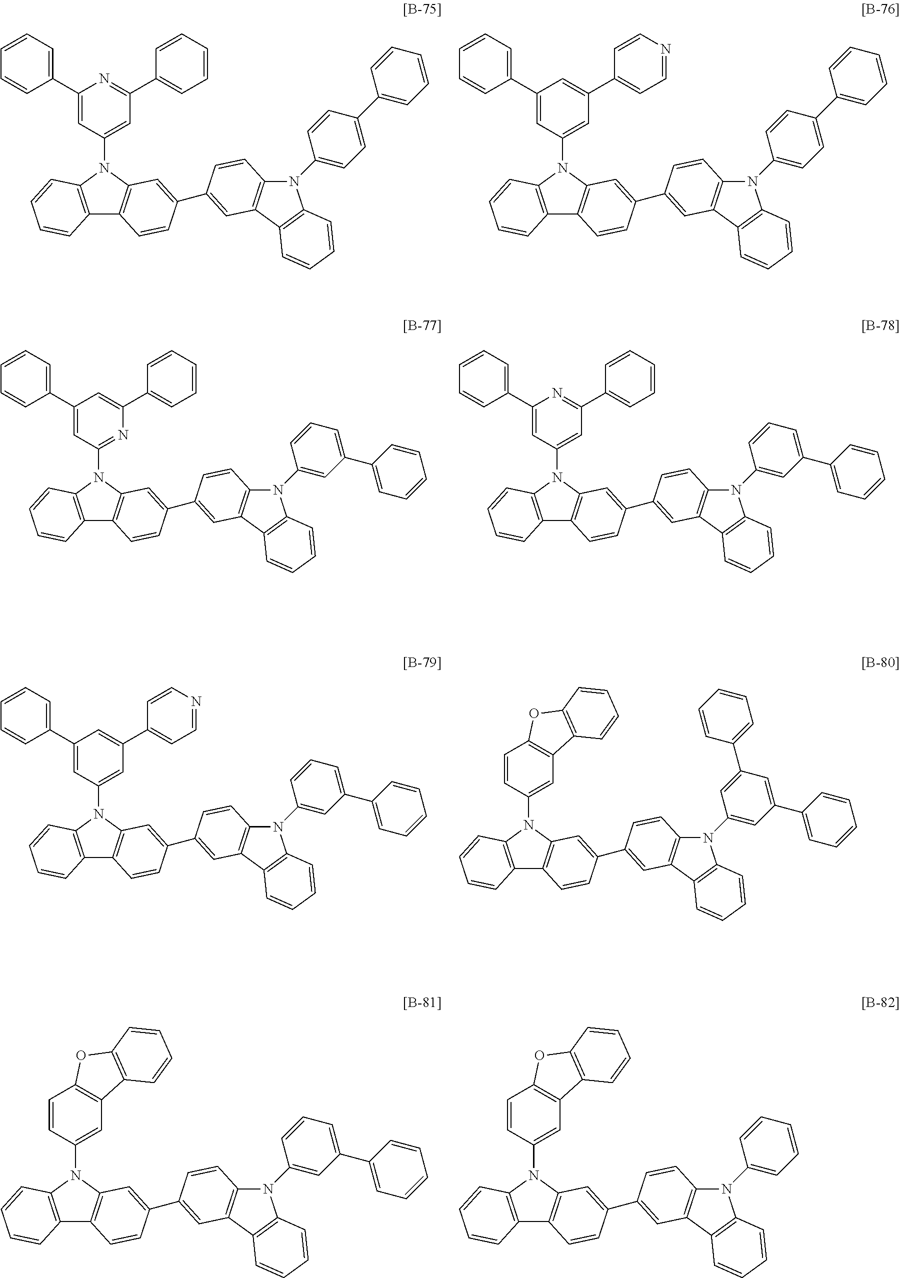 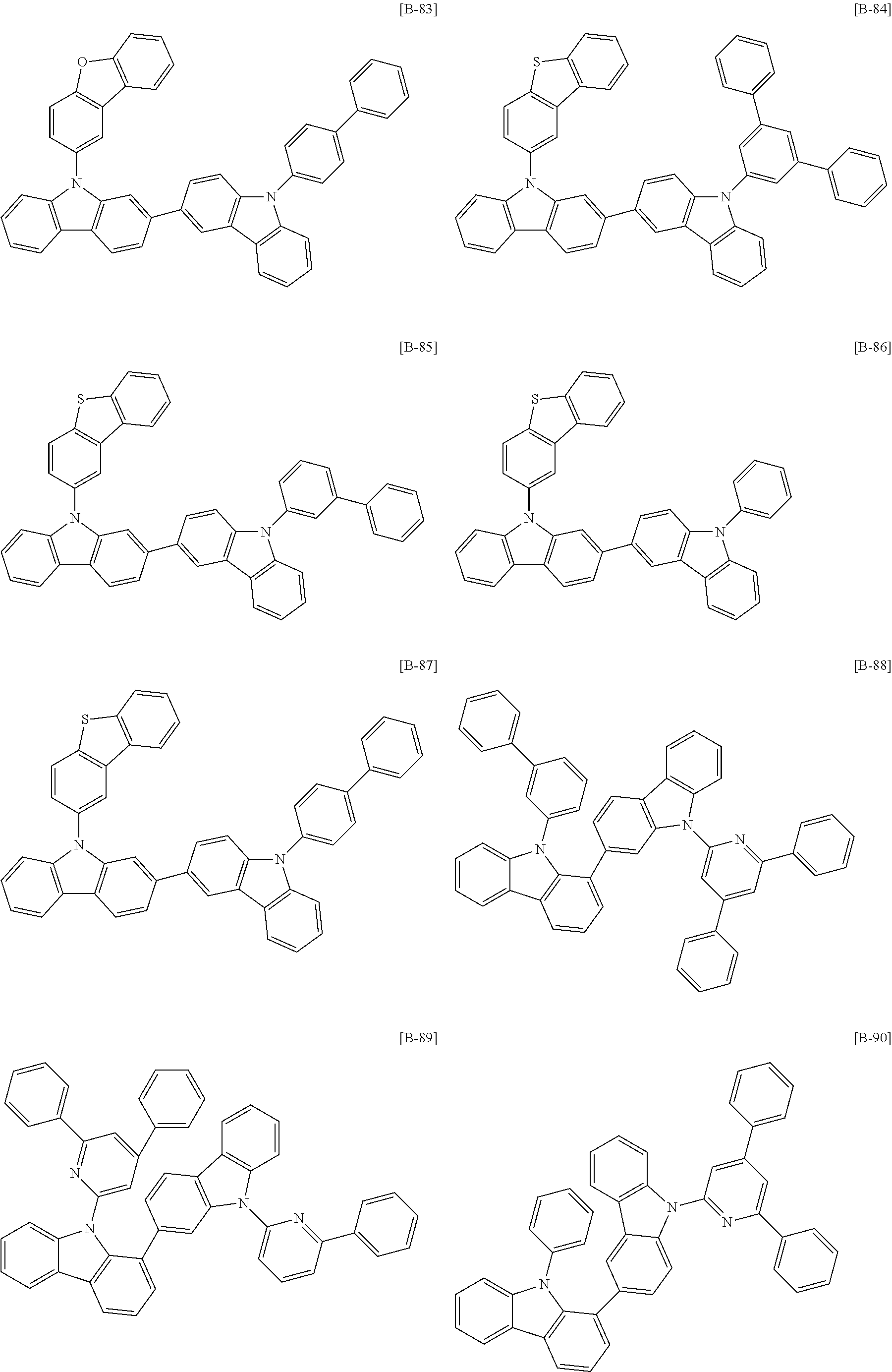 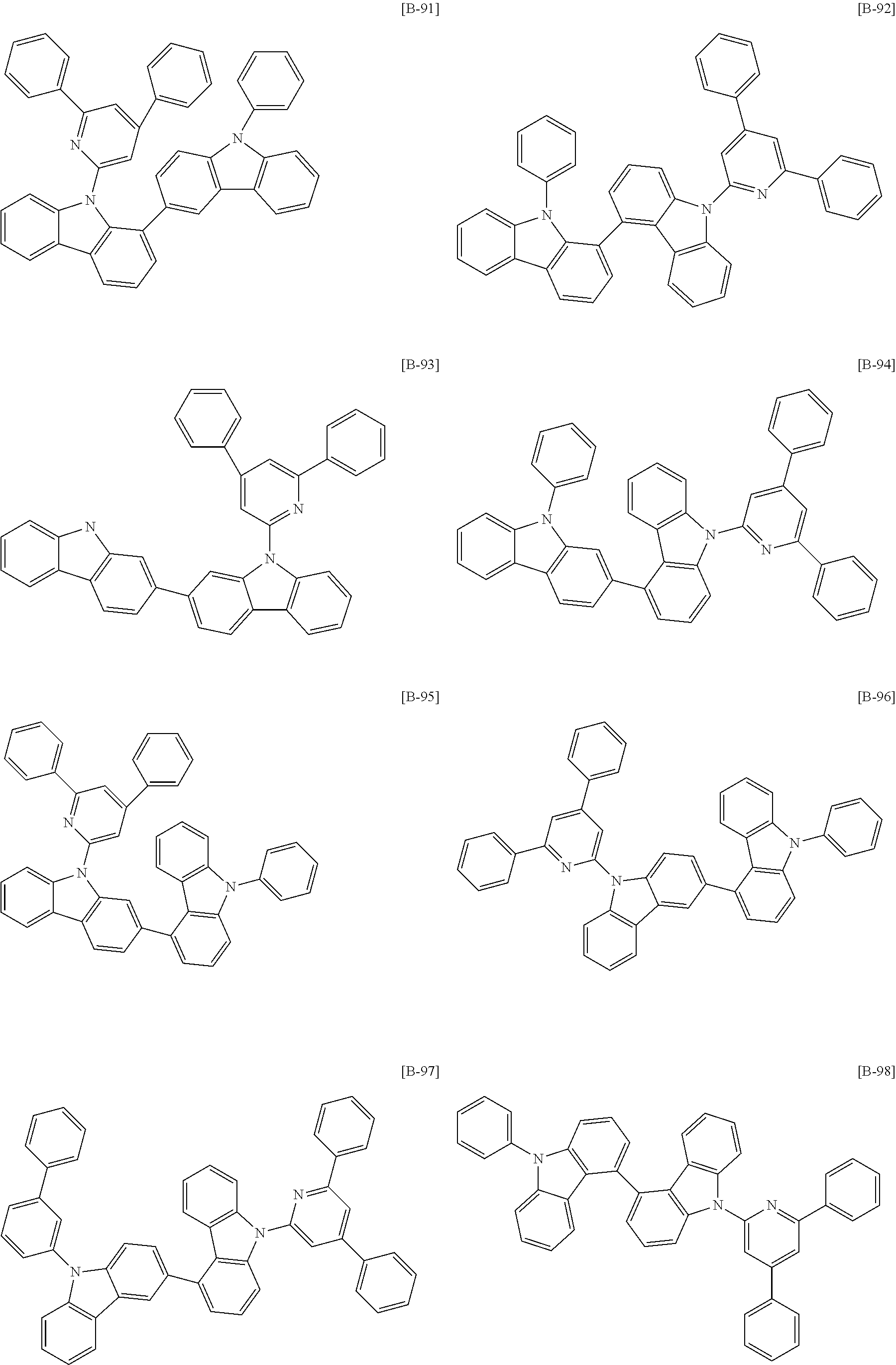 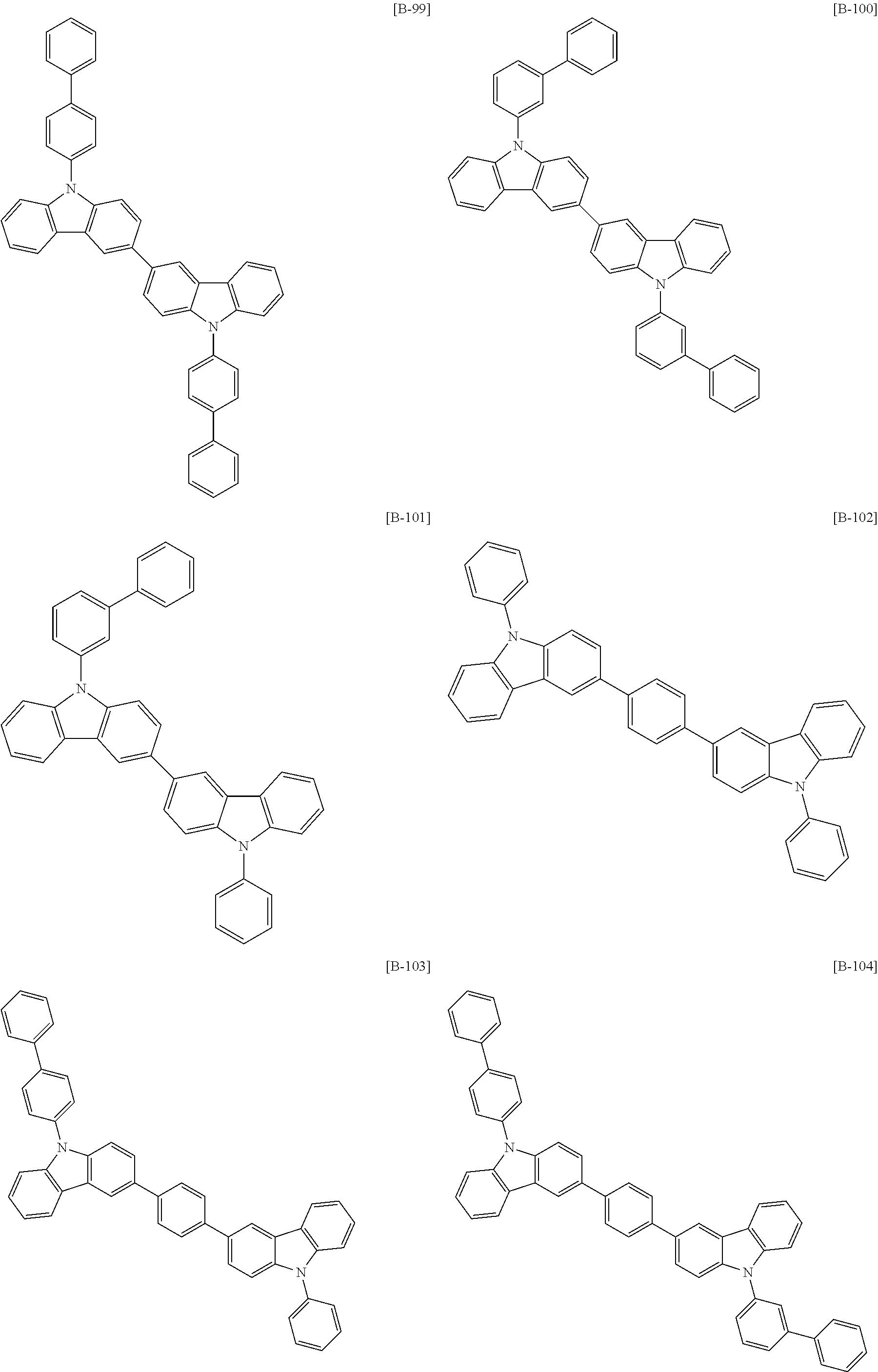 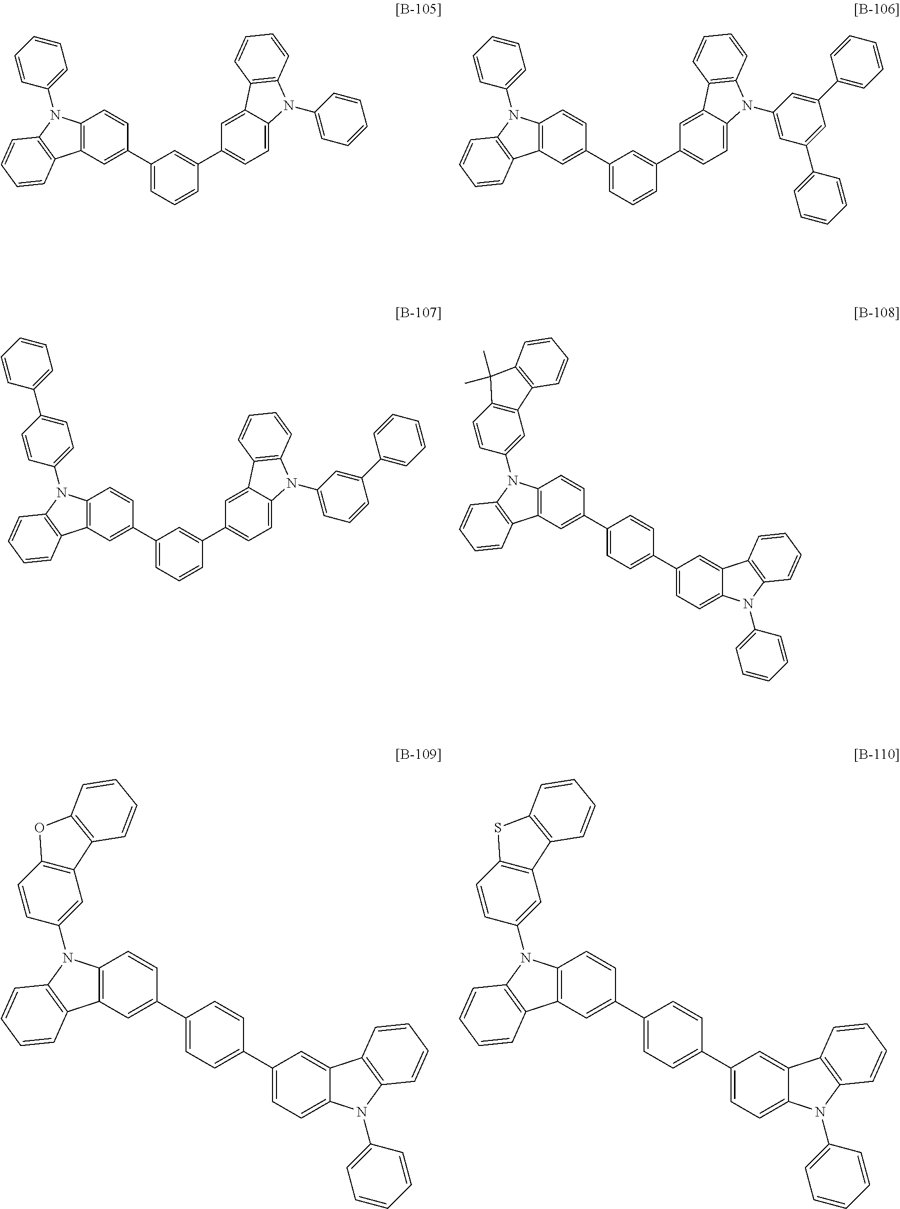 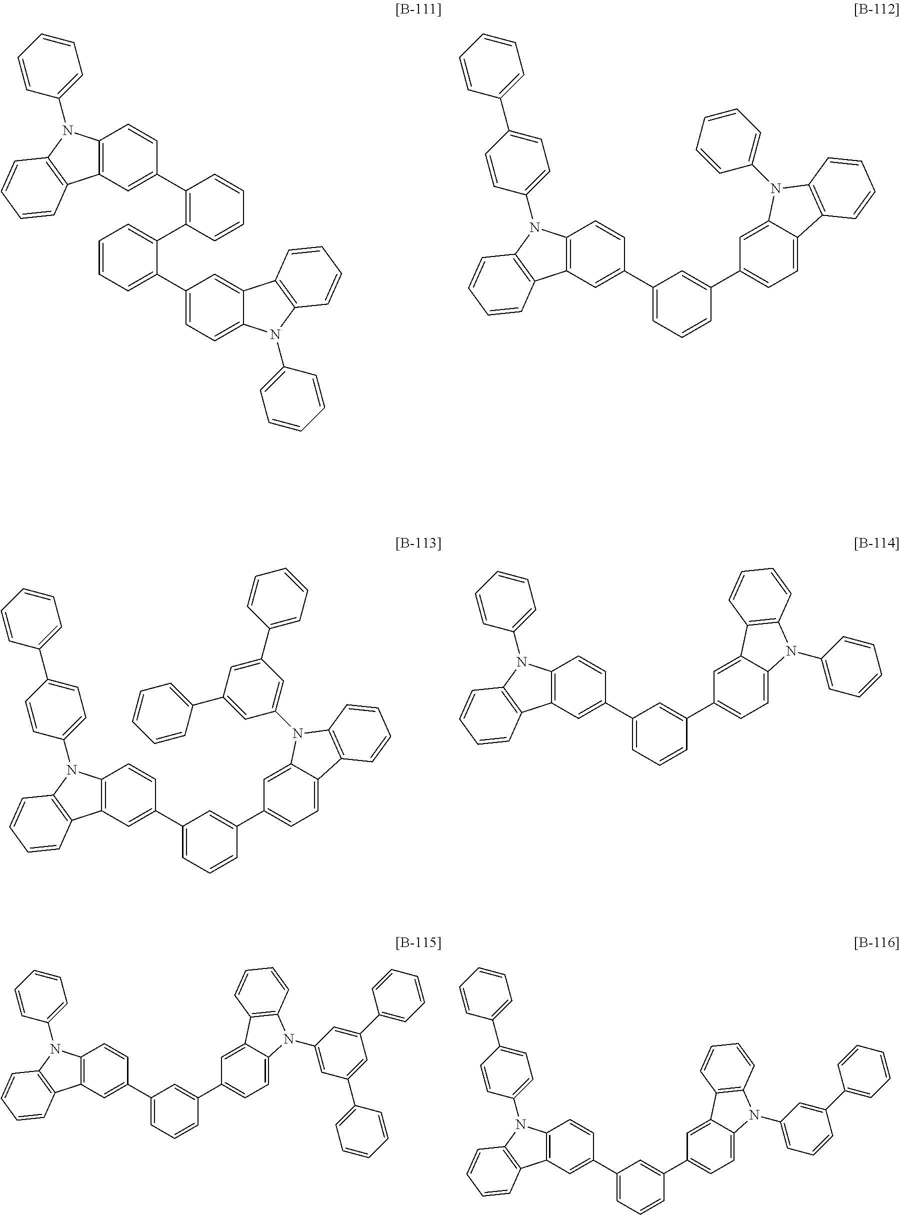 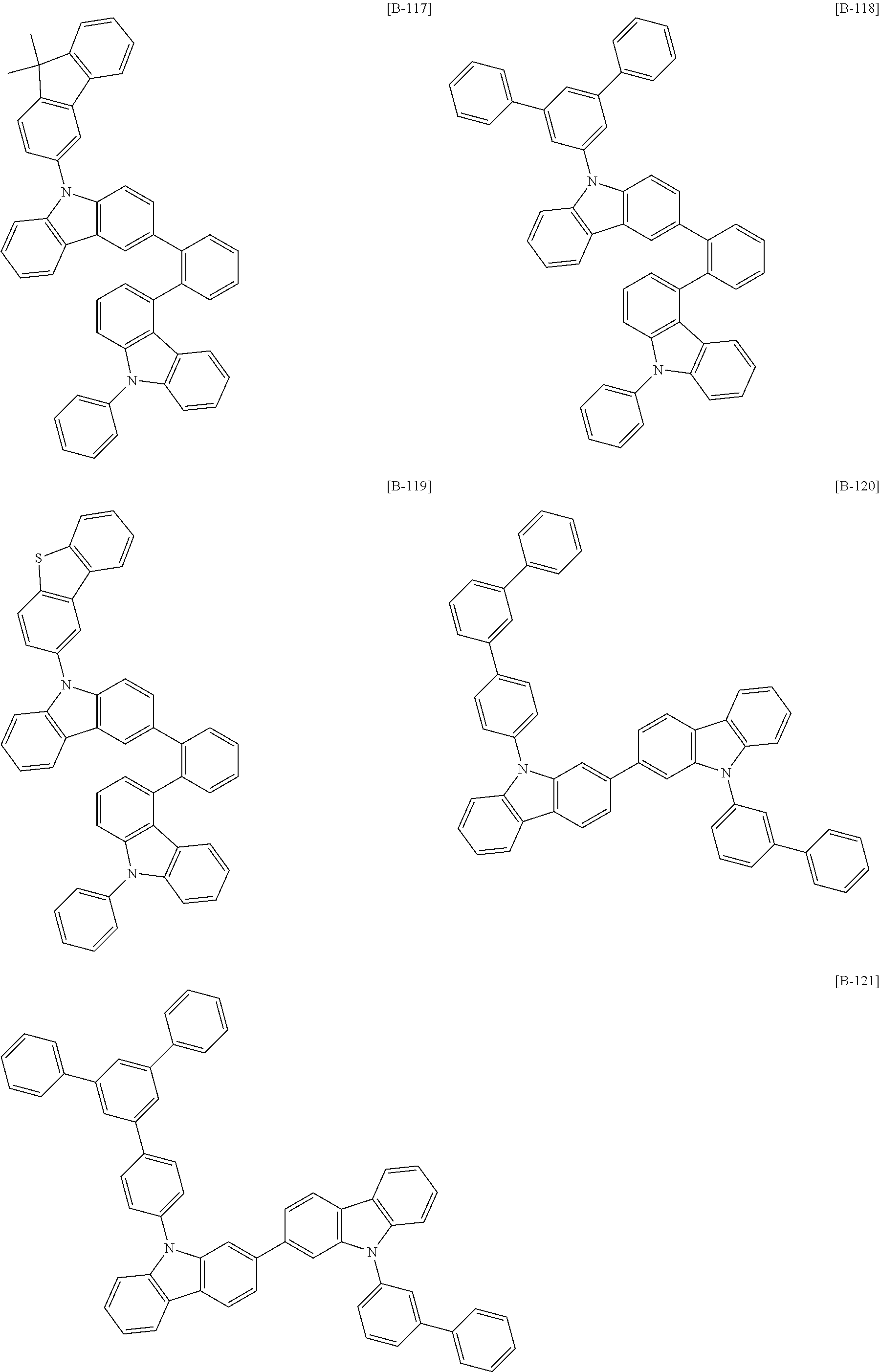 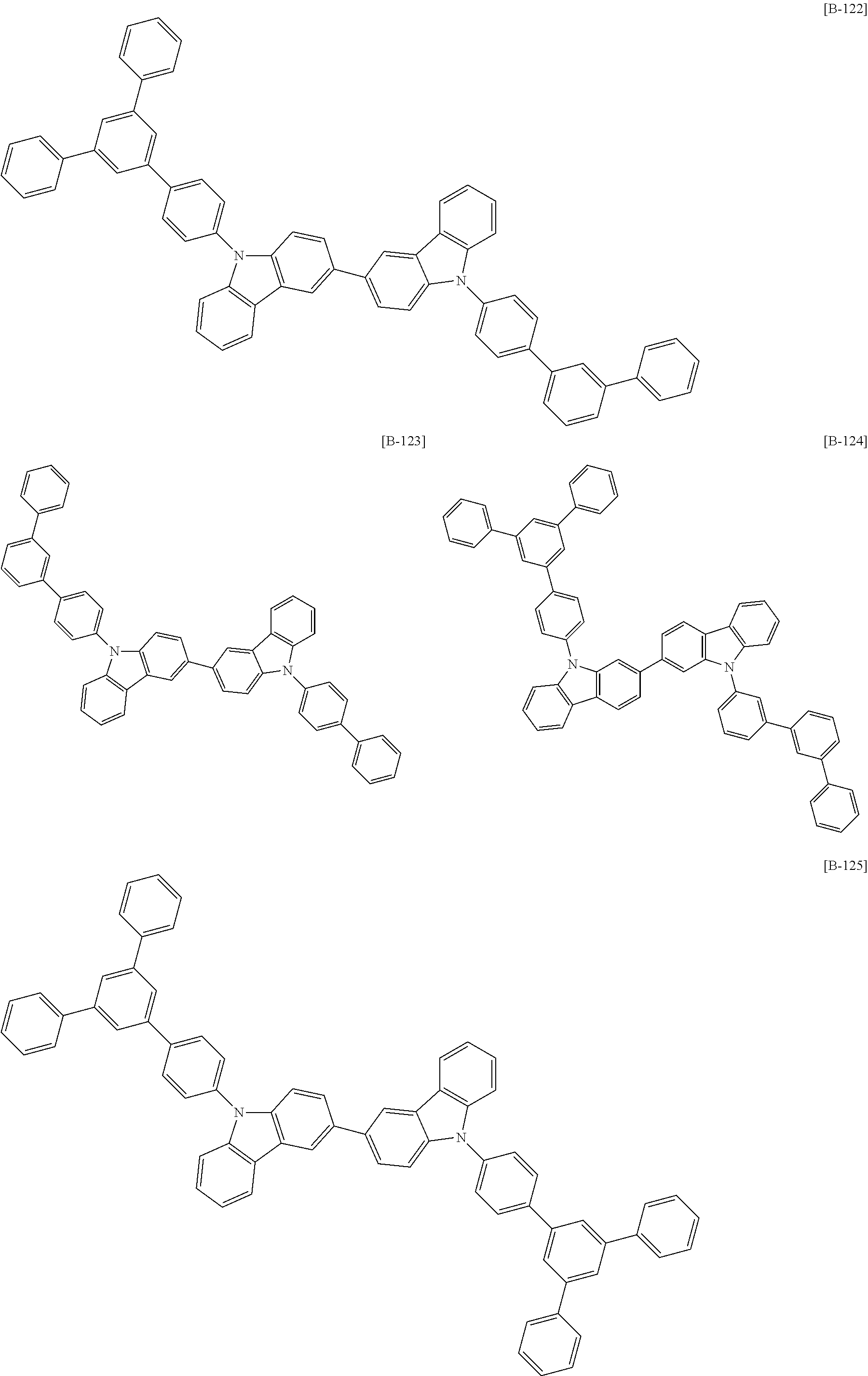 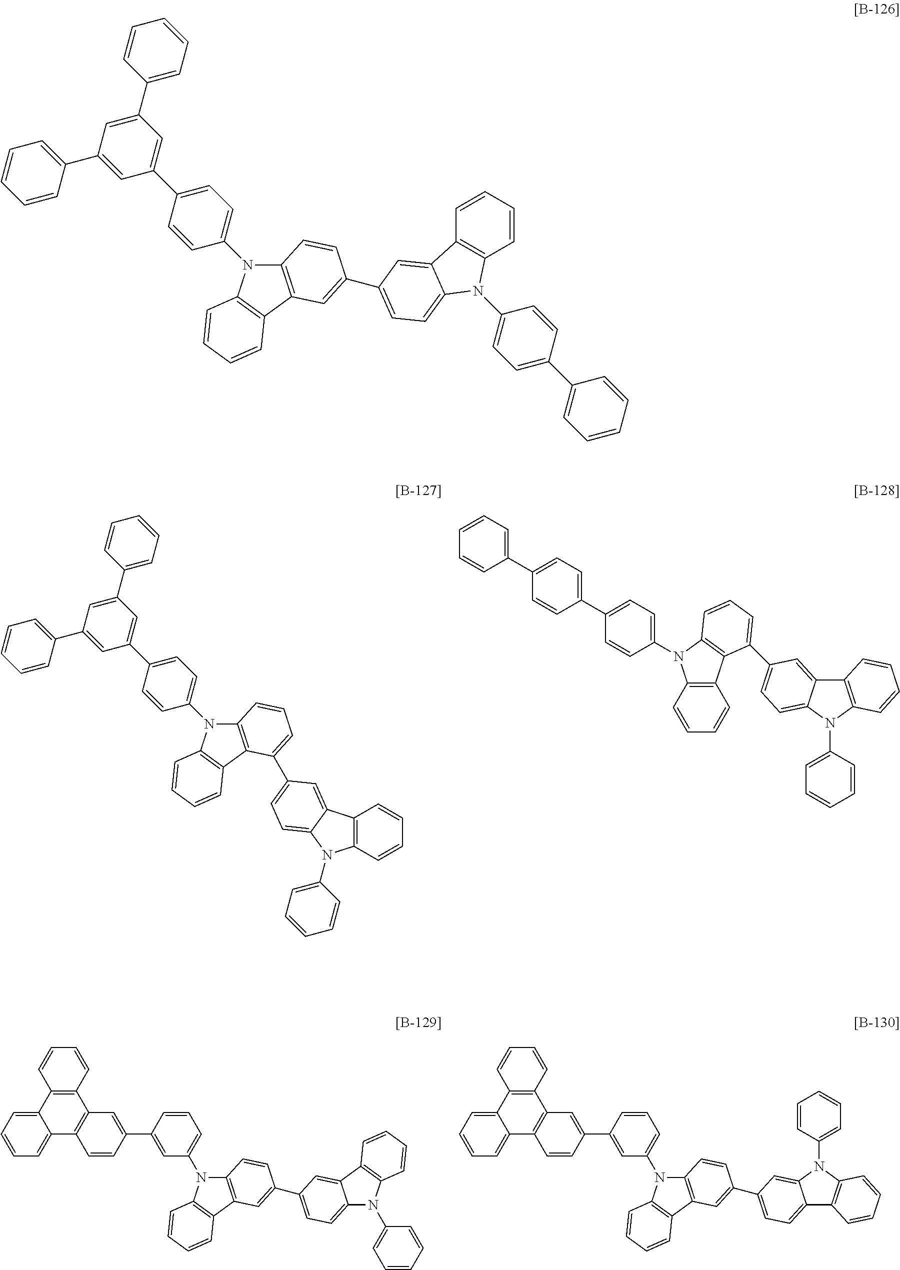 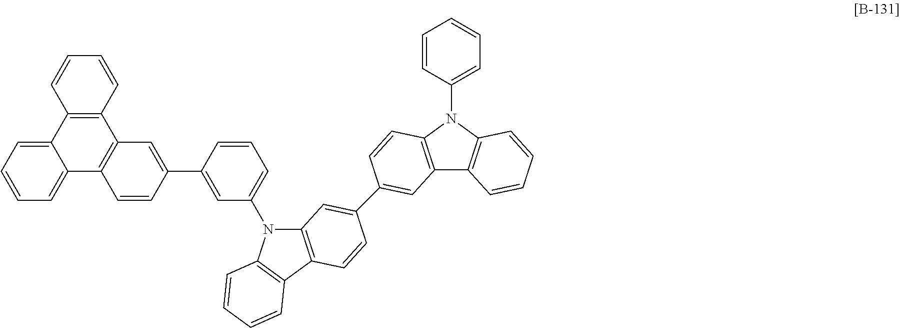
wherein, in Chemical Formula 1-I,
Z is independently N, C, or CR^{a},
at least one of Z is N,
Ar is independently a substituted or unsubstituted C6 to C30 aryl group, a substituted or unsubstituted C2 to C30 heterocyclic group, or a combination thereof,
R¹ to R⁴ and R⁴⁹ to R²⁴ are independently hydrogen, deuterium, a substituted or unsubstituted C1 to C10 alkyl group, a substituted or unsubstituted C6 to C12 aryl group, a substituted or unsubstituted C2 to C30 heterocyclic group, or a combination thereof,
n1 is one of integers of 0 to 3,
n2 and n3 are independently one of integers of 1 to 5, and
n4 is an integer ranging from 0 to 2,
wherein, “substituted” is the same as defined in claim 1.

12. The organic optoelectronic device of claim 1, wherein the hole transport auxiliary layer contacts the hole transport layer and the emission layer, respectively.

13. The organic optoelectronic device of claim 1, wherein the emission layer further includes a phosphorescent dopant.

14. The organic optoelectronic device of claim 1, wherein the organic optoelectronic device is selected from an organic light emitting diode, an organic photoelectric device, an organic solar cell, an organic transistor, an organic photo conductor drum, and an organic memory device.

15. A display device comprising the organic optoelectronic device of claim 1.
